SEQUENCE LISTING (72 sequences)

SEQ ID NO: 1 - protein, 234 aa, Homo sapiens
  misc_feature: Incyte ID No 1888682CD1
MPSGCHSSPPSGLRGDMASLVPLSPYLSPTVLLLVSCDLGFVRADRPPSPVNVTVTHLRANSATVSWDVPEGNIVIGYSISQQRQNGPGQRVIREVNTTTRACALWGLAEDSDYTVQVRSIGLRGESPPGPRVHFRTLKGSDRLPSNSSSPGDITVEGLDGERPLQTGEVVIIVVVLLMWAAVIGLFCRQYDIIKDNDSNNNPKEKGKGPEQSPQGRPVGTRQKKSPSINTIDV

SEQ ID NO: 2 - protein, 443 aa, Homo sapiens
  misc_feature: Incyte ID No 1794980CD1
MGGPRAWALLCLGLLLPGGGAAWSIGAAPFSGRRNWCSYVVTRTISCHVQNGTYLQRVLQNCPWPMSCPGSSYRTVVRPTYKVMYKIVTAREWRCCPGHSGVSCEEVAASSASLEPMWSGSTMRRMALRPTAFSGCLNCSKVSELTERLKVLEAKMTMLTVIEQPVPPTPATPEDPAPLWGPPPAQGSPGDGGLQDQVGAWGLPGPTGPKGDAGSRGPMGMRGPPGPQGPPGSPGRAGAVGTPGERGPPGPPGPPGPPGPPAPVGPPHARISQHGDPLLSNTFTETNNHWPQGPTGPPGPPGPMGPPGPPGPTGVPGSPGHIGPPGPTGPKGISGHPGEKGERGLRGEPGPQGSAGQRGEPGPKGDPGEKSHWGEGLHQLREALKILAERVLILETMIGLYEPELGSGAGPAGTGTPSLLRGKRGGHATNYRIVAPRSRDERG

SEQ ID NO: 3 - protein, 261 aa, Homo sapiens
  misc_feature: Incyte ID No 5533958CD1
MGGAGILLLLLAGAGVVVAWRPPKGKCPLRCSCSKDSALCEGSPDLPVSFSPTLLSLSLVRTGVTQLKAGSFLRIPSLHLLLFTSNSFSVIEDDAFAGLSHLQYLFIEDNEIGSISKNALRGLRSLTHLSLANNHLETLPRFLFRGLDTLTHVDLRGNPFQCDCRVLWLLQWMPTVNASVGTGACAGPASLSHMQLHHLDPKTFKCRAIGGGLSRWGGRREIWGKGCQGQEARLTPCPAISRSGKTLSKQHCLPEPQFSHL

SEQ ID NO: 4 - protein, 643 aa, Homo sapiens
  misc_feature: Incyte ID No 60210196CD1
MEPVPLQDFVRALDPASLPRVLRVCSGVYFEGSIYEISGNECCLSTGDLIKVTQVRLQKVVCENPKTSQTMELAPNFQGYFTPLNTPQSYETLEELVSATTQSSKQLPTCFMSTHRIVTEGRVVTEDQLLMLEAVVMHLGIRSARCVLGMEGQQVILHLPLSQKGPFWTWEPSAPRTLLQVLQDPALKDLVLTCPTLPWHSLILRPQYEIQAIMHMRRTIVKIPSTLEVDVEDVTASSRHVHFIKPLLLSEVLAWEGPFPLSMEILEVPEGRPIFLSPWVGSLQKGQRLCVYGLASPPWRVLASSKGRKVPRHFLVSGGYQGKLRRRPREFPTAYDLLGAFQPGRPLRVVATKDCEGEREENPEFTSLAVGDRLEVLGPGQAHGAQGSDVDVLVCQRLSDQAGEDEEEECKEEAESPERVLLPFHFPGSFVEEMSDSRRYSLADLTAQFSLPCEVKVVAKDTSHPTDPLTSFLGLRLEEKITEPFLVVSLDSEPGMCFEIPPRWLDLTVVKAKGQPDLPEGSLPIATVEELTDTFYYRLRKLPACEIQAPPPRPPKNQGLSKQRRHSSEGGVKSSQVLGLQQHARLPKPKAKTLPEFIKDGSSTYSKIPAHRKGHRPAKPQRQDLDDDEHDYEEILEQFQKTI

SEQ ID NO: 5 - protein, 628 aa, Homo sapiens
  misc_feature: Incyte ID No 815125CD1
MGSCARLLLLWGCTVVAAGLSGVAGVSSRCEKACNPRMGNLALGRKLWADTTCGQNATELYCFYSENTDLTCRQPKCDKCNAAYPHLAHLPSAMADSSFRFPRTWWQSAEDVHREKVQLDLEAEFYFTHLIVMFKSPRPAAMVLDRSQDFGKTWKPYKYFATNCSATFGLEDDVVKKGAICTSKYSSPFPCTGGEVIFKALSPPYDTENPYSAKVQEQLKITNLRVQLLKRQSCPCQRNDLNEEPQHFTHYAIYDFIVKGSCFCNGHADQCIPVHGFRPVKAPGTFHMVHGKCMCKHNTAGSHCQHCAPLYNDRPWEAADGKTGAPNECRTCKCNGHADTCHFDVNVWEASGNRSGGVCDDCQHNTEGQYCQRCKPGFYRDLRRPFSAPDACKPCSCHPVGSAVLPANSVTFCDPSNGDCPCKPGVAGRRCDRCMVGYWGFGDYGCRPCDCAGSCDPITGDCISSHTDIDWYHEVPDFRPVHNKSEPAWEWEDAQGFSALLHSGKCECKEQTLGNAKAFCGMKYSYVLKIKILSAHDKGTHVEVNVKIKKVLKSTKLKIFRGKRTLYPESWTDRGCTCPILNPGLEYLVAGHEDIRTGKLIVNMKSFVQHWKPSLGRKVMDILKRECK

SEQ ID NO: 6 - protein, 686 aa, Homo sapiens
  misc_feature: Incyte ID No 1386915CD1
MLLRGVLLALQALQLAGALDLPAGSCAFEESTCGFDSVLASLPWILNEEGHYIYVDTSFGKQGEKAVLLSPDLQAEEWSCLRLVYQITTSSESLSDPSQLNLYMRFEDESFDRLLWSAKEPSDSWLIASLDLQNSSKKFKILIEGVLGQGNTASIALFEIKMTTGYCIECDFEENHLCGFVNRWNPNVNWFVGGGSIRNVHSILPQDHTFKSELGHYMYVDSVYVKHFQEVAQLISPLTTAPMAGCLSFYYQIQQGNDNVFSLYTRDVAGLYEEIWKADRPGNAAWNLAEVEFNAPYPMEVIFEVAFNGPKGGYVALDDISFSPVHCQNQTELLFSAVEASCNFEQDLCNFYQDKEGPGWTRVKVKPNMYRAGDHTTGLGYYLLANTKFTSQPGYIGRLYGPSLPGNLQYCLRFHYAIYGFLKMSDTLAVYIFEENHVVQEKIWSVLESPRGVWMQAEITFKKPMPTKVVFMSLCKSFWDCGLVALDDITIQLGSCSSSEKLPPPPGECTFEQDECTFTQEKRNRSSWHRRRGETPTSYTGPKGDHTTGVGYYMYIEASHMVYGQKARLLSRPLRGVSGKHCLTFFYHMYGGGTGLLSVYLKKEEDSEESLLWRRRGEQSISWLRALIEYSCERQHQIIFEAIRGVSIRSDIAIDDVKFQAGPCGEMEDTTQQSSGYSEDLNEIEY

SEQ ID NO: 7 - protein, 296 aa, Homo sapiens
  misc_feature: Incyte ID No 1344495CD1
MRHEELLTKTFQGPAVVCGTPTSHVYMFKNGSGDSGDSSEEESHRVVLRPRGKERHKSGVHQPPQAGAGDVVLLQRELAQEDSLNKLALQYGCKVADIKKVNNFIREQDLYALKSVKIPVRNHGILMETHKELKPLLSPSSETTVTVELPEADRAGAGTGAQAGQLMGFFKGIDQDIERAVQSEIFLHESYCMDTSHQPLLPAPPKTPMDGADCGIQWWNAVFIMLLIGIVLPVFYLVYFKIQASGETPNSLNTTVIPNGSMAMGTVPGQAPRLAVAVPAVTSADSQFSQTTQAGS

SEQ ID NO: 8 - protein, 575 aa, Homo sapiens
  misc_feature: Incyte ID No 1485774CD1
MAKPFFRLQKFLRRTQFLLFFLTAAYLMTGSLLLLQRVRVALPQGPRAPGPLQTLPVAAVALGVGLLDSRALHDPRVSPELLLGVDMLQSPLTRPRPGPRWLRSRNSELRQLRRRWFHHFMSDSQGPPALGPEAARPAIHSRGTYIGCFSDDGHERTLKGAVFYDLRKMTVSHCQDACAERSYVYAGLEAGAECYCGNRLPAVSVGLEECNHECKGEKGSVCGAVDRLSVYRVDELQPGSRKRRTATYRGCFRLPENITHAFPSSLIQANVTVGTCSGFCSQKEFPLAILRGWECYCAYPTPRFNLRDAMDSSVCGQDPEAQRLAEYCEVYQTPVQDTRCTDRRFLPNKSKVFVALSSFPGAGNTWARHLIEHATGFYTGSYYFDGTLYNKGFKGEKDHWRSRRTICVKTHESGRREIEMSDSAILLIRNPYRSLVAEFNRKCAGHLGYAADRNWKSKEWPDFVNSYASWWSSHVLDWLKYGKRLLVVHYEELRRSLVPTLREMVAFLNVSVSEERLLCVENNKEGSFRRRGRRSHDPEPFTPEMKDLINGYIRTVDQALRDHNWTGLPREYVPR

SEQ ID NO: 9 - protein, 592 aa, Homo sapiens
  misc_feature: Incyte ID No 7289372CD1
MFPLRALWLVWALLGVAGSCPEPCACVDKYAHQFADCAYKELREVPEGLPANVTTLSLSANKITVLRRGAFADVTQVTSLWLAHNEVRTVEPGALAVLSQLKNLDLSHNFISSFPWSDLRNLSALQLLKMNHNRLGSLPRDALGALPDLRSLRINNNRLRTLAPGTFDALSALSHLQLYHNPFHCGCGLVWLQAWAASTRVSLPEPDSIACASPPALQGVPVYRLPALPCAPPSVHLSAEPPLEAPGTPLRAGLAFVLHCIADGHPTPRLQWQLQIPGGTVVLEPPVLSGEDDGVGAEEGEGEGDGDLLTQTQAQTPTPAPAWPAPPATPRFLALANGSLLVPLLSAKEAGVYTCRAHNELGANSTSIRVAVAATGPPKHAPGAGGEPDGQAPTSERKSTAKGRGNSVLPSKPEGKIKGQGLAKVSILGETETEPEEDTSEGEEAEDQILADPAEEQRCGNGDPSRYVSNHAFNQSAELKPHVFELGVIALDVAEREARVQLTPLAARWGPGPGGAGGAPRPGRRPLRLLYLCPAGGGAAVQWSRVEEGVNAYWFRGLRPGTNYSVCLALAGEACHVQVVFPPRRSSHRCWS

SEQ ID NO: 10 - protein, 255 aa, Homo sapiens [sequence omitted]

SEQ ID NO: 11 - protein, 641 aa, Homo sapiens
  misc_feature: Incyte ID No 184661CD1
MVPGARGGGALARAAGRGLLALLLAVSAPLRLQAEELGDGCGHLVTYQDSGTMTSKNYPGTYPNHTVCEKTITVPKGKRLILRLGDLDIESQTCASDYLLFTSSSDQYGPYCGSMTVPKELLLNTSEVTVRFESGSHISGRGFLLTYASSDHPDLITCLERASHYLKTEYSKFCPAGCRDVAGDISGNMVDGYRDTSLLCKAAIHAGIIADELGGQISVLQRKGISRYEGILANGVLSRDGSLSDKRFLFTSNGCSRSLSFEPDGQIRASSSWQSVNESGDQVHWSPGQARLQDQGPSWASGDSSNNHKPREWLEIDLGEKKKITGIRTTGSTQSNFNFYVKSFVMNFKNNNSKWKTYKGIVNNEEKVFQGNSNFRDPVQNNFIPPIVARYVRVVPQTWHQRIALKVELIGCQITQGNDSLVWRKTSQSTSVSTKKEDETITRPIPSEETSTGINITTVAIPLVLLVVLVFAGMGIFAAFRKKKKKGSPYGSAEAQKTDCWKQIKYPFARHQSAEFTISYDNEKEMTQKLDLITSDMADYQQPLMIGTGTVTRKGSTFRPMDTDAEEAGVSTDAGGHYDCPQRAGRHEYALPWRPRSPSTPRPSWSGTCCAPTRSLRRAATASQGPSPATNTPSPRAASPP

SEQ ID NO: 12 - protein, 924 aa, Homo sapiens [sequence omitted]

SEQ ID NO: 13 - protein, 987 aa, Homo sapiens
  misc_feature: Incyte ID No 5773251CD1
MRISSCSDESSNSNSSRKSDNHSPAVVTTTVSSKKQPSVLVTFPKEERKSVSGKASIKLSETISEGTSNSLSTCTKSGPSPLSSPNGKLTVASPKRGQKREEGWKEVVRRSKKVSVPSTVISRVIGRGGCNINAIREFTGAHIDIDKQKDKTGDRIITIRGGTESTRQATQLINALIKDPDKEIDELIPKNRLKSSSANSKIGSSAPTTTAANTSLMGIKMTTVALSSTSQTATALTVPAISSASTHKTIKNPVNNVRPGFPVSLPLAYPPPQFAHALLAAQTFQQIRPPRLPMTHFGGTFPPAQSTWGPFPVRPLSPARATNSPKPHMVPRHSNQNSSGSQVNSAGSLTSSPTTTTSSSASTVPGTSTNGSPSSPSVRRQLFVTVVKTSNATTTTVTTTASNNNTAPTNATYPMPTAKEHYPVSSPSSPSPPAQPGGVSRNSPLDCGSASPNKVASSSEQEAGSPPVVETTNTRPPNSSSSSGSSSAHSNQQQPPGSVSQEPRPPLQQSQVPPPEVRMTVPPLATSSAPVAVPSTAPVTYPMPQTPMGCPQPTPKMETPAIRPPPHGTTAPHKNSASVQNSSVAVLSVNHIKRPHSVPSSVQLPSTLSTQSACQNSVHPANKPIAPNFSAPLPFGPFSTLFENSPTSAHAFWGGSVVSSQSTPESMLSGKSSYLPNSDPLHQSDTSKAPGFRPPLQRPAPSPSGIVNMDSPYGSVTPSSTHLGNFASNISGGQMYGPGAPLGGAPAAANFNRQHFSPLSLLTPCSSASNDSSAQSVSSGVRAPSPAPSSVPLGSEKPSNVSQDRKVPVPIGTERSARIRQTGTSAPSVIGSNLSTSVGHSGIWSFEGIGGNQDKVDWCNPGMGNPMIHRPMSDPGVFSQHQAMERDSTGIVTPSGTFHQHVPAGYMDFPKVGGMPFSVYGNAMIPPVAPIPDGAGGPIFNGPHAADPSWNSLIKMVSSSTENNGPQTVWTGPWAPHMNSVHMNQLG

SEQ ID NO: 14 - protein, 1028 aa, Homo sapiens
  misc_feature: Incyte ID No 5426470CD1
MMFPWKQLILLSFIGCLGGELLLQGPVFIKEPSNSIFPVGSEDKKITLHCEARGNPSPHYRWQLNGSDIDMSMEHRYKLNGGNLVVINPNRNWDTGTYQCFATNSLGTIVSREAKLQFAYLENFKTKMRSTVSVREGQGVVLLCGPPPHSGELSYAWIFNEYPSFVEEDSRRFVSQETGHLYISKVEPSDVGNYTCVVTSMVTNARVLGSPTPLVLRSDGVMGEYEPKIEVQFPETLPAAKGSTVKLECFALGNPIPQINWRRSDGLPFSSKIKLRKFSGVLEIPNFQQEDAGSYECIAENSRGKNVARGRLTYYAKPHWVQLIKDVEIAVEDSLYWECRASGKPKPSYRWLKNGAALVLEERTQIENGALTISNLSVTDSGMFQCIAENKHGLVYSSAELKVVASAPDFSKNPMKKLVQVQVGSLVSLDCKPRASPRALSSWKKGDVSVQEHERISLLNDGGLKIANVTKADAGTYTCMAENQFGKANGTTHLVVTEPTRITLAPSNMDVSVGESVILPCQVQHDPLLDIIFTWYFNGALADFKKDGSHFEKVGGSSSGDLMIRNIQLKHSGKYVCMVQTGVDSVSSAADLIVRGSPGPPENVKVDEITDTTAQLSWKEGKDNHSPVISYSIQARTPFSVGWQTVTTVPEVIDGKTHTATVVELNPWVEYEFRVVASNKIGGGEPSLPSEKVRTEEAVPEVPPSEVNGGGGSRSELVITWDPVPEELQNGEGFGYVVAFRPLGVTTWIQTVVTSPDTPRYVFRNESIVPYSPYEVKVGVYNNKGEGPFSPVTTVFSAEEEPTVAPSQVSANSLSSSEIEVSWNTIPWKLSNGHLLGYEVRYWNGGGKEESSSKMKVAGNETSARLRGLKSNLAYYTAVRAYNSAGAGPFSATVNVTTKKTPPSQPPGNVVWNATDTKVLLNWEQVKAMENESEVTGYKVFYRTSSQNNVQVLNTNKTSAELVLPIKEDYIIEVKATTDGGDGTSSEQIRIPRITSMDARGSTSAISNVHPMSSYMPIVLFLIVYVLW

SEQ ID NO: 15 - protein, 354 aa, Homo sapiens
  misc_feature: Incyte ID No 7087904CD1
MDMMLLVQGACCSNQWLAAVLLSLCCLLPSCLPAGQSVDFPWAAVDNMMVRKGDTAVLRCYLEDGASKGAWLNRSSIIFAGGDKWSVDPRVSISTLNKRDYSLQIQNVDVTDDGPYTCSVQTQHTPRTMQVHLTVQVPPKIYDISNDMTVNEGTNVTLTCLATGKPEPSISWRHISPSAKPFENGQYLDIYGITRDQAGEYECSAENDVSFPDVRKVKVVVNFAPTIQEIKSGTVTPGRSGLIRCEGAGVPPPAFEWYKGEKKLFNGQQGIIIQNFSTRSILTVTNVTQEHFGNYTCVAANKLGTTNASLPLNPPSTAQYGITGSADVLFSCWYLVLTLSSFTSIFYLKNAILQ

SEQ ID NO: 16 - protein, 1829 aa, Homo sapiens
  misc_feature: Incyte ID No 7477312CD1
MQLSRAAAAAAAAPAEPPEPLSPAPAPAPAPPGPLPRSAADGAPAGGKGGPGRRARSPRALRSPARAAPARAPARGWTAPGPGASAVVVRVGIPDLQQTKCLRLDPAAPVWAAKQRVLCALNHSLQDALNYGLFQPPSRGRAGKFLDEERLLQEYPPNLDTPLPYLEFRYKRRVYAQNLIDDKQFAKLHTKANLKKFMDYVQLHSTDKVARLLDKGLDPNFHDPDSGECPLSLAAQLDNATDLLKVLKNGGAHLDFRTRDGLTAVHCATRQRNAAALTTLLDLGASPDYKDSRGLTPLYHSALGGGDALCCELLLHDHAQLGTTDENGWQEIHQACRFGHVQHLEHLLFYGADMGAQNASGNTALHICALYNQESCARVLLFRGANRDVRNYNSQTAFQVAIIAGNFELAEVIKTHKDSDVGQDSHDLLHPMPTGVPEWGLYTEEELEGGAAFSVPFRETPSYAKRRRLAGPSGLASPRPLQRSASDINLKGEAQPAASPGPSLRSLPHQLLLQRLQEEKDRDRDADQESNISGPLAGRAGQSKIRSCIRIRARFPAPPAPPAPPPRGPKRKLYSAVPGRKFIAVKAHSPQGEGEIPLHRGEAVKVLSIGEGGFWEGTVKGRTGWFPADCVEEVQMRQHDTRPETREDRTKRLFRHYTVGSYDSLTSHSDYVIDDKVAVLQKRDHEGFGFVLRGAKAETPIEEFTPTPAFPALQYLESVDVEGVAWRAGLRTGDFLIEVNGVNVVKVGHKQVVALIRQGGNRLVMKVVSVTRKPEEDGARRRAPPPPKRAPSTTLTLRSKSMTAELEELEKLDEMLAAAAEPTLRPDIADADSRAATVKQRPTSRRITPAEISSLFERQGLPGPEKLPGSLRKGIPRTKSVGEDEKLASLLEGRFPRSTSMQDPVREGRGIPPPPQTAPPPPPAPYYFDSGPPPAFSPPPPPGRAYDTVRSSFKPGLEARLGAGAAGLYEPGAALGPLPYPERQKRARSMIILQDSAPESGDAPRPPPAATPPERPKRRPRPPGPDSPYANLGAFSASLFAPSKPQRRKSPLVKQLQVEDAQERAALAVGSPGPGGGSFAREPSPTHRGPRPGGLDYGAGDGPGLAFGGPGPAKDRRLEERRRSTVFLSVGAIEGSAPGADLPSLQPSRSIDERLLGTGPTAGRDLLLPSPVSALKPLVSGPSLGPSGSTFIHPLTGKPLDPSSPLALALAARERALASQAPSRSPTPVHSPDADRPGPLFVDVQARDPERGSLASPAFSPRSPAWIPVPARREAEKVPREERKSPEDKKSMILSVLDTSLQRPAGLIVVHATSNGQEPSRLGGAEEERPGTPELAPAPMQSAAVAEPLPSPRAQPPGGTPADAGPGQGSSEEEPELVFAVNLPPAQLSSSDEETREELARIGLVPPPEEFANGVLLATPLAGPGPSPTTVPSPASGKPSSEPPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASAGLASAAGPARPRYLFQRRSKLWGDPVESRGLPGPEDDKPTVISELSSRLQQLNKDTRSLGEEPVGGLGSLLDPAKKSPIAAARLFSSLGELSSISAQRSPGGPGGGASYSVRPSGRYPVARRAPSPVKPASLERVEGLGAGAGGAGRPFGLTPPTILKSSSLSIPHEPKEVRFVVRSVSARSRSPSPSPLPSPASGPGPGAPGPRRPFQQKPLQLWSKFDVGDWLESIHLGEHRDRFEDHEIEGAHLPALTKDDFVELGVTRVGHRMNIERALRQLDGS

SEQ ID NO: 17 - protein, 323 aa, Homo sapiens
  misc_feature: Incyte ID No 2739431CD1
MMSPSQASLLFLNVCIFICGEAVQGNCVHHSTDSSVVNIVEDGSNAKDESKSNDTVCKEDCEESCDVKTKITREEKHFMCRNLQNSIVSYTRSTKKLLRNMMDEQQASLDYLSNQVMCDMDYRGGGWTVIQKRIDGIIDFQRLWCDYLDGFGDLLGEFWLGLKKIFYIVNQKNTSFMLYVALESEDDTLAYASYDNFWLEDETRFFKMHLGRYSGNAGDAFRGLKKEDNQNAMPFSTSDVDNDGCRPACLVNGQSVKSCSHLHNKTGWWFNECGLANLNGIHHFSGKLLATGIQWGTWTKNNSPVKIKSVSMKIRRMYNPYFK

SEQ ID NO: 18 - protein, 644 aa, Homo sapiens
  misc_feature: Incyte ID No 7473606CD1
MDGRGAFWTVAIPRARQEGLGRLGLPFPVKRTPPAPQNPGGSTQAPQRVVGKSHSGIRMPAKSRNLRLESKLNRKVVKYKWGKQGSGAGRELVPAFPTNAGLGRRDRCRPPPAGGDVASHGLPGSGVGYSCNQREEGLRGGCGGIPHVPLFLSPLPLDASGQRPSSTYRQSLRRGLGTRAHQSPANEIPELGDLRGSRLAQEPAVLFGLRPSISKRGLLARRLWAQPMLLSGWVVSTTTTIITVTVTFTPTGLLCVKHSRGPLQPTCQESAPENRVGKALITFSKGWRASLRLAPPPSALLLRRHGPGGLPVPGTMCDGALLPPLVLPVLLLLVWGLDPGTGSAPSHSPLHPASCGYLPSAFSRRPGGPGAAAGPLTAPERRRRGPRPEYGNRVAPWQARRRRVSARRCAAPFREVLARLRRRPSPGGAGQRGAVGDAAADVEVVLPWRVRPDDVHLPPLPAAPGPRRRRRPRTPPAAPRARPGERALLLHLPAFGRDLYLQLRRDLRFLSRGFEVEEAGAARRRGRPAELCFYSGRVLGHPGSLVSLSACGAAGGLVGLIQLGQEQVLIQPLNNSQGPFSGREHLIRRKWSLTPSPSAEAQRPEQLCKVLTVPQCLGLTWEDLKSGGWSDLEVPHSCVWPGGG

SEQ ID NO: 19 - protein, 881 aa, Homo sapiens
  misc_feature: Incyte ID No 3534918CD1
MEHGALGSLGEHAAKVVGKVLRQEQDFVITHHQRLVGPTVMEQKHRCKFAMKEIVQFMASGRLGPVGVPVLCHVEEVPDREQGAAPTLCPSMEEGNAKGVMSRVIFATVTLAQVSVGNTHGNWSPWSGWGTCSRTCNGGQMRRYRTCDNPPPSNGGRACGGPDSQIQRCNTDMCPVDGSWGSWHSWSQCSASCGGGEKTRKRLCDHPVPVKGGRPCPGDTTQVTRCNVQACPGGPQRARGSVIGNINDVEFGIAFLNATITDSPNSDTRIIRAKITNVPRSLGSAMRKIVSILNPIYWTTAKEIGEAVNGFTLTNAVFKRETQVEFATGEILQMSHIARGLDSDGSLLLDIVVSGYVLQLQSPAEVTVKDYTEDYIQTGPGQLYAYSTRLFTIDGISIPYTWNHTVFYDQAQGRMPFLVETLHASSVESDYNQIEETLGFKIHASISKGDRSNQCPSGFTLDSVGPFCADEDECAAGNPCSHSCHNAMGTYYCSCPKGLTIAADGRTCQDIDECALGRHTCHAGQDCDNTIGSYRCVVRCGSGFRRTSDGLSCQDINECQESSPCHQRCFNAIGSFHCGCEPGYQLKGRKCMDVNECRQNVCRPDQHCKNTRGGYKCIDLCPNGMTKAENGTCIDIDECKDGTHQCRYNQICENTRGSYRCVCPRGYRSQGVGRPCMDIDECENTDACQHECKNTFGSYQCICPPGYQLTHNGKTCQDIDECLEQNVHCGPNRMCFNMRGSYQCIDTPCPPNYQRDPVSGFCLKNCPPNDLECALSPYALEYKLVSLPFGIATNQDLIRLVAYTQDGVMHPRTTFLMVDEEQTVPFALRDENLKGVVYTTRPLREAETYRMRVRASSYSANGTIEYQTTFIVYIAVSAYPY

SEQ ID NO: 20 - protein, 957 aa, Homo sapiens
  misc_feature: Incyte ID No 2428715CD1
MGAAAVRWHLCVLLALGTRGRLAGGSGLPGSVDVDECSEGTDDCHIDAICQNTPKSYKCLCKPGYKGEGKQCEDIDECENDYYNGGCVHECINIPGNYRCTCFDGFMLAHDGHNCLDVDECQDNNGGCQQICVNAMGSYECQCHSGFFLSDNQHTCIHRSNEGMNCMNKDHGCAHICRETPKGGVACDCRPGFDLAQNQKDCTLTCNYGNGGCQHSCEDTDTGPTCGCHQKYALHSDGRTCIEKDEAAIERSQFNATSVADVDKRVKRRLLMETCAVNNGGCDRTCKDTATGVRCSCPVGFTLQPDGKTCKDINECLVNNGGCDHFCRNTVGSFECGCRKGYKLLTDERTCQDIDECSFERTCDHICINSPGSFQCLCHRGYILYGTTHCGDVDECSMSNGSCDQGCVNTKGSYECVCPPGRRLHWNRKDCVETGKCLSRAKTSPRAQLSCSKAGGVESCFLSCPAHTLFVPDAPTTPIKQKARFKIRDAKCHLRPHSQARAKETARQPLLDHCHVTFVTLKCDSSKKRRRGRKSPSKEVSHITAEFEIETKMEEASDTCEADCLRKRAEQSLQAAIKTLRKSIGRQQFYVQVSGTEYEVAQRPAKALEGQGACGAGQVLQDSKCVACGPGTHFGGELGQCVPCMPGTYQDMEGQLSCTPCPSSDGLGLPGARNVSECGGQCSPGFFSADGFKPCQACPVGTYQPEPGRTGCFPCGGGLLTKHEGTTSFQDCEAKVHCSPGHHYNTTTHRCIRCPVGTYQPEFGQNHCITCPGNTSTDFDGSTNVTHCKNQHCGGELGDYTGYIESPNYPGDYPANAECVWHIAPPPKRRILIVVPEIFLPIEDECGDVLVMRKSASPTSITTYETCQTYERPIAFTSRSRKLWIQFKSNEGNSGKGFQVPYVTYDEDYQQLIEDIVRDGRLYASENHQEILKDKKLIKALFDVLAHPQNRGLVSSC

SEQ ID NO: 21 - protein, 1393 aa, Homo sapiens
  misc_feature: Incyte ID No 3351332CD1
MGASRDRGLAALWCLGLLGGLARVAGTHYRYLWRGCYPCHLGQAGYPVSAGDQRPDVDECRTHNGGCQHRCVNTPGSYLCECKPGFRLHTDSRTCLAINSCALGNGGCQHHCVQLTITRHRCQCRPGFQLQEDGRHCVRRSPCANRNGSCMHRCQVVRGLARCECHVGYQLAADGKACPDVDECAAGLAQCAHGCLNTQGSFKCVCHAGYELGADGRQCYRIEMEIVNSCEANNGGCSHGCSHTSAGPLCTCPRGYELDTDQRTCIDVDDCADSPCCQQVCTNNPGGYECGCYAGYRLSADGCGCEDVDECASSRGGCEHHCTNLAGSFQCSCEAGYRLHEDRRGCSPLEEPMVDLDGELPFVRPLPHIAVLQDELPQLFQDDDVGADEEEAELRGEHTLTEKFVCLDDSFGHDCSLTCDDCRNGGTCLLGLDGCDCPEGWTGLICNETCPPDTFGKNCSFSCSCQNGGTCDSVTGACRCPPGVSGTNCEDGCPKGYYGKHCRKKCNCANRGRCHRLYGACLCDPGLYGRFCHLTCPPWAFGPGCSEECQCVQPHTQSCDKRDGSCSCKAGFRGERCQAECELGYFGPGCWQACTCPVGVACDSVSGECGKRCPAGFQGEDCGQECPVGTFGVNCSSSCSCGGAPCHGVTGQCRCPPGRTGEDCEADCPEGRWGLGCQEICPACQHAARCDPETGACLCLPGFVGSRCQDVCPAGWYGPSCQTRCSCANDGHCHPATGHCSCAPGWTGFSCQRACDTGHWGPDCSHPCNCSAGHGSCDAISGLCLCEAGYVGPRCEQQCPQGHFGPGCEQLCQCQHGAACDHVSGACTCPAGWRGTFCEHACPAGFFGLDCRSACNCTAGAACDAVNGSCLCPAGRRGPRCAETCPAHTYGHNCSQACACFNGASCDPVHGQCHCAPGWMGPSCLQECLPRDVRAGCRHSGGCLNGGLCDPHTGRCLCPAGWTGDKCQSPCLRGWFGEACAQRCSCPPGAACHHVTGACRCPPGFTGSGCEQACPPGSFGEDCAQMCQCPGENPACHPATGTCSCAAGYHGPSCQQRCPPGRYGPGCEQLCGCLNGGSCDAATGACRCPTGFLGTDCNLTCPQGRFGPNCTHVCGCGQGAACDPVTGTCLCPPGRAGVRCERGCPQNRFGVGCEHTCSCRNGGLCHASNGSCSCGLGWTGRHCELACPPGRYGAACHLECSCHNNSTCEPATGTCRCGPGFYGQACEHPCPPGFHGAGCQGLCWCQHGAPCDPISGRCLCPAGFHGHFCERGCEPGSFGEGCHQRCDCDGGAPCDPVTGLCLCPPGRSGATCNLDCRRGQFGPSCTLHCDCGGGADCDPVSGQCHCVDGYMGPTCREGGPLRLPENPSLAQGSAGTLPASSRPTSRSGGPARH

SEQ ID NO: 22 - protein, 3695 aa, Homo sapiens
  misc_feature: Incyte ID No 6382722CD1
MAKRLCAGSALCVRGPRGPAPLLLVGLALLGAARAREEAGGGFSLHPPYFNLAEGARIAASATCGEEAPARGSPRPTEDLYCKLVGGPVAGGDPNQTIRGQYCDICTAANSNKAHPASNAIDGTERWWQSPPLSRGLEYNEVNVTLDLGQVFHVAYVLIKFANSPRPDLWVLERSMDFGRTYQPWQFFASSKRDCLERFGPQTLERITRDDAAICTTEYSRIVPLENGEIVVSLVNGRPGAMNFSYSPLLREFTKATNVRLRFLRTNTLLGHLMGKALRDPTVTRRYYYSIKDISIGGRCVCHGHADACDAKDPTDPFRLQCTCQHNTCGGTCDRCCPGFNQQPWKPATANSANECQSCNCYGHATDCYYDPEVDRRRASQSLDGTYQGGGVCIDCQHHTAGVNCERCLPGFYRSPNHPLDSPHVCRRCNCESDFTDGTCEDLTGRCYCRPNFSGERCDVCAEGFTGFPSCYPTPSSSNDTREQVLPAGQIVNCDCSAAGTQGNACRKDPRVGRCLCKPNFQGTHCELCAPGFYGPGCQPCQCSSPGVADDRCDPDTGQCRCRVGFEGATCDRCAPGYFHFPLCQLCGCSPAGTLPEGCDEAGRCLCQPEFAGPHCDRCRPGYHGFPNCQACTCDPRGALDQLCGAGGLCRCRPGYTGTACQECSPGFHGFPSCVPCHCSAEGSLHAACDPRSGQCSCRPRVTGLRCDTCVPGAYNFPYCEAGSCHPAGLAPVDPALPEAQVPCMCRAHVEGPSCDRCKPGFWGLSPSNPEGCTRCSCDLRGTLGGVAECQPGTGQCFCKPHVCGQACASCKDGFFGLDQADYFGCRSCRCDIGGALGQSCEPRTGVCRCRPNTQGPTCSEPARDHYLPDLHHLRLELEEAATPEGHAVRFGFNPLEFENFSWRGYAQMAPVQPRIVARLNLTSPDLFWLVFRYVNRGAMSVSGRVSVREEGRSAACANCTAQSQPVAFPPSTEPAFITVPQRGFGEPFVLNPGTWALRVEAEGVLLDYVVLLPSAYYEAALLQLRVTEACTYRPSAQQSGDNCLLYTHLPLDGFPSAAGLEALCRQDNSLPRPCPTEQLSPSHPPLITCTGSDVDVQLQVAVPQPGRYALVVEYANEDARQEVGVAVHTPQRAPQQGLLSLHPCLYSTLCRGTARDTQDHLAVFHLDSEASVRLTAEQARFFLHGVTLVPIEEFSPEFVEPRVSCISSHGAFGPNSAACLPSRFPKPPQPIILRDCQVIPLPPGLPLTHAQDLTPATSPAGPRPRPPTAVDPDAEPTLLREPQATVVFTTHVPTLGRYAFLLHGYQPAHPTFPVEVLINAGRVWQGHANASFCPHGYGCRTLVVCEGQALLDVTHSELTVTVRVPEGRWLWLDYVLVVPENVYSFGYLREEPLDKSYDFISHCAAQGYHISPSSSSLFCRNAAASLSLFYNNGARPCGCHEVGATGPTCEPFGGQCPCHAHVIGRDCSRCATGYWGFPNCRPCDCGARLCDELTGQCICPPRTIPPDCLLCQPQTFGCHPLVGCEECNCSGPGIQELTDPTCDTDSGQCKCRPNVTGRRCDTCSPGFHGYPRCRPCDCHEAGTAPGVCDPLTGQCYCKENVQGPKCDQCSLGTFSLDAANPKGCTRCFCFGATERCRSSSYTRQEFVDMEGWVLLSTDRQVVPHERQPGTEMLRADLRHVPEAVPEAFPELYWQAPPSYLGDRVSSYGGTLRYELHSETQRGDVFVPMESRPDVVLQGNQMSITFLEPAYPTPGHVHRGQLQLVEGNFRHTETRNTVSREELMMVLASLEQLQIRALFSQISSAVSLRRVALEVASPAGQGALASNVELCLCPASYRGDSCQECAPGFYRDVKGLFLGRCVPCQCHGHSDRCLPGSGVCVDCQHNTEGAHCERCQAGFMSSRDDPSAPCVSCPCPLSVPSNNFAEGCVLRGGRTQCLCKPGYAGASCERCAPGFFGNPLVLGSSCQPCDCSGNGDPNLLFSDCDPLTGACRGCLRHTTGPRCEICAPGFYGNALLPGNCTRCDCTPCGTEACDPHSGHCLCKAGVTGRRCDRCQEGHFGFNGCGGCRPCACGPAAEGSECHPQSGQCHCRPGTMGPQCRECAPGYWGLPEQGCRRCQCPGGRCDPHTGRCNCPPGLSGERCDTCSQQHQVPVPGGPVGHSIHCEVCDHCVVLLLDDLERAGALLPAIHEQLRGINASSMAWARLHRLNASIADLQSQLRSPLGPRHETAQQLEVLEQQSTSLGQDARRLGGQAVGTRDQASQLLAGTEATLGHAKTLLAAIRAVDRTLSELMSQTGHLGLANASAPSGEQLLRTLAEVERLLWEMRARDLGAPQAAAEAELAAAQRLLARVQEQLSSLWEENQALATQTRDRLAQHEAGLMDLREALNRAVDATREAQELNSRNQERLEEALQRKQELSRDNATLQATLHAARDTLASVFRLLHSLDQAKEELERLAASLDGARTPLLQRMQTFSPAGSKLRLVEAAEAHAQQLGQLALNLSSIILDVNQDRLTQRAIEASNAYSRILQAVQAAEDAAGQALQQADHTWATVVRQGLVDRAQQLLANSTALEEAMLQEQQRLGLVWAALQGARTQLRDVRAKKDQLEAHIQAAQAMLAMDTDETSKKIAHAKAVAAEAQDTATRVQSQLQAMQENVERWQGQYEGLRGQDLGQAVLDAGHSVSTLEKTLPQLLAKLSILENRGVHNASLALSASIGRVRELIAQARGAASKVKVPMKFNGRSGVQLRTPRDLADLAAYTALKFYLQGPEPEPGQGTEDRFVMYMGSRQATGDYMGVSLRDKKVHWVYQLGEAGPAVLSIDEDIGEQFAAVSLDRTLQFGHMSVTVERQMIQETKGDTVAPGAEGLLNLRPDDFVFYVGGYPSTFTPPPLLRFPGYRGCIEMDTLNEEVVSLYNFERTFQLDTAVDRPCARSKSTGDPWLTDGSYLDGTGFARISFDSQISTTKRFEQELRLVSYSGVLFFLKQQSQFLCLAVQEGSLVLLYDFGAGLKKAVPLQPPPPLTSASKAIQVFLLGGSRKRVLVRVERATVYSVEQDNDLELADAYYLGGVPPDQLPPSLRRLFPTGGSVRGCVKGIKALGKYVDLKRLNTTGVSAGCTADLLVGRAMTFHGHGFLRLALSNVAPLTGNVYSGFGFHSAQDSALLYYRASPDGLCQVSLQQGRVSLQLLRTEVKTQAGFADGAPHYVAFYSNATGVWLYVDDQLQQMKPHRGPPPELQPQPEGPPRLLLGGLPESGTIYNFSGCISNVFVQRLLGPQRVFDLQQNLGSVNVSTGCAPALQAQTPGLGPRGLQATARKASRRSRQPARHPACMLPPHLRTTRDSYQFGGSLSSHLEFVGILARHRNWPSLSMHVLPRSSRGLLLFTARLRPGSPSLALFLSNGHFVAQMEGLGTRLRAQSRQRSRPGRWHKVSVRWEKNRILLVTDGARAWSQEGPHRQHQGAEHPQPHTLFVGGLPASSHSSKLPVTVGFSGCVKRLRLHGRPLGAPTRMAGVTPCILGPLEAGLFFPGSGGVITLDLPGATLPDVGLELEVRPLAVTGLIFHLGQARTPPYLQLQVTEKQVLLRADDGAGEFSTSVTRPSVLCDGQWHRLAVMKSGNVLRLEVDAQSNHTVGPLLAAAAGAPAPLYLGGLPEPMAVQPWPPAYCGCMRRLAVNRSPVAMTRSVEVHGAVGASGCPAA

SEQ ID NO: 23 - protein, 1255 aa, Homo sapiens
  misc_feature: Incyte ID No 55022490CD1
MVRGGRWEQAHKKEPLGVWGPLPCVRGAQGTLGDRNGGTGGWRHWGGCEGMPMPSSSQNVCTNSGASVGTTCHSKLDAAVDGTRCGENKWCLSGECVPVGFRPEAVDGGWSGWSAWSICSRSCGMGVQSAERQCTQPTPKYKGRYCVGERKRFRLCNLQACPAGHPSFRHVQCSHFDAMLYKGQLHTWVPVVNDVNPCELHCRPANEYFAEKLRDAVVDGTPCYQVRASRDLCINGICKNVGCDFEIDSGAMEDRCGVCHGNGSTCHTVSGTFEEAEGLGYVDVGLIPAGAREIRIQEVAEAANFLALRSEDPEKYFLNGGWTIQWNGDYQVAGTTFTYARRGNWENLTSPGPTKEPVWIQLLFQESNPGVHYEYTIHREAGGHDEVPPPVFSWHYGPWTKCTVTCGRGVQRQNVYCLERQAGPVDEEHCDPLGRPDDQQRKCSEQPCPARWWAGEWQLCSSSCGPGGLSRRAVLCIRSVGLDEQSALEPPACEHLPRPPTETPCNRHVPCPATWAVGNWSQCSVTCGEGTQRRNVLCTNDTGVPCDEAQQPASEVTCSLPLCRWPLGTLGPEGSGSGSSSHELFNEADFIPHHLAPRPSPASSPKPGTMGNAIEEEAPELDLPGPVFVDDFYYDYNFINFHEDLSYGPSEEPDLDLAGTGDRTPPPHSRPAAPSTGSPVPATEPPAAKEEGVLGPWSPSPWPSQAGRSPPPPSEQTPGNPLINFLPEEDTPIGAPDLGLPSLSWPRVSTDGLQTPATPESQNDFPVGKDSQSQLPPPWRDRTNEVFKDDEEPKGRGAPHLPPRPSSTLPPLSPVGSTHSSPSPDVAELWTGGTVAWEPALEGGLGPVDSELWPTVGVASLLPPPIAPLPEMKVRDSSLEPGTPSFPTPGPGSWDLQTVAVWGTFLPTTLTGLGHMPEPALNPGPKGQPESLSPEVPLSSRLLSTPAWDSPANSHRVPETQPLAPSLAEAGPPADPLVVRNASWQAGNWSECSTTCGLGAVWRPVRCSSGRDEDCAPAGRPQPARRCHLRPCATWHSGNWSKCSRSCGGGSSVRDVQCVDTRDLRPLRPFHCQPGPAKPPAHRPCGAQPCLSWYTSSWRECSEACGGGEQQRLVTCPEPGLCEEALRPNTTRPCNTHPCTQWVVGPWGQCSAPCGGGVQRRLVKCVNTQTGLPEEDSDQCGHEAWPESSRPCGTEDCEPVEPPRCERDRLSFGFCETLRLLGRCQLPTIRTQCCRSCSPPSHGAPSRGHQRVARR

SEQ ID NO: 24 - protein, 911 aa, Homo sapiens
  misc_feature: Incyte ID No 6755002CD1
MAQLFLPLLAALVLAQAPAALADVLEGDSSEDRAFRVRIAGDAPLQGVLGGALTIPCHVHYLRPPPSRRAVLGSPRVKWTFLSRGREAEVLVARGVRVKVNEAYRFRVALPAYPASLTDVSLALSELRPNDSGIYRCEVQHGIDDSSDAVEVKVKGVVFLYREGSARYAFSFSGAQEACARIGAHIATPEQLYAAYLGGYEQCDAGWLSDQTVRYPIQTPREACYGDMDGFPGVRNYGVVDPDDLYDVYCYAEDLNGELFLGDPPEKLTLEEARAYCQERGAEIATTGQLYAAWDGGLDHCSPGWLADGSVRYPIVTPSQRCGGGLPGVKTLFLFPNQTGFPNKHSRFNVYCFRDSAQPSAIPEASNPASNPASDGLEAIVTVTETLEELQLPQEATESESRGAIYSIPIMEDGGGGSSTPEDPAEAPRTLLEFETQSMVPPTGFSEEEGKALEEEEKYEDEEEKEEEEEEEEVEDEALWAWPSELSSPGPEASLPTEPAAQEKSLSQAPARAVLQPGASPLPDGESEASRPPRVHGPPTETLPTPRERNLASPSPSTLVEAREVGEATGGPELSGVPRGESEETGSSEGAPSLLPATRAPEGTRELEAPSEDNSGRTAPAGTSVQAQPVLPTDSASRGGVAVVPASGDCVPSPCHNGGTCLEEEEGVRCLCLPGYGGDLCDVGLRFCNPGWDAFQGACYKHFSTRRSWEEAETQCRMYGAHLASISTPEEQDFINNRYREYQWIGLNDRTIEGDFLWSDGVPLLYENWNPGQPDSYFLSGENCVVMVWHDQGQWSDVPCNYHLSYTCKMGLVSCGPPPELPLAQVFGRPRLRYEVDTVLRYRCREGLAQRNLPLIRCQENGRWEAPQISCVPRRPARALHPEEDPEGRQGRLLGRWKALLIPPSSPMPGP

SEQ ID NO: 25 - protein, 467 aa, Homo sapiens
  misc_feature: Incyte ID No 7350907CD1
MPGRWRWQRDMHPARKLLSLLFLILMGTELTQVLPTNPEESWQVYSSAQDSEGRCICTVVAPQQTMCSRDARTKQLRQLLEKVQNMSQSIEVLDRRTQRDLQYVEKMENQMKGLESKFKQVEESHKQHLARQFKAIKAKMDELRPLIPVLEEYKADAKLVLQFKEEVQNLTSVLNELQEEIGAYDYDELQSRVSNLEERLRACMQKLACGKLTGISDPVTVKTSGSRFGSWMTDPLAPEGDNRVWYMDGYHNNRFVREYKSMVDFMNTDNFTSHRLPHPWSGTGQVVYNGSIYFNKFQSHIIIRFDLKTETILKTRSLDYAGYNNMYHYAWGGHSDIDLMVDESGLWAVYATNQNAGNIVVSRLDPVSLQTLQTWNTSYPKRSAGEAFIICGTLYVTNGYSGGTKVHYAYQTNASTYEYIDIPFQNKYSHISMLDYNPKDRALYAWNNGHQILYNVTLFHVIRSDEL

SEQ ID NO: 26 - protein, 1018 aa, Homo sapiens
  misc_feature: Incyte ID No 7474411CD1
MVSHFMGSLSVLCFLLLLGFQFVCPQPSTQHRKVPQRMAAEGAPEDDGGGGAPGVWGAWGPWSACSRSCSGGVMEQTRPCLPRSYRLRGGQRPGAPARAFADHVVSAVRTSVPLHRSRDETPALAGTDASRQGPTVLRGSRHPQPQGLEVTGDRRSRTRGTIGPGKYGYGKAPYILPLQTDTAHTPQRLRRQKLSSRHSRSQGASSARHGYSSPAHQVPQHGPLYQSDSGPRSGLQAAEAPIYQLPLTHDQGYPAASSLFHSPETSNNHGVGTHGATQSFSQPARSTAISCIGAYRQYKLCNTNVCPESSRSIREVQCASYNNKPFMGRFYEWEPFAEVKGNRKCELNCQAMGYRFYVRQAEKVIDGTPCDQNGTAICVSGQCKSIGCDDYLGSDKVVDKCGVCGGDNTGCQVVSGVFKHALTSLGYHRVVEIPEGATKINITEMYKSNNYLALRSRSGRSIINGNWAIDRPGKYEGGGTMFTYKRPNEISSTAGESFLAEGPTNEILDVYMIHQQPNPGVHYEYVIMGTNAISPQVPPHRRPGEPFNGQMVTEGRSQEEGEQKGRNEEKEDLRGEAPEMFTSESAQTFPVRHPDRFSPHRPDNLVPPAPQPPRRSRDHNWKQLGTTECSTTCGKGSQYPIFRCVHRSTHEEAPESYCDSSMKPTPEEEPCNIFPCPAFWDIGEWSECSKTCGLGMQHRQVLCRQVYANRSLTVQPYRCQHLEKPETTSTCQLKICSEWQIRTDWTSCSVPCGVGQRTRDVKCVSNIGDVVDDEECNMKLRPNDIENCDMGPCAKSWFLTEWSERCSAECGAGVRTRSVVCMTNHVSSLPLEGCGNNRPAEATPCDNGPCTGKVEWFAGSWSQCSIECGSGTQQREVICVRKNADTFEVLDPSECSFLEKPPSQQSCHLKPCGAKWFSTEWSMCSKSCQGGFRVREVRCLSDDMTLSNLCDPQLKPEERESCNPQDCVPEVDENCKDKYYNCNVVVQARLCVYNYYKTACCASCTRVANRQTGFLGSR

SEQ ID NO: 27 - protein, 1458 aa, Homo sapiens [sequence omitted]

SEQ ID NO: 28 - protein, 323 aa, Homo sapiens
  misc_feature: Incyte ID No 379766CD1
MASWTSPWWVLIGMVFMHSPLPQTTAEKSPGAYFLPEFALSPQGSFLEDTTGEQFLTYRYDDQTSRNTRSDEDKDGNWDAWGDWSDCSRTCGGGASYSLRRCLTGRNCEGQNIRYKTCSNHDCPPDAEDFRAQQCSAYNDVQYQGRYYEWLPRYNDPAAPCALKCHAQGQNLVVELAPKVLDGTRCNTDSLDMCISGICQAVGCDRQLGSNAKEDNCGVCAGDGSTCRLVRGQSKSHVSPEKREENVIAVPLGSRSVRITVKGPAYPVAWALAISSNTNCLVLLCKANLASSGPYFALIPVNPTTMALNTAIVSQSAVLIDCL

SEQ ID NO: 29 - protein, 234 aa, Homo sapiens
  misc_feature: Incyte ID No 553744CD1
MMIHSCLFSPFHIAFSTPASQLFSPHGSNPSTPAATPVPTASPVKAINHPSASAAATVSGMNLLNTVLPVFPGQVSSAVHTPQPSIPNPTVIRTPSLPTAPVTSIHSTTTTPVPSIFSGLVSLPGPSATPTAATPTPGPTPRSTLGSSEAFASTSAPFTSLPFSTSSSAASTSNPNSASLSSVFAGLPLPLPPTSQGLSNPTPVIAGGSTPSVAGPLGVNSPSFVCVKRFSDIQ

SEQ ID NO: 30 - protein, 377 aa, Homo sapiens
  misc_feature: Incyte ID No 1825473CD1
MKTLPLFVCICALSACFSFSEGRERDHELRHRRHHHQSPKSHFELPHYPGLLAHQKPFIRKSYKCLHKRCRPKLPPSPNNPPKFPNPHQPPKHPDKNSSVVNPTLVATTQIPSVTFPSASTKITTLPNVTFLPQNATTISSRENVNTSSSVATLAPVNSPAPQDTTAAPPTPSATTPAPPSSSAPPETTAAPPTPSATTQAPPSSSAPPETTAAPPTPPATTPAPPSSSAPPETTAAPPTPSATTPAPLSSSAPPETTAVPPTPSATTLDPSSASAPPETTAAPPTPSATTPAPPSSPAPQETTAAPITTPNSSPTTLAPDTSETSAAPTHQTTTSVTTQTTTTKQPTSAPGQNKISRFLLYMKNLLNRIIDDMVEQ

SEQ ID NO: 31 - protein, 833 aa, Homo sapiens
  misc_feature: Incyte ID No 7950094CD1
MAPHWAVWLLAARLWGLGIGAEVWWNLVPRKTVSSGELATVVRRFSQTGIQDFLTLTLTEPTGLLYVGAREALFAFSMEALELQGAISWEAPVEKKTECIQKGKNNQTECFNFIRFLQPYNASHLYVCGTYAFQPKCTYVNMLTFTLEHGEFEDGKGKCPYDPAKGHAGLLVDGELYSATLNNFLGTEPIILRNMGPHHSMKTEYLAFWLNEPHFVGSAYVPESVGSFTGDDDKVYFFFRERAVESDCYAEQVVARVARVCKGDMGGARTLQRKWTTFLKARLACSAPNWQLYFNQLQAMHTLQDTSWHNTTFFGVFQAQWGDMYLSAICEYQLEEIQRVFEGPYKEYHEEAQKWDRYTDPVPSPRPGSCINNWHRRHGYTSSLELPDNILNFVKKHPLMEEQVGPRWSRPLLVKKGTNFTHLVADRVTGLDGATYTVLFIGTGDGWLLKAVSLGPWVHLIEELQLFDQEPMRSLVLSQSKKLLFAGSRSQLVQLPVADCMKYRSCADCVLARDPYCAWSVNTSRCVAVGGHSGSLLIQHVMTSDTSGICNLRGSKKVRPTPKNITVVAGTDLVLPCHLSSNLAHARWTFGGRDLPAEQPGSFLYDARLQALVVMAAQPRHAGAYHCFSEEQGARLAAEGYLVAVVAGPSVTLEARAPLENLGLVWLAVVALGAVCLVLLLLVLSLRRRLREELEKGAKATERTLVYPLELPKEPTSPPFRPCPEPDEKLWDPVGYYYSDGSLKIVPGHARCQPGGGPPSPPPGIPGQPLPSPTRLHLGGGRNSNANGYVRLQLGGEDRGGLGHPLPELADELRRKLQQRQPLPDSNPEESSV

SEQ ID NO: 32 - protein, 1291 aa, Homo sapiens
  misc_feature: Incyte ID No 7479484CD1
MFRPTTVAVDEDGGEEDKDESSTNSGASAVSSCGFGADFSTDKGGSFTSVQITNTTGLSQAPGLASQGISFGIKNNLGPPLQKLGVSFSFAKKAPVKLESIASVFKDHAEEGSSEDGTKADEKSSDQGVQKVGDTDGTGNLDGKKEDEDPQDGGSLASTLSKLKRMKREEGTGATEPEYYHYIPPAHCKVKPNFPFLLFMRASEQMEGDHSAHSKSAPENRKSSSPKPQGCSKTAASPGAERTVSEASELQKEAAVAGPSEPGGKTETKKGSGGGEDEQSVESRETSESPMCESNPKDISQATPATKAGQGPKHPTGPFFPVLSKDESTALQWPSELLIFTKAEPSISYSCNPLYFDFKLSRNKDAKAKGTEKPKDVAGSSKDHLQSLDPREPNKSQEEEQDVVLSSEGRVDEPASGAACSSLNKQEPGGSHMSETEDTGRSHPSKKEPSGKSHRHKKKKKHKKSSKHKRKHKADTEEKSSKAESGEKSKKRKKRKRKKNKSSAAADSERGPKSEPPGSGSPAPPRRRRRAQDDSQRRSLPAEEGNSGKKDDGGGGSSCQDHSGRKHKGEPPTSSCQRRANTKHSSRSSHRSQPSSGDEDSDDASSHRLHQKSPSQYSEEEEEEEEEEEEEDEDSGSEHSRSRSRSGHRHSSHRSSRRSYSSSSDASSDQSCYSRQHSYSDDSYSDYSDRSRRHSKRSHDSDDSDYTSSKHRSKRHKYSSSDDDYSLSCSQSRSRSRSHTRERSRSRGRSRSSSCSRSRSKRRSRSTTAHSWQRSRSYSRDRSRSTRSPSQRSGSRKGSWGHESPEERRSGRRDFIRSKIYRSQSPHYFQSGRGEGPGKKEDGRGDDSKGAGLPSQNSNTGTGRGSESDCSPEDKNSVTARLLLEKIQSRKVERKPNVCEEVLATPNKAGLKYKNPPQGYFGPKLPPSLGNKPVLPMIGKLPATRKSNKKCEESGLERGEEQEHSEPEEGSPRSSDAPFGHQFSEEAAGPLSDPPPEEPKSEEATADHSVAPLGTPAHTDCYPGDPAISHNYLPDPSDGDTLESLDSGSQPGPVESSLLPIAPDLEHFPNYAPPSGEPSIESTDGTEDASLAPLESQPITFTPEEMEKYSKLQQAAQQHIQQQLLAKQVKAFPASTALAPATPALQPIHIQQPATASATSITTVQHAILQHHAAAAAAAIGIHPHPHPQPLAQVHHIPQPHLTPISLSHLTHSIIPGHPATFLASHPIHIIPASAIHPGPFTFHPVPHAALYPTLLAPRPAAAAATALHLHPLLHPIFSGQDLQHPPSHGT

SEQ ID NO: 33 - protein, 736 aa, Homo sapiens
  misc_feature: Incyte ID No 6780147CD1
MAVRALKLLTTLLAVVAAASQAEVESEAGWGMVTPDLLFAEGTAAYARGDWPGVVLSMERALRSRAALRALRLRCRTQCAADFPWELDPDWSPSPAQASGAAALRDLSFFGGLLRRAACLRRCLGPPAAHSLSEEMELEFRKRSPYNYLQVAYFKINKLEKAVAAAHTFFVGNPEHMEMQQNLDYYQTMSGVKEADFKDLETQPHMQEFRLGVRLYSEEQPQEAVPHLEAALQEYFVAYEECRALCEGPYDYDGYNYLEYNADLFQAITDHYIQVLNCKQNCVTELASHPSREKPFEDFLPSHYNYLQFAYYNIGNYTQAVECAKTYLLFFPNDEVMNQNLAYYAAMLGEEHTRSIGPRESAKEYRQRSLLEKELLFFAYDVFGIPFVDPDSWTPEEVIPKRLQEKQKSERETAVRISQEIGNLMKEIETLVEEKTKESLDVSRLTREGGPLLYEGISLTMNSKLLNGSQRVVMDGVISDHECQELQRLTNVAATSGDGYRGQTSPHTPNEKFYGVTVFKALKLGQEGKVPLQSAHLYYNVTEKVRRIMESYFRLDTPLYFSYSHLVCRTAIEEVQAERKDDSHPVHVDNCILNAETLVCVKEPPAYTFRDYSAILYLNGDFDGGNFYFTELDAKTVTAEVQPQCGRAVGFSSGTENPHGVKAVTRGQRCAIALWFTLDPRHSERDRVQADDLVKMLFSPEEMDLSQEQPLDAQQGPPEPAQESLSGSESKPKDEL

SEQ ID NO: 34 - protein, 1896 aa, Homo sapiens
  misc_feature: Incyte ID No 7204554CD1
MPLPPRSLQVLLLLLLLLLLLPGMWAEAGLPRAGGGSQPPFRTFSASDWGLTHLVVHEQTGEVYVGAVNRIYKLSGNLTLLRAHVTGPVEDNEKCYPPPSVQSCPHGLGSTDNVNKLLLLDYAANRLLACGSASQGICQFLRLDDLFKLGEPHHRKEHYLSSVQEAGSMAGVLIAGPPGQGQAKLFVGTPIDGKSEYFPTLSSRRLMANEEDADMFGFVYQDEFVSSQLKIPSDTLSKFPAFDIYYVYSFRSEQFVYYLTLQLDTQLTSPDAAGEHFFTSKIVRLCVDDPKFYSYVEFPIGCEQAGVEYRLVQDAYLSRPGRALAHQLGLAEDEDVLFTVFAQGQKNRVKPPKESALCLFTLRAIKEKIKERIQSCYRGEGKLSLPWLLNKELGCINSPLQIDDDFCGQDFNQPLGGTVTIEGTPLFVDKDDGLTAVAAYDYRGRTVVFAGTRSGRIRKILVDLSNPGGRPALAYESVVAQEGSPILRDLVLSPNHQYLYAMTEKQVTRVPVESCVQYTSCELCLGSRDPHCGWCVLHSICSRRDACERADEPQRFAADLLQCVQLTVQPRNVSVTMSQVPLVLQAWNVPDLSAGVNCSFEDFTESESVLEDGRIHCRSPSAREVAPITRGQGDQRVVKLYLKSKETGKKFASVDFVFYNCSVHQSCLSCVNGSFPCHWCKYRHVCTHNVADCAFLEGRVNVSEDCPQILPSTQIYVPVGVVKPITLAARNLPQPQSGQRGYECLFHIPGSPARVTALRFNSSSLQCQNSSYSYEGNDVSDLPVNLSVVWNGNFVIDNPQNIQAHLYKCPALRESCGLCLKADPRFECGWCVAERRCSLRHHCAADTPASWMHARHGSSRCTDPKILKLSPETGPRQGGTRLTITGENLGLRFEDVRLGVRVGKVLCSPVESEYISAEQIVCEIGDASSVRAHDALVEVCVRDCSPHYRALSPKRFTFVTPTFYRVSPSRGPLSGGTWIGIEGSHLNAGSDVAVSVGGRPCSFSWRNSREIRCLTPPGQSPGSAPIIININRAQLTNPEVKYNYTEDPTILRIDPEWSINSGGTLLTVTGTNLATVREPRIRAKYGGIERENGCLVYNDTTMVCRAPSVANPVRSPPELGERPDELGFVMDNVRSLLVLNSTSFLYYPDPVLEPLSPTGLLELKPSSPLILKGRNLLPPAPGNSRLNYTVLIGSTPCTLTVSETQLLCEAPNLTGQHKVTVRAGGFEFSPGTLQVYSDSLLTLPAIVGIGGGGGLLLLVIVAVLIAYKRKSRDADRTLKRLQLQMDNLESRVALECKEAFAELQTDIHELTNDLDGAGIPFLDYRTYAMRVLFPGIEDHPVLKEMEVQANVEKSLTLFGQLLTKKHFLLTFIRTLEAQRSFSMRDRGNVASLIMTALQGEMEYATGVLKQLLSDLIEKNLESKNHPKLLLRRTESVAEKMLTNWFTFLLYKFLKECAGEPLFMLYCAIKQQMEKGPIDAITGEARYSLSEDKLIRQQIDYKTLTLNCVNPENENAPEVPVKGLDCDTVTQAKEKLLDAAYKGVPYSQRPKAADMDLEWRQGRMARIILQDEDVTTKIDNDWKRLNTLAHYQVTDGSSVALVPKQTSAYNISNSSTFTKSLSRYESMLRTASSPDSLRSRTPMITPDLESGTKLWHLVKNHDHLDQREGDRGSKMVSEIYLTRLLATKGTLQKFVDDLFETIFSTAHRGSALPLAIKYMFDFLDEQADKHQIHDADVRHTWKSNCLPLRFWVNVIKNPQFVFDIHKNSITDACLSVVAQTFMDSCSTSEHKLGKDSPSNKLLYAKDIPNYKSWVERYYADIAKMPAISDQDMSAYLAEQSRLHLSQFNSMSALHEIYSYITKYKDEILAALEKDEQARRQRLRSKLEQVVDTMALSS

SEQ ID NO: 35 - protein, 215 aa, Homo sapiens
  misc_feature: Incyte ID No 6833247CD1
MGLEKPQSKLEGGMHPQLIPSVIAVVFILLLSVCFIASCLVTHHNFSRCKRGTGVHKLEHHAKLKCIKEKSELKSAEGSTWNCCPIDWRAFQSNCYFPLTDNKTWAESERNCSGMGAHLMTISTEAEQNFIIQFLDRRLSYFLGLRDENAKGQWRWVDQTPFNPRRVFWHKNEPDNSQGENCVVLVYNQDKWAWNDVPCNFEASRICKIPGTTLN

SEQ ID NO: 36 - protein, 579 aa, Homo sapiens
  misc_feature: Incyte ID No 4148119CD1
MGRPTQWPSLLLLLLLPGPPPVAGLEDAAFPHLGESLQPLPRACPLRCSCPRVDTVDCDGLDLRVFPDNITRAAQHLSLQNNQLQELPYNELSRLSGLRTLNLHNNLISSEGLPDEAFESLTQLQHLCVAHNKLSVAPQFLPRSLRVADLAANQVMEIFPLTFGEKPVLRSVYLHNNQLSNAGLPPDAFRGSEAIATLSLSNNQLSYLPPSLPPSLERLHLQNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQMLDLSHNELSFVPPDLPEALEELHLEGNRIGHVGPEAFLSTPRLRALFLRANRLHMTSIAAEAFLGLPNLRVVDTAGNPEQVLIRLPPTTPRGPRAGGP

SEQ ID NO: 37 - DNA, 1211 bp, Homo sapiens
  misc_feature: Incyte ID No 1888682CB1
ccgggacggtcacatcccgctgcaggggcgggcggaggccgccgcactgcctcccgcaccggggacccaggccagcgtccgggcaacgccccctgctcccggacagactccgtggcccgctcgagccctgggggctccgcagacccgcgcccgctccgcccgcagctcggccccgcgctgcccgcgtcgccgggcccgcgccgggatggggtaggggcagcgccaccgagtcgggcgatgggccgccctctgggcaccgagcagccccccgaggcctgaccaaccgcgaggaccggcggaggagccccgcctggatgtcaagcggatgccaagcggatgccacagttccccccccagcggactccgtggggacatggcttcgctggtgcccctttccccatatctaagccccacggtcctcctgctggtcagctgtgacctgggcttcgtgcgagcagaccggcctccctctcctgtgaatgtgacggtcactcacctcagagccaactcggccactgtgtcctgggacgtcccagaaggcaacatcgtcattggctactccatttcccagcaacggcagaatggccccgggcagcgtgtgattcgggaggtgaacaccaccacccgggcctgtgccctctggggcctggctgaagacagtgactacacagtgcaggtcaggagcatcggccttcggggagagagtcccccagggccccgggtgcacttccgaactctcaagggttctgaccggctaccttcaaacagttcaagcccaggtgacatcacagtggaaggtctggatggagagcggccactgcagactggggaagtggtcatcattgtggtggtgttgctcatgtgggctgctgtaattgggctgttctgccgtcagtatgacatcatcaaggacaatgactccaacaacaatcccaaggagaagggaaaggggccggaacagagtcctcagggaaggccagtggggacaagacagaaaaagtcaccatctatcaacaccatcgacgtttgagtgaagaaacacacccagaagagagatgcactaacaactggggatagggatggggtcagggggagcccaagatggtgatctgcccgagactcccagagggtattgccactcccacaatctcaggcctggtacccatcctctttccactgtgagcagagccagaaggtaggtctgttcagag

SEQ ID NO: 38 - DNA, 1523 bp, Homo sapiens
  misc_feature: Incyte ID No 1794980CB1
ggcggctggcggcgcgggcaggcaggcggggaggacaggctgggggcggcgaccgcgaggggccgcgcgcggagggcgcctggtgcagcatgggcggcccgcgggcttgggcgctgctctgcctcgggctcctgctcccgggaggcggcgctgcgtggagcatcggggcagctccgttctccggacgcaggaactggtgctcctatgtggtgacccgcaccatctcatgccatgtgcagaatggcacctaccttcagcgagtgctgcagaactgcccctggcccatgagctgtccggggagcagctacagaactgtggtgagacccacatacaaggtgatgtacaagatagtgaccgcccgtgagtggaggtgctgccctgggcactcaggagtgagctgcgaggaagttgcagcttcctctgcctccttggagcccatgtggtcgggcagtaccatgcggcggatggcgcttcggcccacagccttctcaggttgtctcaactgcagcaaagtgtcagagctgacagagcggctgaaggtgctggaggccaagatgaccatgctgactgtcatagagcagccagtacctccaacaccagctacccctgaggaccctgccccgctctggggtccccctcctgcccagggcagccccggagatggaggcctccaggaccaagtcggtgcttgggggcttcccgggcccaccggccccaagggagatgccggcagtcggggcccaatggggatgagaggcccaccaggtccacagggccccccagggagccctggccgggctggagctgtgggcacccctggagagaggggacctcctgggccaccagggcctcctggcccccctgggcccccagcccctgttgggccaccccatgcccggatctcccagcatggagacccattgctgtccaacaccttcactgagaccaacaaccactggccccagggacccactgggcctccaggccctccagggcccatgggtccccctgggcctcctggccccacaggtgtccctgggagtcctggtcacataggacccccaggccccactggacccaaaggaatctctggccacccaggagagaagggcgagagaggactgcgtggggagcctggcccccaaggctctgctgggcagcggggggaacctggccctaagggagaccctggtgagaagagccactggggggaggggttgcaccagctacgcgaggctttgaagattttagctgagagggttttaatcttggaaacaatgattgggctctatgaaccagagctggggtctggggcgggccctgccggcacaggcacccccagcctccttcggggcaagaggggcggacatgcaaccaactaccggatcgtggcccccaggagccgggacgagagaggctgagttggtggcggcccctgaggcagaccaggccaggcttcccctcctacctggactcggccagctgcctccagggaccgcccgtccataattcaggagcgtccc

SEQ ID NO: 39 - DNA, 1368 bp, Homo sapiens
  misc_feature: Incyte ID No 5533958CB1
ctgccgggtgtgccgggtgtccagcgaacccctttcccaaaccttcggggagaagggaggtgggaggaggcaaagaaactacaggcagggagctggaagggggggtggggggggcaggagacaagaaatcaagacaccaggcagcaggacacacacacactcacatacactcacacacatagagaccaacagatagacagctacctaaagcctgaaagactgacagcaacacagaaaaaaagaaacaggcagaaagagagacaaagacagaaatagaaacagactaacacacagagtcaaaaatacagagacagaaagacagggagaaagagaaacagaaaattagacaccaaagacatacgaacagggaggaaggccgactgaaagaaagacggagaagaggagagagaagccagggccgagcgtgccagcaggcggatggagggcggcctggtggaggaggagacgtagtggcctgggctgagctgggtgggccgggagaagcgggtgcctcagagtgggggtgggggcatgggaggggcaggcattctgctgctgctgctggctggggcgggggtggtggtggcctggagacccccaaagggaaagtgtcccctgcgctgctcctgctctaaagacagcgccctgtgtgagggctccccggacctgcccgtcagcttctctccgaccctgctgtcactctcactcgtcaggacgggagtcacccagctgaaggccggcagcttcctgagaattccgtctctgcacctgctcctcttcacctccaactccttctccgtgattgaggacgatgcatttgcgggcctgtcccacctgcagtacctcttcatcgaggacaatgagattggctccatctctaagaatgccctcagaggacttcgctcgcttacacacctaagcctggccaataaccatctggagaccctccccagattcctgttccgaggcctggacacccttactcacgtggacctccgcgggaacccgttccagtgtgactgccgcgtcctctggctcctgcagtggatgcccaccgtgaatgccagcgtggggaccggcgcctgtgcgggccccgcctccctgagccacatgcagctccaccacctcgaccccaagactttcaagtgcagagccataggtggggggctttcccgatggggtgggaggcgggagatctgggggaaaggctgccagggccaagaggctcgtctcactccctgccctgccatttcccggagtgggaagaccctgagcaagcagcactgccttcctgagccccagttttctcatctgtaaagtgggggtaataaacagtgatataggagtgccatggaaaaaaaaaaaaaa

SEQ ID NO: 40 - DNA, 3157 bp, Homo sapiens
  misc_feature: Incyte ID No 60210196CB1
tggtcgttcctcggtttgccatccattgggcccctgccctccatccccgtggaggccccttgtctgggtgttcgcactcaacgtcgatgtgttgataatggtgcctttttcgtgaagaaactgcctgagtctcacttccagaagtttataggtccacccgttctctccagcgtccgccagcccagatctcgcatgcgcatctgtgtctgcccctctttgccttctgcctgtccctgggtgcccctcagggtcagaatcaccctttccgcccgcactggcccccacatcacctgtcttgtccccactggccttccctgaggactctgttccggcccctttcccttctccttgggattgttgttggagtcattgtccttgatgatgtcatactgacggcagaacagcccaattacagctgaaacacaatacagtctgagcccagagagccgcggggaccatggagccggtgccgctgcaggacttcgtgcgcgccttggaccccgcctccctcccgcgcgtgctgcgggtctgctcgggggtctacttcgagggctccatctatgagatctctgggaatgagtgctgcctctccacgggggacctgatcaaggtcacccaggtccgcctccagaaggtggtctgtgagaacccgaagaccagccagaccatggagctcgcccccaacttccagggctacttcacccccctcaacaccccacagagctatgaaaccctggaggagctggtctctgccacaactcagagctccaagcagctgcccacttgcttcatgtcgacccacaggattgtcacagagggcagggtggtgactgaggaccagctcctcatgcttgaggctgtggtgatgcacctcgggatccgctctgcccgctgtgtcctgggcatggagggtcagcaggtcatcctgcacctgcccctatcccagaaggggcccttctggacatgggagcctagtgcccctcgaactctgctccaggtcctacaggatccagccctgaaagacctcgtcctcacctgccccaccctgccctggcattccctgatcctgcggccccagtatgagatccaagccatcatgcacatgcgcaggaccattgtcaagatcccttctaccctggaggtcgacgtggaggacgtcaccgcctcctcccggcacgtccactttatcaaaccgctgctgctgagcgaggtcctggcctgggaaggccctttccccctgtccatggagatcctggaggttcctgagggccgccccatcttcctcagcccgtgggtgggctccttgcaaaaaggccagaggctttgcgtctatggcctagcctcaccaccctggcgggtcctggcctcaagcaagggccgcaaggtgcccaggcacttcctggtgtcagggggctaccaaggcaagctgcggcggcggccaagggagttccccacggcctatgacctcctaggtgctttccagccaggccggccactccgggtggtggccacaaaggactgtgagggcgagagggaggagaatcccgagttcacgtccctggctgtgggtgaccggctggaggtgctggggcctggccaggcccatggggcccagggcagtgacgtggatgtcttggtttgtcagcggctgagtgaccaggctggggaggatgaggaggaagagtgcaaagaggaggcagagagcccagagcgggtcctgctgcccttccacttccctggcagtttcgtggaggagatgagtgacagccggcgctacagcctggcagatctgactgcccagttttcactgccttgtgaggtcaaggtggtggccaaggacaccagccaccccactgaccctctgacctccttcctgggcctgcggctggaggagaagatcacagagccattcttggtggtgagcctagactctgagcctgggatgtgctttgagatccctccccggtggctggacctgactgttgtgaaggccaaggggcagccagacttgccagaggggtctctccccatagccacagtggaggagctgacagacaccttctattatcgtcttcggaagttaccagcctgtgagatccaagcccccccacccaggccccctaaaaatcagggcctcagcaagcagaggagacacagcagtgagggaggcgtcaagtcttctcaagtcttaggattgcagcaacacgctcggctgcccaaacccaaggcgaagaccttgccagagttcatcaaggatggctccagtacgtacagcaagattcctgcccacaggaagggccacaggcccgctaagccccaaaggcaggatctagatgatgatgaacatgattatgaagaaatacttgagcaatttcagaaaaccatctaagtgctggaggaaccacgcttcctaactgctgcttctcagggaatccgacaccagccaaccattttaagcctctaaaagacctcgggcaagtctcacagaaactgagctgcagacggggagtagctttgtggaaactgatttgatggacactgcaccagcttccttcaggttctagattcttgctacttagggcgggctggtttggacctaacatctcgcacgtgactccctcagcctcagagccttgggatgcagagcagctggcagggttcctctcaatcctgcaaccccagctgtcccaccggtggatgcagaggggaatccgaggccatcaaccttggtgacagcagcgcagtgccaatgctgatcacactgcatgggagattttgttaacgtctgccacccccactctcacccccaagctctaagcccccgggaggcctggactgtcttcctcatctctgtagcaccaagcctgatagatctgtatatggtaaacaggggtttaaccacatgtggttaacatggattaatgtgggaatttggcttcaagaacacaaccttaggaccttgggccccaaaagctggtggtgaaatgagaggagccaatttaagaagacccttatggagacctgaggctgcagaaactggtaggtttcatcaggtggttaaagtcgtcaaagttgtaagtgactaaccaagattatttcattttaaaaccacagaataaaaatgacacctgagcttctctaaaaaaaaaaaaaaaaa

SEQ ID NO: 41 - DNA, 3264 bp, Homo sapiens
  misc_feature: Incyte ID No 815125CB1
ggagccggggcagccagaagaggtgggaaaagcggaggaggacgcccaggaggaggcggcggcggcggccgggaagtgaaaggtctcgcaaagttcagcggcggctgcgggcgccgagccccgggctagcggcagacgagcccgcagggccgctccgcggggcagcgcagccaggccggctatggtcccggggctcccgccgccccccaggtgcccgggacccgccaggccgggtgcgcgagggtcaccccacctccccgcgcggtcccggcccctggctcccagctgccggcgaccgctgaccgagcccggcgccccaggaggaggaagaaaccagggccccgttccctcccgaggacggcggcgcttcatcccgcagcccagaggtctcggctccctccggcacccgcccggcccggctgctcccggctcctcccggccatggggagctgcgcgcggctgctgctgctctggggctgcacggtggtggccgcaggactgagtggagtagctggagtgagttcccgctgtgaaaaagcctgcaaccctcggatgggaaatttggctttggggcgaaaactctgggcagacaccacctgcggtcagaatgctaccgaactgtactgcttctacagtgagaacacggatctgacttgtcggcagcccaaatgtgacaagtgcaatgctgcctatcctcacctggctcacctgccatctgccatggcagactcatccttccggtttcctcgcacatggtggcagtctgcggaggatgtgcacagagaaaaggtccagttagacctggaagctgaattctacttcactcacctaattgtgatgttcaagtcccccaggccagctgccatggtgctggaccgctcccaggactttgggaaaacatggaagccttataagtactttgcgactaactgctccgctacatttggcctggaagatgatgttgtcaagaagggcgctatttgtacttctaaatactccagtccttttccatgcactggaggagaggttattttcaaagctttgtcaccaccatacgatacagagaacccttacagtgccaaagttcaggagcagctgaagatcaccaaccttcgcgtgcagctgctgaaacgacagtcttgtccctgtcagagaaatgacctgaacgaagagcctcaacattttacacactatgcaatctatgatttcattgtcaagggcagctgcttctgcaatggccacgctgatcaatgcatacctgttcatggcttcagacctgtcaaggccccaggaacattccacatggtccatgggaagtgtatgtgtaagcacaacacagcaggcagccactgccagcactgtgccccgttatacaatgaccggccatgggaggcagctgatggcaaaacgggggctcccaacgagtgcagaacctgcaagtgtaatgggcatgctgatacctgtcacttcgacgttaatgtgtgggaggcatcagggaatcgtagtggtggtgtctgtgatgactgtcagcacaacacagaaggacagtattgccagaggtgcaagccaggcttctatcgtgacctgcggagacccttctcagctccagatgcttgcaaaccgtgttcctgccatccagtaggatcagctgtccttcctgccaactcagtgaccttctgcgaccccagcaatggtgactgcccttgcaagcctggggtggcagggcgacgttgtgacaggtgcatggtgggatactggggcttcggagactatggctgtcgaccatgtgactgtgcagggagctgtgaccctatcaccggagactgcatcagcagccacacagacatagactggtatcatgaagttcctgacttccgtcccgtgcacaataagagcgaaccagcctgggagtgggaggatgcgcaggggttttctgcacttctacactcaggtaaatgcgaatgtaaggaacagacattaggaaatgccaaggcattctgtggaatgaaatattcatatgtgctaaaaataaagattttatcagctcatgataaaggtactcatgttgaggtcaatgtgaagattaaaaaggtcttaaaatctaccaaactgaagattttccgaggaaagcgaacattatatccagaatcatggacggacagaggatgcacttgtccaatcctcaatcctggtttggaataccttgtagcaggacatgaggatataagaacaggcaaactaattgtgaatatgaaaagctttgtccagcactggaaaccttctcttggaagaaaagtcatggatattttaaaaagagagtgcaagtagcattaagatggatagcacataatggcacttgtctatgtacaaaacacaaactttagagcaagaagacctcagacaggaaactggaattttttaaagtgccaaaacatatagaaatgtttgaatgcatgggtcttatctaatttatctcttctggacccatgtttaaatacagttttatttcatgaagagaaatgaaaacccctacactgatatctgttttctatgggactgattctgaaattcttaactattaagaatattttaatagcagcatgacatttagcagtaatccattaagggcagtacctctaacaaggacgccttccagcttcagctatgttacttacgtttgatgctacttaaagtaatgaatgacgttttaaggaatccctaaccctactatcagaaaaggtgtttgttaaagagccttctcttgtgtgttacgcatgaactttggtctgtaggtgttaaatggaacctctccatgtgtatatagtatttccttgtataaagcactttactacctaccacttgtgttgtgaacgtttggtgactgctgttgaaagaaggaaaagggtgtgtgagaaagcctactgaagcagcagcactgccactacatgtggacaaaagtgaacatataaaagaagttgtgctatttaactctgaatacttggagaaactaggtgaagatgcaaccagaaaggagaatatgtatgcgtgaagtctcagctttgagctggaggctagattccaagatgacagccatgatgaaactttttaaaaaactaaaccagaagagactttaaaataagagaaagaaatcataaatgtagacatatgcttggctaaaggggaaatggactttaaattttaaagagctcatttgcaatgcacttgtatacacttcaaaaattattgtagacacagcatttgtta

SEQ ID NO: 42 - DNA, 3383 bp, Homo sapiens
  misc_feature: Incyte ID No 1386915CB1
tgcgatctagaacgtccgacctctcctctcccagccagtcgtggctggcctttcaaagtgtgcagttgtctcctccctgtccagccccatcgtcgcccaggaccagctgggccgcggtctgacctgaggctgctgctcagcgccggggcgctggcgctctccattcgagcaccttccagcataccgctcggctccgggagccgctctgcaaagttgagcagctcagagcgcaagctttgcctctcgacttctccctccttgggtccccggcgcccccgcctcccacgatccctttcactaggagcagccagtcccagcgggctggcaacttgcaccccttcctagtcatcctccctgaaacgcgaccatgctgttaaggggcgtcctcctggcgttgcaagccctgcagctcgccggtgccctcgacctgcccgctgggtcctgtgcctttgaagagagcacttgcggctttgactccgtgttggcctctctgccgtggattttaaatgaggaaggccattacatttatgtggatacctcctttggcaagcagggggagaaagctgtgctgctaagtcctgacttacaggctgaggaatggagctgcctccgtttggtctaccagataaccacatcttcggagtctctgtcagatcccagccagctgaacctctacatgagatttgaagatgaaagctttgatcgcttgctttggtcagctaaggaaccttcagacagctggctcatagccagcttggatttgcaaaacagttccaagaaattcaagattttaatagaaggtgtactaggacagggaaacacagccagcatcgcactatttgaaatcaagatgacaaccggctactgtattgaatgtgactttgaagaaaatcatctctgtggctttgtgaaccgctggaatcccaatgtgaactggtttgttggaggaggaagtattcggaatgtccactccattctcccacaggatcacaccttcaagagtgaactgggccactacatgtacgtggactcagtttatgtgaagcacttccaggaggtggcacagctcatctccccgttgaccacggcccccatggctggctgcctgtcattttattaccagatccagcaggggaatgacaatgtcttttccctttacactcgggatgtggctggcctttacgaggaaatctggaaagcagacaggccagggaatgctgcctggaaccttgcggaggtcgagttcaatgctccttaccccatggaggttatttttgaagttgctttcaatggtcccaagggaggttatgttgccctggatgatatttcattctctcctgttcactgccagaatcagacagaacttctgttcagtgccgtggaagccagctgcaattttgagcaagatctctgcaacttttaccaagataaagaaggtccaggttggacccgagtgaaagtaaaaccaaacatgtatcgggctggagaccacactacaggcttagggtattacctgctagccaacacaaagttcacatctcagcctggctacattggaaggctctatgggccctccctaccaggaaacttgcagtattgtctgcgttttcattatgccatctatggatttttaaaaatgagtgacaccctagcagtttacatctttgaagagaaccatgtggttcaagagaagatctggtctgtgttggagtccccaaggggtgtttggatgcaagctgaaatcacctttaagaagcccatgcctaccaaggtggttttcatgagcctatgcaaaagtttctgggactgtgggcttgtagccctggatgacattacaatacaattgggaagctgctcatcttcagagaaacttccacctccacctggagagtgtactttcgagcaagatgaatgtacatttactcaggagaaaagaaaccggagcagctggcacaggaggaggggagaaactcccacttcctacacaggaccaaagggagatcacactactggggtaggctactacatgtacattgaggcctcccatatggtgtatggacaaaaagcacgcctcttgtccaggcctctgcgaggagtctctggaaaacactgcttgacctttttctaccacatgtatggagggggcactggcctgctgagtgtttatctgaaaaaggaagaagacagtgaagagtccctcttatggaggagaagaggtgaacagagcatttcctggctacgagcactgattgaatacagctgtgagaggcaacaccagataatttttgaagccattcgaggagtatcaataagaagtgatattgccattgatgatgttaaatttcaggcaggaccctgtggagaaatggaagatacaactcaacaatcatcaggatattctgaggacttaaatgaaattgagtattaagaaatgatctgcattggatttactagacgaaaaccatacctctcttcaatcaaaatgaaaacaaagcaaatgaatactggacagtcttaacaattttataagttataaaatgactttagagcaccctccttcattacttttgcaaaaacatactgactcagggctctttttttctttttgcatatgacaactgttactagaaatacaggctactggttttgcatagatcattcatcttaattttggtaccagttaaaaatacaaatgtactatattgtagtcattttaaagtacacaaagggcacaatcaaaatgagatgcactcatttaaatctgcattcagtgaatgtattgggagaaaaataggtcttgcaggtttccttttgaattttaagtatcataaatattttttaagtaaataatacggggtgtcagtaatatctgcagaatgaatgcagtctttcatgctaatgagttagtctggaaaaataaagtcttattttctatgttttattcatagaaatggagtattaatttttaatattttcaccatatgtgataacaaaggatctttcatgaatgtccaagggtaagtcagtattaattaatgctgtattacaaggcaatgctaccttctttattccccctttgaactacctttgaagtcactatgagcacatggatagaaatttaacttttttttgtaaagcaagcttaaaatgtttatgtatacatacccagcaacttttataaatgtgttaaacaattttactgatttttataataaatattttggtaagattttgaataatatgaattcaggcagatatactaaactgcttttatttacttgtttagaaaattgtatatatatgtttgtgtatcctaacagctgctatgaaattataaaattacctaataaaaataatttgaaaatcttaaaaaaaaaaaaaaa

SEQ ID NO: 43 - DNA, 2741 bp, Homo sapiens
  misc_feature: Incyte ID No 1344495CB1
ggcagctgcgggtcgcgggtcgcgggtcgcgagtcgccggtcgccggtcgcggcggagcctgggcgctgagtgaagaaaatgaggcacgaggaattgttaaccaagaccttccaaggcccagctgttgtgtgtgggactccgaccagccacgtatacatgtttaagaatggcagtggggactcgggggactcttctgaagaagagtctcaccgtgtggttttgcggccccggggcaaggagcgccacaagagcggtgtccaccagcctccccaggcgggagcaggtgacgtggtgctgctgcagcgggagctggcccaggaggacagcctcaacaagctggcgctgcagtatggctgcaaagttgcagatatcaagaaagtcaacaacttcatcagagaacaagacttatatgctttgaaatctgttaagattccagtgagaaaccatgggatcctgatggagacccacaaagaactgaaaccccttctgagcccgtcttccgagaccacagtgaccgtggaactgccagaggcagacagagcaggcgcgggcaccggtgcccaggccggccaactgatgggcttctttaaggggattgaccaggatattgagcgtgcagtgcagtcagaaatctttctacatgaaagttactgcatggacacctcccatcagccactgctcccggcacctccgaagacgcctatggatggtgcagattgtggcattcagtggtggaatgctgttttcatcatgctgctgattggtattgtcttgcctgtcttttatttggtctactttaaaatacaagctagtggtgagacccctaatagcttgaacacaactgtcatccccaatggctcgatggcaatgggtacagttccagggcaagcccccagactagcagttgcagtgccagccgtcacttctgcagacagccagttcagtcagaccacccaagcggggagctaagctttgtttttaaagactcggcccagctttagcaattggctgttgatgtgcctcagctgtcactggcgatgtcctaggggtgctgcattttgcttccggggaaggatggacacttttcagaagtcactgcagtattcccaattgcactggccctgggcatggccttacccagtctaagctggcaggatctaaaacagcagcgacctcggcccctatccagagaggtgcagcaagagagccatttccctgtgacatttagtggactggccagttcatagcagcactgtgaggacccccaagttggacgtgctcggagggaaagatttatggcctctgtcgagggacctgcagcgtgagagccagtggcatctgcgcggcttgcctggctcttgctgtatcctcacttcctgtggagcggggattggctctgagaaggagtgttctctgtctgcctggcaaaggtgctgtggaataggcttggcatgccaccctgttttagagagtgacagttacagttgtaacaagcctacttcatattggccccctcagttagcctttttgaggcaatgccatttctagagttgaaaaagccctggacccaaactgcggcactgttgaataaagggcagtcctactcctgtccttttagagtggcttagtgtgacacacaggcatctcccaggccaagcacacacaggctgcgcccagttccgcaggagccgtcccacagcgtggctctctggattctcccacttgtcctccttggaaggagctcttgctggccagtgtttggaggggaggatgagtgcctgtcactgaggcctcactatggttggcgtctgaagctgggcggtcgtcaggcctgtgctgagagccgcagcccctgtgcacacctaacacagggcgctccccctgctgcttccctggctcagttcttcggagctccagagtgagaaggccgcttcgtcctttttctctgggtgatgcccttagaataacactatatgcaatgtaactcacaatgttccaggaccaaagacttgatggaggggctagaggcgacccttgttgtaaaaggcgatcagaacacctgagggaggaaggggcttgcagttttcccagcccttctcgctgccaaggcagcagtggtgctgtggatgggctggggactgcgggacagagcctgctactacttgggagttggtgctgccctgtggcatggaggggtgggaggggctgagatggctgctggcccggcctccaagagttctggacaggaggcagacactgcccagatgctcggtggagggacagtgatggcctttgactcatgaggcctggagaaaagtatcaaaggtctcaccatgtaagagtgatttccgatttctctcctttcagttgtgtgaaaaaacagctggcctgggttccattagcaaattaaatcatcttcaatcttaaattagagaccagaatgatcttcaggataaaaagaacttctgaatctctgcaataggaaatgtttcgatcatgcaagtgctttcccagccaaatgtctgtgctctctgtgtcactgagggccacaggttcctctaacatctgtcactgtcacttcaccaggcaggccttggagttccatgacaaaatcacttttgtcagacaaagaatgtatcctttacttttctcaaatggaataaaattatttcttctgtggaggaaaattgattccccctttttatttaatttttttttggagacagtctcactccttccc

SEQ ID NO: 44 - DNA, 2076 bp, Homo sapiens
  misc_feature: Incyte ID No 1485774CB1
cggggctccagccaggagccctgctgcccagggcatggccaaacctttcttccgactccagaagtttctccgccgaacacagttcctgctgttcttcctcacggctgcctacctgatgaccggcagcctgctgctgctgcagcgggtccgcgtggctctcccacagggcccccgggcacccggccccctgcagaccttgccagtggccgccgtggcgctgggcgtgggcttgctggacagcagagccctgcacgaccctcgagtcagcccagagctgctgctgggtgtggacatgctgcagagccccctgacccggccccggcccggcccccgctggctccggagccgcaactcggagctgcgtcagttgcgtcgccgctggttccaccacttcatgagtgactcccagggaccgcccgccctgggccccgaggctgccaggcccgccatccacagccgaggcacctacattggatgcttcagtgacgatggccatgagaggactctgaaaggagctgtgttttatgacttgagaaagatgactgtctcccactgccaggatgcgtgtgctgagcggtcctatgtctacgccggcttggaggccggggcggagtgttactgcgggaaccggctgccagcggtgagcgtggggctggaagagtgtaaccatgagtgcaaaggcgagaagggctctgtgtgcggggctgtggaccggctctccgtgtaccgtgtggacgagctgcagccgggctccaggaagcggcggaccgccacctaccgcggatgcttccgactgccagagaacatcacacatgccttccccagctccctgatacaggccaatgtgaccgtggggacttgctcgggcttttgttcccagaaagagttccccttggccattctcaggggctgggaatgctactgtgcttaccctaccccccggttcaacctgcgggatgccatggacagctcagtatgtggccaggaccctgaggcacagaggctggcagaatactgtgaggtctaccagacacctgtgcaagacactcgttgtacagacaggaggttcctgcctaacaaatccaaagtgtttgtggctttgtcaagcttcccaggagccgggaacacgtgggcacggcacctcattgagcatgccactggcttctatacagggagctactactttgatggaaccctctacaacaaagggttcaagggcgaaaaggaccactggcggagccgacgcaccatctgtgtcaaaacccacgagagtggcaggagggagattgagatgtctgattcagccatcctgctaatccggaacccatacaggtccctggtggcagaattcaacagaaaatgtgccgggcacctgggatatgcagctgaccgcaactggaagagcaaagagtggccggactttgtcaacagctacgcctcgtggtggtcctcgcacgtcctggactggctcaagtacgggaagcggctgctggtggtgcactacgaggagctgcggcgcagcctggtgcccacgttacgggagatggtggccttcctcaacgtgtctgtgagcgaggagcggctgctctgcgtggagaacaacaaggagggcagcttccggcggcgcggccggcgctcccacgaccctgagcccttcaccccggagatgaaagacttgatcaatggctacatccggacggtggaccaagccctgcgtgaccacaactggacggggctgcccagggagtatgtgcccagatgataggcctggcccacgccgccgcccccgctgagtgacgcaatcgcaccacggggctgcgctccccactctgatgctcaggcccgtggcctcactgggacgaacggtgggtggggggctcaccctggtgctgcctcccgcacaaggagacctggacacaacagacacacatcacaaggcgaacacaaatggacacacatacctggccatgaacccacacctcctcagacactcagacaccactccaggctcatagcccgtcttgatgcagagaagcccccacgtgggtgtgccaggcaccccagatacaaatgttt

SEQ ID NO: 45 - DNA, 2957 bp, Homo sapiens
  misc_feature: Incyte ID No 7289372CB1
cataagattgctttacaccagggttcctgaaatcaaggaaaatactggccaaaatcaccgaccccattctaccttcaattacctagattctgcgtccccggcgctaggtacccaatcctggctgtccgaccacaggatccccggcagggacgggtcacagtgctctcacccctcgaccattttcgaaaaaaccttcctctgcaaacgcattgcgccctccccatgggtccgcgggcggggactccaggcccgagcagtcggtgtgaagttctgtgttctgaactggggctgagcaagatgcgatggtctcntaccgctgggccgcccgtagcgacggcaggagtaggggtattgatctccacggaagccccaaaccctcgccatcgagagacccccatggcccggggtgatggctgtggggcttggtgctcccagagagctcagtggctacagaatgggtggggattctgcgtgtctcccggagcctgaacccctttcctggttatggccggtagctgtctccagggactaacgtgggcagcgcaggggggcggaaaccgggttttagccaaatgcctcgacatcgccgcgcctccgcctcctcgtcgctgaaagaaatgtcggggtttcatcagagctagggagcgacagtcgggaacagcgagtctgccgaagccggctgttgtgtgagggtgtgagacggcggggcggtgaggggccaccgcggcttgggggatagtgcgtgtggggttgaccgtgtgtctgcttgagaggctgtgaagatatggggggcagatatgggagaaatgctcgggcctgaagtccccagcccaccgtgctcaagagtagcggacgttttgccaccatccttgtctgtgctactgtctgctgcagcttccgtgccccgttctcctggagcagggcgtaaaagcggcttgcattcaattagcagcgaagctcgcgggcgctggcgggacaggcgcgtgaggccacaacacatgcgtgtatcttgcttgggctatcttccctgctctgccacgccgggtctggagaaggggtttcagccccaggacatttactgagagtcggcgaatattgggagccgcgatgttcccccttcgggccctgtggttggtctgggcgcttctaggagtggccggatcatgcccggagccgtgcgcctgcgtggacaagtacgctcaccagttcgcggactgcgcttacaaagagttgcgtgaggtgccggaaggactgcctgccaacgtgacgacgcttagtctgtccgcgaacaagatcactgtgctgcggcgcggggccttcgccgacgtcacacaggtcacgtcgctgtggctggcgcacaatgaggtgcgcaccgtggagccaggcgcactggccgtgctgagtcagctcaagaacctcgatctgagccacaacttcatatccagctttccgtggagcgacctgcgcaacctgagcgcgctgcagctgctcaaaatgaaccacaaccgcctgggctctctgccccgggacgcactcggtgcgctacccgacctgcgttccctgcgcatcaacaacaaccggctgcgtacgctggcgcctggcaccttcgacgcgcttagcgcgctgtcacacttgcaactctatcacaatcccttccactgcggctgcggccttgtgtggctgcaggcctgggccgcgagcacccgggtgtccttacccgagcccgactccattgcttgtgcctcgcctcccgcgctgcagggggtgccggtgtaccgcctgcccgccctgccctgtgcaccgcccagcgtgcatctgagtgccgagccaccgcttgaagcacccggcaccccactgcgcgcaggactggcgttcgtgttacactgcatcgccgacggccaccctacgcctcgcctgcaatggcaacttcagatccccggtggcaccgtagtcttagagccaccggttctgagcggggaggacgacggggttggggcggaggaaggagagggagaaggagatggggatttgctgacgcagacccaagcccaaacgccgactccagcacccgcttggccggcgcccccagccacaccgcgcttcctggccctcgcaaatggctccctgttggtgcccctcctgagtgccaaggaggcgggcgtctacacttgccgtgcacacaatgagctgggcgccaactctacgtcaatacgcgtggcggtggcagcaaccgggcccccaaaacacgcgcctggcgccgggggagaacccgacggacaggccccgacctctgagcgcaagtccacagccaagggccggggcaacagcgtcctgccttccaaacccgagggcaaaatcaaaggccaaggcctggccaaggtcagcattctcggggagaccgagacggagccggaggaggacacaagtgagggagaggaggccgaagaccagatcctcgcggacccggcggaggagcagcgctgtggcaacggggacccctctcggtacgtttctaaccacgcgttcaaccagagcgcagagctcaagccgcacgtcttcgagctgggcgtcatcgcgctggatgtggcggagcgcgaggcgcgggtgcagctgactccgctggctgcgcgctggggccctgggcccggcggggctggcggagccccgcgacccgggcggcgacccctgcgcctactctatctgtgtccagcggggggcggcgcggcagtgcagtggtcccgcgtagaggaaggcgtcaacgcctactggttccgcggcctgcggccgggtaccaactactccgtgtgcctggcgctggcgggcgaagcctgccacgtgcaagtggtgtttccaccaagaaggagctcccatcgctgctggtcatagtggcagtgagcgtatccctcctggtgctggccacagtgccccttctgggcgccgcctgttgccatctgctg

SEQ ID NO: 46 - DNA, 1223 bp, Homo sapiens
  misc_feature: Incyte ID No 1672338CB1
ggcacctggagggccgcactcccgttccagccaggctgagccttctgtcccctgcctctggggcctgggaaccccccttcttctttctcctgaatggcacccccgccctagaatccagacaccgagtttcccactgtggctggttcaagggtatgtgagagctccctggtgacagtctgtggctgagcatggccctcccagccctgggcctggacccctggagcctcctgggccttttcctcttccaactgcttcagctgctgctgccgacgacgaccgcggggggaggcgggcaggggcccatgcccagggtcagatactatgcaggggatgaacgtagggcacttagcttcttccaccagaagggcctccaggattttgacactctgctcctgagtggtgatggaaatactctctacgtgggggctcgagaagccattctggccttggatatccaggatccaggggtccccaggctaaagaacatgataccgtggccagccagtgacagaaaaaagagtgaatgtgcctttaagaagaagagcaatgagacacagtgtttcaacttcatccgtgtcctggtttcttacaatgtcacccatctctacacctgcggcaccttcgccttcagccctgcttgtaccttcattgtgagttctctggtgcccagcgctcaggcccccaagcatcccttctcacatctacccacgactttcctctgtagctctggaaaactctggccttccagatgcaggaccctcatgaacttcctggccccagaccaatttccctctatgtccctttcccttccttcctcaagcccctcatttcccagatgtgagaccttggcgttctggccccccagcctctctccccatttaggaacttcaagattcctacctgttgcccatctcggaggacaaggtcatggagggaaaaggccaaagcccctttgaccccgctcacaagcatacggctgtcttggtgggtgagtatcaggtttcccacttcatcccaacatctactttctccagtcacgctgtgaaatatggaatattacagagttttccaaaaggcaggggaaactgggtgtggtgatgcgtgcatatggtcccagttatttggaagctgaagttgaaggatgcttgagtttaggggtttgagtctagcctgggcaacacagcgagatcgtctcaaaaaaaaaaaaaaaaaaaaaaaaaaaa

SEQ ID NO: 47 - DNA, 2888 bp, Homo sapiens
  misc_feature: Incyte ID No 184661CB1
cgcacgacgtaaatcctgcggctacatgagcggctcggaattcggctcgagcccggcccgggcagctgcggctcgggatccgtcgaggggaggccgagcttgccaagctggcgcccagcggggtcatggtgcccggcgcccgcggcggcggcgcactggcgcgggctgccgggcggggcctcctggctttgctgctcgcggtctccgccccgctccggctgcaggcggaggagctgggtgatggctgtggacacctagtgacttatcaggatagtggcacaatgacatctaagaattatcccgggacctaccccaatcacactgtttgcgaaaagacaattacagtaccaaaggggaaaagactgattctgaggttgggagatttggatatcgaatcccagacctgtgcttctgactatcttctcttcaccagctcttcagatcaatatggtccatactgtggaagtatgactgttcccaaagaactcttgttgaacacaagtgaagtaaccgtccgctttgagagtggatcccacatttctggccggggttttttgctgacctatgcgagcagcgaccatccagatttaataacatgtttggaacgagctagccattatttgaagacagaatacagcaaattctgcccagctggttgtagagacgtagcaggagacatttctgggaatatggtagatggatatagagatacctctttattgtgcaaagctgccatccatgcaggaataattgctgatgaactaggtggccagatcagtgtgcttcagcgcaaagggatcagtcgatatgaagggattctggccaatggtgttctttcgagggatggttccctgtcagacaagcgatttctgtttacctccaatggttgcagcagatccttgagttttgaacctgacgggcaaatcagagcttcttcctcatggcagtcggtcaatgagagtggagaccaagttcactggtctcctggccaagcccgacttcaggaccaaggcccatcatgggcttcgggcgacagtagcaacaaccacaaaccacgagagtggctggagatcgatttgggggagaaaaagaaaataacaggaattaggaccacaggatctacacagtcgaacttcaacttttatgttaagagttttgtgatgaacttcaaaaacaataattctaagtggaagacctataaaggaattgtgaataatgaagaaaaggtgtttcagggtaactctaactttcgggacccagtgcaaaacaatttcatccctcccatcgtggccagatatgtgcgggttgtcccccagacatggcaccagaggatagccttgaaggtggagctcattggttgccagattacacaaggtaatgattcattggtgtggcgcaagacaagtcaaagcaccagtgtttcaactaagaaagaagatgagacaatcacaaggcccatcccctcggaagaaacatccacaggaataaacattacaacggtggctattccattggtgctccttgttgtcctggtgtttgctggaatggggatctttgcagcctttagaaagaagaagaagaaaggaagtccgtatggatcagcagaggctcagaaaacagactgttggaagcagattaaatatccctttgccagacatcagtcagctgagtttaccatcagctatgataatgagaaggagatgacacaaaagttagatctcatcacaagtgatatggcagattaccagcagcccctcatgattggcaccgggacagtcacgaggaagggctccaccttccggcccatggacacggatgccgaggaggcaggggtgagcaccgatgccggcggccactatgactgcccgcagcgggccggccgccacgagtacgcgctgccctggcgcccccggagcccgagtacgccacgcccatcgtggagcggcacgtgctgcgcgcccacacgttctctgcgcagagcggctaccgcgtcccagggccccagcccggccacaaacactccctctcctcgggcggcttctcccccgtagcgggtgtgggcgcccaggacggagactatcaaaggccacacagcgcacagcctgcgggcaggggctacgaccggcccaaagctgtcagcgccctcgccaccgaaagcggacaccctgactctcagaagcccccaacgcatcccgggacgagtgacagctattctgcccccagagactgcctcacacccctcaaccagacggccatgactgcccttttgtgaacacaatgtgaaagaagcctgctgtggtactgagcgtcgggctgtcacaaggcactggaagaagggagcctgctggtccagagtgtgcgtgtgtatcggtgtgtgtgtacacttgcatgtgtgtgtgtgatccagtaggatcctagagacaacctgtcatactgtttacaaaattgtgcagctggtttcgtgctgacccttagggtgcgtctgttgggttttgttgggctagaaaaatgaaaatttttagatggcgttttcattcctctgactgatattgagctgctttggtgttaaaggtgtaatgtgtacagagttgtatttaacaataataaaagtaacttaagtttgctctatcagattttagttctgcacagaggttaagtgggaaaatgcagctgttgcaaaatgtatataaatagtatgttcatttttttcagtatattatctgatactgtgttagcagcaggtctgtcttaaacctagtcttgttgttatttgagtcatttcctctcctttgataactagaactgaaagcatttttaacattcttctcctggaagaaatgaattacttgaagcatgaaaagcacaccagggtggttgtttatttagca

SEQ ID NO: 48 - DNA, 3142 bp, Homo sapiens
  misc_feature: Incyte ID No 3719737CB1
tgcgcgcaggctcacaggccctgggagtgagctggtgcccggcgacctggcacccgcgcctggatatggggcgtctacatcgtcccaggagcagcaccagctacaggaacctgccgcatctgtttctgtttttcctcttcgtgggacccttcagctgcctcgggagttacagccgggccaccgagcttctgtacagcctaaacgagggactacccgcgggggtgctcatcggcagcctggccgaggacctgcggctgctgcccaggtctgcagggaggccggacccgcagtcgcagctgccagagcgcaccggtgctgagtggaacccccctctctccttcagcctggcctcccggggactgagtggccagtacgtgaccctagacaaccgctctggggagctgcacacttcagctcaggagatcgacagggaggccctgtgtgttgaagggggtggagggactgcgtggagcggcagcgtttccatctcctcctctccttctgactcttgtcttttgctgctggatgtgcttgtcctgcctcaggaatacttcaggtttgtgaaggtgaagatcgccatcagagacatcaatgacaacgccccgcagttccctgtttcccagatctcggtgtgggtcccggaaaatgcacctgtaaacacccgactggccatagagcatcctgctgtggacccagatgtaggcattaatggggtacagacctatcgcttactggactaccatggtatgttcaccctggacgtggaggagaatgagaatggggagcgcaccccctacctaattgtcatgggtgctttggacagggaaacccaggaccagtatgtgagcatcatcatagctgaggatggtgggtctccaccacttttgggcagtgccactctcaccattggcatcagtgacattaatgacaattgccctctcttcacagactcacaaatcaatgtcactgtgtatgggaatgctacagtgggcaccccaattgcagctgtccaggctgtggataaagacttggggaccaatgctcaaattacttattcttacagtcagaaagttccacaagcatctaaggatttatttcacctggatgaaaacactggagtcattaaacttttcagtaagattggaggaagtgttctggagtcccacaagctcaccatccttgctaatggaccaggctgcatccctgctgtaatcactgctcttgtgtccattattaaagttattttcagaccccctgaaattgtccctcgttacatagcaaacgagatagatggtgttgtttatctgaaagaactggaacccgttaacactcccattgcgtttttcaccataagagatccagaaggtaaatacaaggttaactgctacctggatggtgaagggccgtttaggttatcaccttacaaaccatacaataatgaatatttactagagaccacaaaacctatggactatgagctacagcagttctatgaagtagctgtggtggcttggaactctgagggatttcatgtcaaaagggtcattaaagtgcaacttttagatgacaatgataatgctccaattttccttcaacccttaatagaactaaccatcgaagagaacaactcacccaatgcctttttgactaagctgtatgctacagatgccgacagcgaggagagaggccaagtttcatattttctgggacctgatgctccatcatatttttccttagacagtgtcacaggaattctgacagtttctactcagctggaccgagaagagaaagaaaagtacagatacactgtcagagctgttgactgtgggaagccacccagagaatcagtagccactgtggccctcacagtgttggataaaaatgacaacagtcctcggtttatcaacaaggacttcagcttttttgtgcctgaaaactttccaggctatggtgagattggagtaattagtgtaacagatgctgacgctggacgaaatggatgggtcgccctctctgtggtgaaccagagtgatatttttgtcatagatacaggaaagggtatgctgagggctaaagtctctttggacagagagcagcaaagctcctatactttgtgggttgaagctgttgatgggggtgagcctgccctctcctctacagcaaaaatcacaattctccttctagatatcaatgacaaccctcctcttgttttgtttcctcagtctaatatgtcttatctgttagtactgccttctactctgccaggctccccggttacagaagtctatgctgtcgacaaagacacaggcatgaatgctgtcatagcttacagcatcatagggagaagaggtcctaggcctgagtccttcaggattgaccctaaaactggcaacattactttggaagaggcattgctgcagacagattatgggctccatcgcttactggtgaaagtgagtgatcatggttatcccgagcctctccactccacagtcatggtgaacctatttgtcaatgacactgtcagtaatgagagttacattgagagtcttttaagaaaagaaccagagattaatatagaggagaaagaaccacaaatctcaatagaaccgactcataggaaggtagaatctgtgtcttgtatgcccaccttagtagctctgtctgtaataagcttgggttccatcacactggtcacagggatgggcatatacatctgtttaaggaaaggggaaaagcatcccagggaagatgaaaatttggaagtacagattccactgaaaggaaaaattgacttgcatatgcgagagagaaagccaatggatatttctaatatttgatatttcatggtggaataacacagagaaatgttttaactgactttggatcttcatcacctaaaaaagagtgtgttgatggcagttccaatgaaggacaactaatttataacttgttctatattgtaaatagctgtttacaggtttttaaatttaaattcagaggttataaaatgtgtacagcatttttaagtgaaaattagtactaacagctataggacttgtatttaaaaaaaaaaaaaaaaagcttggacatggtttgcagctttcatacaccaagcagatgtttgataaaacctgggggt

SEQ ID NO: 49 - DNA, 4749 bp, Homo sapiens
  misc_feature: Incyte ID No 5773251CB1
gtgcttgcagtggtggaattcctagagcgttaaatattcagtgataagtgggcagggttgaggctgaaaagatagataggggccaggtcaaagtgggttaatttatgtgctgtttgtaccaacaagcttggactttggtattgtgacaggcagttaggaggcattcaagacttaagcagagtgacatggtaaaattactccagaaaacgttcaaattatatttgatgatccactaccaatttcatacagtcagccagagaaggtgaatggagagtccaagagcagcagtaccagcgagagtggggacagtgataacatgaggatttccagctgcagcgatgaaagtagtaacagcaacagcagtcgtaagagtgacaatcattcaccagctgtggtcactaccactgtgagcagcaaaaagcagccatcagttcttgttacatttccaaaggaagagagaaaatctgtttctggcaaggcttcaataaaattgtcagaaactatcagtgaagggaccagtaattctctatctacttgtacaaaatctggtccatctcccctttcttctccaaatgggaagttaacagtagcaagtcctaagcgtgggcaaaagagggaagaaggatggaaagaagttgtaagaaggtcaaagaaagtcggaggctggctttggctgcgaaaagagaaaaaagaaaagagaagagaaggaagaaaaaggaagaacaaagaaggaaactagaagaaattgaagccaaaaataaaagagaactttgaactccaagctgctcaagaaaaagaaaagcttaaagttgaagatgagcctgaagtcttgacagaacctccaagtgccacaaccactactaccataggtatatctgcaacctggacaactttggcaggttctcatggtaaaagaaataataccataactacaaccagttcaaagaggaaaaacaggaaaaataaaattactccagaaaacgttcaaattatatttgatgatccactaccaatttcatacagtcagccagagaaggtgaatggagagtccaagagcagcagtaccagcgagagtggggacagtgataacatgaggatttccagctgcagcgatgaaagtagtaacagcaacagcagtcgtaagagtgacaatcattcaccagctgtggtcactaccactgtgagcagcaaaaagcagccatcagttcttgttacatttccaaaggaagagagaaaatctgtttctggcaaggcttcaataaaattgtcagaaactatcagtgaagggaccagtaattctctatctacttgtacaaaatctggtccatctcccctttcttctccaaatgggaagttaacagtagcaagtcctaagcgtgggcaaaagagggaagaaggatggaaagaagttgtaagaaggtcaaagaaagtatctgttccatcaactgtgatatccagagtgattggaagaggaggctgtaatatcaatgctattcgggagtttactggtgcacacatagatattgataaacagaaagacaagactggagaccggataatcactataaggggtggcactgaatcaacaagacaagcaactcaattgattaatgctttgatcaaggatccagacaaagaaattgatgaacttattccaaagaatcgtttgaaaagctcctcagcaaattccaaaatagggtcatcagcacctaccaccactgctgctaacacttccttaatgggaattaaaatgacaactgtagctctgtcatcaacatctcaaactgccacagcactcactgtgcctgcaatttcttctgcatccactcacaaaaccattaagaacccagtgaataatgtgaggcctggttttccagtttctcttccattagcatatcctcctccacagtttgcacatgctttgcttgctgctcagactttccagcagatccgtccaccaaggttgcccatgacccactttggaggtacttttccaccagctcaatccacttggggtccgtttcctgtcaggcctttgagccctgccagagctactaactcgcctaagcctcacatggtgcctcgccatagcaatcagaatagcagtggttctcaggtgaattcagcaggttctttaacttcaagcccaacaactacaaccagttcatcagcttcaacggtgcctggtacatctacaaatggcagtccaagttcaccttctgtccgaaggcagctttttgtcacagttgtgaagacatccaatgccaccacaacaacagtcacaaccacggcaagcaacaacaacactgcacccacaaatgccacatatcctatgcctactgccaaagaacactatccagtatcatccccatcttccccatcaccaccagcccagccaggaggggtttctagaaacagccctttggattgtggatcagcatctccaaataaagtggcatcttcctccgaacaggaagcaggtagtccaccagtagtagaaacaacaaacactagacctccaaacagcagcagttcttctgggagttcatcagctcattctaatcagcaacaacctccgggatctgtttctcaggaaccaagaccacctcttcagcagtctcaggttcctcccccggaagttagaatgactgttcctcctttagcaacaagttctgctccagtggcggtgccttctactgccccagtgacttaccctatgcctcagacaccaatgggatgcccccagcctactcctaaaatggaaacccctgctattagaccaccccctcatggcacaactgcccctcacaagaattcagcttcagtgcaaaattcatctgttgcagtccttagtgtcaatcacattaaaagacctcacagtgttccctcttctgtccagctaccttcgaccttaagtacacaaagtgcttgtcagaattcagtacatccagcaaataagcctattgctcccaatttcagtgcccccttaccatttgggccctttagcacattgtttgaaaacagccctacttctgctcatgccttctggggaggatctgttgtttcatctcagtcaacaccagaatctatgctatcaggaaaatcctcatatttgccaaattcagatcctttacatcagtctgatacttccaaagctccaggttttagaccaccattacagagacctgctccaagtccctcaggtattgtcaatatggactcgccatatggttctgtaacaccttcttcaacacatttgggaaactttgcttcaaacatttcaggaggtcagatgtacggacctggggcaccccttggaggagcacccgcagctgctaactttaacagacaacatttttccccgcttagtttgttgactccgtgttcatcagcatcaaatgattcttctgcacagtcagtatcctcgggagttcgtgcaccatctcctgccccatcatcagtaccgttagggtcagaaaagcccagcaatgtgtctcaggacaggaaagttccagtccctattgggactgaacgttctgcacgtatcaggcaaactggaacgtcagctccatctgttattgggagcaatttgtctacatcagtaggacatagtggcatctggtcctttgaagggattggtggcaatcaagacaaagtagactggtgtaaccctgggatgggaaatcctatgatccacagaccgatgtctgacccaggagtattttcacaacatcaagcaatggagcgagatagtacaggaattgtaactccttctggtacattccatcagcatgttcctgcaggctacatggactttcctaaagttgggggtatgcctttttctgtgtatgggaatgcaatgattcctccagtagcacctatccctgatggtgctggaggacccatatttaatggccctcatgctgcagacccttcttggaactcactgataaagatggtttccagctccacggaaaataatggccctcaaacggtgtggactggaccctgggcacctcacatgaacagtgtgcatatgaaccagcttggctgatgaggatcagcttgttagcctgcagattccttttcatttggaggaaatcacaagtggccgaaaaaaaaaattatgctcccaaatcattctactgatgtgcttgactgaagtgtgtaggctttttgcagaagatcttactaactgacctattttctgtgaacatttgtgactgcccattccccatcatcatccgttttaccttagttagcatttttcttatcatttttctttttttctttccctcttcccctttggacataactttctgttgaagctgttctttggctggttggttttagtactgtaaactgcttctgagcaaacacggaaatttagcaaaattatgtaaacttgatcctgaagttttagaatggcaaataaatgtacaattgtttacataacagaaaaggctaagcagaaagtaaatttcaatatgtcagtatagaggctctactttatgtagacttaaattaatgtgagatatgtaccttcatattcagaaatctggatgtttccttcatacattaaactattaataagcataacttttctactggtgtaatttaagtataaagtaaaataatgggcattatcattggatgtttccccacattggcttttaaaatacccatcttgctttctttttggtttatttgtagcaaggcacatatagaagaagaaatttctggcttttc

SEQ ID NO: 50 - DNA, 4155 bp, Homo sapiens
  misc_feature: Incyte ID No 5426470CB1
gccgtcggggcgggcgtctggcgagctgcagagaccagattaaaggatttacctgaagagaaagcattctattcatcagagactggacaagagttactcttgcatttggcaattaaagatgatgtttccatggaaacagttgatcctgctttcattcattggctgcttaggaggtgagcttctcttacaaggccctgtatttatcaaagaacccagcaacagcattttccctgttggttcagaagataaaaaaataactttgcattgtgaagcaagaggcaatccatcacctcattacagatggcagctgaatggaagtgatattgatatgagtatggaacatcgttataagttgaatggaggaaatcttgtggttattaatcccaacagaaattgggatacaggaacttaccaatgttttgcaacaaattcacttggaacaattgtcagcagagaagccaaacttcagtttgcctatcttgaaaattttaaaaccaaaatgaggagtacagtgtctgtgcgtgaaggccagggagttgtgctgctctgcggccccccaccacactctggagaactgtcatatgcttggatcttcaatgaatacccatcgtttgttgaagaagatagtcggagatttgtctcccaggagacagggcacctctacatatctaaggtggagccgtctgatgtgggaaattacacatgtgtggtgacaagtatggtgacaaatgcccgagtgctgggctctccaactcctttggtgctacgttctgatggtgtgatgggtgaatatgaacctaaaatagaagttcagtttccagaaactcttccagcagctaaaggttcgactgtgaaattggaatgttttgcccttggaaatcccatacctcagattaattggagaagaagtgatgggctgccattttccagcaaaattaaattaaggaagttcagtggtgtgcttgaaatccccaacttccaacaggaagatgcaggttcctatgaatgcattgctgagaattcacgaggaaaaaatgttgccagagggcgtctcacttactatgcaaagccccattgggttcaactcataaaggatgtggaaatagccgtggaggacagtctttattgggaatgcagggcaagcggcaagcccaagccttcctaccgatggctgaaaaatggagcagccctggtgctagaggagagaacacagatagaaaatggtgcccttacaatatcaaacctaagtgtgactgattctggcatgttccaatgcatagcagaaaacaaacatggccttgtttattccagtgctgagctcaaagttgttgcttctgctccagatttttcaaagaatccaatgaagaagttggttcaggtgcaggtgggcagcctggtcagcttggattgtaaacccagagcctccccaagggcactctcttcctggaagaagggggatgtgagcgtgcaggagcatgaaagaatttctttgttaaacgatggaggactcaaaatagccaatgtgactaaagctgatgctggaacttacacctgcatggcagaaaaccagtttgggaaagcaaatggcacaacacatttggttgttacggaaccaacaagaataactttggcaccatctaacatggatgtttctgttggtgaaagcgtcatattgccctgccaggtacaacatgacccgctgttagacatcatctttacctggtatttcaatggggcccttgcagattttaagaaagatggatctcactttgagaaagttggtgggagttcatctggtgatttaatgatcagaaacattcagctgaaacacagtgggaaatatgtttgtatggtgcaaacgggggtggacagtgtttcatctgctgctgacctcatagtaagaggttcacctggaccaccagaaaatgtgaaggtagatgaaattacagacacaacagcccaactctcttggaaagaaggtaaagacaaccatagcccagttatatcctattctatccaggctcggacacctttctccgtgggttggcaaaccgtcacaacagtgcctgaggtcatcgatgggaagacgcacacagccactgtagttgagttaaacccatgggtggaatatgaatttcgggttgtagccagtaacaaaattggaggtggagaaccaagtttaccctcagaaaaagtaagaactgaagaggcagttccagaagtgcctccttctgaagtcaatggaggaggcggaagccggtctgaacttgtgataacctgggatccagtccctgaagaactacagaatggtgaaggttttgggtatgttgttgctttccgccctcttggggttaccacctggatccagacagtggtgacatcccctgacaccccaagatatgtctttaggaatgaaagcatcgtgccatattcaccatatgaagttaaagtgggtgtttataataacaaaggtgaaggaccatttagcccagtgacaacagtgttctctgcagaagaagagcctacagtggccccatctcaagtctctgcaaatagcctatcttcctcagaaattgaggtttcatggaacaccattccttggaagttgagcaatggacatttactgggctatgaggtgcggtactggaatgggggtggaaaggaggaatcatccagtaagatgaaagtggcaggaaatgagacatcagccagactacggggcctgaagagcaacctggcctattacacggctgtccgggcttacaacagtgccggcgctgggccttttagcgccacagttaatgtaaccaccaagaaaacgcctcccagtcagccaccaggaaatgttgtttggaatgccacagacactaaagtgttacttaattgggagcaagttaaagccatggagaatgagtcagaagtaacaggatataaagttttctataggactagcagtcaaaataacgtacaagtactgaacacaaataaaacttcagctgaacttgtgctgcccattaaagaggactacattattgaagtcaaggccacaacagatggaggggatgggaccagtagtgaacagatcaggattccacgaataaccagtatggatgcaagaggatccacttcagccatctcgaatgtccaccctatgtcaagttatatgcctatagtactgttcttaattgtatatgtcctgtggtgatattaactcctttttattatttattggaaagttatttggttaccaaaaaaagtgctttcatgaaatgcagtgattatgcatgtttttttcaactcttatttttaactttctacttcattataggtaaatatgaatataattaaaaaaacagtaaatccttttaggggaatctgaaatgccttaatattaacttgataaaccaaaggaatttacatattacatacttcagacttttgatataaatgttcttaaactatgagtttaagcactgcctatggataaagactcacacactctcacatgtacacacacacgcatgagaatttctttttacattgaaaaactctttcatttaattcaaatgctattttcccattataatagcattatttggaagacttaaccagtatcaatttgaaatgctgatttaagtccccaaggatgaaaaatacattttaaaaattattttgttggagaggagtggcatgtgattcaaaagagcattgttggaaaatgctactgtggggcttagaagaatgatgtttggtttggtatgctgctaactagttgtaagactttacaaatcactttgccatctgtacctctcaattattcctctataaaatatggagataataatacctatctgatcagactttgccccatgaattagtttttaaaagataaagactgaagtatgaaagtgcttttgtcaccccaaatgcaattgacccatgcaaaatattagcatgaatttatttaatcacataaaagtcatgaagaccagccagattttcaagcttcattctgtttcattcagttatattccaaaattcaaatgatcacattttattctttctcaaaaaaaaaaaagtttttttaaattaaaaaaggaattgtttccttcacagctatgaataagctttcaggttttattaaaacctagagga

SEQ ID NO: 51 - DNA, 1327 bp, Homo sapiens
  misc_feature: Incyte ID No 7087904CB1
gcgagctgaaagctgctggagagtgagcagccctagcagggatggacatgatgctgttggtgcagggtgcttgttgctcgaaccagtggctggcggcggtgctcctcagcctgtgctgcctgctaccctcctgcctcccggctggacagagtgtggacttcccctgggcggccgtggacaacatgatggtcagaaaaggggacacggcggtgcttaggtgttatttggaagatggagcttcaaagggtgcctggctgaaccggtcaagtattatttttgcgggaggtgataagtggtcagtggatcctcgagtttcaatttcaacattgaataaaagggactacagcctccagatacagaatgtagatgtgacagatgatggcccatacacgtgttctgttcagactcaacatacacccagaacaatgcaggtgcatctaactgtgcaagttcctcctaagatatatgacatctcaaatgatatgaccgtcaatgaaggaaccaacgtcactcttacttgtttggccactgggaaaccagagccttccatttcttggcgacacatctccccatcagcaaaaccatttgaaaatggacaatatttggacatttatggaattacaagggaccaggctggggaatatgaatgcagtgcggaaaatgatgtgtcattcccagatgtgaggaaagtaaaagttgttgtcaactttgctcctactattcaggaaattaaatctggcaccgtgacccccggacgcagtggcctgataagatgtgaaggtgcaggtgtgccgcctccagcctttgaatggtacaaaggagagaagaagctcttcaatggccaacaaggaattattattcaaaattttagcacaagatccattctcactgttaccaacgtgacacaggagcacttcggcaattatacttgtgtggctgccaacaagctaggcacaaccaatgcgagcctgcctcttaaccctccaagtacagcccagtatggaattaccgggagcgctgatgttcttttctcctgctggtaccttgtgttgacactgtcctctttcaccagcatattctacctgaagaatgccattctacaataaattcaaagacccataaaaggcttttaaggattctctgaaagtgctgatggctggatccaatctggtacagttgttaaaagcgcgtgggatttatcagcagtgctacctgggatgaccgctttggaaaatgcccttatttatccttatccacccttttgaaagaactccttgaggcgacattgcctttaaacgacgcgaatctaagatacggccgttgcacc

SEQ ID NO: 52 - DNA, 5529 bp, Homo sapiens
  misc_feature: Incyte ID No 7477312CB1
atgcagctgagccgcgccgccgccgccgccgccgccgcccctgcggagcccccggagccgctgtcccccgcgccggccccggccccggccccccccggccccctcccgcgcagcgcggccgacggggctccggcgggggggaagggggggccggggcgccgcgcgcggagtccccgggcgctccgttccccggcgcgagcggccccggcccgggccccggcgcggggatggacggccccggggccaggggccagcgccgtggtcgtgcgcgtcggcatcccggacctgcagcagacgaagtgcctgcgcctggacccggccgcgcccgtgtgggccgccaagcagcgcgtgctctgcgccctcaaccacagcctccaggacgcgctcaactatgggcttttccagccgccctcccggggccgcgccggcaagttcctggatgaggagcggctcctgcaggagtacccgcccaacctggacacgcccctgccctacctggagtttcgatacaagcggcgagtttatgcccagaacctcatcgatgataagcagtttgcaaagcttcacacaaaggcgaacctgaagaagttcatggactacgtccagctgcatagcacggacaaggtggcacgcctgttggacaaggggctggaccccaacttccatgaccctgactcaggagagtgccccctgagcctcgcagcccagctggacaacgccacggacctgctaaaggtgctgaagaatggtggtgcccacctggacttccgcactcgcgatgggctcactgccgtgcactgtgccacacgccagcggaatgcggcagcactgacgaccctgctggacctgggggcttcacctgactacaaggacagccgcggcttgacacccctctaccacagcgccctggggggtggggatgccctctgctgtgagctgcttctccacgaccacgctcagctggggaccaccgacgagaatggctggcaggagatccaccaggcctgccgctttgggcacgtgcagcatctggagcacctgctgttctatggggcagacatgggggcccagaacgcctcggggaacacagccctgcacatctgtgccctctacaaccaggagagctgtgctcgtgtcctgctcttccgtggagctaacagggatgtccgcaactacaacagccagacagccttccaggtggccatcatcgcagggaactttgagcttgcagaggttatcaagacccacaaagactcggatgttggacaggacagtcatgacttgctacatcctatgcccactggggtcccagagtggggcctgtacacagaagaggaactggaaggaggtgccgccttctctgtaccattcagggaaacccccagctatgcgaagcggcggcgactggctggccccagtggcttggcatcccctcggcctctgcagcgctcagccagcgatatcaacctgaagggggaggcacagccagcagcttctcctggaccctcgctgagaagcctcccccaccagctgctgctccagcggctgcaagaggagaaagatcgtgaccgggatgccgaccaggagagcaacatcagtggccctttagcaggcagggccggccaaagcaagatcaggagctgtattcgaattcgagctcggttccccgcgccccctgcgccccccgcaccgccgccccggggcccgaagcggaaactttacagcgccgtccccggccgcaagttcatcgccgtgaaggcgcacagcccgcagggtgaaggcgagatcccgctgcaccgcggcgaggccgtgaaggtgctcagcattggggagggcggtttctgggagggaaccgtgaaaggccgcacgggctggttcccggccgactgcgtggaggaagtgcagatgaggcagcatgacacacggcctgaaacgcgggaggaccggacgaagcggctctttcggcactacacagtgggctcctacgacagcctcacctcacacagcgattatgtcattgatgacaaagtggctgtcctgcagaaacgggaccacgagggctttggttttgtgctccggggagccaaagcagagacccccatcgaggagttcacgcccacgccagccttcccggcgctgcagtatctcgagtcggtggacgtggagggtgtggcctggagggccgggctgcgcacgggagacttcctcatcgaggtgaacggggtgaacgtggtgaaggtcggacacaagcaggtggtggctctgattcgccagggtggcaaccgcctcgtcatgaaggttgtgtctgtgacaaggaagccagaagaggacggggctcggcgcagagccccaccgccccccaagagggcccccagcaccacactgaccctgcgctccaagtccatgacagctgagctcgaggaacttgagaagctggacgagatgctggcagccgccgcagagccaacgctgcggccagacatcgcagacgcagactccagagccgccaccgtcaaacagaggcccaccagtcggaggatcacacccgccgagattagctcattgtttgaacgccagggcctcccaggcccagagaagctgccgggctccttgcggaaggggattccacggaccaagtctgtaggggaggacgagaagctggcgtccctgctggaagggcgcttcccgcggagcacctcgatgcaagacccggtgcgcgagggtcgcggcatcccgcccccgccgcagaccgcgccgcctcccccgcccgcgccctactacttcgactcggggccgcccccggccttctcgccgccgcccccgccgggccgcgcctacgacacggtgcgctccagcttcaagcccggcctggaggcgcgcctgggcgcgggcgctgccggcctgtacgagccgggcgcggccctcggcccgctgccgtatcccgagcggcagaagcgcgcgcgctccatgatcatcctgcaggactcggcgcccgagtcgggcgacgcccctcgacccccgcccgcggccaccccgcccgagcgacccaagcgccggccgcggccgcccggccccgacagcccctacgccaacctgggcgccttcagcgccagcctcttcgctccgtccaagccgcagcgccgcaagagccccctggtgaagcagctgcaggtggaggacgcgcaggagcgcgcggccctggccgtgggcagccccggtcccggcggcggcagcttcgcccgcgagccctccccgacccaccgcggtccgcgcccgggtggcctcgactacggcgcgggcgatggcccggggctcgcgttcggcggcccgggcccggccaaggaccggcggctggaggagcggcgccgctccactgtgttcctgtccgtgggggccatcgagggcagcgcccccggcgcggatctgccatccctacagccctcccgctccatcgacgagcgcctcctggggaccggccccaccgccggccgcgacctgctgctgccctccccggtgtctgccctgaagccgttggtcagcggcccgagcctggggccctcgggttccaccttcatccacccactcaccggcaaacccctggaccccagctcacccctggcccttgccctggctgcccgagagcgagctctggcctcccaggcgccctcccggtcccccacacccgtgcacagtcccgacgccgaccgccccggacccctgtttgtggatgtacaggcccgggacccagagcgagggtccctggcttccccggctttctccccacggagcccagcctggattcctgtgcctgctcgcagggaggcagagaaggtcccccgggaggagcggaagtcacccgaggacaagaagtccatgatcctcagcgtcctggacacatccctgcagcggccagctggcctcatcgttgtgcacgccaccagcaacgggcaggagcccagcaggctggggggggccgaagaggagcgcccgggcaccccggagttggccccggcccccatgcagtcagcggctgtggcagagcccctgcccagcccccgggcccagccccctggtggcaccccggcagacgccgggccaggccagggcagctcagaggaagagccagagctggtgtttgctgtgaacctgccacctgcccagctgtcgtccagcgatgaggagaccagggaggagctggcccgaattgggttggtgccaccccctgaagagtttgccaacggggtcctgctggccaccccactcgctggcccgggcccctcgcccaccacggtgcccagcccggcctcagggaagcccagcagtgagccaccccctgcccctgagtctgcagccgactctggggtggaggaggctgacacacgcagctccagcgacccccacctggagaccacaagcaccatctccacggtgtccagcatgtccaccttgagctcggagagcggggaactcactgacacccacacctccttcgctgacggacacacttttctactcgagaagccaccagtgcctcccaagcccaagctcaagtccccgctggggaaggggccggtgaccttcagggacccgctgctgaagcagtcctcggacagcgagctcatggcccagcagcaccacgccgcctctgccgggctggcctctgccgccgggcctgcccgccctcgctacctcttccagagaaggtccaagctatggggggaccccgtggagagccgggggctccctgggcctgaagacgacaaaccaactgtgatcagtgagctcagctcccgcctgcagcagctgaacaaggacacgcgttccctgggggaggaaccagttggtggcctgggcagcctgctggaccctgccaagaagtcgcccatcgcagcagctcggctcttcagcagcctcggtgagctgagctccatttcagcgcagcgcagccccgggggcccgggcggcggggcctcgtactcggtgaggcccagtggccgctaccccgtggcgagacgcgccccgagcccggtgaagcccgcgtcgctggagcgggtggaggggctgggggcgggcgcggggggcgcagggcggcccttcggcctcacgccccccaccatcctcaagtcgtccagcctctccatcccgcacgagcccaaggaggtgcgcttcgtggtgcgcagcgtgagcgcgcgcagtcgctccccctcgccgtcgccgctgccctcgcccgcgtccggccccggccccggcgcccccggcccacgccgacccttccagcagaagccgctgcagctctggagcaagttcgacgtgggcgactggctggagagcatccacctaggcgagcaccgcgaccgcttcgaggaccatgagatagaaggcgcgcacctacccgcgcttaccaaggacgacttcgtggagctgggcgtcacgcgcgtgggccaccgcatgaacatcgagcgcgcgctcaggcagctggacggcagctgacgccccacccccactcccgccccaggccgagcccgcggc

SEQ ID NO: 53 - DNA, 1623 bp, Homo sapiens
  misc_feature: Incyte ID No 2739431CB1
tgatatttgaagaagtgttttcatctatccaagaaaaatatgatgtctccatcccaagcctcactcttattcttaaatgtatgtatttttatttgtggagaagctgtacaaggtaactgtgtacatcattctacggactcttcagtagttaacattgtagaagatggatctaatgcaaaagatgaaagtaaaagtaatgatactgtttgtaaggaagactgtgaggaatcatgtgatgttaaaactaaaattacacgagaagaaaaacatttcatgtgtagaaatttgcaaaattctattgtttcctacacaagaagtaccaaaaaactactaaggaatatgatggatgagcaacaagcttccttggattatttatctaatcaggtaatgtgtgacatggattacagaggaggtggatggactgtgatacagaaaagaattgatgggataattgatttccagaggttgtggtgtgattatctggatggatttggagatcttctaggagaattttggctaggactgaaaaagattttttatatagtaaatcagaaaaataccagttttatgctgtatgtggctttggaatctgaagatgacactcttgcttatgcatcatatgataatttttggctagaggatgaaacgagattttttaaaatgcacttaggacggtattcaggaaatgctggtgatgcattccggggtctcaaaaaagaagataatcaaaatgcaatgccttttagcacatcagatgttgataatgatgggtgtcgccctgcatgcctggtcaatggtcagtctgtgaagagctgcagtcacctccataacaagaccggctggtggtttaacgagtgtggtctagcaaatctaaatggcattcatcacttctctggaaaattgcttgcaactggaattcaatggggcacgtggaccaaaaacaactcacctgtcaagattaaatctgtttcaatgaaaattagaagaatgtacaatccatattttaaataatctcatttaacattgtaatgcaagttctacaatgataatatattaaagatttttaaaagtttatcttttcacttagtgtttcaaacatattaggcaaaatttaactgtagatggcatttagatgttatgagtttaattagaaaacttcaattttgtagtattctataaaagaaaacatggcttattgtatgtttttacttctgactatattaacaatatacaatgaaatttgtttcaagtgaactacaacttgtcttcctaaaatttatagtgattttaaaggattttgccttttctttgaagcatttttaaaccataatatgttgtaaggaaaattgaagggaatattttacttatttttatactttatatgattatataatctacagataatttctactgaagacagttacaataaataactttatgcagattaatatataagctacacatgatgtaaaaaccttactatttctaggtgatgccataccattttaaaagtagtaagagtttgctgcccaaatagtttttcttgttttcatatctaatcatggttaactattttgttattgtttgtaataaatatatgtacttttatatcctgaaaaaaaa

SEQ ID NO: 54 - DNA, 2242 bp, Homo sapiens
  misc_feature: Incyte ID No 7473606CB1
ccacaagggcctgactgagcgagcgagcatggacggccgcggggctttctggacagtggccattcccagagccaggcaggaaggcctcgggaggctggggctcccgttcccggtgaagcggacgccgccagcgccccagaacccaggaggaagcacacaggccccacagagagtggttggcaagagtcactcggggattaggatgccggccaaatcgcggaatttgaggctggaatccaagctcaacaggaaagtagtgaaatacaaatggggaaaacagggctctggagcggggagggagctggtgccggcatttcccaccaacgccggtttaggaagacgggaccgatgccggccgccccctgctggaggggatgtggcatctcacgggctgccagggagcggggttggctactcctgcaaccagcgtgaagagggtctcaggggaggctgtggtgggatcccccacgtgcccttgttcctctcaccgttacctctggatgcctcggggcaaaggccttcttccacctatagacagagtctacgcaggggtcttggaacccgggcacaccagtccccagctaacgaaatccccgagttgggggatttgagagggtcacgtttggcccaagaacccgcagtcctctttggtcttcggccctctatttctaagcgtgggcttctggcacggcggctctgggcacagcccatgctgctttcgggctgggtggtttcaacgacgacaacaattatcacagtgacggtgaccttcaccccaacaggactgctgtgtgtgaagcactcaagagggcccctacaaccaacctgccaggagtcggctcctgaaaacagggtcggaaaagcgctaattactttttccaaaggctggagggcttcactccggctggcgccgccgcctagcgcgctcctgcttcgccgccacggtccgggggggctgccggtcccgggtaccatgtgtgacggcgccctgctgcctccgctcgtcctgcccgtgctgctgctgctggtttggggactggacccgggcacaggtagcgccccctcccacagccctcttcaccccgcgtcctgcggctaccttccctctgcgttctcgcggcgtcctggcggcccgggggcggcggcgggaccgctgacggcgcccgagcggaggaggcgcgggccgcggccggagtacgggaatcgggtggctccgtggcaggcgcgccgccgccgggtctccgctcgccgatgcgcggcgccgttccgggaggtgctcgcgcggctgcgccggagaccctccccgggtggcgcgggccagcgtggagctgtcggcgacgcggcggccgacgtggaggtggtgctcccgtggcgggtgcgccccgacgacgtgcacctgccgccgctgcccgcagcccccgggccccgacggcggcgacgcccccgcacgcccccagccgccccgcgcgcccggcccggagagcgcgccctgctgctgcacctgccggccttcgggcgcgacctgtaccttcagctgcgccgcgacctgcgcttcctgtcccgaggcttcgaggtggaggaggcgggcgcggcccggcgccgcggccgccccgccgagctgtgcttctactcgggccgtgtgctcggccaccccggctccctcgtctcgctcagcgcctgcggcgccgccggcggcctggttggcctcattcagcttgggcaggagcaggtgctaatccagcccctcaacaactcccagggcccattcagtggacgagaacatctgatcaggcgcaaatggtccttgacccccagcccttctgctgaggcccagagacctgagcagctctgcaaggttctaacagttccacagtgtctgggcctcacctgggaggacttgaaatctggaggctggagtgatctggaggtgcctcattcatgtgtctggcctggaggtggatgacttgaagacaaggacaacaacgtggagcgtctacctgtggcctctgcagcttggcgtccataccttggtggcagacatacttcttctatggccaccagggctcccaatgcaagagttcccgcaagcccagcaggagctgtgctgcctttgaggatcagcctcagaaatcctagagcatcactctgaaggtactctgttggctgaagcggttagaaacctacccaggttcaagggcagagagatagaccccaccgctcaatgtcaaagaatttggggggc

SEQ ID NO: 55 - DNA, 3751 bp, Homo sapiens
  misc_feature: Incyte ID No 3534918CB1
ttcatggagcatggagcgcttggcagccttggggaacatgcagcgaaagttgtgggaaaggtactcagacaagagcaagactttgtaataacccaccaccagcgtttggtgggtcctactgtgatggagcagaaacacagatgcaagtttgcaatgaaagaaattgtccaattcatggcaagtgggcgacttgggccagttggagtgcctgttctgtgtcatgtggaggaggtgccagacagagaacaaggggctgctccgaccctgtgccccagtatggaggaaggaaatgcgaagggagtgatgtccagagtgatttttgcaacagtgacccttgcccaagtgagtgttggaaatacccatggtaactggagtccttggagtggctggggaacatgcagccggacgtgtaacggagggcagatgcggcggtaccgcacatgtgataaccctcctccctccaatgggggaagagcttgtgggggaccagactcccagatccagaggtgcaacactgacatgtgtcctgtggatggaagttggggaagctggcatagttggagccagtgctctgcctcctgtggaggaggtgaaaagactcggaagcggctgtgcgaccatcctgtgccagttaaaggtggccgtccctgtcccggagacactactcaggtgaccaggtgcaatgtacaagcatgtccaggtgggccccagcgagccagaggaagtgttattggaaatattaatgatgttgaatttggaattgctttccttaatgccacaataactgatagccctaactctgatactagaataatacgtgccaaaattaccaatgtacctcgtagtcttggttcagcaatgagaaagatagtttctattctaaatcccatttattggacaacagcaaaggaaataggagaagcagtcaatggctttaccctcaccaatgcagtcttcaaaagagaaactcaagtggaatttgcaactggagaaatcttgcagatgagtcatattgcccggggcttggattccgatggttctttgctgctagatatcgttgtgagtggctatgtcctacagcttcagtcacctgctgaagtcactgtaaaggattacacagaggactacattcaaacaggtcctgggcagctgtacgcctactcaacccggctgttcaccattgatggcatcagcatcccatacacatggaaccacaccgttttctatgatcaggcacagggaagaatgcctttcttggttgaaacacttcatgcatcctctgtggaatctgactataaccagatagaagagacactgggttttaaaattcatgcttcaatatccaaaggagatcgcagtaatcagtgcccctccgggtttaccttagactcagttggacctttttgtgctgatgaggatgaatgtgcagcagggaatccctgctcccatagctgccacaatgccatggggacttactactgctcctgccctaaaggcctcaccatagctgcagatggaagaacttgtcaagatattgatgagtgtgctttgggtaggcatacctgccacgctggtcaggactgtgacaatacgattggatcttatcgctgtgtggtccgttgtggaagtggctttcgaagaacctctgatgggctgagttgtcaagatattaatgaatgtcaagaatccagcccctgtcaccagcgctgtttcaatgccataggaagtttccattgtggatgtgaacctgggtatcagctcaaaggcagaaaatgcatggatgtgaacgagtgtagacaaaatgtatgcagaccagatcagcactgtaagaacacccgtggtggctataagtgcattgatctttgtccaaatggaatgaccaaggcagaaaatggaacctgtattgatattgatgaatgtaaagatgggacccatcagtgcagatataaccagatatgtgagaatacaagaggcagctatcgttgtgtatgcccaagaggttatcggtctcaaggagttggaagaccctgcatggatattgatgaatgtgaaaatacagatgcctgccagcatgagtgtaagaatacctttggaagttatcagtgcatctgcccacctggctatcaactcacacacaatggaaagacatgccaagatatcgatgaatgtctggagcagaatgtgcactgtggacccaatcgcatgtgcttcaacatgagaggaagctaccagtgcatcgatacaccctgtccacccaactaccaacgggatcctgtttcagggttctgcctcaagaactgtccacccaatgatttggaatgtgccttgagcccatatgccttggaatacaaactcgtctccctcccatttggaatagccaccaatcaagatttaatccggctggttgcatacacacaggatggagtgatgcatcccaggacaactttcctcatggtagatgaggaacagactgttccttttgccttgagggatgaaaacctgaaaggagtggtgtatacaacacgaccactacgagaagcagagacctaccgcatgagggtccgagcctcatcctacagtgccaatgggaccattgaatatcagaccacattcatagtttatatagctgtgtccgcctatccatactaaggaactctccaaagcctattccacatatttaaaccgcattaatcatggcaatcaagcccccttccagattactgtctcttgaacagttgcaatcttggcagcttgaaaatggtgctacactctgttttgtgtgccttccttggtacttctgaggtattttcatgatcccaccatggtcatatcttgaagtatggtctagaaaagtcccttattattttatttattacactggagcagttacttcccaaagattattctgaacatctaacaggacatatcagtgatggtttacagtagtgtagtacctaagatcattttcctgaaagccaaaccaaacaacgaaaaacaagaacaactaattcagaatcaaatagagtttttgagcatttgactatttttagaatcataaaattagttactaagtattttgatcaaagcttataaaataacttacggagatttttgtaagtattgatacattataataggacttgcctattttcatttttaagaagaaaaacaccactcattttataaaatatagtacagctactataaggcttgtttgatcccaaatggtgcttatcttgattgaacattcagaacaaggatattattttcagtgattttgtgagatcagctgaaccacttatgataataataataaaaaagactgctttgccctcacgtcagttgtacatggcatggaactttaaaaattttaatataaactttcatccagttagcttcataacttttacgttccagaattttgtttattttcctgtcaatgaaagcaatttttaaagataccagtgggacaggtttggttttttaaaaatctcatgtgttcaaattaacataaatattacacgtcaatacactgtacatggtggtaatagactctaagcaattgccaagatgtattctatttttatgaagtgtatatatattaccttagtgtgcattttctatataatatcttgatggactcttttataaaattattttataaaaaacaatgttacactaaaatcagcctaaataaattttcacaactttttttcataaccaaaaacaacaaacaacaaaaccggggccg

SEQ ID NO: 56 - DNA, 3579 bp, Homo sapiens
  misc_feature: Incyte ID No 2428715CB1
gggtcgaccacgcgtcggggcaggtggcgagcagcgagcagcgcctgcgggagcggccggtcggtcgggtccccgcgccccgcacgcccgcacgcccagcggggcccgcattgagcatgggcgcggcggccgtgcgctggcacttgtgcgtgctgctggccctgggcacacgcgggcggctggccgggggcagcgggctcccagggtcagtcgacgtggatgagtgctcagagggcacagatgactgccacatcgatgccatctgtcagaacacgcccaagtcctacaaatgcctctgcaagccaggctacaagggggaaggcaagcagtgtgaagacattgacgagtgtgagaatgactactacaatgggggctgtgtccacgagtgcatcaacatcccggggaactacaggtgtacctgctttgatggcttcatgctggcacacgatggacacaactgcctggatgtggacgagtgtcaggacaataatggtggctgccagcagatctgcgtcaatgccatgggcagctacgagtgtcagtgccacagtggcttcttccttagtgacaaccagcatacctgcatccaccgctccaatgagggtatgaactgcatgaacaaagaccatggctgtgcccacatctgccgggagacgcccaaaggtggggtggcctgcgactgcaggcccggctttgaccttgcccaaaaccagaaggactgcacactaacctgtaattatggaaacggaggctgccagcacagctgtgaggacacagacacaggccccacgtgtggttgccaccagaagtacgccctccactcagacggtcgcacgtgcatcgagaaggatgaggctgcaattgagcgctctcagttcaatgccacgtcagtagctgatgtggacaagcgggtgaaacggcggctactcatggagacgtgcgcagtcaataacggaggctgcgaccggacatgcaaggacacagccactggcgtgcgatgcagctgccccgttggattcacactgcagccggacgggaagacatgcaaagacatcaacgagtgcctggtcaacaacggaggctgcgaccacttctgccgcaacaccgtgggcagcttcgagtgcggctgccggaagggctacaagctgctcaccgacgagcgcacctgccaggacatcgacgagtgctccttcgagcggacctgtgaccacatctgcatcaactccccgggcagcttccagtgcctgtgtcaccgcggctacatcctctacgggacaacccactgcggagatgtggacgagtgcagcatgagcaacgggagctgtgaccagggctgcgtcaacaccaagggcagctacgagtgcgtctgtcccccggggaggcggctccactggaaccggaaggattgcgtggagacaggcaagtgtctttctcgtgccaagacctccccccgggcccagctgtcctgcagcaaggcaggcggtgtggagagctgcttcctttcctgcccggctcacacactcttcgtgccagatgcccccaccacccccatcaaacagaaggcccgcttcaagatccgagatgccaagtgccacctccggccccacagccaggcacgagcaaaggagaccgccaggcagccgctgctggaccactgccatgtgactttcgtgaccctcaagtgtgactcctccaagaagaggcgccgtggccgcaagtccccatccaaggaggtgtcccacatcacagcagagtttgagatcgagacaaagatggaagaggcctcagacacatgcgaagcggactgcttgcggaagcgagcagaacagagcctgcaggccgccatcaagaccctgcgcaagtccatcggccggcagcagttctatgtccaggtctcaggcactgagtacgaggtagcccagaggccagccaaggcgctggaggggcagggggcatgtggcgcaggccaggtgctacaggacagcaaatgcgttgcctgtgggcctggcacccacttcggtggtgagctcggccagtgtgtgccatgtatgccaggaacataccaggacatggaaggccagctcagttgcacaccgtgccccagcagcgacgggcttggtctgcctggtgcccgcaacgtgtcggaatgtggaggccagtgttctccaggcttcttctcggccgatggcttcaagccctgccaggcctgccccgtgggcacgtaccagcctgagcccgggcgcaccggctgcttcccctgtggagggggtttgctcaccaaacacgaaggcaccacctccttccaggactgcgaggctaaagtgcactgctcccccggccaccactacaacaccaccacccaccgctgcatccgctgccccgtcggcacctaccagcccgagtttggccagaaccactgcatcacctgtccgggcaacaccagcacagacttcgatggctccaccaacgtcacacactgcaaaaaccagcactgcggcggcgagcttggtgactacaccggctacatcgagtcccccaactaccctggcgactacccagccaacgctgaatgcgtctggcacatcgcacctcccccaaagcgcaggatcctcatcgtggtccctgagatcttcctgcccatcgaggatgagtgcggcgatgttctggtcatgaggaagagtgcctctcccacgtccatcaccacctatgagacctgccagacctacgagaggcccatcgccttcacctcccgctcccgcaagctctggatccagttcaaatccaatgaaggcaacagcggcaaaggcttccaagtgccctatgtcacctacgatgaggactaccagcaactcatagaggacatcgtgcgcgatgggcgcctgtacgcctcggagaaccaccaggaaattttgaaagacaagaagctgatcaaggccctcttcgacgtgctggcgcatccccagaaccgcggcttagtttcctcatgttaaaagaaaatacttatcctccctgtggcacagggttttgtttaaaagattagacaagatgatacaaccattttggaaataatttggcagcttcttataaacatatactttacaggtaagccagcaattgcactcctagctgcacccacgagaaatgaaaatatgtccatacaaagatttatacacaaatgtttatagcagctttattcataataatcaaaaactgaaaacaactcaaacatccatcaacaggcagatggataaacaaattatggtatgtccatgcaacggaatacaactcactgatgaaaaggaataaaccacaaatgcctgcaacgccatgatgaatctcaaaacatgctgagtgtagagaagccagacacaagagtagatactcctatacaattccacttacatggaaatctagaaaagacaattcgaatatatagtggcaaaaagcagaagagtggttgcctggaaccagggtgggaatgaagattaactgcccagaggcataaaaaatggggtggtgggggcggtgatggaaaagtgctatgccttcactgtacctttgtcaaaacttgttgaactg

SEQ ID NO: 57 - DNA, 5178 bp, Homo sapiens
  misc_feature: Incyte ID No 3351332CB1
gggaacccagaaccagcccgagccgcctgccccgtcgcccggccgccggcttagggcgcagcgcggttggtcctcgccccctcccgcccgccggcctaccaggccatgggggcgtcccgggaccgcgggctggccgcgctctggtgccttgggctcctggggggcctggcgcgcgtcgcgggcacgcactaccgctacctctggaggggctgctacccatgtcacctgggccaggccggctaccccgtgagcgccggtgaccagaggccagatgtggacgaatgccgaacccacaacggtggctgccagcaccggtgcgtgaacaccccaggctcctacctctgtgagtgcaagcccggcttccggctccacactgacagcaggacctgcctggccattaactcctgcgccctgggcaatggcggctgccagcaccactgtgtccagctcacaatcactcggcatcgctgccagtgccggcccgggttccagctccaggaggacggcaggcattgtgtccgtagaagcccgtgtgccaacaggaacggcagctgcatgcacaggtgccaggtggtccggggcctcgcccgctgtgagtgccacgtgggctatcagctagcagcggacggcaaggcctgtccagatgtggacgaatgtgccgcagggctggcccagtgtgcccatggctgcctcaacacccaggggtccttcaagtgcgtgtgtcacgcgggctatgagctgggcgccgatggccggcagtgctaccggattgagatggaaatcgtgaacagctgtgaggccaacaacggcggctgctcccatggctgcagccacaccagtgctgggcccctgtgcacatgtccccgcggctacgagctggacacagatcagaggacctgcatcgatgtcgacgactgtgcagacagcccgtgctgccagcaggtgtgcaccaacaaccctggcgggtacgagtgcggctgctacgccggctaccggctcagtgccgatggctgcggctgtgaggatgtggatgagtgcgcctccagccgtggcggctgcgagcaccactgcaccaacctggccggctccttccagtgctcctgcgaggccggctaccggctgcacgaggaccgtaggggctgcagccccctggaggagccgatggtggacctggacggcgagctgcctttcgtgcggcccctgccccacattgccgtgctccaggacgagctgccgcaactcttccaggatgacgacgtcggggccgatgaggaagaggcagagttgcggggcgaacacacgctcacagagaagtttgtctgcctggatgactcctttggccatgactgcagcttgacctgtgatgactgcaggaacggagggacctgcctcctgggcctggatggctgtgattgccccgagggctggactgggctcatctgcaatgagacttgtcctccggacacctttgggaagaactgcagcttctcctgcagctgtcagaatggtgggacctgcgactctgtcacgggggcctgccgctgccccccgggtgtcagtggaactaactgtgaggatggctgccccaagggctactatggcaagcactgtcgcaagaaatgcaactgtgccaaccggggccggtgccaccgcctctacggggcctgcctctgcgacccagggctctacggccgcttctgccacctcacctgcccgccgtgggcctttgggccgggctgctcggaggagtgccagtgtgtgcagccccacacgcagtcctgtgacaagagggatggcagctgctcctgcaaggctggcttccggggcgagcgctgtcaggcagagtgtgagctgggctactttgggccggggtgctggcaggcatgcacctgcccagtgggcgtggcctgtgactccgtgagcggcgagtgtgggaagcggtgtcctgctggcttccagggagaggactgtggccaagagtgcccggtggggacgtttggcgtgaactgctcgagctcctgctcctgtgggggggccccctgccacggggtcacggggcagtgccggtgtccgccggggaggactggggaagactgtgaggcagattgtcccgagggccgctgggggctgggctgccaggagatctgcccagcatgccagcacgctgcccgctgcgaccctgagaccggagcctgcctgtgcctccctggcttcgtcggcagccgctgccaggacgtgtgcccagcaggctggtatggtcccagctgccagacaaggtgctcttgtgccaatgatgggcactgccacccagccaccggacactgcagctgtgcccccgggtggaccggctttagctgccagagagcctgtgatactgggcactggggacctgactgcagccacccctgcaactgcagcgctggccacgggagctgtgatgccatcagcggcctgtgtctgtgtgaggctggctacgtgggcccgcggtgcgagcagcagtgtccccagggccactttgggcccggctgtgagcagctgtgccagtgtcagcatggagcagcctgtgaccacgtcagcggggcctgcacctgcccggccggctggaggggcaccttctgcgagcatgcctgcccggccggcttctttggattggactgtcgcagtgcctgcaactgcaccgccggagctgcctgtgatgccgtgaatggctcctgcctctgccccgctggccgccggggcccccgctgtgccgagacctgcccagcccacacctacgggcacaattgcagccaggcctgtgcctgctttaacggggcctcctgtgaccctgtccacgggcagtgccactgtgcccctggctggatggggccctcctgcctgcaggagtgcctcccccgggacgtcagagctggctgccggcacagcggcggttgcctcaacgggggcctgtgtgacccgcacacgggccgctgcctctgcccagccggctggactggggacaagtgtcagagcccctgcctgcggggctggtttggagaggcctgtgcccagcgctgcagctgcccgcctggcgctgcctgccaccacgtcactggggcctgccgctgtccccctggcttcactggctccggctgcgagcaggcctgcccacccggcagctttggggaggactgtgcgcagatgtgccagtgtcccggtgagaacccggcctgccaccctgccaccgggacctgctcatgtgctgctggctaccacggccccagctgccagcaacgatgtccgcccgggcggtatgggccaggctgtgaacagctgtgtgggtgtctcaacgggggctcctgtgatgcggccacgggggcctgccgctgccccactgggttcctcgggacggactgcaacctcacctgtccgcagggccgcttcggccccaactgcacccacgtgtgtgggtgtgggcagggggcggcctgcgaccctgtgaccggcacctgcctctgccccccggggagagccggcgtccgctgtgagcgaggctgcccccagaaccggtttggcgtgggctgcgagcacacctgctcctgcagaaatgggggcctgtgccacgccagcaacggcagctgctcctgtggcctgggctggacggggcggcactgcgagctggcctgtccccctgggcgctacggagccgcctgccatctggagtgctcctgccacaacaacagcacgtgtgagcctgccacgggcacctgccgctgcggccccggcttctatggccaggcctgcgagcacccctgtccccctggcttccacggggctggctgccaggggttgtgctggtgtcaacatggagccccctgcgaccccatcagtggccgatgcctctgccctgccggcttccacggccacttctgtgagagggggtgtgagccaggttcatttggagagggctgccaccagcgctgtgactgtgacgggggggcaccctgtgaccctgtcaccggtctctgcctttgcccaccagggcgctcaggagccacctgtaacctggattgcagaaggggccagtttgggcccagctgcaccctgcactgtgactgcgggggtggggctgactgcgaccctgtcagtgggcagtgtcactgtgtggatggctacatggggcccacgtgccgggaaggtgggcccctccggctccccgagaacccgtccttagcccagggctcagcgggcacactgcccgcctccagcagacccacatcccggagcggtggaccagcgaggcactagtagaggcagtcccgtggagcccgcctctccagtcccagccagaggggaccctggcctttggtgaccactgagaaggacacttcacgggcccagagctcctggtactgcccttcctttgagggccgtggagggctgtggacagcccagcaacctgtcgctcttggaggctggtgtggccttgaggagggaagcctcgcatggccgctggaagagaggcgcctcctggcctggctctgcagaacccaggggcacgctctgggcctgggctgaggaagtcccgctctccccgcggctctgagttggactgaggacaggtgtgggcgccagtgtgggtgcaggcgcaggtgcaggcacagggccactgtcctccaggcaggctttttggtgctaggccctgggactggaagtcgcccagcccgtatttatgtaaaggtatttatgggccactgcacatgcccgctgcagccctgggatcagctggaagctgcctgtcatctcctgcccaatccccagaaaccctgattcaggtctgcaggctcctgcgggctcaccaggctgctggctccggtaccatgtaaacctaggaaggtaaaggagcaggcaacctcctcgtggcctgtgtgtttgctgtgttacgtggactctgtgtgggctcctccctggggcccggccagcataacggtgcacccagggacctcccagtgcacccggggccctttgcaggggtgggggtgccacacaagtgaagaagttgggactcatctcagttcccagtgctattgaggagaacgctggggctgcattcattaccgctgagacccagagactggctgttcccagagaatggcccagggggaggagggctggtgtggaagggcaacttggactgag

SEQ ID NO: 58 - DNA, 11367 bp, Homo sapiens
  misc_feature: Incyte ID No 6382722CB1
atggcgaagcggctctgcgcggggagcgcactgtgtgttcgcggcccccggggccccgcgccgctgctgctggtcgggctggcgctgctgggcgcggcgcgggcgcgggaggaggcgggcggcggcttcagcctgcacccgccctacttcaacctggccgagggcgcccgcatcgccgcctccgcgacctgcggagaggaggccccggcgcgcggctccccgcgccccaccgaggacctttactgcaagctggtagggggccccgtggccggcggcgaccccaaccagaccatccggggccagtactgcgacatctgcacggctgccaacagcaacaaggcacaccccgcgagcaatgccatcgatggcacggagcgctggtggcagagtccaccgctgtcccgcggcctggagtacaacgaggtcaacgtcaccctggacctgggccaggtcttccacgtggcctacgtcctcatcaagtttgccaactcaccccggccggacctctgggtgctggagcggtccatggacttcggccgcacctaccagccctggcagttctttgcctcctctaagagggactgtctggagcggttcgggccacagacgctggagcgcatcacacgggacgacgcggccatctgcaccaccgagtactcacgcatcgtgcccctggagaacggagagatcgtggtgtccctggtgaacggacgtccgggcgccatgaatttctcctactcgccgctgctacgtgagttcaccaaggccaccaacgtccgcctgcgcttcctgcgtaccaacacgctgctgggccatctcatggggaaggcgctgcgggaccccacggtcacccgccggtattattacagcatcaaggatatcagcatcggaggccgctgtgtctgccacggccacgcggatgcctgcgatgccaaagaccccacggacccgttcaggctgcagtgcacctgccagcacaacacctgcgggggcacctgcgaccgctgctgccccggcttcaatcagcagccgtggaagcctgcgactgccaacagtgccaacgagtgccagtcctgtaactgctacggccatgccaccgactgttactacgaccctgaggtggaccggcgccgcgccagccagagcctggatggcacctatcagggtgggggtgtctgtatcgactgccagcaccacaccgccggcgtcaactgtgagcgctgcctgcccggcttctaccgctctcccaaccaccctctcgactcgccccacgtctgccgccgctgcaactgcgagtccgacttcacggatggcacctgcgaggacctgacgggtcgatgctactgccggcccaacttctctggggagcggtgtgacgtgtgtgccgagggcttcacgggcttcccaagctgctacccgacgccctcgtcctccaatgacaccagggagcaggtgctgccagctggccagattgtgaattgtgactgcagcgcggcagggacccagggcaacgcctgccggaaggacccaagggtgggacgctgtctgtgcaaacccaacttccaaggcacccattgtgagctctgcgcgccagggttctacggccccggctgccagccctgccagtgttccagccctggagtggccgatgaccgctgtgaccctgacacaggccagtgcaggtgccgagtgggcttcgagggggccacatgtgatcgctgtgcccccggctactttcacttccctctctgccagttgtgtggctgcagccctgcaggaaccttgcccgagggctgcgatgaggccggccgctgcctatgccagcctgagtttgctggacctcattgtgaccggtgccgccctggctaccatggtttccccaactgccaagcatgcacctgcgaccctcggggagccctggaccagctctgtggggcgggaggtttgtgccgctgccgccccggctacacaggcactgcctgccaggaatgcagccccggctttcacggcttccccagctgtgtcccctgccactgctctgctgaaggctccctgcacgcagcctgtgacccccggagtgggcagtgcagctgccggccccgtgtgacggggctgcggtgtgacacgtgtgtgcccggtgcctacaacttcccctactgcgaagctggctcttgccaccctgccggtctggccccagtggatcctgcccttcctgaggcacaggttccctgtatgtgccgggctcacgtggaggggccgagctgtgaccgctgcaaacctgggttctggggactgagccccagcaaccccgagggctgtacccgctgcagctgcgacctcaggggcacactgggtggagttgctgagtgccagccgggcaccggccagtgcttctgcaagccccacgtgtgcggccaggcctgcgcgtcctgcaaggatggcttctttggactggatcaggctgactattttggctgccgcagctgccggtgtgacattggcggtgcactgggccagagctgtgaaccgaggacgggcgtctgccggtgccgccccaacacccagggccccacctgcagcgagcctgcgagggaccactacctcccggacctgcaccacctgcgcctggagctggaggaggctgccacacctgagggtcacgccgtgcgctttggcttcaaccccctcgagttcgagaacttcagctggaggggctacgcgcagatggcacctgtccagcccaggatcgtggccaggctgaacctgacctcccccgaccttttctggctcgtcttccgatacgtcaaccggggggccatgagtgtgagcgggcgggtctctgtgcgagaggagggcaggtcggccgcctgtgccaactgcacagcacagagtcagcccgtggccttcccacccagcacggagcctgccttcatcaccgtgccccagaggggcttcggagagccctttgtgctgaaccctggcacctgggccctgcgtgtggaggccgaaggggtgctcctggactacgtggttctgctgcctagcgcatactacgaggcggcgctcctgcagctgcgggtgactgaggcctgcacataccgtccctctgcccagcagtctggcgacaactgcctcctctacacacacctccccctggatggcttcccctcggccgccgggctggaggccctgtgtcgccaggacaacagcctgccccggccctgccccacggagcagctcagcccgtcgcacccgccactgatcacctgcacgggcagtgatgtggacgtccagcttcaagtggcagtgccacagccaggccgctatgccctagtggtggagtacgccaatgaggatgcccgccaggaggtgggcgtggctgtgcacaccccacagcgggccccccagcaggggctgctctccctgcacccctgcctgtacagcaccctgtgccggggcactgcccgggatacccaggaccacctggctgtcttccacctggactcggaggccagcgtgaggctcacagccgagcaggcacgcttcttcctgcacggggtcactctggtgcccattgaggagttcagcccggagttcgtggagccccgggtcagctgcatcagcagccacggcgcctttggccccaacagtgccgcctgtctgccctcgcgcttcccaaagccgccccagcccatcatcctcagggactgccaggtgatcccgctgccgcccggcctcccgctgacccacgcgcaggatctcactccagccacgtccccagctggaccccgacctcggccccccaccgctgtggaccctgatgcagagcccaccctgctgcgtgagccccaggccaccgtggtcttcaccacccatgtgcccacgctgggccgctatgccttcctgctgcacggctaccagccagcccaccccaccttccccgtggaagtcctcatcaacgccggccgcgtgtggcagggccacgccaacgccagcttctgtccacatggctacggctgccgcaccctggtggtgtgtgagggccaggccctgctggacgtgacccacagcgagctcactgtgaccgtgcgtgtgcccgagggccggtggctctggctggattatgtactcgtggtccctgagaacgtctacagctttggctacctccgggaggagcccctggataaatcctatgacttcatcagccactgcgcagcccagggctaccacatcagccccagcagctcatccctgttctgccgaaacgctgctgcttccctctccctcttctataacaacggagcccgtccatgtggctgccacgaagtaggtgctacaggccccacgtgtgagcccttcgggggccagtgtccctgccatgcccatgtcattggccgtgactgctcccgctgtgccaccggatactggggcttccccaactgcaggccctgtgactgcggtgcccgcctctgtgacgagctcacgggccagtgcatctgcccgccacgcaccatcccgcccgactgcctgctgtgccagccccagacctttggctgccaccccctggtcggctgtgaggagtgtaactgctcagggcccggcatccaggagctcacagaccctacctgtgacacagacagcggccagtgcaagtgcagacccaacgtgactgggcgccgctgtgatacctgctctccgggcttccatggctacccccgctgccgcccctgtgactgtcacgaggcgggcactgcgcctggcgtgtgtgaccccctcacagggcagtgctactgtaaggagaacgtgcagggccccaaatgtgaccagtgcagccttgggaccttctcactggatgctgccaaccccaaaggttgcacccgctgcttctgctttggggccacggagcgctgccggagctcgtcctacacccgccaggagttcgtggatatggagggatgggtgctgctgagcactgaccggcaggtggtgccccacgagcggcagccagggacggagatgctccgtgcagacctgcggcacgtgcctgaggctgtgcccgaggctttccccgagctgtactggcaggccccaccctcctacctgggggaccgggtgtcatcctacggtgggaccctccgttatgaactgcactcagagacccagcggggagatgtctttgtccccatggagagcaggccggatgtggtgctgcagggcaaccagatgagcatcacattcctggagccggcataccccacgcctggccacgttcaccgtgggcagctgcagctggtggaggggaacttccggcatacggagactcgcaacactgtgtcccgcgaggagctcatgatggtgctggccagcctggagcagctgcagatccgtgccctcttctcacagatctcctcggctgtctccctgcgcagggtggcactggaggtggccagcccagcaggccagggggccctggccagcaatgtggagctgtgcctgtgccccgccagctaccggggggactcatgccaggaatgtgcccccggcttctatcgggacgtcaaaggtctcttcctgggccgatgtgtcccttgtcagtgccatggacactcagaccgctgcctccctggctctggcgtctgtgtggactgccagcacaacaccgaaggggcccactgtgagcgctgccaggctggcttcatgagcagcagggacgaccccagcgccccctgtgtcagctgcccctgccccctctcagtgccttccaacaacttcgccgagggctgtgtcctgcgaggcggccgcacccagtgcctctgcaaacctggttatgcaggtgcctcctgcgagcggtgtgcgcccggattctttgggaacccactggtgctgggcagctcctgccagccatgcgactgcagcggcaacggtgaccccaacttgctcttcagcgactgcgaccccctgacgggcgcctgccgtggctgcctgcgccacaccactgggccccgctgcgagatctgtgcccccggcttctacggcaacgccctgctgcccggcaactgcacccggtgcgactgtaccccatgtgggacagaggcctgcgacccccacagcgggcactgcctgtgcaaggcgggcgtgactgggcggcgctgtgaccgctgccaggagggacattttggtttcaatggctgcgggggctgccgcccgtgtgcttgtggaccggccgccgagggctccgagtgccacccccagagcggacagtgccactgccgaccagggaccatgggaccccagtgccgcgagtgtgcccctggctactgggggctccctgagcagggctgcaggcgctgccagtgccctgggggccgctgtgaccctcacacgggccgctgcaactgccccccggggctcagcggggagcgctgcgacacctgcagccagcagcatcaggtgcctgttccaggcgggcctgtgggccacagcatccactgtgaagtgtgtgaccactgtgtggtcctgctcctggatgacctggaacgggccggcgccctcctccccgccattcacgagcaactgcgtggcatcaatgccagctccatggcctgggcccgtctgcacaggctgaacgcctccatcgctgacctgcagagccagctccggagccccctgggcccccgccatgagacggcacagcagctggaggtgctggagcagcagagcacaagcctcgggcaggacgcacggcggctaggcggccaggccgtggggacccgagaccaggcgagccaattgctggccggcaccgaggccacactgggccatgcgaagacgctgttggcggccatccgggctgtggaccgcaccctgagcgagctcatgtcccagacgggccacctggggctggccaatgcctcggctccatcaggtgagcagctgctccggacactggccgaggtggagcggctgctctgggagatgcgggcccgggacctgggggccccgcaggcagcagctgaggctgagttggctgcagcacagagattgctggcccgggtgcaggagcagctgagcagcctctgggaggagaaccaggcactggccacacaaacgcgcgaccggctggcccagcacgaggccggcctcatggacctgcgagaggctttgaaccgggcagtggacgccacacgggaggcccaggagctcaacagccgcaaccaggagcgcctggaggaagccctgcaaaggaagcaggagctgtcccgggacaatgccaccctgcaggccactctgcatgcggctagggacaccctggccagcgtcttcagattgctgcacagcctggaccaggctaaggaggagctggagcgcctcgccgccagcctggacggggctcggaccccactgctgcagaggatgcagaccttctccccggcgggcagcaagctgcgtctagtggaggccgccgaggcccacgcacagcagctgggccagctggcactcaatctgtccagcatcatcctggacgtcaaccaggaccgcctcacccagagggccatcgaggcctccaacgcctacagccgcatcctgcaggccgtgcaggctgccgaggatgctgctggccaggccctgcagcaggcggaccacacgtgggcgacggtggtgcggcagggcctggtggaccgagcccagcagctcctggccaacagcactgcactagaagaggccatgctccaggaacagcagaggctgggccttgtgtgggctgccctccagggtgccaggacccagctccgagatgtccgggccaagaaggaccagctggaggcgcacatccaggcggcgcaggccatgcttgccatggacacagacgagacaagcaagaagatcgcacatgccaaggctgtggctgctgaagcccaggacaccgccacccgtgtgcagtcccagctgcaggccatgcaggagaatgtggagcggtggcagggccagtacgagggcctgcggggccaggacctgggccaggcagtgcttgacgcaggccactcagtgtccaccctggagaagacgctgccccagctgctggccaagctgagcatcctggagaaccgtggggtgcacaacgccagcctggccctgtccgccagcattggccgcgtgcgagagctcattgcccaggcccggggggctgccagtaaggtcaaggtgcccatgaagttcaacgggcgctcaggggtgcagctgcgcaccccacgggatcttgccgaccttgctgcctacactgccctcaagttctacctgcagggcccagagcctgagcctgggcagggtaccgaggatcgctttgtgatgtacatgggcagccgccaggccactggggactacatgggtgtgtctctgcgtgacaagaaggtgcactgggtgtatcagctgggtgaggcgggccctgcagtcctaagcatcgatgaggacattggggagcagttcgcagctgtcagcctggacaggactctccagtttggccacatgtccgtcacagtggagagacagatgatccaggaaaccaagggtgacacggtggcccctggggcagaggggctgctcaacctgcggccagacgacttcgtcttctacgtcggggggtaccccagtaccttcacgccccctcccctgcttcgcttccccggctaccggggctgcatcgagatggacacgctgaatgaggaggtggtcagcctctacaacttcgagaggaccttccagctggacacggctgtggacaggccttgtgcccgctccaagtcgaccggggacccgtggctcacggacggctcctacctggacggcaccggcttcgcccgcatcagcttcgacagtcagatcagcaccaccaagcgcttcgagcaggagctgcggctcgtgtcctacagcggggtgctcttcttcctgaagcagcagagccagttcctgtgcttggccgtgcaagaaggcagcctcgtgctgttgtatgactttggggctggcctgaaaaaggccgtcccactgcagcccccaccgcccctgacctcggccagcaaggcgatccaggtgttcctgctggggggcagccgcaagcgtgtgctggtgcgtgtggagcgggccacggtgtacagcgtggagcaggacaatgatctggagctggccgacgcctactacctggggggcgtgccgcccgaccagctgcccccgagcctgcgacggctcttccccaccggaggctcagtccgtggctgcgtcaaaggcatcaaggccctgggcaagtatgtggacctcaagcggctgaacacgacaggcgtgagcgccggctgcaccgccgacctgctggtggggcgcgccatgactttccatggccacggcttccttcgcctggcgctctcgaacgtggcaccgctcactggcaacgtctactccggcttcggcttccacagcgcccaggacagtgccctgctctactaccgggcgtccccggatgggctatgccaggtgtccctgcagcagggccgtgtgagcctacagctcctgaggactgaagtgaaaactcaagcgggcttcgccgatggtgccccccattacgtcgccttctacagcaatgccacgggagtctggctgtatgtcgatgaccagctccagcagatgaagccccaccggggaccaccccccgagctccagccgcagcctgaggggcccccgaggctcctcctgggaggcctgcctgagtctggcaccatttacaacttcagtggctgcatcagcaacgtcttcgtgcagcggctcctgggcccacagcgcgtatttgatctgcagcagaacctgggcagcgtcaatgtgagcacgggctgtgcacccgccctgcaagcccagaccccgggcctggggcctagaggactgcaggccaccgcccggaaggcctcccgccgcagccgtcagcccgcccggcatcctgcctgcatgctgcccccacacctcaggaccacccgagactcctaccagtttgggggttccctgtccagtcacctggagtttgtgggcatcctggcccgacataggaactggcccagtctctccatgcacgtcctcccgcgaagctcccgaggcctcctcctcttcactgcccgtctgaggcccggcagcccctccctggcgctcttcctgagcaatggccacttcgttgcacagatggaaggcctcgggactcggctccgcgcccagagccgccagcgctcccggcctggccgctggcacaaggtctccgtgcgctgggagaagaaccggatcctgctggtgacggacggggcccgggcctggagccaggaggggccgcaccggcagcaccagggggcagagcacccccagccccacaccctctttgtgggcggcctcccggccagcagccacagctccaaacttccggtgaccgtcgggttcagcggctgtgtgaagagactgaggctgcacgggaggcccctgggggcccccacacggatggcaggggtcacaccctgcatcttgggccccctggaggcgggcctgttcttcccaggcagcgggggagttatcactttagacctcccaggagctacactgcctgatgtgggcctggaactggaggtgcggcccctggcagtcaccggactgatcttccacttgggccaggcccggacgcccccctacttgcagttgcaggtgaccgagaagcaagtcctgctgcgggcggatgacggagcaggggagttctccacgtcagtgacccgcccctcagtgctgtgtgatggccagtggcaccggctagcggtgatgaaaagcgggaatgtgctccggctggaggtggacgcgcagagcaaccacaccgtgggccccttgctggcggctgcagctggtgccccagcccctctgtacctcgggggcctgcctgagcccatggccgtgcagccctggccccccgcctactgcggctgcatgaggaggctggcggtgaaccggtcccccgtcgccatgactcgctctgtggaggtccacggggcagtgggggccagtggctgcccagccgcctaggacacagccaaccccggcccctggtcaggcccctgcagctgcctcacaccgccccttgtgctcgcctcataggtgtctatttggactctaagctctacgggtgacagatcttgtttctgaagatggtttaagttatagcttcttaaacgaaagaataaaatactgcaaaatgtttttatatttggcccttccacccatttttaattgtgagagatttgtcaccaatcatcactggttcctccttaaaaattaaaaagtaacttctgtgtaaaaaaaaaa

SEQ ID NO: 59 - DNA, 4255 bp, Homo sapiens
  misc_feature: Incyte ID No 55022490CB1
gcggcacagacccagctctgaattctgggtcaaccatggaccaactgtgacccttcggacaagtcccttcgcctctctggagctggcctataagagggaaaaggaacccctgtggagagggtctatttatcctggcgaagatcgcctgaagtgatcttctaacaggagtgtttccagaggaggggctgggccgggagaggtgtggacagctggggaccgctctgagcagcgcagccccgggcgccccacaccaccacatggtccggggaggaaggtgggagcaggcacacaagaaggaacctctgggggtctgggggcccctgccatgtgtgaggggtgcccaggggacccttggggacaggaacgggggcacgggtgggtggcggcactggggagggtgtgagggtatgcccatgccctcctcctcgcagaatgtctgcacaaactctggtgcatctgtggggaccacctgtcactccaagctggatgcagctgtggacggcacccggtgtggggagaataagtggtgtctcagtggggagtgcgtacccgtgggcttccggcccgaggccgtggatggtggctggtctggctggagcgcctggtccatctgctcacggagctgtggcatgggcgtacagagcgccgagcggcagtgcacgcagcctacgcccaaatacaaaggcagatactgtgtgggtgagcgcaagcgcttccgcctctgcaacctgcaggcctgccctgctggccacccctccttccgccacgtccagtgcagccactttgacgctatgctctacaagggccagctgcacacatgggtgcccgtggtcaatgacgtgaacccctgcgagctgcactgccggcccgcgaatgagtactttgccgagaagctgcgggacgccgtggtcgatggcaccccctgctaccaggtccgagccagccgggacctctgcatcaacggcatctgtaagaacgtgggctgtgacttcgagattgactccggtgctatggaggaccgctgtggtgtgtgccacggcaacggctccacctgccacaccgtgagcgggaccttcgaggaggccgagggcctggggtatgtggatgtggggctgatcccagccggcgcacgcgagatccgcatccaagaggttgccgaggctgccaacttcctggcactgcggagtgaggacccggagaagtacttcctcaatggtggctggaccatccagtggaacggggactaccaggtggcagggaccaccttcacatacgcacgcaggggcaactgggagaacctcacgtccccgggtcccaccaaggagcctgtctggatccagctgctgttccaggagagcaaccctggggtgcactacgagtacaccatccacagggaggcaggtggccacgacgaggtcccgccgcccgtgttctcctggcattatgggccctggaccaagtgcacagtcacctgcggcagaggtgtgcagaggcagaatgtgtactgcttggagcggcaggcagggcccgtggacgaggagcactgtgaccccctgggccggcctgatgaccaacagaggaagtgcagcgagcagccctgccctgccaggtggtgggcaggtgagtggcagctgtgctccagctcctgcgggcctgggggcctctcccgccgggccgtgctctgcatccgcagcgtggggctggatgagcagagcgccctggagccacccgcctgtgaacaccttccccggccccctactgaaaccccttgcaaccgccatgtaccctgtccggccacctgggctgtggggaactggtctcagtgctcagtgacatgtggggagggcactcagcgccgaaatgtcctctgcaccaatgacaccggtgtcccctgtgacgaggcccagcagccagccagcgaagtcacctgctctctgccactctgtcggtggcccctgggcacactgggccctgaaggctcaggcagcggctcctccagccacgagctcttcaacgaggctgacttcatcccgcaccacctggccccacgcccttcacccgcctcatcacccaagccaggcaccatgggcaacgccattgaggaggaggctccagagctggacctgccggggcccgtgtttgtggacgacttctactacgactacaatttcatcaatttccacgaggatctgtcctacgggccctctgaggagcccgatctagacctggcggggacaggggaccggacacccccaccacacagccgtcctgctgcgccctccacgggtagccctgtgcctgccacagagcctcctgcagccaaggaggagggggtactgggaccttggtccccgagcccttggcctagccaggccggccgctccccacccccaccctcagagcagacccctgggaaccctttgatcaatttcctgcctgaggaagacacccccataggggccccagatcttgggctccccagcctgtcctggcccagggtttccactgatggcctgcagacacctgccacccctgagagccaaaatgatttcccagttggcaaggacagccagagccagctgccccctccatggcgggacaggaccaatgaggttttcaaggatgatgaggaacccaagggccgcggagcaccccacctgcccccgagacccagctccacgctgccccctttgtcccctgttggcagcacccactcctctcctagtcctgacgtggcggagctgtggacaggaggcacagtggcctgggagccagctctggagggtggcctggggcctgtggacagtgaactgtggcccactgttggggtggcttctctccttcctcctcccatagcccctctgccagagatgaaggtcagggacagttccctggagccggggactccctccttcccaaccccaggaccaggctcatgggacctgcagactgtggcagtgtgggggaccttcctccccacaaccctgactggcctcgggcacatgcctgagcctgccctgaacccaggacccaagggtcagcctgagtccctcagccctgaggtgcccctgagctctaggctgctgtccacaccagcttgggacagccccgccaacagccacagagtccctgagacccagccgctggctcccagcctggctgaagcggggccccccgcggacccgttggttgtcaggaacgccagctggcaagcgggaaactggagcgagtgctctaccacctgtggcctgggtgcggtctggaggccggtgcgctgtagctccggccgggatgaggactgcgcccccgctggccggccccagcctgcccgccgctgccacctgcggccctgtgccacctggcactcaggcaactggagtaagtgctcccgcagctgcggcggaggttcctcagtgcgggacgtgcagtgtgtggacacacgggacctccggccactgcggcccttccattgtcagcccgggcctgccaagccgcctgcgcaccggccctgcggggcccagccctgcctcagctggtacacatcttcctggagggagtgctccgaggcctgtggcggtggtgagcagcagcgtctagtgacctgcccggagccaggcctctgcgaggaggcgctgagacccaacaccacccggccctgcaacacccacccctgcacgcagtgggtggtggggccctggggccagtgctcagccccctgtggtggtggtgtccagcggcgcctggtcaagtgtgtcaacacccagacagggctgcccgaggaagacagtgaccagtgtggccacgaggcctggcctgagagctcccggccgtgtggcaccgaggattgtgagcccgtcgagcctccccgctgtgagcgggaccgcctgtccttcgggttctgcgagacgctgcgcctactgggccgctgccagctgcccaccatccgcacccagtgctgccgctcgtgctctccgcccagccacggcgccccctcccgaggccatcagcgggttgcccgccgctgactgtgccaggatgcacagaccgaccgacagacctcagtgcccaccacgggctgtggcggagctcccgccccctgcgccctaatggtgctaaccccctctcactacccagcagcaggctggggacctcctccccctcaaaaaaggtatttttttattctaacagtttgtgtaacatttattatgattttacataaatgagcatctaccattccaaaaaaaaaaaaaaaaaa

SEQ ID NO: 60 - DNA, 3438 bp, Homo sapiens
  misc_feature: Incyte ID No 6755002CB1
tgtgcgccgggagggccggcgccctcttccgaatgtcctgcggccccagcctctcctcacgctcgcgcagtctccgccgcagtctcagctgcagctgcaggactgagccgtgcacccggaggagacccccggaggaggcgacaaacttcgcagtgccgcgacccaaccccagccctgggtagcctgcagcatggcccagctgttcctgcccctgctggcagccctggtcctggcccaggctcctgcagctttagcagatgttctggaaggagacagctcagaggaccgcgcttttcgcgtgcgcatcgcgggcgacgcgccactgcagggcgtgctcggcggcgccctcaccatcccttgccacgtccactacctgcggccaccgccgagccgccgggctgtgctgggctctccgcgggtcaagtggactttcctgtcccggggccgggaggcagaggtgctggtggcgcggggagtgcgcgtcaaggtgaacgaggcctaccggttccgcgtggcactgcctgcgtacccagcgtcgctcaccgacgtctccctggcgctgagcgagctgcgccccaacgactcaggtatctatcgctgtgaggtccagcacggcatcgatgacagcagcgacgctgtggaggtcaaggtcaaaggggtcgtctttctctaccgagagggctctgcccgctatgctttctccttttctggggcccaggaggcctgtgcccgcattggagcccacatcgccaccccggagcagctctatgccgcctaccttgggggctatgagcaatgtgatgctggctggctgtcggatcagaccgtgaggtatcccatccagaccccacgagaggcctgttacggagacatggatggcttccccggggtccggaactatggtgtggtggacccggatgacctctatgatgtgtactgttatgctgaagacctaaatggagaactgttcctgggtgaccctccagagaagctgacattggaggaagcacgggcgtactgccaggagcggggtgcagagattgccaccacgggccaactgtatgcagcctgggatggtggcctggaccactgcagcccagggtggctagctgatggcagtgtgcgctaccccatcgtcacacccagccagcgctgtggtgggggcttgcctggtgtcaagactctcttcctcttccccaaccagactggcttccccaataagcacagccgcttcaacgtctactgcttccgagactcggcccagccttctgccatccctgaggcctccaacccagcctccaacccagcctctgatggactagaggctatcgtcacagtgacagagaccctggaggaactgcagctgcctcaggaagccacagagagtgaatcccgtggggccatctactccatccccatcatggaggacggaggaggtggaagctccactccagaagacccagcagaggcccctaggacgctcctagaatttgaaacacaatccatggtaccgcccacggggttctcagaagaggaaggtaaggcattggaggaagaagagaaatatgaagatgaagaagagaaagaggaggaagaagaagaggaggaggtggaggatgaggctctgtgggcatggcccagcgagctcagcagcccgggccctgaggcctctctccccactgagccagcagcccaggagaagtcactctcccaggcgccagcaagggcagtcctgcagcctggtgcatcaccacttcctgatggagagtcagaagcttccaggcctccaagggtccatggaccacctactgagactctgcccactcccagggagaggaacctagcatccccatcaccttccactctggttgaggcaagagaggtgggggaggcaactggtggtcctgagctatctggggtccctcgaggagagagcgaggagacaggaagctccgagggtgccccttccctgcttccagccacacgggcccctgagggtaccagggagctggaggccccctctgaagataattctggaagaactgccccagcagggacctcagtgcaggcccagccagtgctgcccactgacagcgccagccgaggtggagtggccgtggtccccgcatcaggtgactgtgtccccagcccctgccacaatggtgggacatgcttggaggaggaggaaggggtccgctgcctatgtctgcctggctatgggggggacctgtgcgatgttggcctccgcttctgcaaccccggctgggacgccttccagggcgcctgctacaagcacttttccacacgaaggagctgggaggaggcagagacccagtgccggatgtacggcgcgcatctggccagcatcagcacacccgaggaacaggacttcatcaacaaccggtaccgggagtaccagtggatcggactcaacgacaggaccatcgaaggcgacttcttgtggtcggatggcgtccccctgctctatgagaactggaaccctgggcagcctgacagctacttcctgtctggagagaactgcgtggtcatggtgtggcatgatcagggacaatggagtgacgtgccctgcaactaccacctgtcctacacctgcaagatggggctggtgtcctgtgggccgccaccggagctgcccctggctcaagtgttcggccgcccacggctgcgctatgaggtggacactgtgcttcgctaccggtgccgggaaggactggcccagcgcaatctgccgctgatccgatgccaagagaacggtcgttgggaggccccccagatctcctgtgtgcccagaagacctgcccgagctctgcacccagaggaggacccagaaggacgtcaggggaggctactgggacgctggaaggcgctgttgatccccccttccagccccatgccaggtccctagggggcaaggccttgaacactgccggccacagcactgccctgtcacccaaattttccctcacaccctgcgctcccgccaccacaggaagtgacaacatgacgaggggtggtgctggagtccaggtgacagttcctgaaggggcttctgggaaatacctaggaggctccagcccagcccaggccctctccccctaccctgggcaccagatcttccatcagggccggagtaaatccctaagtgcctcaactgccctctccctggcagccatcttgtcccctctattcctctagggagcactgtgcccactctttctgggttttccaagggaatgggcttgcaggatggagtgtctgtaaaatcaacaggaaataaaactgtgtatgagcccagggtagggggagagggcctgggctgggctggagcctcctaggtatttcccagaagccccttcaggaactgtcacctggactccagcaccacccctcgtcatgttgtcacttcctgtggtggcg

SEQ ID NO: 61 - DNA, 1683 bp, Homo sapiens
  misc_feature: Incyte ID No 7350907CB1
ggcgtggggacacgagccaggcgccgccgccggagccagcggagccggggccagagccggagcgcgtccgcgtccacgcagccgccggccggccagcacccagggccctgcatgccaggtcgttggaggtggcagcgagacatgcacccggcccggaagctcctcagcctcctcttcctcatcctgatgggcactgaactcactcaagtgctgcccaccaaccctgaggagagctggcaggtgtacagctctgcccaggacagcgagggcaggtgtatctgcacagtggtcgccccacagcagaccatgtgttcacgggatgcccgcacaaaacagctgaggcagctactggagaaggtgcagaacatgtctcaatccatagaggtcttggacaggcggacccagagagacttgcagtacgtggagaagatggagaaccaaatgaaaggactggagtccaagttcaaacaggtggaggagagtcataagcaacacctggccaggcagtttaaggcgataaaagcgaaaatggatgaacttaggcctttgatacctgtgttggaagagtacaaggccgatgccaaattggtattgcagtttaaagaggaggtccagaatctgacgtcagtgcttaacgagctgcaagaggaaattggcgcctatgactacgatgaacttcagagcagagtgtccaatcttgaagaaaggctccgtgcatgcatgcaaaaactagcttgcgggaagttgacgggcatcagtgaccccgtgactgtcaagacctccggctcgaggttcggatcctggatgacagaccctctcgcccctgaaggcgataaccgggtgtggtacatggacggctatcacaacaaccgcttcgtacgtgagtacaagtccatggttgacttcatgaacacggacaatttcacctcccaccgtctcccccacccctggtcgggcacggggcaggtggtctacaacggttctatctacttcaacaagttccagagccacatcatcatcaggtttgacctgaagacagagaccatcctcaagacccgcagcctggactatgccggttacaacaacatgtaccactacgcctggggtggccactcggacatcgacctcatggtggacgagagcgggctgtgggccgtgtacgccaccaaccagaacgctggcaacatcgtggtcagtaggctggaccccgtgtccctgcagaccctgcagacctggaacacgagctaccccaagcgcagcgccggggaggccttcatcatctgcggcacgctgtacgtcaccaacggctactcagggggtaccaaggtccactatgcataccagaccaatgcctccacctatgaatacatcgacatcccattccagaacaaatactcccacatctccatgctggactacaaccccaaggaccgggccctgtatgcctggaacaacggccaccagatcctctacaacgtgaccctcttccacgtcatccgctccgacgagttgtagctccctcctcctggaagccaagggcccacgtcctcaccacaaagggactcctgtgaaactgctgccaaaaagataccaataacactaacaataccgatcttgaaaaatcatcagcagtgcggattctgacatcgagggatggcattacctccgtgtttctccctttcg

SEQ ID NO: 62 - DNA, 6886 bp, Homo sapiens
  misc_feature: Incyte ID No 7474411CB1
cggggcacagcgggagcccgtgcaggcgggcgcggggcggctgggcggcggtggcggccgtccatgcggcggcgctcggggctgcccggcgccgggaaccacgcgggggcgaggcgaggcgaggcggccgccggtcgctccgggacgcggaccgccaggacttgaacgcaactcccaattgcagaaaattggcaacgtctctgaagagcccttgcttttgcctggacccccagcatcatggtttcccatttcatggggtctctcagtgtcctgtgtttccttctgctgcttggattccagttcgtctgcccacagccctccactcaacacaggaaggtcccgcagcggatggcggcggagggcgcccccgaggacgacggcggcggcggcgccccgggagtgtggggcgcctggggcccctggtcggcctgctcgcgtagctgcagcggcggcgtgatggagcagacgcggccctgcctgccccgctcctaccgcctgcgcggcggccagcggcctggcgcccctgcgcgcgccttcgcggaccacgtggtgtcggcggtgcgcacgtcggtgccactgcaccggagccgcgacgagacgccagcgctggccggtacggacgccagccgccagggccccacggtgctgcgaggcagccggcacccacagccccagggcctcgaagtcactggggacagaaggagcaggacccgtggtaccattggccctggcaagtatggctatggtaaggccccatatatcttaccactgcagacagacactgcacacacgccacagaggctccggagacagaagctctcatcccgccattccaggtcccagggagcatcttctgctaggcatggctacagttcaccagcccaccaggtcccccaacatgggcctttgtaccaaagtgacagtggccctcgctctggactgcaggctgcggaggcccccatctaccagctacctttgacccatgatcaaggctaccctgcagcttcaagtctctttcacagcccagaaacaagcaacaaccacggtgtggggacccatggggcaactcagagcttctctcagcctgcccgatctacagcaatctcatgcatcggggcctatcggcagtacaagctgtgcaacaccaacgtatgtccagaaagcagtagaagtatccgggaggtacagtgtgcatcctacaacaacaagccattcatgggccggttttatgagtgggaaccatttgcagaagtaaaaggcaatcgcaaatgtgagttgaactgccaggcaatgggctaccgcttctatgtacggcaagctgagaaagtcatcgatggcaccccctgtgaccagaacggcacggccatctgtgtgtctgggcagtgcaagagcattggctgtgatgactacttaggctccgacaaagtcgtggacaaatgtggggtgtgtggaggagacaacacgggctgtcaggttgtgtcgggcgtgtttaagcatgccctcaccagcctgggctaccaccgcgtcgtggagattcccgagggagccacgaaaatcaacatcacggagatgtacaagagcaacaactatttggccctgagaagtcgttctggacgctccatcatcaatgggaactgggcaattgatcgaccaggaaaatacgagggcggagggaccatgttcacctacaagcgtccaaatgagatttcgagcactgccggagagtcctttttggcggaaggtcccaccaacgagatcttggatgtctacatgatacaccagcagccaaacccaggcgtgcactacgagtacgtgatcatggggaccaacgccatcagcccccaggtgccaccccacaggagaccaggggaacccttcaatggccagatggtgacagaaggcaggagccaggaggagggagaacagaaagggaggaacgaggagaaggaagacttgcgtggggaggcccctgagatgttcacctcagaatcggcacagaccttcccagtcaggcatccagacagattttctccccatcgaccggacaacttggtgccaccagcaccgcagcccccacggcgcagccgggatcacaactggaagcagcttgggacaacagaatgttccacgacctgtgggaaaggatcgcagtaccctattttccgctgtgtgcacagaagcactcatgaagaggctcctgagagttactgtgactccagcatgaagccgacccccgaggaggagccctgcaacatcttcccttgcccagccttctgggacatcggggagtggtctgagtgcagcaagacctgtggcctgggcatgcagcaccgccaggttctgtgccgccaggtgtacgccaaccgcagcctgacggtgcagccctaccgctgccagcacctggagaaacctgagaccaccagcacctgccaactcaagatctgcagcgagtggcagatccggaccgactggacctcgtgctcggtgccctgtggcgtgggacagaggacccgtgatgtgaagtgtgtgagcaacattggggatgtggttgacgatgaggaatgcaacatgaagctccggccgaatgacattgagaactgcgacatgggaccctgtgccaagagctggttcctcaccgagtggagcgaaaggtgctcagcggagtgtggggccggagtgcggacacgctcggtggtgtgcatgaccaaccatgtcagcagcctgcccctggagggctgtgggaacaaccggccggcagaggccaccccatgtgacaacggaccctgcacgggcaaggtggagtggtttgccgggagctggagtcagtgttccatcgagtgtgggagcgggacgcaacagagggaggtgatttgtgttagaaagaatgcagacacctttgaagtgttggacccctctgaatgttctttcctggagaaaccccccagccagcaatcctgccacctcaagccttgcggagccaaatggtttagcaccgaatggagcatgtgttccaagagctgccagggtggctttcgggtccgggaagtgcggtgtctgtctgatgacatgactctaagtaacctctgtgaccctcagttgaaaccagaagagagagaatcttgtaaccctcaggactgtgtccctgaagttgatgaaaactgcaaggacaagtactacaactgcaacgtggtggtccaggcaagactctgtgtctacaactactacaagaccgcctgctgtgcctcctgcacccgtgtggccaacaggcagacgggcttcctggggagcagataacactcctgcacccccatcagtagggcagcatcactgccttcccgggggcttcagcagtgcgcctggctggctgctgctccaccacgggccccctggcccaggcgctgccaaccaacttagtcaccacccctgcctccggtgaatgcaccccgtggtacccaggggctttttacacaagatgtttgaaagccacagtcagtcctttaagcatcaccatgtactgatgatcccctccttggacctggcatctgctaatggtgccctttgaaagtcaagcagtgggaagtacatggagctctcagccctgctcccatctggcaccttcaagtcagcagatgggccactgactgagcactgccccgtccctggtgctactggtctttctaaacttagcaccctggagagtccaaggaggcagcgcccccaacccagcgccccactaagccttgctgacacgcgtgcatccctctgtgacctcagcccagatgtgcctgttttcattctcaaagacattagactgttttcctgccctatgacacagatagctcacatgaatattgtgctttatttagcaggtgtactcacagatactagctccttagcagctcacaacatcccagaatgggaggcagggggtgactcattatccccattttactgacagggaaactgaggctcaacttaagtaattgacctgccaggtatattcacccatccagtggaagagctgagtccccgccccagtcatctaccagtatccagcctggggcctgtacttagatgtgaaaggtgctgcttcatttctgaccaagagactgagaagtttcccagaatgcaaacaaagcccaggcccctgaaatctttccggtcaagcctttatcccagcactcagttgttttggatgtctgttcctacttgcccttacccccaaagttacagatcctagttacaggactctgccagctttgttaaactgtccgtgagacaagaaagccattggggaaaccaggtgattgcctgaaattcttactccgttccaagtgctgttcctcccaggaaatcaaaggccagggtccttatggccgtggagccttcccgaccacagagccaacttgtgaagcacacagctctgcagcctgggctctgccctgcctcagccgcctcccccacgctcttcaccacgttcctggagagtccggccaacctgtcccagccaaaacactgctgtattagaaaaagtctctttctggtctttctggttttgtttatgaatttccctctgtggccacaaattcctcccctcccccatgactcacagtccatatggcccacccccagacttgagcaccaagctctgcattaatgcagttggcctgcgacaaggagctgtggacccttccccatctcttccaattcactttccccaactatccagttccagaggccgcaggcctggaaggatgcagtgcatattgaaaggtggaccctctgaaaacagttaagaggaatatatgtatgttttgcccattaagaaaaaatggcaagctaaacaaatgttaaacttacagaaaatttgtcttatggtcctgagcatatttcccttttagagcaagcctggattcttagcaaagtgtttcccccatttgctcttttagctgacaaatctgccactgtgatgatggtttgcagcttttggaagcagtatggcaacctggcctgacatgctctttaggcttccactaacctggggctttcagaaattctatttggcctttctgtgggtagctttccagcttctcttctagggagccccaggcatcatttcccaaaagcatccccatctcctgattctcttggaactcctacagataagcatcctggcagaggcccaggctcccaaaccgacaaagtgaaaagagaccagagaggccaagcatattgactggtgctgttcagggcctgctcttttccactcaccacttgttttgctgcttgtcacgaggagagttgttcctgtatgtggctgctctcagatctttccaagcaagccagtcatttgaagaggttttcttttcatgctggagggcaggctaagatcaatgagtggaagagagaaaggctgttttagctcaagttaaaggaacaccttctagccatcaaagccgcccaacagaggcaagggccaccacacatgagagagcgctctgtccttaaagggaattctctgttgagtgggaggtgaacaccctggttcttccaactcaggaattctcgtggctgggctgggtcagtgatggctttgtctctttatgtctaaagtgccctatggctgctgaaggttacctaaccattctttaaaaggagaatgaccctccatgggaatggccagcctgccaactgtgcaattgaagaagacccgatggatcaaccccatgtctcccttggggagaaagtgcataaaccaggggtctcttttttttttttttcaacaaaccattgagctgttcttggagttcatctctggagaggttatacattattagaagtttgattattattatagtttgatcaatttatttgtcttagagatccaatttttactaattccctagttttttatttcagcatctgaatgtctttctccctagcacagtgcatacaatcagggccttgggtatttccagtgataactttccttggagaggatctaagaaaagcccagatttcggtagccatctccctccaaatatgtctctttctgctttcttagtgcccattatttccccttctcctttcttctgtcactgccatctccttcttggtcttcccattgttctttaactggctgtaatgtggaattgatatttacattttgatacggtttttttcttggcctgtgtacgggattgcctcatttcctgctctgaattttaaaattagatattaaagctgtcatatggtttcctcacaaaagtcaacaaagtccaaacaaaaatagtttgccgttttactttcatccattgaaaaaggaaattgtgcctcttgcagcctaggcaaaggacatttagtactatcgattctttccaccctcacgatgacttgcggttctctctgtagaaaagggatggcctaagaaatacaactaaaaaaacaaacaaaaacaccaaaagaaaaaaaaaagccatttaaagccagccactagagggagtcagttcagttccgtaaaggtatgctcagtgcccgctgcctgcaagctgttggggaccccagggagggcaaggcagcctgtccccgcccccagggaactagaacatgacaagaattctccgcactgtgcctacctgtccctttaacttacctctctggcccagagttcttggagggtaaaccttctatttctcttatgtactcatctacttattctcaaagtatttagcattcaacactcttttggctttaaaaagaatgggccttacaaagggacagaacacgagaagacacgagctaggtgtatttcatcaagtatgtggcacgagaaatccagatattaccaggacctgtctaaccaatgtggggttactttcatcggatg

SEQ ID NO: 63 - DNA, 4457 bp, Homo sapiens
  misc_feature: Incyte ID No 4755911CB1
atggggaaggagcaggagctggtgcaggcggtgaaggcggaggacgtagggaccgcgcagaggctgctgcagaggccgcggcccgggaaggccacgcgtagcctccctgggggccgccggagatggatggatgggcgtgtggaccagccgcgtgtgcggctgcgcacgtatagccgtgtcagtgtgtcagggcacctgtgcgggcacggacagggctctgcagagctcctgggttccaccaagaagatcaatgtcaacttccaggacccggatggggttgggtttggggtcaagggtcagctcccagcatcccctcgccccccaggcatgcggccgctgcactatgcggcctggcagggccggaaggagcccatgaagctggtgctgaaggcgggctcggccgtgaacatcccgtctgatgagggccacatccccctgcacctggcggcccagcatggtcactatgatgtgtctgagatgctgctacagcaccagtctaacccgtgcatggtggacaactcggggaagacgcccctggacctggcctgcgagttcggccgcgttggggtggtccagctgctcctcagcagcaatatgtgtgcggcgctgctggagccccggccgggagacgccaccgaccccaacggcaccagccctttgcacctcgcagctaaaaacggccacatcgacatcatcaggctcctcctccaagccggcatcgacattaaccgccagaccaagtccggcacggccctgcacgaggctgcgctctgcggaaagacagaggtggtgcggctgctgctggatagcgggatcaatgcccacgtgaggaacacctacagccagacagccctggacatcgtgcaccagttcaccacgtcccaggccagcagggagatcaagcagctgttgcgagaggcctcagcggccctgcaggtccgggcgaccaaggattattgcaacaattacgacctgaccagcctcaacgtgaaggcaggggacatcatcacagtcctcgagcagcatccggatggccggtggaagggctgcatccatgacaaccggacgggcaatgaccgggtgggctacttcccgtcctccctgggcgaggccattgtcaagcgagcaggttcccgagcaggcactgaaccaagcctgccccagggaagcagctcatcgggaccctctgcacccccagaggagatctgggtgctgaggaagccttttgcaggtggggaccgaagcggcagcattagcggcatggctggcggccggggcagcgggggtcacgccctacacgcgggctctgaaggcgtcaagctcctggcaacggtgctttcccagaagtccgtctctgagtccggcccgggggacagccccgccaagcctccggaaggctctgcaggtgtggcccggtcccagcctccagtggcccacgccgggcaggtctatggggagcagccgcccaagaagctggagccagcatcggagggcaagagctctgaggccgtgagccagtggctcaccgcgttccagctgcagctctacgcccccaacttcatcagcgccggctacgacctgcccaccatcagccgcatgactcccgaggacctcacggccattggtgtcaccaagccgggccaccggaagaagatcgcggcagagatcagcggcctaagcatccctgactggctgcctgagcacaaacccgctaacctggccgtgtggctgtccatgatcggcctggcccagtactacaaggtgttggtggacaatggctacgagaacattgatttcatcaccgacatcacctgggaggacctgcaggagatcggcatcaccaagctggggcaccagaagaagctgatgctcgctgtgaggaagctggcagagctgcagaaggctgaatacgccaagtatgaggggggccccctgcgccggaaggcgccccagtctcttgaagtgatggccatcgagtcgccgcccccgcctgagcccacaccggccgactgccagtcccctaaaatgaccaccttccaggacagcgagctcagtgacgagctgcaggctgccatgactggcccggctgaggtggggcccaccactgagaagccctccagccacctgccacccaccccgagggccaccacgcggcaggactccagcctgggtggtcgggcacggcacatgagcagctcgcaggagctgctgggagatgggccccctgggcccagcagccccatgtctcgaagccaggagtacctcctggatgagggccccgcccccggcaccccgcccagggaggcccggcccggccgccacggccacagcatcaagagggccagcgtgccccccgtgcctggcaagccacggcaggtcctcccaccaggcactagccacttcacgcccccccagacgcccaccaaaacccgaccaggctctccccaggcccttgggggacctcatggtccagccccagctacggccaaggtgaagcccaccccgcagctgctgccgccgacagagcgccccatgtcaccccgctccctgcctcagtcaccgacgcaccgcggctttgcctacgtgctgccccagcccgtggagggcgaggtggggccggctgccccggggcctgcgcccccacccgtgccgacggctgtgcccacactgtgcctgccccctgaggccgacgcggagccggggcggcccaagaagcgggcccacagcctgaatcgctatgcggcgtccgacagcgagccggagcgggacgagctgctggtgcctgcggctgccggcccctatgccacggtccagcggcgcgtgggccgcagccactcagtgagggcgcccgcaggtgccgacaagaacgtcaaccgcagccagtcctttgccgtgcggccccgaaagaaggggcccccgccgcccccacccaagcgctccagctcggccctggctagtgccaacctggcggatgagccggtgcctgacgccgagcctgaggatggcctgctgggggtccgggcacagtgccggcgggccagtgacctggccggcagcgtggacacgggtagtgccggcagtgtgaagagcatcgcggccatgctggagctgtcctccattgggggtgggggccgggctgcccgcaggcctcctgagggccaccccactccccgccctgccagcccagagccgggccgggtggccaccgtgctggcctcagtgaaacacaaagaggccatcgggcctggcggggaggtggtgaaccggcgccgcacgctcagcgggccagtcaccggacttctggccactgcccgccgggggcctggggagtcggcagacccaggcccctttgtggaggatggcactggccggcagcggcctcggggtccctccaagggcgaggcgggtgtcgaaggcccgcccttggccaaggtggaagccagcgccacactcaagaggcgcatccgggccaagcagaaccagcaggagaacgtcaagttcatcctgaccgagtctgacacggtcaagcgcaggcccaaggccaaggagcgggaggccgggcctgagccaccaccgccactgtccgtgtaccataatggcactggcaccgtgcgccgccgaccggcctcggagcaggctgggcctccggagctgcctccaccgcccccgcctgccgaacccccgcccaccgacctggcgcacctacccccattgcccccgcccgagggcgaagcccggaagccggccaagccgcctgtctctcccaagcccgtcctgacgcagcctgtgcccaagctccagggctcgcccacacccacctccaagaaggtgccgctgccaggccctggcagcccagaggtgaagcgcgcccacggcacgccaccgcccgtgtctcccaagccgccgccgccgcccacagcgcccaagcccgtcaaggcggtcgcggggctgccttcgggcagcgccggcccttcacccgcaccctcgcccgcgcgacagccgcccgccgccctcgccaagccgcccggtacgccgccctcgctgggcgccagccccgccaagcccccgtcccccggcgcgcccgcgctgcacgtgcccgccaagcccccgcgagccgccgccgccgccgccgccgccgccgccgcgccccccgccccgcccgaaggcgcctcgccaggggacagcgcccggcagaaactggaggagacaagcgcgtgcctggccgcggcgctgcaggcggtggaggagaagatccggcaggaggacgcgcagggcccgcgcgactcggcggcggaaaagagcactggcagcatcctggacgacatcggcagcatgttcgacgacctggccgaccagctggatgccatgctggagtgaacgccgcctggccgggccctcccgcgccgcccgggccctccccgcacactgacctatacctcaggatgggcgcgtctggg

SEQ ID NO: 64 - DNA, 1943 bp, Homo sapiens
  misc_feature: Incyte ID No 379766CB1
ggcggcggcgactgcggcgccgcgggctggaggccggcgtcggggaaggtcctggtgccggattccgcacgaggtgttgacgggcggcttctgccaacttctccccagcgcgcgccgagcccgcgcggccccggggctgcacgtcccagatacttctgcggcgcaaggctacaactgagacccggaggagactagaccccatggcttcctggacgagcccctggtgggtgctgatagggatggtcttcatgcactctcccctcccgcagaccacagctgagaaatctcctggagcctatttccttcccgagtttgcactttctcctcagggaagttttctggaagacacaacaggggagcagttcctcacttatcgctatgatgaccagacctcaagaaacactcgttcagatgaagacaaagatggcaactgggatgcttggggcgactggagtgactgctcccggacctgtgggggaggagcatcatattctctgcggagatgtttgactggaaggaattgtgaagggcagaacattcggtacaagacatgcagcaatcatgactgccctccagatgcagaagatttcagagcccagcagtgctcagcctacaatgatgtccagtatcaggggcgttactatgaatggcttccacgatataatgatcctgctgccccgtgtgcactcaagtgtcatgcacaaggacaaaacttggtggtggagctggcacctaaggtactggatggaactcgttgcaacacggactccttggacatgtgtatcagtggcatctgtcaggcagtgggctgcgatcggcaactgggaagcaatgccaaggaggacaactgtggagtctgtgccggcgatggctccacctgcaggcttgtacggggacaatcaaagtcacacgtttctcctgaaaaaagagaagaaaatgtaattgctgttcctttgggaagtcgaagtgtgagaattacagtgaaaggacctgcttatcctgtggcctgggctttagccatctcttccaataccaattgcctagtgttattatgtaaagctaatttggccagctctggtccttattttgcactcattccagtaaacccaaccactatggcacttaatactgccattgtcagtcagtctgcagtattgattgactgcctttagagctctcttttgtgtgccttgtccactcttcagtcactgagagagagaccaaagaacagagaccaacaccctgtacttggcatggccattagtcactggagttagatgaatcacactgtatctaagagagaagactcagggagaagggcttagcacaacacagaaaagctttaaacactcttacctttgactggaattacacacacacacacacacacacacacatacacacacacacatacacacacacacactaaggctttcccacaaagccatgatgcatccttaaaaataacacacagctctgaaaagtgaatgtcgggggtgaagagagccctcctacactccttttcctagtgatgacaaggttgtgggggcatggctgactgtgaggagcagaagatgagagggagatatcattttacttctttgtactgcaataataaaaagaacagatagaatggaaggaagaggccaggggcagtggctcatacctgtaatcccagcactttgggaggctgaggcaggtggatcacctgaggtcaggagttcgagaccagcctggccaacatggagaaactccgtctctattaagaatacaaaaattagccaggcgtggtggtgggcacctataatcacagctacccgggaggttgaggcaggagaatcacttgaacttgtggggcggaggtcgcagtgagccaagattgcaccacttcactccagcctgggcgagaaagtgaactctgtctcaaaaaaaaaaaaaaaaaa

SEQ ID NO: 65 - DNA, 4111 bp, Homo sapiens
  misc_feature: Incyte ID No 553744CB1
gcgatctagggcggggcaactgtacagatgaacaatctggaatattaaattcaactcacagagtggcaacaaacaatactggagagctgggcatgaatcctggaaagccttgtatggccctgcaagggaagccatgttctgaaagctcctatgagggggactgactgactctagataagtagattgaaagaattagaggtaaagtcagtgcagcagcactaatcacaattaaaataaatgctttaatttttaaaaagcggaaaaacatgaaaggacacttcgcaagcttctcaaaacttcctgtgatttggagggctattttgagcagagtctagacgaaaactggatctgactggctccagagtaaagatctgagaaatggaacccagcaatttagatattacaagagcctgttatacttgtcattttttatttggtatttgttaaatattacaaaaatggttatgctttttaattcaaaatttgacgtttcagcatgatgatacattcttgccttttttcccccttccatatagcattttccaccccagcaagtcaactcttttctcctcatggttctaatccttcaacacctgctgcaactcctgttcctactgcatccccagtcaaggcaattaatcatccatcagcatcagcagctgccaccgtttctggaatgaacctgctgaatactgtccttcctgtgttcccagggcaggtctcctcagccgttcacacacctcagccatcaataccaaacccaacagttatcagaaccccttcattgcccactgcacctgttacatccatccacagtacaaccaccactcctgttccttccattttttctggcctagtgtcactgccaggtccttctgccactcctaccgcagccactcctaccccaggacctacaccacggtccactcttggttccagtgaagcatttgcttctacttctgcacctttcactagcctccccttttccaccagctcttctgctgcttctaccagcaacccaaattctgcttcattgtcatcagtttttgcagggctccctttgcccttaccaccaacatcccaaggcctatccaacccgactcctgtaattgctggtggctctactcccagcgttgccggtccacttggtgtgaacagtccatcttttgtctgcgttaaaaggttttctgacatccaatgacaccaatttaatcaactcctctgctttatcctctgctgtcacaagtgggctggcttcactatcttctcttactcttcagaactctgactcttctgcttcagcccctaacaagtgctatgccccatcagccatccctaccccacagaggacttccactccagggttggccctgttcccaggcctgccgtctcccgtggctaactcaacttccactcccctgacattgcctgtacagtctcctttagccactgctgcatcagcttccacgtcagtgccagttagctgtggctcctcagcctcccttttgcgtggcccccacccaggtacctcagatctgcatatttcatctacccctgctgcaacaactcttcctgttatgatcaaaactgagcccacaagtcctactccctcggccttcaaaggtccatctcattctgggaatccctctcatggcactttaggtttgtcagggacattgggccgtgcatatacttcaacatccgtgcccatcagtttatctgcttgccttaatcctgcattgtcaggtctctccagcttgagtactcctttaaatggttcaaatcctctttcctctatttcccttccaccacatggttcctccactcccattgcaccagtattcactgctcttccttcttttacttctttgaccaacaattttcctttaactggcaacccatctcttaatccgtcagtatctctcccagggtcattaatagccacctcatctaccgctgccacctccacatctctccctcatcctagctcaacggcagctgttctctcagggctttctgcttcagcaccagtctcagcagcacctttccccctcaacctgtccactgctgttccctcacttttctctgttactcaaggacctctgtcatcttcaaatccctcctatccaggcttttctgtctctaataccccaagcgttacccctgctcttccctcattcccggggctgcaggcgccctctacagtcgcagctgtcacaccactacctgtggctgccacagccccatccccagctccagtcctcccaggattcgcctcagcattcagttccaatttcaactccgctcttgttgcacaagccggtttatcatctggacttcaagctgcaggcagttctgtttttccaggccttttgtccctcccgggtatccctgggtttcctcagaatccttcacaatcatccttgcaagaattacagcataatgcggctgcgcagtcagcattgttacagcaggtccattcagcttcggctctggaaagctatccagctcagcctgatgggtttcctagttatccttcagcgccaggaacaccattttctttgcaaccaagcctgtcccagagtgggtggcagtgaatacttttaacttttattctccttcagagcaacatcagaattgcctgagaactgcaatgaacaatctgacaaatgtgaagctggccaaaagtcggaaaatgagaatgagggtaatcctggagaaattgtgacaacaatttgaaaattgtggttgcattttaaagtgtgaacactcccctatgtaaatatgctgacaataaattgtatggagaatggtatttaaaaagtgtttggagacttttcacctgtcctataaaattttgaattgtgtatgtgatctacatagaaagaatattaaagagtaggttgaactctttatagccgaatacagccttaaatatgcttgtatagcatccactggcagaagtaatagttgtgcctcagacttgggggttgcatgtggccctgggggagttactacccttggtatgcatgagcggttcctattagcatcagtgggaactcagtactctgtatgtatccacaaaagggaacttgagacccacagttattcttaatttctgatattaacaaccgtacatactgctgaatttaactcaaaatatttcaggtaagtgaaagtggtgcttaatgtagactatagaatgactttcaggtgttttcaactgaaagtatatatccagaactgcatccttatagaaatacaagtaagacttaggataatttgccttcaaaacagttttcctaatctcagcagtatccagtgagtgaagaacacttgactgactcttgggccacctctgttacttactgtactatggaagctcctggtgaatgtttacaattatgggatgtagtatttctatttgtactttaagtcaaatgcttatatgaaatatgtgacaacaaatagagaagactggctctgttagtaattatgcagtatgtactctatttaaggatctgtggtagtataacatgagtgaatgtcattaattttgaagtaataactgccacatgtgggaagtaggggagtaaggagaatgaattccaatctgtgattaaaagtgtaaactatagactctactgtagtacatttcaggatctagaagttttacttttataaagatggtgtccggaagatgttgctaatgtattttacttcaacatagggaacaaactttttaagtatattaataaacctgtatggttagtttttaacagttttttaaaataaactatggatatgacaaatattctgtgttttactaagtgcttggataggctttctaattttgtatacgtgctagagttaattattgaacatttttatccaaatttagttgtaactctgtttatactactgattgctcattcgtttaaatgatattttaatgtaaaagtcataaccaacatatgaacagacagatttatgtctttaaacacagaatgtaagctatagtttaatctgataccagttgctggaagttgccatttgtttttcttaaatctatacccataaaacttcttttaagattaaaaaaaaaaaaaaa

SEQ ID NO: 66 - DNA, 1604 bp, Homo sapiens
  misc_feature: Incyte ID No 1825473CB1
gatcttaaatctaatagatttcctatttccaaaaagctcgactggagtgttataaaacctgaaaattctcttgtgctttctcttcttttgcttctagttaccatcctcaaaggattggctaaaagcaagcaactggattgaacaccctaagaagaaagattcacactgcaccaggagacatcagaaagaatgaaaactctgccgctgtttgtgtgcatctgtgcactgagtgcttgcttctcgttcagtgaaggtcgagaaagggatcatgaactacgtcacagaaggcatcatcaccaatcacccaaatctcactttgaattaccacattatcctggactgctagctcaccagaagccgttcattagaaagtcctataaatgtctgcacaaacgctgtaggcctaagcttccaccttcacctaataacccccccaaattcccaaatcctcaccagccacctaaacatccagataaaaatagcagtgtggtcaaccctaccttagtggctacaacccaaattccatctgtgactttcccatcagcttccaccaaaattactacccttccaaatgtgacttttcttccccagaatgccaccaccatatcttcaagagaaaatgttaacacaagctcttctgtagctacattagcaccagtgaattccccagctccacaagacaccacagctgccccacccacaccttctgcaactacaccagctccaccatcttcctcagctccaccagagaccacagctgccccacccacaccttctgcaactacacaagctccaccatcttcctcagctccaccagagaccacagctgccccacccacacctcctgcaactacaccagctccaccatcttcctcagctccaccagagaccacagctgccccacccacaccttctgcaactacaccagctccactatcttcctcagctccaccagagaccacagctgtcccacccacaccttctgcaactaccctagacccatcatccgcctcagctccaccagagaccacagctgccccacccacaccttctgcaactacaccagctccaccgtcttccccagctccacaagagaccacagctgccccaattaccacacctaattcttccccaactactcttgcacctgacacttctgaaacttcagctgcacccacacaccagactactacttcggtcactactcaaactactactactaaacaaccaacttcagctcctggccaaaataaaatttctcgatttcttttatatatgaagaatctactaaacagaattattgacgacatggtggagcaatagtatattgtatgttgtaaagtgttctgtcatttacaagatgtgattcatgagtgcagaactaccacctttcttttagcaccaatcccaacatgaaattatattactcagatttaaagcactatcattaatctttcaatctaattattcaccaccacaagacctattaacaagacaaaatgcctctatcccacaagccagatgcaggtctggggttcaaaataactctttggatcctacagagatagcctactgagggcagagaaagtccttagataaagaga

SEQ ID NO: 67 - DNA, 2646 bp, Homo sapiens
  misc_feature: Incyte ID No 7950094CB1
gcgagcgcgggcaggcggcgacgcgggggcaggggtggacggcggtcagagccgaacgcgagggcggcgcccggggactggagctgcgcgcaataggacagctggcctgaagctcagagccggggcgtgcgccatggccccacactgggctgtctggctgctggcagcaaggctgtggggcctgggcattggggctgaggtgtggtggaaccttgtgccgcgtaagacagtgtcttctggggagctggccacggtagtacggcggttctcccagaccggcatccaggacttcctgacactgacgctgacggagcccactgggcttctgtacgtgggcgcccgagaggccctgtttgccttcagcatggaggccctggagctgcaaggagcgatctcctgggaggcccccgtggagaagaagactgagtgtatccagaaagggaagaacaaccagaccgagtgcttcaacttcatccgcttcctgcagccctacaatgcctcccacctgtacgtctgtggcacctacgccttccagcccaagtgcacctacgtcaacatgctcaccttcactttggagcatggagagtttgaagatgggaagggcaagtgtccctatgacccagctaagggccatgctggccttcttgtggatggtgagctgtactcggccacactcaacaacttcctgggcacggaacccattatcctgcgtaacatggggccccaccactccatgaagacagagtacctggccttttggctcaacgaacctcactttgtaggctctgcctatgtacctgagagtgtgggcagcttcacgggggacgacgacaaggtctacttcttcttcagggagcgggcagtggagtccgactgctatgccgagcaggtggtggctcgtgtggcccgtgtctgcaagggcgatatggggggcgcacggaccctgcagaggaagtggaccacgttcctgaaggcgcggctggcatgctctgccccgaactggcagctctacttcaaccagctgcaggcgatgcacaccctgcaggacacctcctggcacaacaccaccttctttggggtttttcaagcacagtggggtgacatgtacctgtcggccatctgtgagtaccagttggaagagatccagcgggtgtttgagggcccctataaggagtaccatgaggaagcccagaagtgggaccgctacactgaccctgtacccagccctcggcctggctcgtgcattaacaactggcatcggcgccacggctacaccagctccctggagctacccgacaacatcctcaacttcgtcaagaagcacccgctgatggaggagcaggtggggcctcggtggagccgccccctgctcgtgaagaagggcaccaacttcacccacctggtggccgaccgggttacaggacttgatggagccacctatacagtgctgttcattggcacaggagacggctggctgctcaaggctgtgagcctggggccctgggttcacctgattgaggagctgcagctgtttgaccaggagcccatgagaagcctggtgctatctcagagcaagaagctgctctttgccggctcccgctctcagctggtgcagctgcccgtggccgactgcatgaagtatcgctcctgtgcagactgtgtcctcgcccgggacccctattgcgcctggagcgtcaacaccagccgctgtgtggccgtgggtggccactctggatctctactgatccagcatgtgatgacctcggacacttcaggcatctgcaacctccgtggcagtaagaaagtcaggcccactcccaaaaacatcacggtggtggcgggcacagacctggtgctgccctgccacctctcctccaacttggcccatgcccgctggacctttgggggccgggacctgcctgcggaacagcccgggtccttcctctacgatgcccggctccaggccctggttgtgatggctgcccagccccgccatgccggggcctaccactgcttttcagaggagcagggggcgcggctggctgctgaaggctaccttgtggctgtcgtggcaggcccgtcggtgaccttggaggcccgggcccccctggaaaacctggggctggtgtggctggcggtggtggccctgggggctgtgtgcctggtgctgctgctgctggtgctgtcattgcgccggcggctgcgggaagagctggagaaaggggccaaggctactgagaggaccttggtgtaccccctggagctgcccaaggagcccaccagtccccccttccggccctgtcctgaaccagatgagaaactttgggatcctgtcggttactactattcagatggctcccttaagatagtacctgggcatgcccggtgccagcccggtggggggcccccttcgccacctccaggcatcccaggccagcctctgccttctccaactcggcttcacctggggggtgggcggaactcaaatgccaatggttacgtgcgcttacaactaggaggggaggaccggggagggctcgggcaccccctgcctgagctcgcggatgaactgagacgcaaactgcagcaacgccagccactgcccgactccaaccccgaggagtcatcagtatgaggggaaccccc

SEQ ID NO: 68 - DNA, 3876 bp, Homo sapiens
  misc_feature: Incyte ID No 7479484CB1
atgttccgaccaaccacggtggctgtagacgaggatggtggagaagaggataaagatgagtcatcaaccaacagtggtgcaagtgctgtttcttcttgtggctttggagccgacttctccacagataaagggggctccttcacgtcagtacagatcactaataccactggactgtcacaggctcctggcttagcctcccaaggtattagctttggcattaagaataatctgggacccccactgcagaaattgggagtatcattttcctttgccaagaaagctcccgtcaaacttgaatcaatagcatccgttttcaaggaccatgcagaggaagggagctcagaagatggaacgaaggctgatgagaagagctctgaccaaggggtgcagaaggtgggagatactgatggcactggtaatcttgatggaaagaaagaagatgaagaccctcaggatggagggtcccttgcctcaacactgtccaagttgaaaaggatgaaacgggaagaaggaacaggggctacagagccagaatattaccactacatccccccagcacactgcaaggtcaaacctaatttccccttcttactctttatgagagccagtgaacagatggaaggggatcatagtgcacactcaaagagtgcccccgagaacagaaaaagcagctctcccaagccgcaaggctgtagtaagacagcagcaagcccaggggcagaaagaacagtgagtgaagcttctgagctgcaaaaggaagccgctgtggctgggccttcagagcctggaggtaaaactgaaacaaagaaaggctccggaggaggggaagatgagcagagtgtagagagtagggagacgtcagagagcccgatgtgtgagtccaatcctaaagacatttctcaggccaccccagcaacaaaagcaggccagggacccaagcatcctactggtccattctttccagttttaagcaaggatgaaagcactgccctccagtggccatcagaactactcattttcaccaaagcagagccttccatctcctacagttgtaatcctttatactttgactttaaactttcaagaaacaaagatgctaaagctaaagggacagaaaagccaaaagatgtcgcaggctcctcaaaggatcatctccagagccttgatcctagagaaccgaataaaagccaggaagaggagcaggatgtagtgctctcttcagaaggcagagtggatgaacctgcatcaggggctgcctgtagcagcctgaacaagcaggagcctgggggtagccatatgtcagaaactgaagacactgggagaagccatcctagcaagaaagaaccatcaggcaagtctcacagacacaagaagaaaaagaaacacaaaaaatccagcaagcacaaacgtaaacacaaggctgacacggaagagaagagttctaaggcagagtctggggagaaatctaagaagcgcaagaaacgaaaacggaagaagaacaaatcatcagccgcagctgattctgaacgcggacccaaatcagaacctcctggaagcggcagcccggcaccaccgagaaggcggcgccgagctcaagatgattcccagcggagatcccttcctgctgaagaaggaaacagtggcaagaaggatgatggtgggggtggtagcagttgccaagatcacagtgggaggaaacacaaaggtgaaccaccaacttcctcctgccagcggagagctaacaccaaacatagcagccggtccagccatcggagccaacccagtagtggtgatgaggatagtgatgatgcttcctcacaccgactgcaccagaagtctccatcccagtacagtgaggaggaggaagaggaggaggaggaagaagaggaggaagatgaagactccggtagtgagcattctcgtagccgctctcggtctggccatcgccattcctcacatcgttcctcccggcgctcttattctagcagctctgatgcctcttcagaccagagctgctatagtagacagcacagttactctgatgatagctatagtgactatagcgaccgatcacgaaggcactctaagcgctctcacgattcagatgattcagactataccagctccaaacacaggtctaaacgacacaaatactcatcatctgatgatgactatagcctcagttgcagccagtcccgaagccgatctcggagtcatacaagggagcgatcaagatcccggggtcgaagccgcagtagcagctgtagtcgcagtcgaagcaagaggagaagtcgcagcaccacagcccacagctggcagcgaagccgaagctatagccgggaccggagccgcagcaccaggagcccttctcagagatcaggctccagaaagggctcatggggtcatgagagcccagaggaaaggcgctctggccgccgggatttcattcgttcaaagatctaccgctctcaatccccccactatttccaatcaggtcggggagaaggtcctggaaagaaagaagatggcagaggagatgacagtaaaggagcaggcctgccctcccagaatagcaatactggcacaggaagggggtcagaaagtgactgcagtcctgaagataagaattctgttactgccagactgctgctagagaagatccagtccaggaaagtggagaggaaacccaatgtgtgcgaggaggtgctggccacccctaataaggctgggctcaagtacaagaaccccccacaaggttactttgggcctaagctccccccgtctcttggtaataagcctgttcttccaatgatagggaagcttccagctacccggaagtccaataagaaatgtgaagagtctggcttagaaaggggagaagagcaggaacattcagagccagaagaagggtccccaaggagtagtgatgctccatttgggcatcagttctcagaggaagcagctggtcccttatcagaccctcccccagaagagccaaagtctgaagaagctactgctgatcactctgtggctccgctaggcaccccagcccacactgactgctaccctggggatccagccatctcccataactacctcccggaccccagtgatggggatactctggagtctctggatagtggcagtcaaccaggccctgtggaatccagcttgctgcctatagccccagaccttgagcacttccccaattatgcacctcccagtggggaacctagtattgaatcaacagatgggactgaggatgcctccttggctcctctcgagagccagcccatcaccttcacccctgaggagatggagaagtacagcaagctccagcaggctgcacagcagcacatccagcagcagcttctggccaaacaagtgaaggcctttccagcctccaccgccctagctccagccacaccagccctgcagcccatccacattcagcagccagccacagcctctgccacctccatcaccactgttcagcatgccatcctacagcatcatgctgcagccgctgctgccgccattggcattcaccctcacccccaccctcagccgcttgctcaagtacatcatattccccagccccatctaacccctatttctttgtcccatctcactcactcaattatccctggccaccctgccacctttcttgctagccaccctatccatataattcctgcctcagccatccatcctgggcccttcacctttcatcccgtcccacacgctgccctctaccccaccctgcttgccccacggcctgctgcagcagctgccacagccctccatcttcacccactgcttcaccccatcttctcaggtcaggacctgcagcaccctcccagccatgggacttga

SEQ ID NO: 69 - DNA, 2583 bp, Homo sapiens
  misc_feature: Incyte ID No 6780147CB1
ggtggcgggtggctggcggttccgttaggtctgagggagcgatggcggtacgcgcgttgaagctgctgaccacactgctggctgtcgtggccgctgcctcccaagccgaggtcgagtccgaggcaggatggggcatggtgacgcctgatctgctcttcgccgaggggaccgcagcctacgcgcgcggggactggcccggggtggtcctgagcatggaacgggcgctgcgctcccgggcagccctccgcgcccttcgcctgcgctgccgcacccagtgtgccgccgacttcccgtgggagctggaccccgactggtcccccagcccggcccaggcctcgggcgccgccgccctgcgcgacctgagcttcttcgggggccttctgcgtcgcgctgcctgcctgcgccgctgcctcgggccgccggccgcccactcgctcagcgaagagatggagctggagttccgcaagcggagcccctacaactacctgcaggtcgcctacttcaagatcaacaagttggagaaagctgttgctgcagcacacaccttcttcgtgggcaatcctgagcacatggaaatgcagcagaacctagactattaccaaaccatgtctggagtgaaggaggccgacttcaaggatcttgagactcaaccccatatgcaagaatttcgactgggagtgcgactctactcagaggaacagccacaggaagctgtgccccacctagaggcggcgctgcaagaatactttgtggcctatgaggagtgccgtgccctctgcgaagggccctatgactacgatggctacaactaccttgagtacaacgctgacctcttccaggccatcacagatcattacatccaggtcctcaactgtaagcagaactgtgtcacggagcttgcttcccacccaagtcgagagaagccctttgaagacttcctcccatcgcattataattatctgcagtttgcctactataacattgggaattatacacaggctgttgaatgtgccaagacctatcttctcttcttccccaatgacgaggtgatgaaccaaaatttggcctattatgcagctatgcttggagaagaacacaccagatccatcggcccccgtgagagtgccaaggagtaccgacagcgaagcctactggaaaaagaactgcttttcttcgcttatgatgtttttggaattccctttgtggatccggattcatggactccagaagaagtgattcccaagagattgcaagagaaacagaagtcagaacgggaaacagccgtacgcatctcccaggagattgggaaccttatgaaggaaatcgagacccttgtggaagagaagaccaaggagtcactggatgtgagcagactgacccgggaaggtggccccctgctgtatgaaggcatcagtctcaccatgaactccaaactcctgaatggttcccagcgggtggtgatggacggcgtaatctctgaccacgagtgtcaggagctgcagagactgaccaatgtggcagcaacctcaggagatggctaccggggtcagacctccccacatactcccaatgaaaagttctatggtgtcactgtcttcaaagccctcaagctggggcaagaaggcaaagttcctctgcagagtgcccacctgtactacaacgtgacggagaaggtgcggcgcatcatggagtcctacttccgcctggatacgcccctctacttttcctactctcatctggtgtgccgcactgccatcgaagaggtccaggcagagaggaaggatgatagtcatccagtccacgtggacaactgcatcctgaatgccgagaccctcgtgtgtgtcaaagagcccccagcctacaccttccgcgactacagcgccatcctttacctaaatggggacttcgatggcggaaacttttatttcactgaactggatgccaagaccgtgacggcagaggtgcagcctcagtgtggaagagccgtgggattctcttcaggcactgaaaacccacatggagtgaaggctgtcaccagggggcagcgctgtgccatcgccctgtggttcaccctggaccctcgacacagcgagcgggacagggtgcaggcagatgacctggtgaagatgctcttcagcccagaagagatggacctctcccaggagcagcccctggatgcccagcagggcccccccgaacctgcacaagagtctctctcaggcagtgaatcgaagcccaaggatgagctatgacagcgtccaggtcagacggatgggtgactagacccatggagaggaactcttctgcactctgagctggccagcccctcggggctgcagagcagtgagcctacatctgccactcagccgaggggaccctgctcacagccttctacatggtgctactgctcttggagtggacatgaccagacaccgcaccccctggatctggctgagggctcaggacacaggcccagccacccccaggggcctccacaggccgctgcataacagcgatacagtacttaagtgtctgtgtagacaaccaaagaataaatgattcatggttttttttaaaaaaaaa

SEQ ID NO: 70 - DNA, 6147 bp, Homo sapiens
  misc_feature: Incyte ID No 7204554CB1
tgatatatagggccatgcttatctagcatgcatgctcgagacgcgcgcatatgtgctggaaagggcggggcgcgccccggggcggcggggctgaagctcctggcaccatgatgctcaccccagcaggaccagagcaccgaggcccaaggccccagcctgccatgccgctgccaccgcggagcctgcaggtgctcctgctgctgctgctgttgctgctgctgctgccgggcatgtgggctgaggcaggcttgcccagggcaggcgggggttcacagccccccttccgcaccttctcggccagcgactggggcctcacccacctagtggtgcatgagcagacaggcgaggtgtatgtgggcgcagtgaaccgcatctataagctgtcggggaacctgacactgctgcgggcccacgtcacgggccctgtggaggacaacgagaagtgctacccgccgcccagcgtgcagtcctgcccccacggcctgggcagtactgacaacgtcaacaagctgctgctgctggactatgccgctaaccgcctgctggcctgtggcagcgcctcccagggcatctgccagttcctgcgtctggacgatctcttcaaactgggtgagccacaccaccgtaaggagcactacctgtccagcgtgcaggaggcaggcagcatggcgggcgtgctcattgccgggccaccgggccagggccaggccaagctcttcgtgggcacacccatcgatggcaagtccgagtacttccccacactgtccagccgtcggctcatggccaacgaggaggatgccgacatgttcggcttcgtgtaccaggatgagtttgtgtcatcacagctcaagatcccttcggacacgctgtccaagttcccggcctttgacatctactatgtgtacagcttccgcagcgagcagtttgtctactacctcacgctgcagctagacacacagctgacctcgcctgatgccgccggcgagcacttcttcacgtccaagatcgtgcggctctgtgtggacgaccccaaattctactcgtacgttgagttccccattggctgcgagcaggcgggtgtggagtaccgcctggtgcaggatgcctacctgagccggcccggccgtgccctggcccaccagctgggcctggctgaggacgaggacgtgctgttcactgtgttcgcccagggccagaagaaccgcgtgaagccaccaaaggagtcagcactgtgcctgttcacgctcagggccatcaaggagaagattaaggagcgcatccagtcctgctaccgtggtgagggcaagctctccctgccgtggctgctcaacaaggagctgggctgcatcaactcgcccctgcagatcgatgacgacttctgcgggcaggacttcaaccagcccctggggggcacagtcaccattgaggggacgcccctgttcgtggacaaggatgatggcctgaccgccgtggctgcctatgactatcggggccgcactgtggtattcgccggcacgcgaagtggccgcatccgcaagatcctggtggacctctcaaaccccggtggccggcctgccctggcctacgagagcgtcgtggcccaggagggcagccccatcctgcgagacctcgtcctcagccccaaccaccagtacctctacgccatgaccgagaagcaggtgacgcgggtgcctgtggagagctgtgtgcagtacacgtcctgtgagctgtgtctggggtcacgggacccccactgtggctggtgtgtcctgcacagcatctgctcgcggcgggacgcctgtgagcgagcagacgagccccagcgctttgctgcggacctgctgcagtgtgtgcagctgactgtgcagccccgcaatgtgtctgtcaccatgtcccaggtcccacttgtgctgcaggcctggaacgtgcctgacctctcagctggcgtcaactgctccttcgaggacttcacggaatctgagagcgtcctggaggatggccggatccactgccgctcaccctccgcccgggaggtggcgcccatcacgcggggccagggagaccagcgggtggtgaaactctacctaaagtccaaggagacagggaagaagtttgcgtctgtggacttcgtcttctacaactgcagcgtccaccagtcctgcctgtcctgtgtcaacggctcctttccctgccactggtgcaaataccgccacgtgtgcacacacaacgtggctgactgcgccttcctggagggccgtgtcaacgtgtctgaggactgcccacagatcctgccctccacgcagatctacgtgccagtgggagtggtaaaacccatcaccctggccgcacggaacctgccacagccacagtcaggccagcgtggatatgagtgcctcttccacatcccgggcagcccggcccgtgtcaccgccctgcgcttcaacagctccagcctgcagtgccagaattcctcgtactcctacgaggggaacgatgtcagcgacctgccagtgaacctgtcagtcgtgtggaacggcaactttgtcattgacaacccacagaacatccaggcgcacctctacaagtgcccggccctgcgcgagagctgcggcctctgcctcaaggccgacccgcgcttcgagtgcggatggtgcgtggccgagcgccgctgctccctgcgacaccactgcgctgccgacacacctgcatcgtggatgcacgcgcgtcacggcagcagtcgctgcaccgaccccaagatcctcaagctgtcccccgagacgggcccgaggcagggcggcacgcggctcactatcacaggcgagaacctgggcctgcgattcgaagacgtgcgtctgggcgtgcgcgtgggcaaggtgctgtgcagccctgtggagagcgagtacatcagtgcggagcagatcgtctgtgagatcggggacgccagctccgtgcgtgcccatgacgccctggtggaggtgtgtgtgcgggactgctcaccacactaccgcgccctgtcacccaagcgcttcaccttcgtgacaccaaccttctaccgtgtgagcccctcccgtgggcctctgtcagggggcacctggattggcatcgagggaagccacctgaacgcaggcagtgatgtggctgtgtcggtcggtggccggccctgctccttctcctggaggaactcccgtgagatccggtgcctgacaccccccgggcagagccctggcagcgctcccatcatcatcaacatcaaccgcgcccagctcaccaaccctgaggtgaagtacaactacaccgaggaccccaccatcctgaggatcgaccccgagtggagcatcaacagcggtgggaccctcctgacggtcacaggcaccaacctggccactgtccgtgaaccccgaatccgggccaagtatggaggcattgagagggagaacggctgcctggtgtacaatgacaccaccatggtatgccgcgccccgtctgtggccaaccctgtgcgcagcccaccagagctgggggagcggccggatgagctgggcttcgtcatggacaacgtgcgctccctgcttgtgctcaactccacctccttcctctactaccctgaccccgtactggagccactcagccccactggcctgctggagctgaagcccagctccccactcatcctcaagggccggaacctcttgccacctgcacccggcaactcccgactcaactacacggtgctcatcggctccacaccctgtaccctcaccgtgtcggagacgcaactgctgtgcgaggcgcccaacctcactgggcagcacaaggtcacggtgcgggcaggtggcttcgagttctcgccagggacactgcaggtgtactcggacagcctgctgacgctgcctgccattgtgggcattggcggaggcgggggtctcctgctgctggtcatcgtggctgtgctcatcgcctacaagcgcaagtcacgagatgctgaccgcacactcaagcggctgcagctccagatggacaacctggagtcccgcgtggccctcgaatgcaaggaagcctttgcagagctgcagacagacatccacgagctgaccaatgacctggacggtgccggcatccccttccttgactaccggacatatgccatgcgggtgctctttcctgggatcgaggaccaccctgtgctcaaggagatggaggtgcaggccaatgtggagaagtcgctgacactgttcgggcagctgctgaccaagaagcacttcctgctgaccttcatccgcacgctggaggcacagcgcagcttctccatgcgcgaccgcgggaatgtggcctcgctcatcatgacggccctgcagggcgagatggaatacgccacaggcgtgctcaagcagctgctttccgacctcatcgagaagaacctggagagcaagaaccaccccaagctgctactgcgccggactgagtcggtggcagagaagatgctaactaactggttcaccttcctcttgtataagttcctcaaggagtgcgctggggagccgctgttcatgctgtactgcgccatcaagcagcagatggagaagggccccattgacgccatcacgggtgaggcacgctactccctgagtgaggacaagctcatccggcagcagattgactacaagacactgaccctgaactgtgtgaaccctgagaatgagaatgcacctgaggtgccggtgaaggggctggactgtgacacggtcacccaggccaaggagaagctgctggacgctgcctacaagggcgtgccctactcccagcggcccaaggccgcggacatggacctggagtggcgccagggccgcatggcgcgcatcatcctgcaggacgaggacgtcaccaccaagattgacaacgattggaagaggctgaacacactggctcactaccaggtgacagacgggtcctcggtggcactggtgcccaagcagacgtccgcctacaacatctccaactcctccaccttcaccaagtccctcagcagatacgagagcatgctgcgcacggccagcagccccgacagcctgcgctcgcgcacgcccatgatcacgcccgacctggagagcggcaccaagctgtggcacctggtgaagaaccacgaccacctggaccagcgtgagggtgaccgcggcagcaagatggtctcggagatctacttgacacggctactggccaccaagggcacactgcagaagtttgtggacgacctgtttgagaccatcttcagcacggcacaccggggctcagccctgccgctggccatcaagtacatgttcgacttcctggatgagcaggccgacaagcaccagatccacgatgctgacgtgcgccacacctggaagagcaactgcctgcccctgcgcttctgggtgaacgtgatcaagaacccacagtttgtgttcgacattcacaagaacagcatcacggacgcctgcttgtcggtggtggcccagaccttcatggactcctgctccacctctgagcacaagctgggcaaggactcaccctccaacaagctgctctacgccaaggacatccccaactacaagagctgggtggagaggtactatgcagacatcgccaagatgccagccatcagcgaccaggacatgagtgcgtatctggctgagcagtcccgcctgcacctgagccagttcaacagcatgagcgccttgcacgagatctactcctacatcaccaagtacaaggatgagatcctggcagccctggagaaggatgagcaggcgcggcggcagcggctgcggagcaagctggagcaggtggtggacacgatggccctgagcagctgagccccagctgtgatcatccagcatgatgcagcgtgaggacagctgagcagggaccgggacagccctcaccgcatgcgtgtggagtgtccggtggtgctcgggccgccgcagtgcagcgactgcccggccctccctcccctgcctcacccggtcgggtcccggctcttcctgtgtggaggtgatggtacctgccacaccacagctgcgcacacagctgcttgctcaggggccgggacagcactgggtgctcaggctggccaaggaccttcattgcctgggcaagagctgcccagtg

SEQ ID NO: 71 - DNA, 888 bp, Homo sapiens
  misc_feature: Incyte ID No 6833247CB1
cgagcttacttatactggtacctttctaatctcactacaatatgtaacattggtgttcgatctcaagtatttctgaatatattcccctatccacagaaatatactctgggggaaaaaaaatagaacaaattcttgccgtcctgaccattgaacaagagactaattagacaatggggctagaaaaacctcaaagtaaactggaaggaggcatgcatccccagctgataccttcggttattgctgtagttttcatcttacttctcagtgtctgttttattgcaagttgtttggtgactcatcacaacttttcacgctgtaagagaggcacaggagtgcacaagttagagcaccatgcaaagctcaaatgcatcaaagagaaatcagaactgaaaagtgctgaagggagcacctggaactgttgtcctattgactggagagccttccagtccaactgctattttcctcttactgacaacaagacgtgggctgagagtgaaaggaactgttcagggatgggggcccatctgatgaccatcagcacggaagctgagcagaactttattattcagtttctggatagacggctttcctatttccttggacttagagatgagaatgccaaaggtcagtggcgttgggtggaccagacgccatttaacccacgcagagtattctggcataagaatgaacccgacaactctcagggagaaaactgtgttgttcttgtttataaccaagataaatgggcctggaatgatgttccttgtaactttgaagcaagtaggatttgtaaaatacctggaacaacattgaactagaaactcagaaagtggtccttgtgatggaaagagaaaagaaaaaccaattagaataaggcagaatgtacgt

SEQ ID NO: 72 - DNA, 3582 bp, Homo sapiens
  misc_feature: Incyte ID No 4148119CB1
ctggccacaccgaaagagagcccacccaagtctgaggaaccacacccggagcagttccgaaaagcgagggagacttcccctgatctggaaagggtgccaggcacttcagcaccatcggcagccccagcccctcacccacggcaagagaggcagagcggctctcaccgggtccagaccggcccagcccaccataatggtcaaaagaaggccaagctctggaaggtgggaacaactgaggcagatggggacagtaaagccagatggaccgcgcttgagccttcatctccaccagggacccaggccaccttcccagttccatacctcttcagcctcaggtccctcttgactgagcccagcgtcaccgcccccaagaaagttccccaatcaccctgtcccacgctgggccacctgcttccctccctgaaggctgtccctaagctgtgggtatttatcctactaaggaggctgggacagagtcctgacctgcacccatctgttcccaacccaccagccttcaaacaagggggctcctcctccccggggcagctctggcctctgctgggggccagtcgaggcagatagaagggtgagcctaaccaatgacccaacgcccccttcccaattaactaccgccttccaacggaatccaagggaatccctcatccccaaaacttcaccgcggaaggatctgcggggactgacaggcagaagccaggcacagggatatccgaacagcctcagtcttgctacccaactctgccttcaagacattccaatctgatgggaagagtcctgtctgggaagctccctgtctgatgggagacagccctgtccacatggaggtctcagtctgacggaggaaacagcctggccagccaggcccaggccgacaggggagacacagtccctgcccaaggagcttccaagctaagggcggaaccacagccaagcccagggagctcccaggctaagggcggagactgtcccagcccagggagctcccagtcaaaagggggagacacagcactgcccttacaaagctaccagcctcacggagaaggcgcagtccctgtccacagagacacagccttgccccagttactgcccacctgcaagagatccacatttctgccctccagagctcccaaactgatgggggagagagacatcatcccccagctctgggagtttccagtctgatggaggagacagagcctgcttcggtaactctcactctgcgggggaagactcagccctatccagggagctcccacactttcaatggggaggcccacccagtggccgagcctgctgctgctcctgctgttgccggggcccccgcccgtcgccggcttggaagacgctgccttcccccacctgggggagagcttgcagcccctgccccgggcctgtcccctgcgctgctcctgcccccgagtcgacactgtggactgtgatggcttggaccttcgagtgttcccggacaacatcaccagagccgctcagcacctctccctgcagaacaaccagctccaggaactcccctacaatgagctgtcccgcctcagtggcctgcgaaccctcaacctccacaacaacctcatctcctccgaaggcctgcctgacgaggccttcgagtccctcacccagctgcagcacctctgcgtggctcacaacaagctctcagtggcccctcagtttctgccccggtccctccgtgtcgcggatctggctgccaaccaagtgatggagatcttccccctcacctttggggagaagccggtactcaggtccgtgtacctccacaacaaccagctgagcaacgctggcctgccccccgacgccttccgcggctccgaggccatcgccaccctcagcctctccaacaaccagctcagctacctgccgcccagcctgccgccctcactcgagcggctccacctgcagaacaatctcatctccaaggtgccccgaggagccctgagccgccagactcaactccgtgagctctacctccagcacaaccagctgacagacagtggcctggatgccaccaccttcagcaagctgcatagccttgaatacctggatctctcccacaaccagctgaccacagtgcccgccggcctgccccggaccctggctatcctgcacctgggccgcaaccgcatccggcaggtggaggcggctcggctgcacggggcgcgtggtctgcgctatttgttgctgcagcacaaccagctggggagctcagggctgcccgccggggctctgcggccgctgcggggcctgcacacgctgcacctctatggcaatgggctggaccgcgtgcctccagccctgccccgccgcctgcgtgccctggtgctgccccacaaccacgtggccgcgctgggtgcccgtgacctggtcgccacaccgggcctgacggagcttaacctggcctataaccgcctggccagcgcccgtgtgcaccaccgggccttccgccggttgcgtgccctgcgcagcctcgacctggcagggaatcagctaacccggctgcccatgggcctgcccactggcctgcgcaccctgcagctgcaacgcaaccagctgcggatgctcgagcccgagcctctggccggcctggaccaactgcgggagctcagcctggcgcacaaccggctccgggtcggcgacatcgggccaggcacctggcatgagctccaagccctccagatgctggacctcagccacaatgagctgtcctttgtgcccccggacctgcctgaggccctagaggagctgcacctcgagggcaaccgcatcggccacgtgggccccgaggccttcctcagcacaccccgcctgcgtgccctcttcctcagggccaacaggcttcacatgacgagcatcgcggctgaggccttcctggggctcccaaacctgcgtgtggtggacacggcagggaatccggagcaggtcctgatccggctgcctcccaccaccccacgtgggccacgggcagggggcccctgatcctagagaggcccagcagagcagctcagactcctgggactccgctgggccgtggactgaggagacaacgcccaccaggggcccttggtctggctctcctgggcctccagggctgggcctgctctgcctgccactggccgagacacagaggcacacagctggcatactccaggctcacagaccacgccggcctggcgggacacaccctaccccaaactcccaacacagatggaggcagcaacaataaagccaaacccttccagcactcagcacggaccaggcacccttcgggggctctgtccacggactcctccccacaaccagtccagctggggaaactgaggctctgggatgctaagtgggtcaggactgaattttgaggtcttgaggcacacactggggtcaccaaacagcaccctgtgcgacctagccacgtgtgattgcagggacgcccaaggccacccactgaaaaaacactgggtgacagatatagggaccctcacatgtatccccccccacagcaagcatgggaatgaaatgcatccttcaaaaaaaaaaaaa

(19) United States
(12) Patent Application Publication
(10) Pub. No.: US 2004/0053824 A1
(43) Pub. Date: Mar. 18, 2004

(54) Extracellular matrix and cell adhesion molecules

(76) Inventors: Y Tom Tang, San Jose, CA (US); Henry Yue, Sunnyvale, CA (US); Yalda Azimzai, Oakland, CA (US); Ann He, San Jose, CA (US); Terence P Lo, Foster City, CA (US); Daniel B Nguyen, San Jose, CA (US); John D Burrill, Redwood City, CA (US); Gregory A Marcus, San Carlos, CA (US); Kurt A Zingler, San Francisco, CA (US); Ameena R Gandhi, San Francisco, CA (US); Liam Kearney, San Francisco, CA (US); Neil Burford, Durham, CT (US); Monique G Yao, Carmel, IN (US); Narinder K Chawla, Union City, CA (US); Vicki S Elliott, San Jose, CA (US); Chandra S Arvizu, San Jose, CA (US); Mariah R Baughn, San Leandro, CA (US); April J A Hafalia, Santa Clara, CA (US); Jennifer L Policky, San Jose, CA (US); Janice K Au-Young, Brisbane, CA (US); Yan Lu, Mountain View, CA (US); Mark L Borowsky, Redwood City, CA (US); Dyung Aina M Lu, San Jose, CA (US); Jayalaxmi Ramkumar, Fremont, CA (US); Junming Yang, San Jose, CA (US); Rajagopal Gururajan, San Jose, CA (US); Bridget A Warren, Encinitas, CA (US); Kimberly J Gietzen, San Jose, CA (US); Yuming Xu, Mountain View, CA (US); Deborah A Kallick, Portola Valley, CA (US); Ernestine A Lee, Castro Valley, CA (US); Kavitha Thangavelu, Sunnyvale, CA (US); Angelo M Delegeane, Milpitas, CA (US); Sally Lee, San Jose, CA (US); Sajeev Batra, Oakland, CA (US); Preetr G. Lal, Santa Clara, CA (US); Farrah A. Khan, Des Plaines, IL (US)

Correspondence Address: INCYTE CORPORATION (formerly known as Incyte, Genomics, Inc.), 3160 PORTER DRIVE, PALO ALTO, CA, 94304, US

(21) Appl. No.: 10/312,352

(22) PCT Filed: Jun. 29, 2001
(86) PCT No.: PCT/US01/21067

(51) Int. Cl. C07K 14/705; C12Q 1/68; C07H 21/04; C12P 21/02; C12N 5/06; A61K 38/17

(52) U.S. Cl. 514/12; 435/6; 435/69.1; 435/320.1; 435/325; 530/350; 536/23.5; 530/388.22

(57) ABSTRACT

The invention provides human extracellular matrix and cell adhesion molecules (ECMCAD) and polynucleotides which identity and encode ECMCAD. The invention also provides expression vectors, host cells, antibodies, agonists, and antagonists. The invention also provides methods for diagnosing, treating, or preventing disorders associated with aberrant expression of ECMCAD.

### TECHNICAL FIELD

This invention relates to nucleic acid and amino acid sequences of extracellular matrix and cell adhesion molecules and to the use of these sequences in the diagnosis, treatment, and prevention of genetic, immune/inflammatory, developmental, neurological, connective tissue, and cell proliferative disorders, including cancer and in the assessment of the effects of exogenous compounds on the expression of nucleic acid and amino acid sequences of extracellular matrix and cell adhesion molecules.

### BACKGROUND OF THE INVENTION

### Extracellular Matrix Proteins

The extracellular matrix (ECM) is a complex network of glycoproteins, polysaccharides, proteoglycans, and other macromolecules that are secreted from the cell into the extracellular space. The ECM remains in close association with the cell surface and provides a supportive meshwork that profoundly influences cell shape, motility, strength, flexibility, and adhesion. In fact, adhesion of a cell to its surrounding matrix is required for cell survival except in the case of metastatic tumor cells, which have overcome the need for cell-ECM anchorage. This phenomenon suggests that the ECM plays a critical role in the molecular mechanisms of growth control and metastasis. (Reviewed in Ruoslahti, E. (I 996) Sci. Am. 275:72-77.) Furthermore, the ECM determines the structure and physical properties of connective tissue and is particularly important for morphogenesis and other processes associated with embryonic development and pattern formation.

The collagens comprise a family of ECM proteins that provide structure to bone, teeth, skin, ligaments, tendons, cartilage, blood vessels, and basement membranes. Multiple collagen proteins have been identified. Three collagen molecules fold together in a triple helix stabilized by interchain disulfide bonds. Bundles of these triple helices then associate to form fibrils.

Elastin and related proteins confer elasticity to tissues such as skin, blood vessels, and lungs. Elastin is a highly hydrophobic protein of about 750 amino acids that is rich in proline and glycine residues. Elastin molecules are highly cross-linked, forming an extensive extracellular network of fibers and sheets. Elastin fibers are surrounded by a sheath of microfibrilins which are composed of a number of glycoproteins, including fibrillin.

Fibronectin is a large ECM glycoprotein found in all vertebrates. Fibronectin exists as a dimer of two subunits, each containing about 2,500 amino acids. Each subunit folds into a rod-like structure containing multiple domains. The domains each contain multiple repeated modules, the most common of which is the type III fibronectin repeat. The type III fibronectin repeat is about 90 amino acids in length and is also found in other ECM proteins and in some plasma membrane and cytoplasmic proteins. Furthermore, some type III fibronectin repeats contain a characteristic tripeptide consisting of Arginine-Glycine-Aspartic acid (RGD). The RGD sequence is recognized by the integrin family of cell surface receptors and is also found in other ECM proteins. (Reviewed in Alberts, et al. (1994) Molecular Biology of the Cell, Garland Publishing, New York, N.Y., pp. 986-987.)

Laminin is a major glycoprotein component of the basal lamina which underlies and supports epithelial cell sheets. Laminin is one of the first ECM proteins synthesized in the developing embryo. Laminin is an 850 kilodalton protein composed of three polypeptide chains joined in the shape of a cross by disulfide bonds. Laminin is especially important for angiogenesis and, in particular, for guiding the formation of capillaries. (Reviewed in Alberts, supra, pp. 990-991.)

Many proteinaceous ECM components are proteoglycans. Proteoglycans are composed of unibranched polysaccharide chains (glycosaminoglycans) attached to protein cores. Common proteoglycans include aggrecan, betaglycan, decorin, perlecan, serglycin, and syndecan-1. Some of these molecules not only provide mechanical support, but also bind to extracellular signaling molecules, such as fibroblast growth factor and transforming growth factor β, suggesting a role for proteoglycans in cell-cell communication. (Reviewed in Alberts, supra, pp. 973-978.)

Dentin phosphoryn (DPP) is a major component of the dentin ECM. DPP is a proteoglycan that is synthesized and expressed by odontoblasts (Gu, K., et al. (1998) Eur. J. Oral Sci. 106:1043-1047). DPP is believed to nucleate or modulate the formation of hydroxyapatite crystals.

Mucins are highly glycosylated glycoproteins that are the major structural component of the mucus gel. The physiological functions of mucins are cytoprotection, mechanical protection, maintenance of viscosity in secretions, and cellular recognition. MUC6 is a human gastric mucin that is also found in gall bladder, pancreas, seminal vesicles, and female reproductive tract (Toribara, N. W., et al. (1997) J. Biol. Chem. 272:16398-16403). The MUC6 gene has been mapped to human chromosome 11 (Toribara, N. W., et al. (1993) J. Biol. Chem. 268:5879-5885). Hemomucin is a novel Drosophila surface mucin that may be involved in the induction of antibacterial effector molecules (Theopold, U., et al. (1996) J. Biol. Chem. 217:12708-12715).

Olfactomedin was originally identified as the major component of the mucus layer surrounding the chemosensory dendrites of olfactory neurons. Olfactomedin-related proteins are secreted glycoproteins with conserved C-terminal motifs. The TIGR/myocilin protein, an olfactomedin-related protein expressed in the eye, is associated with the pathogenesis of glaucoma (Kulkarni, N. H. et al. (2000) Genet. Res. 76:41-50).

Ankyrin (ANK) repeats mediate protein-protein interactions associated with diverse intracellular functions. ANK repeats are composed of about 33 amino acids that form a helix-turn-helix core preceded by a protruding “tip.” These tips are of variable sequence and may play a role in protein-protein interactions. The helix-turn-helix region of the ANK repeats stack on top of one another and are stabilized by hydrophobic interactions (Yang, Y. et al. (I 998) Structure 6:619-626).

Sushi repeats, also called short consensus repeats (SCR), are found in a number of proteins that share the common feature of binding to other proteins. For example, in the C-terminal domain of versican, the sushi domain is important for heparin binding. Sushi domains contain basic amino acid residues, which may play a role in binding (Oleszewski, M. et al. (2000) J. Biol. Chem. 275:34478-34485).

Link, or X-link, modules are hyaluronan-binding domains found in proteins involved in the assembly of extracellular matrix, cell adhesion, and migration. The Link module superfamily includes CD44, cartilage link protein, and aggrecan. There is close similarity between the Link module and the C-type lectin domain, with the predicted hyaluronan-binding site at an analogous position to the carbohydrate-binding pocket in E-selectin (Kohda, D. et al. (1996) Cell, Vol. 86, 767-775).

Multidomain or mosaic proteins play an important role in the diverse functions of the extracellular matrix (Engel, J. et al. (1994) Development (Camb.) S35-42). ECM proteins are frequently characterized by the presence of one or more domains which may contain a number of potential intracellular disulfide bridge motifs. For example, domains which match the epidermal growth factor (EGF) tandem repeat consensus are present within several known extracellular proteins that promote cell growth, development, and cell signaling. This signature sequence is about forty amino acid residues in length and includes six conserved cysteine residues, and a calcium-binding site near the N-terminus of the signature sequence. The main structure is a two-stranded beta-sheet followed by a loop to a C-terminal short two-stranded sheet. Subdomains between the conserved cysteines vary in length (Davis, C. G. New Biol (1990) May;2(5):410-9). Post-translational hydroxylation of aspartic acid or asparagine residues has been associated with EGF-like domains in several proteins (Prosite PDOC00010 Aspartic acid and asparagine hydroxylation site).

A number of proteins that contain calcium-binding EGF-like domain signature sequences are involved in growth and differentiation. Examples include bone morphogenic protein 1, which induces the formation of cartilage and bone; crumbs, which is a Drosophila epithelial development protein; Notch and a number of its homologs, which are involved in neural growth and differentiation, and transforming growth factor beta-1 binding protein (Expasy PROSITE document PDOC00913; Soler, C. and Carpenter, G., in Nicola, N. A. (1994) The Cytokine Facts Book, Oxford University Press, Oxford, UK, pp 193-197). EGF-like domains mediate protein-protein interactions for a variety of proteins. For example, EGF-like domains in the ECM glycoprotein fibulin-1 have been shown to mediate both self-association and binding to fibronectin (Tran, H. et al. (1997) J. Biol. Chem. 272:22600-22606). Point mutations in the EGF-like domains of ECM proteins have been identified as the cause of human disorders such as Marfan syndrome and pseudochondroplasia (Maurer, P. et al. (1996) Curr. Opin. Cell Biol. 8:609-617).

The CUB domain is an extracellular domain of approximately 110 amino acid residues found mostly in developmentally regulated proteins. The CUB domain contains four conserved cysteine residues and is predicted to have a structure similar to that of immunoglobulins. Vertebrate bone morphogenic protein 1, which induces cartilage and bone formation, and fibropellins I and III from sea urchin, which form the apical lamina component of the ECM, are examples of proteins that contain both CUB and EGF domains (PROSITE PDOC00908 CUB domain profile).

Other ECM proteins are members of the type A domain of von Willebrand factor (vWFA)-like module superfamily, a diverse group of proteins with a module sharing high sequence similarity. The vWFA-like module is found not only in plasma proteins but also in plasma membrane and ECM proteins (Colombatti, A. and Bonaldo, P. (1991) Blood 77:2305-2315). Crystal structure analysis of an integrin vWFA-like module shows a classic “Rossmann” fold and suggests a metal ion-dependent adhesion site for binding protein ligands (Lee, J.-O. et al. (1995) Cell 80:631-638). This family includes the protein matrilin-2, an extracellular matrix protein that is expressed in a broad range of mammalian tissues and organs. Matrilin-2 is thought to play a role in ECM assembly by bridging collagen fibrils and the aggrecan network (Deak, F. et al. (1997) J. Biol. Chem. 272:9268-9274).

The thrombospondins are multimeric, calcium-binding extracellular glycoproteins found widely in the embryonic extracellular matrix. These proteins are expressed in the developing nervous system or at specific sites in the adult nervous system after injury. Thrombospondins contain multiple EGF-type repeats, as well as a motif known as the thrombospondin type 1 repeat (TSR). The TSR is approximately 60 amino acids in length and contains six conserved cysteine residues. Motifs within TSR domains are involved in mediating cell adhesion through binding to proteoglycans and sulfated glycolipids. Thrombospondin-1 inhibits angiogenesis and modulates endothelial cell adhesion, motility, and growth. TSR domains are found in a diverse group of other proteins, most of which are expressed in the developing nervous system and have potential roles in the guidance of cell and growth cone migration Proteins that share TSRs include the F-spondin gene family, the semapholin 5 family, UNC-5, and SCO-spondin. The TSR superfamily includes the ADAMTS proteins which contain an ADAM (A Disintegin and Metalloproteinase) domain as well as one or more TSRS. The ADAMTS proteins have roles in regulating the turnover of cartilage, matrix, regulation of blood vessel garowth, and possibly development of the nervous system. (Reviewed in Adams, J. C. and Tucker, R. P. (2000) Dev. Dyn. 218:280-299).

Fibrinogen, the principle protein of vertebrate blood clotting, is a hexamer consisting of two sets of three different chains (alpha, beta, and gamma). The C-terminal domain of the beta and gamma chains comprises about 270 amino acid residues and contains four cysteines involved in two disulfide bonds. This domain has also been found in mammalian tenascin-X, an ECM protein that appears to be involved in cell adhesion (Pro site PDOC00445 Fibrinogen beta and gamma chains C-terminal domain signature).

### Adhesion-Associated Proteins

The surface of a cell is rich in transmembrane proteoglycans, glycoproteins, glycolipids, and receptors. These macromolecules mediate adhesion with other cells and with components of the ECM. The interaction of the cell with its surroundings profoundly influences cell shape, strength, flexibility, motility, and adhesion. These dynamic properties are intimately associated with signal transduction pathways controlling cell proliferation and differentiation, tissue construction, and embryonic development. Families of cell adhesion molecules include the cadherins, integrins, lectins, neural cell adhesion proteins, and some members of the proline-rich proteins.

Cadherins comprise a family of calcium-dependent glycoproteins that function in mediating cell-cell adhesion in virtually all solid tissues of multicellular organisms. These proteins share multiple repeats of a cadherin-specific motif, and the repeats form the folding units of the cadherin extracellular domain. Cadherin molecules cooperate to form focal contacts, or adhesion plaques, between adjacent epithelial cells. The cadherin family includes the classical cadherins and protoeadherins. Classical cadherins include the E-cadherin, N-cadherin, and P-cadherin subfamilies. E-cadherin is present on many types of epithelial cells and is especially important for embryonic

[symbol]

Integrins are ubiquitous transmembrane adhesion molecules that link the ECM to the internal cytoskeleton. Integrins are composed of two noncovalently associated transmembrane glycoprotein subunits called α and β. Integrins function as receptors that play a role in signal transduction. For example, binding of integrin to its extracellular ligand may stimulate changes in intracellular calcium levels or protein kinase activity (Sjaastad, M. D. and Nelson, W. J. (1997) BioEssays 19:47-55). At least ten cell surface receptors of the integrin family recognize the ECM component fibronectin, which is involved in many different biological processes including cell migration and embryogenesis (Johansson, S. et al. (1997) Front. Biosci. 2:D126-D146).

Lectins comprise a ubiquitous family of extracellular glycoproteins which bind cell surface carbohydrates specifically and reversibly, resulting in the agglutination of cells (reviewed in Drickamer, K. and Taylor, M. E. (1993) Annu. Rev. Cell Biol. 9:237-264). This function is particularly important for activation of the immune response. Lectins mediate the agglutination and mitogenic stimulation of lymphocytes at sites of inflammation (Lasky, L. A. (1991) J. Cell. Biochem. 45:139-146; Paietta, E. et al. (1989) J. Immunol. 143:2850-2857).

Lectins are further classified into subfamilies based on carbohydrate-binding specificity and other criteria. The galectin subfamily, in particular, includes lectins that bind β-galactoside carbohydrate moieties in a thiol-dependent manner (reviewed in Hadari, Y. R. et al. (1998) J. Biol. Chem. 270:3447-3453). Galectins are widely expressed and developmentally regulated. Galectins contain a characteristic carbohydrate recognition domain (CRD). The CRD is about 140 amino acids and contains several stretches of about 1 - 10 amino acids which are highly conserved among all galectins. A particular 6-amino acid motif within the CRD contains conserved tryptophan and arginine residues which are critical for carbohydrate binding. The CRD of some galectins also contains cysteine residues which may be important for disulfide bond formation. Secondary structure predictions indicate that the CRD forms several β-sheets.

Galectins play a number of roles in diseases and conditions associated with cell-cell and cell-matrix interactions. For example, certain galectins associate with sites of inflammation and bind to cell surface immunoglobulin E molecules. In addition, galectins may play an important role in cancer metastasis. Galectin overexpression is correlated with the metastatic potential of cancers in humans and mice. Moreover, anti-galectin antibodies inhibit processes associated with cell transformation, such as cell aggregation and anchorage-independent growth (see, for example, Su, Z.-Z. et al. (1996) Proc. Natl. Acad. Sci. USA 93:7252-7257).

Selectins, or LEC-CAMs, comprise a specialized lectin subfamily involved primarily in inflammation and leukocyte adhesion (Reviewed in Lasky, supra). Selectins mediate the recruitment of leukocytes from the circulation to sites of acute inflammation and are expressed on the surface of vascular endothelial cells in response to cytokine signaling. Selectins bind to specific ligands on the leukocyte cell membrane and enable the leukocyte to adhere to and migrate along the endothelial surface. Binding of selectin to its ligand leads to polarized rearrangement of the actin cytoskeleton and stimulates signal transduction within the leukocyte (Breiner, B. et al. (1997) Biochem. Biophys. Res. Commun. 231:802-807; Hidati, K. I. et al. (1997) J. Biol. Chem. 272:28750-28756). Members of the selectin family possess three characteristic motifs: a lectin or carbohydrate recognition domain; an epidermal growth factor-like domain; and a variable number of short consensus repeats (scr or “sushi” repeats) which are also present in complement regulatory proteins.

Neural cell adhesion proteins (NCAPs) play roles in the establishment of neural networks during development and regeneration of the nervous system (Uyemura et al. (1996) Essays Biochem. 31:37-48; Blummendorf and Rathjen (1996) Curr. Opin. Neurobiol. 6:584-593). NCAP participates in neuronal cell migration, cell adhesion, neurite outgrowth, axonal fasciculation, pathfinding, synaptic target-recognition. synaptic formation, myelination and regeneration. NCAPs are expressed on the surfaces of neurons associated with learning and memory. Mutations in genes encoding NCAPS are linked with neurological diseases, including hereditary neuropathy Charcot-Marie-Tooth disease, Dejerine-Sottas disease, X-linked hydrocephalus, MASA syndrome (mental retardation, aphasia, shuffling gait and adducted thumbs), and spastic paraplegia type I. In some cases, expression of NCAP is not restricted to the nervous system. L1, for example, is expressed in melanoma cells and hematopoiatic tumor cells where it is implicated in cell spreading and migration, and may play a role in tumor progression (Montgomery et al. (1996) J. Cell Biol. 132:475-485).

NCAPs have at least one immunoglobulin constant or variable domain (Uyemura et al., supra). They are generally linked to the plasma membrane through a transmembrane domain and/or a glycosyl-phosphatidylinositol (GPI) anchor. The GPI linkage can be cleaved by GPI phospholipase C. Most NCAPs consist of an extracellular region made up of one or more immunoglobulin domains, a membrane spanning domain, and an intracellular region. Many NCAPs contain post-translational modifications including covalently attached oligosaccharide, glucuronic acid, and sulfate. NCAPs fall into three subgroups: simple-type, complex-type, and mixed-type. Simple-type NCAPs contain one or more variable or constant immunoglobulin domains, but lack other types of domains. Members of the simple-type subgroup include Schwann cell myclin protein (SMP), limbic system-associated membrane protein (LAMP), opiate-binding cell-adhesion molecule (OBCAM), and myelin-associated glycoprotein (MAG). The complex-type NCAPs contain fibronectin type III domains in addition to the immunoglobulin domains. The complex-type subgroup includes neural cell-adhesion molecule (NCAM), axonin-1, F11, Bravo, and L1. Mixed-type NCAPs contain a combination of immunoglobulin domains and other motifs such as tyrosine kinase and epidermal growth factor-like domains. This subgroup includes Trk receptors of nerve growth factors such as nerve growth factor (NGF) and neurotropin 4 (NT4), Neu differentiation factors such as glial growth factor II (GGFUI) and acctylcholinc receptor-inducing factor (ARIA), and the semaphorin/collapsin family such as semaphorin B and collapsin.

Semaphorins are a large group of axonal guidance molecules consisting of at least 30 different members and are found in vertebrates, invertebrates, and even certain viruses. All semaphorins contain the soma domain which is approximately 500 amino acids in length. Neuropilin, a semaphorin receptor has been shown to promote neurite outgrowth in vitro. The extracellular region of neuropilins consists of three different domains: CUB, discoidin, and MAM domains. The CUB and the MAM motifs of neuropilin have been suggested as having roles in protein-protein interactions and are suggested to be involved in the binding of semaphorins through the sema and the C-terminal domains (reviewed in Raper, J. A. (2000) Curr. Opin. Neurobiol. 10:88-94).

An NCAP subfamily, the NCAP-LON subgroup, includes cell adhesion proteins expressed on distinct subpopulations of brain neurons. Members of the NCAP-LON subgroup possess three immunoglobulin domains and bind to cell membranes through GPI anchors. Kilon (a kindred of NCAP-LON), for example, is expressed in the brain cerebral cortex and hippocampus (Funatsu et al. (1999) J. Biol. Chem. 274:8224-8230). Immunostaining localizes Kilon to the dendrites and soma of pyramidal neurons. Kilon has three C2 type immunoglobulin-like domains, six predicted glycosylation sites, and a GPI anchor. Expression of Kilon is developmentally regulated. It is expressed at higher levels in adult brain in comparison to embryonic and early postnatal brains. Confocal microscopy shows the presence of Kilon in dendrites of hypothalamic magnocellular neurons secreting neuropeptides, oxytocin or arginine vasopressin (Miyata et al. (2000) J. Comp. Neurol. 424:74-85). Arginine vasopressin regulates body fluid homeostasis, extracellular osmolarity and intravascular volume. Oxytocin induces contractions of uterine smooth muscle during child birth and of myoepithelial cells in mammary glands during lactation. In magnocellular neurons, Kilon is proposed to play roles in the reorganization of dendritic connections during neuropeptide secretion.

Cell adhesion proteins also include some members of the proline-rich proteins (PRPs). PRPs are defined by a high frequency of proline, ranging from 20-50% of the total amuino acid content. Some PRPs have short domains which are rich in proline. These proline-rich regions are associated with protein-protein interactions. One family of PRPs are the proline-rich synapse-associated proteins (ProSAPs) which have been shown to bind to members of the postsynaptic density (PSD) protein family and subtypes of the somatostatin receptor (Yao, I. et al. (1999) J. Biol. Chem. 274: 27463-27466; Zitzer, H. et al. (1999) J. Biol. Chem. 274:32997-33001). Members of ProSAP contain at the N-terminus six to seven ankyrin repeats, followed by an SH3 domain, a PDZ domain, then by seven proline-rich regions and a SAM domain at the C terminus. Several groups of ProSAP are important structural constituents of synaptic structures in human brain (Ziter et al., supra). Another member of PRP is the HLA-B-associated transcript 2 protein (BAT2) which is rich in proline and include short tracts of polyproline, polyglycine, and charged amino acids. BAT2 also contains four RGD (Arg-Gly-Asp) motifs typical of integrins (Bancrji, J. et al. (1990) Proc. Natl. Acad. Sci. USA 87:2374-2378).

There are additional specific domains characteristic of cell adhesion proteins. One such domain is the MAM domain, a domain of about 170 amino acids found in the extracellular region of diverse proteins. These proteins all share a receptor-like architecture comprising a signal peptide, followed by a large N-terminal extracellular domain, a transmembrane region, and an intracellular domain. (PROSITE document PDOC00604 MAM domain signature and profile). MAM domain proteins include zonadhesin, a sperm-specific membrane protein that binds to the zona pellucida of the egg; neuropilin, a cell adhesion molecule that functions during the formation of certain neuronal circuits, and Xenonus laevis thyroid hormone induced protein B, which contains four MAM domains and is involved in metamorphosis (Brown, D. D. et al. (1996) Proc. Natl. Acad. Sci. USA 93:1924-1929).

The WSC domain was originally found in the yeast WSC (cell-wall integrity and stress response component) proteins which act as sensors of environmental stress. The WSC domains are extracellular and are thought to possess a carbohydrate binding role (Ponting, C. P. et al. (1999) Curr. Biol. 9:S1-S2). A WSC domain has recently been identified in polycystin-1, a human plasma membrane protein. Mutations in polycystin-1 are the cause of the commonest form of autosonial dominant polycystic kidney disease (Ponting, C. P. et al. (1999) Curr. Biol. 9:R585-R588).

Toposome is a cell-adhesion glycoprotein isolated from mesenchyme-blastula embryos. Toposome precursors including vitellogenin promote cell adhesion of dissociated blastula cells.

Leucine rich repeats (LRR) are short motifs found in numerous proteins from a wide range of species. LRR motifs are of variable length, most commonly 20-29 amino acids and multiple repeats are typically present in tandem. LRR is important for protein/protein interactions and cell adhesion, and LRR proteins are involved in cell/cell interactions, morphogenesis, and development (Kobe, B. and Deisenhofer, J. (1 995) Curr. Opin. Struct. Biol. 5:409-416). The human ISLR (immunoglobulin superfamnily containing leucine-rich repeat) protein contains a C2-type immunoglobulin domain as well as LRR. The ISLR gene is linked to the critical region for Bardet-Biedl syndrome, a developmental disorder of which the most common feature is retinal dystrophy (Nagasawa, A. et al. (1999) Genomics 61:37-43).

The sterile alpha motif (SAM) domain is a conserved protein binding domain, approximately 70 amino acids in length, and is involved in the regulation of many developmental processes in many eukaryotes. The SAM domain can potentially function as a protein interaction module through its ability to form homo- or hetero-oligomers with other SAM domains (Schultz, J. et al. (1997) Protein Sci. 6:249-253).

The discovery of new extracellular matrix and cell adhesion molecules and the polynucleotides encoding them satisfies a need in the art by providing new compositions which are useful in the diagnosis, prevention, and treatment of genetic, immune/inflammatory, developmental, neurological, connective tissue, and cell proliferative disorders, including cancer, and in the assessment of the effects of exogenous compounds on the expression of nucleic acid and amino acid sequences of extracellular matrix and cell adhesion molecules.

### SUMMARY OF THE INVENTION

The invention features purified polypeptides, extracellular matrix and cell adhesion molecules, referred to collectively as “ECMCAD” and individually as “ECMCAD-1,” “ECMCAD-2,” “ECMCAD-3,” “ECMCAD-4,” “ECMCAD-5,” “ECMCAD-6,” “ECMCAD-7,” “ECMCAD-8,” “ECMCAD-9,” “ECMCAD-10,” “ECMCAD-11,” “ECMCAD-12,” “ECMCAD-13,” “ECMCAD-14,” “ECMCAD-15,” “ECMCAD-16,” “ECMCAD-17,” “ECMCAD-18,” “ECMCAD-19,” “ECMCAD-20,” “ECMCAD-21,” “ECMCAD-22,” “ECMCAD-23,” “ECMCAD-24,” “ECMCAD-25,” “ECMCAD-26,” “ECMCAD-27,” “ECMCAD-28,” “ECMCAD-29,” “ECMCAD-30,” “ECMCAD-31,” “ECMCAD-32,” “ECMCAD-33,” “ECMCAD-34,” “ECMCAD-35,” and “ECMCAD-36.” In one aspect, the invention provides an isolated polypeptide selected from the group consisting of a) a polypeptide comprising an amino acid sequence selected from the group consisting of SEQ ID NO:1-36, b) a naturally occurring polypeptide comprising an amino acid sequence at least 90% identical to an amino acid sequence selected from the group consisting of SEQ ID NO:1-36, c) a biologically active fragment of a polypeptide having an amino acid sequence selected from the group consisting of SEQ ID NO:1-36, and d) an immunogenic fragment of a polypeptide having an amino acid sequence selected from the group consisting of SEQ ID NO:1-36. In one alternative, the invention provides an isolated polypeptide comprising the amino acid sequence of SEQ ID NO:1-36.

The invention further provides an isolated polynucleotide encoding a polypeptide selected from the group consisting of a) a polypeptide comprising an amino acid sequence selected from the group consisting of SEQ ID NO:1-36, b) a naturally occurring polypeptide comprising an amino acid sequence at least 90% identical to an amino acid sequence selected from the group consisting of SEQ ID NO:1-36, c) a biologically active fragment of a polypeptide having an amino acid sequence selected from the group consisting of SEQ ID NO:1-36, and d) an immunogenic fragment of a polypeptide having an amino acid sequence selected from the group consisting of SEQ ID NO:1-36. In one alternative, the polynucleotide encodes a polypeptide selected from the group consisting of SEQ ID NO:1-36. In another alternative, the polynucleotide is selected from the group consisting of SEQ ID NO:37-72.

Additionally, the invention provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-36, b) a naturally occurring polypeptide comprising an amino acid sequence at least 90% identical to an amino acid sequence selected from the group consisting of SEQ ID NO:1-36, c) a biologically active fragment of a polypeptide having an amino acid sequence selected from the group consisting of SEQ ID NO:1-36, and d) an immunogenic fragment of a polypeptide having an amino acid sequence selected from the group consisting of SEQ ID NO:1-36. In one alternative, the invention provides a cell transformed with the recombinant polynucleotide. In another alternative, the invention provides a transgenic organism comprising the recombinant polynucleotide.

The invention also provides a method for producing a polypeptide selected from the group consisting of a) a polypeptide comprising an amino acid sequence selected from the group consisting of SEQ ID NO:1-36, b) a naturally occurring polypeptide comprising an amino acid sequence at least 90% identical to an amino acid sequence selected from the group consisting of SEQ ID NO:1-36, c) a biologically active fragment of a polypeptide having an amino acid sequence selected from the group consisting of SEQ ID NO:1-36, and d) an immunogenic fragment of a polypeptide having an amino acid sequence selected from the group consisting of SEQ ID NO:1-36. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Additionally, the invention provides an isolated antibody which specifically binds to a polypeptide selected from ide group consisting of a) a polypeptide comprising an amino acid sequence selected from the group consisting of SEQ ID NO: 1-36, b) a naturally occurring polypeptide comprising an amino acid sequence at least 90% identical to an amino acid sequence selected from the group consisting of SEQ ID NO:1-36, c) a biologically active fragment of a polypeptide having an amino acid sequence selected from the group consisting of SEQ ID NO:1-36, and d) an immunogenic fragment of a polypeptide having an amino acid sequence selected from the group consisting of SEQ ID NO:1-36.

The invention further provides an isolated polynucleotide selected from the group consisting of a) a polynucleotide comprising a polynucleotide sequence selected forms the group consisting of SEQ ID NO:37-72, b) a naturally occurring polynucleotide comprising a polynucleotide sequence at least 90% identical to a polynucleotide sequence selected from the group consisting of SEQ ID NO:37-72, c) a polynucleotide complementary to the polynucleotide of a), d) a polynucleotide complementary to the polynucleotide of b), and e) an RNA equivalent of a)-d). In one alternative, the polynucleotide comprises at least 60 contiguous nucleotides.

Additionally, the invention provides a method for detecting a target polynucleotide in a sample, said target polynucleotide having a sequence of a polynucleotide selected from the group consisting of a) a polynucleotide comprising a polynucleotide sequence selected from the group consisting of SEQ ID, NO:37-72, b) a naturally occurring polynucleotide comprising a polynucleotide sequence at least 90% identical to a polynucleotide sequence selected from the group consisting of SEQ ID NO:37-72, c) a polynucleotide complementary to the polynucleotide of a), d) a polynucleotide complementary to the polynucleotide of b), and c) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex, and optionally, if present, the amount thereof. In one alternative, the probe comprises at least 60 contiguous nucleotides.

The invention further provides a method for detecting a target polynucleotide in a sample, said target polynucleotide having a sequence of a polynucleotide selected from the group consisting of a) a polynucleotide comprising a polynucleotide sequence selected from the group consisting of SEQ ID NO:37-72, b) a naturally occurring polynucleotide comprising a polynucleotide sequence at least 90% identical to a polynucleotide sequence selected from the group consisting of SEQ ID NO:37-72, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof, and, optionally, if present, the amount thereof.

The invention further provides a composition comprising an effective amount of a polypeptide selected from the group consisting of a) a polypeptide comprising an amino acid sequence selected from the group consisting of SEQ ID NO: 1-36, b) a naturally occurring polypeptide comprising an amino acid sequence at least 90% identical to an amino acid sequence selected from the group consisting of SEQ ID NO:1-36, c) a biologically active fragment of a polypeptide having an amino acid sequence selected from the group consisting of SEQ ID NO:1 -36, and d) an immunogenic fragment of a polypeptide having an amino acid sequence selected from the group consisting of SEQ ID NO: 1 -36, and a pharmaceutically acceptable excipient. In one embodiment, the composition comprises an amino acid sequence selected from the group consisting of SEQ ID NO:1-36. The invention additionally provides a method of treating a disease or condition associated with decreased expression of functional ECMCAD, comprising administering to a patient in need of such treatment the composition.

The invention also provides a method for screening a compound for effectiveness as an agonist of a polypeptide selected from the group consisting of a) a polypeptide comprising an amino acid sequence selected from the group consisting of SEQ ID NO:1-36, b) a naturally occurring polypeptide comprising an amino acid sequence at least 90% identical to an amino acid sequence selected from the group consisting of SEQ ID NO:1-36, c) a biologically active fragment of a polypeptide having an amino acid sequence selected from the group consisting of SEQ ID NO:1 -36, and d) an immunogenic fragment of a polypeptide having an amino acid sequence selected from the group consisting of SEQ ID NO:1-36. The method comprises a) exposing a sample comprising the polypeptide to a compound, and b) detecting agonist activity in the sample. In one alternative, the invention provides a composition comprising an agonist compound identified by the method and a pharmaceutically acceptable excipient. In another alternative, the invention provides a method of treating a disease or condition associated with decreased expression of functional ECMCAD, comprising administering to a patient in need of such treatment the composition.

Additionally, the invention provides a method for screening a compound for effectiveness as an antagonist of a polypeptide selected from the group consisting of a) a polypeptide comprising an amino acid sequence selected from the group consisting of SEQ ID NO:1-36, b) a naturally occurring polypeptide comprising an amino acid sequence at least 90% identical to an amino acid sequence selected from the group consisting of SEQ ID NO:1-36, c) a biologically active fragment of a polypeptide having an amino acid sequence selected from the group consisting of SEQ ID NO:1-36, and d) an immunogenic fragment of a polypeptide having an amino acid sequence selected from the group consisting of SEQ ID NO:1-36. The method comprises a) exposing a sample comprising the polypeptide to a compound, and b) detecting antagonist activity in the sample. In one alternative, the invention provides a composition comprising an antagonist compound identified by the method and a pharmaceutically acceptable excipient. In another alternative, the invention provides a method of treating a disease or condition associated with overexpression of functional ECMCAD, comprising administering to a patient in need of such treatment the composition.

The invention further provides a method of screening for a compound that specifically binds to a polypeptide selected from the group consisting of a) a polypeptide comprising an amino acid sequence selected from the, group consisting of SEQ ID NO: 1-36, b) a naturally occurring polypeptide comprising an amino acid sequence at least 90% identical to an amino acid sequence selected from the group consisting of SEQ ID NO:1-36, c) a biologically active fragment of a polypeptide having an amino acid sequence selected from the group consisting of SEQ ID NO:1-36, and d) an immunogenic fragment of a polypeptide having an amino acid sequence selected from the group consisting of SEQ ID NO:1-36. The method comprises a) combining the polypeptide with at least one test compound under suitable conditions, and b) detecting binding of the polypeptide to the test compound, thereby identifying a compound that specifically binds to the polypeptide.

The invention further provides a method of screening for a compound that modulates the activity of a polypeptide selected from the group consisting of a) a polypeptide comprising an amino acid sequence selected from the group consisting of SEQ ID NO: 1-36, b) a naturally occurring polypeptide comprising an amino acid sequence at least 90% identical to an amino acid sequence selected from the group consisting of SEQ ID NO:1-36, c) a biologically active fragment of a polypeptide having an amino acid sequence selected from the group consisting of SEQ ID NO:1-36, and d) an immunogenic fragment of a polypeptide having an amino acid sequence selected from the group consisting of SEQ ID NO:1-36. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

The invention further provides a method for screening a compound for effectiveness in altering expression of a target polynucleotide, wherein said target polynucleotide comprises a sequence selected from the group consisting of SEQ ID NO:37-72, the method comprising a) exposing a sample comprising the target polynucleotide to a compound, and b) detecting altered expression of the target polynucleotide.

The invention further provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:37-72, ii) a naturally occurring polynucleotide comprising a polynucleotide sequence at least 90% identical to a polynucleotide sequence selected from the group consisting of SEQ ID NO:37-72, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:37-72, ii) a naturally occurring polynucleotide comprising a polynucleotide sequence at least 90% identical to a polynucleotide sequence selected from the group consisting of SEQ ID NO:37-72, iii) a polynucleotide complementary to the polynucleotide of i), iv) a polynucleotide complementary to the polynucleotide of ii), and v) an RNA equivalent of i)-iv). Alternatively, the target polynucleotide comprises a fragment of a polynucleotide sequence selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for the full length polynucleotide and polypeptide sequences of the present invention.

Table 2 shows the GenBank identification number and annotation of the nearest GenBank homolog for polypeptides of the invention. The probability score for the match between each polypeptide and its GenBank homolog is also shown.

Table 3 shows structural features of polypeptide sequences of the invention, including predicted motifs and domains, along with the methods, algorithms, and searchable databases used for analysis of the polypeptides.

Table 4 lists the cDNA and/or genomic DNA fragments which were used to assemble polynucleotide sequences of the invention, along with selected fragments of the polynucleotide sequences.

Table 5 shows the representative cDNA library for polynucleotides of the invention.

Table 6 provides an appendix which describes the tissues and vectors used for construction of the cDNA libraries shown in Table 5.

Table 7 shows the tools, programs, and algorithims used to analyze the polynucleotides and polypeptides of the invention, along with applicable descriptions, references, and threshold parameters.

### DESCRIPTION OF THE INVENTION

Before the present proteins, nucleotide sequences, and methods are described, it is understood that this invention is not limited to the particular machines, materials and methods described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

### DEFINITIONS

“ECMCAD” refers to the amino acid sequences of substantially purified ECMCAD obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of ECMCAD. Agonists may include proteins, nucleic acids, carbohydrates, small molecules, or any other compound or composition which modulates the activity of ECMCAD either by directly interacting with ECMCAD or by acting oil components of the biological pathway in which ECMCAD participates.

An “allelic variant” is an alternative form of the gene encoding ECMCAD. Allelic variants may result from at least one mutation in the nucleic acid sequence and may result in altered mRNAs or in polypeptidcs whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding ECMCAD include those sequences with deletions, insertions, or substitutions of different nucleotides, resulting in a polypeptide the same as ECMCAD or a polypeptide with at least one functional characteristic of ECMCAD. Included within this definition are polymorphisms which may or may not be readily detectable using a particular oligonucleotide probe of the polynucleotide encoding ECMCAD, and improper or unexpected hybridization to allelic variants, with a locus other than the normal chromosomal locus for the polynucleotide sequence encoding ECMCAD. The encoded protein may also be “altered,” and may contain deletions, insertions, or substitutions of amino acid residues which produce a silent change and result in a functionally equivalent ECMCAD. Deliberate amino acid substitutions may be made on the basis of similarity in polarity, charge, solubility, hydrophobicity, hydrophilicity, and/or the amphipathic nature of the residues, as long as the biological or immunological activity of ECMCAD is retained. For example, negatively charged amino acids may include aspailtic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” refer to an oligopeptide, peptide, polypeptide, or protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid sequence. Amplification is generally carried out using polymerase chain reaction (PCR) technologies well known in the art.

The term “antagonist” refers to a molecule which inhibits or attenuates the biological activity of ECMCAD. Antagonists may include proteins such as antibodies, nucleic acids, carbohydrates, small molecules, or any other compound or composition which modulates the activity of ECMCAD either by directly interacting with ECMCAD or by acting on components of the biological pathway in which ECMCAD participates.

The term “antibody” refers to intact immunoglobulin molecules as well as to fragments thereof, such as Fab, F(ab′)₂, and Fv fragments, which are capable of binding an epitopic determinant. Antibodies that bind ECMCAD polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epilope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “antisense” refers to any composition capable of base-pairing with the “sense” (coding) strand of a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic ECMCAD, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide sequence” and a “composition comprising a given amino acid sequence” refer broadly to any composition containing the given polynucleotide or amino acid sequence. The composition may comprise a dry formulation or an aqueous solution. Compositions comprising polynucleotide sequences encoding ECMCAD or fragments of ECMCAD may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

A “fragment” is a unique portion of ECMCAD or the polynucleotide encoding ECMCAD which is identical in sequence to but shorter in length than the parent sequence. A fragment may comprise up to the entire length of the defined sequence, minus one nucleotide/amino acid residue. For example, a fragment may comprise from 5 to 1000 continuous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:37-72 comprises a region of unique polynucleotide sequence that specifically identifies SEQ ID NO :37-72, for example, as distinct from any other sequence in the genome from which the fragment was obtained. A fragment of SEQ ID NO:37-72 is useful, for example, in hybridization and amplification technologies and in analogous methods that distinguish SEQ ID NO:37-72 from related polynucleotide sequences. The precise length of a fragment of SEQ ID NO:37-72 and the region of SEQ ID NO:37-72 to which the fragment corresponds are routinely determinable by one of ordinary skill in the art based on the intended purpose for the fragment.

A fragment of SEQ ID NO:1-36 is encoded by a fragment of SEQ ID NO:37-72. A fragment of SEQ ID NO:1-36 comprises a region of unique amino acid sequence that specifically identifies SEQ ID NO:1-36. For example, a fragment of SEQ ID NO:1-36 is useful as an immunogenic peptide for the development of antibodies that specifically recognize SEQ ID NO :1-36. The precise length of a fragment of SEQ ID NO:1-36 and the region of SEQ ID NO:1-36 to which the fragment corresponds are routinely determinable by one of ordinary skill in the art based on the intended purpose for the fragment.

A “full length” polynucleotide sequence is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighied” residue weight table is selected as the default. Percent identity is reported by CLUSTAL V as the “percent similarity” between aligned polynucleotide sequences.

Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:

Matrix: BLOSUM62

Reward for match: 1

Penalty for mismatch: −2

Open Gap: 5 and Extension Gap: 2 penalties

Gap x drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:

Matrix: BLOSUM62

Open Gap: 11 and Extension Gap: 1 penalties

Gap x drop-off: 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formaride at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded potypeptides.

The term “hybridization complex” refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” and “addition” refer to changes in an amino acid or nucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of ECMCAD which is capable of eliciting an immune response when introduced into a living organism, for example, a mammal. The term “immunogenic fragment” also includes any polypeptide or oligopeptide fragment of ECMCAD which is useful in any of the antibody production methods disclosed herein or known in the art.

The term “microarray” refers to an arrangement of a plurality of polynucleotides, polypeptides, or other chemical compounds on a substrate.

The terms “element” and “array element” refer to a polynucleotide, polypeptide, or other chemical compound having a unique and defined position on a microarray.

The term “modulate” refers to a change in the activity of ECMCAD. For example, modulation may cause an increase or a decrease in protein activity, binding characteristics, or any other biological, functional, or immunological properties of ECMCAD.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an ECMCAD may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of ECMCAD.

“Probe” refers to nucleic acid sequences encoding ECMCAD, their complements, or fragments thereof, which are used to detect identical, allelic or related nucleic acid sequences. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid sequence, e.g., by the polymerase chain reaction (PCR). Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook, J. et al. (1989) Molecular Cloning: A Laboratory Manual, 2^{nd} ed., vol. 1-3, Cold Spring Harbor Press, Plainview N.Y.; Ausubel, F. M. et al. (1987) Current Protocols in Molecular Biolog, Greene Publ. Assoc. & Wiley-Intersciences, New York N.Y.; Innis, M. et al. (1990) PCR Protocols, A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genonie Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose.

The term “sample” is used in its broadest sense. A sample suspected of containing ECMCAD, nucleic acids encoding ECMCAD, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least 60% free, preferably at least 75% free, and most preferably at least 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism. including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternative splicing of exons during mRNA processing. The corresponding polypeptide may possess additional functional domains or lack domains that are present in the reference molecule. Species variants arc polynucleotide sequences that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptidc sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 91 %, at least 92%, at least 93%, at least 94%, at least 95%, at least 96%, at least 97%, at least 98%, or at least 99% or greater sequence identity over a certain defined length of one of the polypeptides.

### THE INVENTION

The invention is based on the discovery of new human extracellular matrix and cell adhesion molecules (ECMCAD), the polynucleotides encoding ECMCAD, and the use of these compositions for the diagnosis, treatment, or prevention of genetic, immune/inflammatory, developmental, neurological, connective tissue, and cell proliferative disorders, including cancer.

Table 1 summarizes the nomenclatrre for the full length polynucleotide and polypeptide sequences of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown.

Table 2 shows sequences with homology to the polypeptides of the invention as identified by BLAST analysis against the GenBank protein (genpept) database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (Genbank ID NO:) of the nearest GenBank homolog. Column 4 shows the probability score for the match between each polypeptide and its GenBank homolog. Column 5 shows the annotation of the GenBank homolog along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Genetics Computer Group, Madison Wis.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are extracellular matrix and cell adhesion molecules. For example, SEQ ID NO:2 is 48% identical over 46% of its length to mouse procollagen type I alpha chain, (GenBank ID g192264) as determined by the Basic Local Alignment Search Tool (BLAST). (See Table 2.) The BLAST probability score is 6.9e-46, which indicates the probability of obtaining the observed polypeptide sequence alignment by chance. SEQ ID NO:2 also contains a collagen triple helix repeat, as determined by searching for statistically significant matches in the PFAM database. (See Table 3.) HMMER and SPSCAN analyses indicate the presence of a signal peptide at the N-terminus of SEQ ID NO:2. Data from BLAST analysis of the PRODOM and DOMO databases, as well as MOTIFS analysis, provide further corroborative evidence that SEQ ID NO:2 is a cellular matrix protein associated with cell adhesion. In an alternative example, SEQ ID NO:6 is 64% identical to frog MAM domain protein (GenBank ID g1234793) as determined by the Basic Local Alignment Search Tool (BLAST). (See Table 2.) The BLAST probability score is 4.2e-254, which indicates the probability of obtaining the observed polypeptide sequence alignment by chance. SEQ ID NO:6 also contains four MAM domains as determined by searching for statistically significant matches in the hidden Markov model (HMM)-based PFAM database of conserved protein family domains. (See Table 3.) Data from MOTIFS analysis provide further corroborative evidence that SEQ ID NO:6 is a MAM domain cell adhesion protein. In an alternative example, SEQ ID NO:10 is 80% identical to murine semaphorin B (GenBank ID g854326) as determined by the Basic Local Alignment Search Tool (BLAST). (See Table 2.) The BLAST probability score is 6.0e-66, which indicates the probability of obtaining the observed polypeptide sequence alignment by chance. SEQ ID NO:10 also contains a soma domain as determined by searching for statistically significant matches in the hidden Markov model (HMM)-based PFAM database of conserved protein family domains. (See Table 3.) The BLAST and HMMER analyses provide evidence that SEQ ID NO:10 is a semaphorin. SEQ ID NO:12 is 44% identical to human cadherin superfamily protein VR4-11 (GenBank ID g9622240) as determined by the Basic Local Alignment Search Tool (BLAST). (See Table 2.) The BLAST probability score is 9.9e-170, which indicates the probability of obtaining the observed polypeptide sequence alignment by chance. SEQ ID NO:12 also contains a cadherin domain as determined by searching for statistically significant matches in the hidden Markov model (HMM)-based PFAM database of conserved protein family domains. (See Table 3.) Data from BLIMPS, MOTIFS, and PROFLES CAN analyses provide further corroborative evidence that SEQ ID NO: 12 is a cadherin. SEQ ID NO: 14 is 91% identical to mutin neuronal glycoprotein (GenBank ID g200057) as determined by the Basic Local Alignment Search Tool (BLAST). (See Table 2.) The BLAST probability score is 0.0, which indicates the probability of obtaining the observed polypeptide sequence alignment by chance, SEQ ID NO: 14 also contains fibronectin type III and immunoglobulin domains as determined by searching for statistically significant matches in the hidden Markov model (HMM)-based PFAM database of conserved protein family domains. (See Table 3.) The BLAST and HMMER analyses provide evidence that SEQ ID NO:14 is a cell adhesion molecule. In an alternative example, SEQ ID NO:22 is 79% identical to mouse lamidn 5 alpha chain (GenBank ID g2599232) as determined by the Basic Local Alignment Search Tool (BLAST), (See Table 2.) The BLAST probability score is 0.0, which indicates the probability of obtaining the observed polypeptide sequence alignment by chance. SEQ ID NO:22 also contains a laminin N-terminal domain, multiple laminin EGF-like domains, a laminin B domain, and laminin G domains, as determined by searching for statistically significant matches in the hidden Markov model (HMM)-based PFAM database of conserved protein family domains. (See Table 3.) Data from BLIMPS, and MOTIFS analyses provide further corroborative evidence that SEQ ID NO:22 is a laminin. In an alternative example, SEQ ID NO:24 is 89% identical to Bos taurus brevican (GenBank ID g452821) as determined by the Basic Local Alignment Search Tool (BLAST). (See Table 2.) The BLAST probability score is 0.0, which indicates the probability of obtaining the observed polypeptide sequence alignment by chance. SEQ ID NO:24 also contains a lectin C-type domain, an extracellular link domain, an EGF-fike domain, a sushi domain, and an immunoglobulin domain as determined by searching for statistically significant matches in the hidden Markov model (HMM)-based PFAM database of conserved protein family domains. (See Table 3.) Data from BLIMPS, MOTIFS, and PROFILESCAN analyses provide further corroborative evidence that SEQ ID NO:24 is a c-type lectin. In an alternative example, SEQ ID NO:31 is 87% identical lo a mouse semaphorin homolog (GenBank ID g1110599) as determined by the Basic Local Alignment Search Tool (BLAST). (See Table 2.) The BLAST probability score is 0.0, which indicates the probability of obtaining the observed polypeptide sequence alignment by chance. SEQ ID NO:31 also contains a Sema domain and a plexin repeat as determined by searching for statistically significant matches in the hidden Markov model (HMM)-based PFAM database of conserved protein family domains. (See Table 3.) Data from BLAST analyses against the DOMO and PRODOM databases provide further corroborative evidence that SEQ ID NO:31 is a semaphorin. In an alternative example, SEQ ID NO:35 is 61% identical to murine C-type lectin (GenBank ID g4159801) as determined by the Basic local Alignment Search Tool (BLAST). (See Table 2.) The BLAST probability score is 2.9e-75, which indicates the probability of obtaining the observed polypeptide sequence alignment by chance. SEQ ID NO:35 also contains a lectin C-type domain as determined by searching for statistically significant matches in the hidden Markov model (HMM)-based PFAM database of conserved protein family domains. (See Table 3.) Data from BLIMPS and PROFILESCAN analyses provide further corroborative evidence that SEQ ID NO:35 is a lectin. SEQ ID NO:1, SEQ ID NO:3-5, SEQ ID NO:7-9, SEQ ID NO:11, SEQ ID NO: 13, SEQ ID NO:15-21, SEQ ID NO:23, SEQ ID NO:25-30, SEQ ID NO:32-34 and SEQ ID NO:36 were analyzed and annotated in a similar manner. The algorithims and parameters for the analysis of SEQ ID NO:1-36 are described in Table 7.

As shown in Table 4, the full length polynucleotide sequences of the present invention were assembled using cDNA sequences or coding (exon) sequences derived from genomic DNA, or any combination of these two types of sequences. Columns 1 and 2 list the polynucleotide sequence identification number (Polynucleotide SEQ ID NO:) and the corresponding Incyte polynucleotide consensus sequence number (Incyte Polynucleotide ID) for each polynucleotide of the invention. Column 3 shows the length of each polynucleotide sequence in basepairs. Column 4 lists fragments of the polynucleotide sequences which are useful, for example, in hybridization or amplification technologies that identify SEQ ID NO:37-72 or that distinguish between SEQ ID NO:37-72 and related polynucleotide sequences. Column 5 shows identification numbers corresponding to cDNA sequences, coding sequences (exons) predicted from genomic DNA, and/or sequence assemblages comprised of both cDNA and genomic DNA. These sequences were used to assemble the full length polynucleotide sequences of the invention. Columns 6 and 7 of Table 4 show the nucleotide start (5′) and stop (3′) positions of the cDNA and/or genomic sequences in column 5 relative to their respective full length sequences.

The identification numbers in Column 5 of Table 4 may refer specifically, for example, to Incyte cDNAs along with their corresponding cDNA libraries. For example, 7347284H1 is the identification number of an Incyte cDNA sequence, and LUNLTUE01 is the cDNA library from which it is derived. Incyte cDNAs for which cDNA libraries are not indicated were derived from pooled cDNA libraries (e.g., 71699406V1). Alternatively, the identification numbers in column 5 may refer to GenBank cDNAs or ESTs (e.g., g1242437) which contributed to the assembly of the full length polynucleotide sequences. Alternatively, the identification numbers in column 5 may refer to coding regions predicted by Genscan analysis of genomic DNA. For example, GNN.g7923864_{—}002 is the identification number of a Genscan-predicted coding sequence, with g7923864 being the GenBank identification number of the sequence to which Genscan was applied. The Genscan-predicted coding sequences may have been edited prior to assembly. (See Example IV.) Alternatively, the identification numbers in column 5 may refer to assemblages of both cDNA and Genscan-predicted exons brought together by an “exon stitching” algorithm. (See Example V.) Alternatively, the identification numbers in column 5 may refer to assemblages of both cDNA and Genscan-predicted exons brought together by an “exon-stretching” algorithm. For example, FL2428715_g6815043_{—}000026_g8052237_{—}1_{—}3_{—}4.edit is the identification number of a “stretched” sequence, with 2428715 being the Incyte project identification number, g6815043 being the GenBank identification number of the human genomic sequence to which the “exon-stretching” algorithm was applied, and g8052237 being the GenBank identification number of the nearest GenBank protein homolog. (See Example V.) In some cases, Incyte cDNA coverage redundant with the sequence coverage shown in column 5 was obtained to confirm the final consensus polynucleotide sequence, but the relevant Incyte cDNA identification numbers are not shown.

Table 5 shows the representative cDNA libraries for those fill length polynucleotide sequences which were assembled using Incyte cDNA sequences. The representative cDNA library is the Incyte cDNA library which is most frequently represented by the Incyte cDNA sequences which were used to assemble and confirm the above polynucleotide sequences. The tissues and vectors which were used to construct the cDNA libraries shown in Table 5 are described in Table 6.

The invention also encompasses ECMCAD variants. A preferred ECMCAD variant is one which has at least about 80%, or alternatively at least about 90%, or even at least about 95% amnino acid sequence identity to the ECMCAD amino acid sequence, and which contains at least one functional or structural characteristic of ECMCAD.

The invention also encompasses polynucleotides which encode ECMCAD. In a particular embodiment, the invention encompasses a polynucleotide sequence comprising a sequence selected from the group consisting of SEQ ID NO:37-72, which encodes ECMCAD. The polynucleotide sequences of SEQ ID NO:37-72, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses a variant of a polynucleotide sequence encoding ECMCAD. In particular, such a variant polynucleotide sequence will have at least about 70%, or alternatively at least about 85%, or even at least about 95% polynucleotide sequence identity to the polynucleotide sequence encoding ECMCAD. A particular aspect of the invention encompasses a variant of a polynucleotide sequence comprising a sequence selected from the group consisting of SEQ ID NO:37-72 which has at least about 70%, or alternatively at least about 85%, or even at least about 95% polynuclcotidc sequence identity to a nucleic acid sequence selected from the group consisting of SEQ ID NO:37-72. Any one of the polynucleotide variants described above can encode an amino acid sequence which contains at least one functional or structural characteristic of ECMCAD.

It will be appreciated by those skilled in the art that as a result of the degeneracy of the genetic code, a multitude of polynucleotide sequences encoding ECMCAD, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring ECMCAD, and all such variations arc to be considered as being specifically disclosed.

Although nucleotide sequences which encode ECMCAD and its variants are generally capable of hybridizing to the nucleotide sequence of the naturally occurring ECMCAD under appropriately selected conditions of stringency, it may be advantageous to produce nucleotide sequences encoding ECMCAD or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding ECMCAD and its derivatives without altering the encoded amino acid sequences include the production of RNA transcripts having more desirable properties, such as a greater half-life, than transcripts produced from the naturally occurring sequence.

The invention also encompasses production of DNA sequences which encode ECMCAD and ECMCAD derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding ECMCAD or any fragment thereof.

Also encompassed by the invention are polynucleotide sequences that are capable of hybridizing to the claimed polynucleotide sequences, and, in particular, to those shown in SEQ ID NO:37-72 and fragments thereof under various conditions of stringency. (See, e.g., Wahl, G. M. and S. L. Berger (1987) Methods Enzymol. 152:399407; Kimmel, A. R. (1987) Methods Enzymol. 152:507-511.) Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase 1, SEQUENASE (US Biochemical, Cleveland Ohio), Taq polymerase (Applied Biosystems), thermostable T7 polymerase (Amersham Pharmacia Biotech, Piscataway N.J.), or combinations of polymerases and proofreading exonucleases such as those found in the ELONGASE amplification system (Life Technologies, Gaithersburg Md.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems), Sequencing is then carried out using either the ABI 373 or 377 DNA sequencing system (Applied Biosystems), the MEGABACE 1000 DNA sequencing system (Molecular Dynamics, Sunnyvale Calif.), or other systems known in the art. The resulting sequences are analyzed using a variety of algorithms which are well known in the art. (See, e.g., Ausubel, F. M. (1997) Short Protocols in Molecular Biology, John Wiley & Sons, New York N.Y., unit 7.7; Meyers, R. A. (1995) Molecular Biology and Biotechnology, Wiley VCH, New York N.Y., pp. 856-853.)

The nucleic acid sequences encoding ECMCAD may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, T. et al. (1988) Nucleic Acids Res. 16:8186.) A third method, capture PCR, involves PCR amplification of DNA fragments adjacent to known sequences in human and yeast artificial chromosome DNA. (See, e.g., Lagerstrom, M. et al. (1991) PCR Methods Applic. 1: 111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genornic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotide sequences or fragments thereof which encode ECMCAD may be cloned in recombinant DNA molecules that direct expression of ECMCAD, or fragments or functional equivalents thereof, in appropriate host cells. Due to the inherent degeneracy of the genetic code, other DNA sequences which encode substantially the same or a functionally equivalent amino acid sequence may be produced and used to express ECMCAD.

The nucleotide sequences of the present invention can be engineered using methods generally known in the art in order to alter ECMCAD-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458: Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of ECMCAD, such as its biological or enzymatic activity or its ability to bind to other molecules or compounds. DNA shuffling is a process by which a library of gene variants is produced using PCR-moediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, sequences encoding ECMCAD may be synthesized, in whole or in part, using chemical methods well known in the art. (See, e.g., Caruthers, M. H. et al. (1980) Nucleic Acids Symp. Ser. 7:215-223; and Horn, T. et al. (1980) Nucleic Acids Symp. Ser. 7:225-232.) Alternatively, ECMCAD itself or a fragment thereof may be synthesized using chemical methods. For example, peptide synthesis can be performed using various solution-phase of solid-phase techniques. (See, e.g., Creighton, T. (1984) Proteins, Structures and Molecular Properties, WH, Freeman, New York N.Y., pp. 55-60; and Roberge, J. Y. et al. (1995) Science 269:202-204.) Automated synthesis may be achieved using the ABI 431A peptide synthesizer (Applied Biosystems). Additionally, the amino acid sequence of ECMCAD, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography. (See, e.g., Chiez, R. M. and F. Z. Regnier (1990) Methods Enzymol. 182:392-421.) The composition of the synthetic peptides may be confirmed by amino acid analysis or by sequencing. (See, e.g., Creighton, supra, pp. 28-53.)

In order to express a biologically active ECMCAD, the nucleotide sequences encoding ECMCAD or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′untranslated regions in the vector and in polynucleotide sequences encoding ECMCAD. Such elements may vary in their strength and specificity. Specific initiation signals may also be used to achieve more efficient translation of sequences encoding ECMCAD. Such signals include the ATG initiation codon and adjacent sequences, e.g. the Kozak sequence. In cases where sequences encoding ECMCAD and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probl. Cell Differ. 20:125-162.)

Methods which are well known to those skilled in the art may be used to construct expression vectors containing sequences encoding ECMCAD and appropriate transcriptional and translational control elements. These methods include in vitro recombinant DNA tecluiques, synthetic techniques, and in vivo genetic recombination. (See, e.g., Sambrook J. et al. (1989) Molecular Cloning, A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16.)

A variety of expression vector/host systems may be utilized to contain and express sequences encoding ECMCAD. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plan( cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. (See, e.g., Sambrook, supra; Ausubel, supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet. 15:345-355.) Expression vectors derived from retroviruses, adenoviruses, or herpes or vaccinia viruses, or from various bacterial plasmids, may be used for delivery of nucleotide sequences to the targeted organ, tissue, or cell population. (See, e.g., Di Nicola, M. et al. (1998) Cancer Gen. Ther. 5(6):350-356; Yu, M. et al. (1993) Proc. Natl. Acad. Sci. USA 90(13):6340-6344; Buller, R. M. et al. (1985) Nature 317(6040):813-815; McGregor, D. P. et al. (1994) Mol. Immunol. 31(3):219-226; and Verma, I. M. and N. Somia (1997) Nature 389:239-242.) The invention is not limited by the host cell employed.

In bacterial systems, a number of cloning and expression vectors may be selected depending upon the use intended for polynucleotide sequences encoding ECMCAD. For example, routine cloning, subcloning, and propagation of polynucleotide sequences encoding ECMCAD can be achieved using a multifunctional E. coli vector such as PBLUESCRIPT (Stratagene, La Jolla Calif.) or PSPORT1 plasmid (Life Technologies). Ligation of sequences encoding ECMCAD into the vector's multiple cloning site disrupts the lacZ gene, allowing a calorimetric screening procedure for identification of transformed bacteria containing recombinant molecules. In addition, these vectors may be useful for in vitro transcription, didcoxy sequencing, single strand rescue with helper phage, and creation of nested deletions in the cloned sequence. (See, e.g., Van Heeke, G. and S. M. Schuster (1989) J. Biol. Chem. 264:5503-5509.) When large quantities of ECMCAD are needed, e.g. for the production of antibodies, vectors which direct high level expression of ECMCAD may be used. For example, vectors containing the strong, inducible SP6 or T7 bacteriophage promoter may be used.

Yeast expression systems may be used for production of ECMCAD. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign sequences into the host genome for stable propagation. (See, e.g., Ausubel, 1995, supra; Bitter, G. A. et al. (1987) Methods Enzymol. 153:516-544; and Scorer, C. A. et al. (1994) Bio/Technology 12:181-184.)

Plant systems may also be used for expression of ECMCAD. Transcription of sequences encoding ECMCAD may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., The McGraw Hill Yearbook of Science and Technology (1992) McGraw Hill, New York N.Y., pp. 191-196.)

In mammalian cells, a number of viral-based expression systems may be utilized. In cases where an adenovirus is used as an expression vector, sequences encoding ECMCAD may be ligated into an adenovirus transcription/translation complex consisting of the late promoter and tripartite leader sequence. Insertion in a non-essential E1 or E3 region of the viral genome may be used to obtain infective virus which expresses ECMCAD in host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. RACs of about 6 kb to 10 Mb are constructed and delivered via conventional delivery methods (liposomes, polycationic amino polymers, or vesicles) for therapeutic purposes. (See, e.g., Harrington, J. J. et al. (I 997) Nat. Genet. 15:345-355.)

For long term production of recombinant proteins in mammalian systems, stable expression of ECMCAD in cell lines is preferred. For example, sequences encoding ECMCAD can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk and apr cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfluon and phosphinotricin acetyltransferase, respectively. (See, e.g., Wigler, M. et al. (1980) Proc. Natl. Acad. Sci. USA 77:3567-3570; Colbere-Garapin, F. et al. (1981) J. Mol. Biol. 150:1-14.) Additional selectable genes have been described, e.g., trpB and hisD, which alter cellular requirements for metabolites. (See, e.g., Haitman, S. C. and R. C. Mulligan (1988) Proc. Natl. Acad. Sci. USA 85:8047-8051.) Visible markers, e.g., anthocyanins, green fluorescent proteins (GFP; Clontech), β glucuronidase and its substrate β-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may need to be confirmed. For example, if the sequence encoding ECMCAD is inserted within a marker gene sequence, transformed cells containing sequences encoding ECMCAD can be identified by the absence of marker gene function. Alternatively, a marker gene can be placed in tandem with a sequence encoding ECMCAD under the control of a single promoter. Expression of the marker gene in response to induction or selection usually indicates expression of the tandem gene as well.

In general, host cells that contain the nucleic acid sequence encoding ECMCAD and that express ECMCAD may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of ECMCAD using either specific polyclonal or monoclonal antibodies are known in the art. Examples of such techniques include enzyme-linked immunosorbent assays (ELISAs), radioimmunoassays (RfAs), and fluorescence activated cell sorting (FACS). A two-site, monoclonal-based immunoassay utilizing monoclonal antibodies reactive to two non-interfering epitopes on ECMCAD is preferred, but a competitive binding assay may be employed. These and other assays are well known in the art. (See, e.g., Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and may be used in various nucleic acid and amino acid assays. Means for producing labeled hybridization or PCR probes for detecting sequences related to polynucleotides encoding ECMCAD include oligolabeling, nick translation, end-labeling, or PCR amplification using a labeled nucleotide. Alternatively, the sequences encoding ECMCAD, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Pharmacia Biotech, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding ECMCAD may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode ECMCAD may be designed to contain signal sequences which direct secretion of ECMCAD through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and W138) are available from the American Type Culture Collection (ATCC, Manassas Va.) and may be chosen to ensure the correct modification and processing of the foreign protein.

In another embodiment of the invention, natural, modified, or tecombinant nucleic acid sequences encoding ECMCAD may be ligated to a heterologous sequence resulting in translation of a fusion protein in any of the aforementioned host systems. For example, a chimeric ECMCAD protein containing a heterologous moiety that can be recognized by a commercially available antibody may facilitate the screening of peptide libraries for inhibitors of ECMCAD activity. Heterologous protein and peptide moieties may also facilitate purification of fusion proteins using commercially available affinity matrices. Such moieties include, but are not limited to, glutathione S-transferase (GST), maltose binding protein (MBP), tioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the ECMCAD encoding sequence and the heterologous protein sequence, so that ECMCAD may be cleaved away from the heterologous moiety following purification. Methods for fusion protein expression and purification are discussed in Ausubel (1995, supra, ch. 10). A variety of commercially available kits may also be used to facilitate expression and purification of fusion proteins.

In a further embodiment of the invention, synthesis of radiolabeled ECMCAD may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

ECMCAD of the present invention or fragments thereof may be used to screen for compounds that specifically bind to ECMCAD. At least one and up to a plurality of test compounds may be screened for specific binding to ECMCAD. Examples of test compounds include antibodies, oligonucleotides, proteins (e.g., receptors), or small molecules.

In one embodiment, the compound thus identified is closely related to the natural ligand of ECMCAD. e.g., a ligand or fragment thereof, a natural substrate, a structural or functional mimetic, or a natural binding partner. (See, e.g., Coligan, J. E. et al. (1991) Current Protocols in Immunology 1(2): Chapter 5.) Similarly, the compound can be closely related to the natural receptor to which ECMCAD binds, or to at least a fragment of the receptor, e.g., the ligand binding site. In either case, the compound can be rationally designed using known techniques. In one embodiment, screening for these compounds involves producing appropriate cells which express ECMCAD, either as a secreted protein or on the cell membrane. Preferred cells include cells from mammals, yeast, Drosophila, or E. coli. Cells expressing ECMCAD or cell membrane fractions which contain ECMCAD are then contacted with a test compound and binding, stimulation, or inhibition of activity of either ECMCAD or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with ECMCAD, either in solution or affixed to a solid support, and detecting the binding of ECMCAD to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

ECMCAD of the present invention or fragments thereof may be used to screen for compounds that modulate the activity of ECMCAD. Such compounds may include agonists, antagonists, or partial or inverse agonists. In one embodiment, an assay is performed under conditions permissive for ECMCAD activity, wherein ECMCAD is combined with at least one test compound, and the activity of ECMCAD in the presence of a test compound is compared with the activity of ECMCAD in the absence of the test compound. A change in the activity of ECMCAD in the presence of the test compound is indicative of a compound that modulates the activity of ECMCAD. Alternatively, a test compound is combined with an in vitro or cell-free system comprising ECMCAD under conditions suitable for ECMCAD activity, and the assay is performed. In either of these assays, a test compound which modulates the activity of ECMCAD may do so indirectly and need not come in direct contact with the test compound. At least one and up to a plurality of test compounds may be screened.

In another embodiment, polynucleotides encoding ECMCAD or their mammalian homologs may be “knocked out” in an animal model system using homologous recombination in embryonic stem (ES) cells. Such techniques arc well known in the art and are useful for the generation of animal models of human disease. (See, e.g., U.S. Pat. No. 5,175,383 and U.S. Pat. No. 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Maith, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding ECMCAD may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding ECMCAD can also be used to create “knockin” humanized animals (pigs) or transgenic animals (mice or rats) to model human disease. With knockin technology, a region of a polynucleotide encoding ECMCAD is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress ECMCAD, e.g., by secreting ECMCAD in its milk, may also serve as a convenient source of that protein (Janne, J. et al. (1998) Biotechnol. Annu. Rev. 4:55-74).

### THERAPEUTICS

Chemical and structural similarity, e.g., in the context of sequences and motifs, exists between regions of ECMCAD and extracellular matrix and cell adhesion molecules In addition, the expression of ECMCAD is closely associated with brain, prostate, atrial myxoma, cerebellum, cervical dorsal root ganglion, cardiac muscle, mesentel fat, kidney epithelium, thymus, endothelium, ovary, placenta, smooth muscle, fallopian tube, breast, cartilage, bladder, rib, colon, spine, gall bladder, blood granulocytes, submandibular gland, seminal vesicle, and intestine tissues; with tumors of the brain, prostate, rib, and fallopian tube; and with dermal microvascular endothelial cells, hNT2 cells derived from a human teratocarcinoma, and 293-EBNA transformed embryonal cells derived from kidney epithelial tissue. Therefore, ECMCAD appears to play a role in genetic, immune/inflammatory, developmental, neurological, connective tissue, and cell proliferative disorders, including cancer. In the treatment of disorders associated with increased ECMCAD expression or activity, it is desirable to decrease the expression or activity of ECMCAD. In the treatment of disorders associated with decreased ECMCAD expression or activity, it is desirable to increase the expression or activity of ECMCAD.

Therefore, in one embodiment, ECMCAD or a fragment or derivative thereof may be administered to a subject to treat or prevent a disorder associated with decreased expression or activity of ECMCAD. Examples of such disorders include, but are not limited to, a genetic disorder such as adrenoleukodystrophy, Alport's syndrome, choroideremia, Duchenne and Becker muscular dystrophy, Down's syndrome, cystic fibrosis, chronic granulomatous disease, Gaucher's disease, Huntington's chorea, Marfan's syndrome, muscular dystrophy, myotonic dystrophy, pyenodysostosis, Refsum's syndrome, retinoblastoma, sickle cell anemia, thalassemia, Werner syndrome, von Willebrand's disease, Wilms' tumor, Zellweger syndrome, peroxisomal acyl-CoA oxidase deficiency, peroxisomal thiolase deficiency, peroxisomal bifunctional protein deficiency, mitochondrial carnitine palmitoyl transferase and carnitine deficiency, mitochondrial very-long-chain acyl-CoA dehydrogenase deficiency, mitochondrial medium-chain acyl-CoA dehydrogenase deficiency, mitochondrial short-chain acyl-CoA dehydrogenase deficiency, mitochondrial electron transport flavoprotein and electron transport flavoprotein:ubiquinone oxidoreductase deficiency, mitochondrial trifunctional protein deficiency, and mitochondrial short-chain 3-hydroxyacyl-CoA dehydrogenase deficiency; an immune/inflammatory disorder such as acquired immunodeficiency syndrome (AIDS), X-linked agammaglobinemia of Bruton, common variable immunodeficiency (CVI), DiGeorge's syndrome (thymic hypoplasia), thymic dysplasia, isolated IgA deficiency, severe combined immunodeficiency disease (SCID), immunodeficiency with thrombocytopenia and eczema (Wiskott-Aldrich syndrome), Chediak-Higashi syndrome, chronic granulomatous diseases, hereditary angioneurotic edema, immunodeficiency associated with Cushing's disease, Addison's disease, adult respiratory distress syndrome, allergies, ankylosinig spondylitis, aimyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, eiytlioblastosis fetalis, erythema nodosum, atrophic gasthitis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjogren's syndrome, systemic anaphylaxis, systemic lupus erythematosus, systernic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; a neurological disorder such as epilepsy, ischemic cerebrovascular disease, stroke, cerebral neoplasms, Alzheimer's disease, Pick's disease, Huntinigton's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyoma, epidural abscess, suppurative intracranial thrombophlebitis, myelitis and radiculitis, viral central nervous system disease, prion diseases including kuru, Creutzfeldt-Jakob disease, and Gerstmann-Straussler-Scheiniker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postheipetic neuralgia, Tourette's disorder, progressive supranuclear palsy, corticobasal degeneration, and familial frontotemporal dementia; a connective tissue disorder such as osteogenesis imperfecta, Ehlers-Danlos syndrome, chondrodysplasias, Marfan syndrome, Alport syndrome, familial aortic aneurysm, achondroplasia, mucopolysaccharidoses, osteoporosis, osteopetrosis, Paget's disease, rickets, osteomalacia, hyperparathyroidism, renal osteodystrophy, osteoneciosis, osteomyelitis, osteoma, osteoid osteoma, osteoblastoma, osteosarcoma, osteochondroma, chondroma, chondroblastoma, chondromyxoid fibroma, chondrosarcoma, fibrous cortical defect, nonossifying fibroma, fibrous dysplasia, fibrosarcoma, malignant fibrous histiocytoma, Ewing's sarcoma, primitive neuroectodermal tumor, giant cell tumor, osteoarthritis, rheumatoid arthritis, ankylosing spondyloarthritis, Reiter's syndrome, psoriatic arthritis, enteropathic arthritis, infectious arthritis, gout, gouty arthritis, calcium pyrophosphate crystal deposition disease, ganglion, synovial cyst, villonodular synovitis, systemic sclerosis, Dupuytren's contracture, hepatic fibrosis, lupus erythematosus, mixed connective tissue disease, epidermolysis bullosa simplex, bullous congenital ichthyosiform erythroderma (epiderniolytic hyperkeratosis), non-epidermolytic and epidermolytic palmoplantar keratoderina, ichthyosis bullosa of Siemens, pachyonychia congenital and white sponge nevus; and a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycytheima vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovaty, pancreas, parathyroid, penis, prostate, salivary glands, skin, spleen, testis, thymus, thyroid, and uterus.

In another embodiment, a vector capable of expressing ECMCAD or a fragment or derivative thereof may be administered to a subject to treat or prevent a disorder associated with decreased expression or activity of ECMCAD including, but not limited to, those described above.

In a further embodiment, a composition comprising a substantially purified ECMCAD in conjunction with a suitable pharmaceutical carrier may be administered to a subject to treat or prevent a disorder associated with decreased expression or activity of ECMCAD including, but not limited to, those provided above.

In still another embodiment, an agonist which modulates the activity of ECMCAD may be administered to a subject to treat or prevent a disorder associated with decreased expression or activity of ECMCAD including, but not limited to, those listed above.

In a further embodiment, an antagonist of ECMCAD may be administered to a subject to treat or prevent a disorder associated with increased expression or activity of ECMCAD. Examples of such disorders include, but are not limited to, those genetic, immunelinflammatory, developmental, neurological, connective tissue, and cell proliferative disorders, including cancer described above. In one aspect, an antibody which specifically binds ECMCAD may be used directly as an antagonist or indirectly as a targeting or delivery mechanism for bringing a pharmaceutical agent to cells or tissues which express ECMCAD.

In an additional embodiment, a vector expressing the complement of the polynucleotide encoding ECMCAD may be administered to a subject to treat or prevent a disorder associated with increased expression or activity of ECMCAD including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to aclueve therapeutic efficacy with lower dosages of each agent, thus reducing the potential for adverse side effects.

An antagonist of ECMCAD may be produced using methods which are generally known in the art. In particular, purified ECMCAD may be used to produce antibodies or to screen libraries of pharmaceutical agents to identify those which specifically bind ECMCAD. Antibodies to ECMCAD may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use.

For the production of antibodies, various hosts including goats, rabbits, rats, mice, humans, and others may be immunized by injection with ECMCAD or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Cornebacterium parvum are especially preferable.

It is preferred that the oligopeptides, peptides, or fragments used to induce antibodies to ECMCAD have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of ECMCAD amino acids may be fused with those of another protein, such as KLH, and antibodies to the chimeric molecule may be produced.

Monoclonal antibodies to ECMCAD may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce ECMCAD-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for ECMCAD may also be generated. For example, such fragments include, but are not limited to, F(ab′)₂ fragments produced by pepsin digestion of the antibody molecule and Fab fragments generated by reducing the disulfide bridges of the F(ab)2 fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between ECMCAD and its specific antibody. A two-site, monoclonal-based immunoassay utilizing monoclonal antibodies reactive to two non-interfering ECMCAD epitopes is generally used, but a competitive binding assay may also be employed (Pound, supra).

Various methods such as Scatchard analysis in conjunction with radioimmunoassay techniques may be used to assess the affinity of antibodies for ECMCAD. Affinity is expressed as an association constant Kₐ, which is defined as the molar concentration of ECMCAD-antibody complex divided by the molar concentrations of free antigen and free antibody under equilibrium conditions. The Kₐ determined for a preparation of polyclonal antibodies, which are heterogeneous in their affinitics for multiple ECMCAD epitopes, represents the average affinity, or avidity, of the antibodies for ECMCAD. The Kₐ determined for a preparation of monoclonal antibodies, which are monospecific for a particular ECMCAD epitope, represents a true measure of affinity. High-affinity antibody preparations with Kₐ ranging from about 10⁹ to 10¹² L/mole are preferred for use in immunoassays in which the ECMCAD-antibody complex must withstand rigorous manipulations. Low-affinity antibody preparations with Kₐ ranging from about 10⁶ to 10⁷ L/mole are preferred for use in immunopurification and similar procedures which ultimately require dissociation of ECMCAD, preferably in active form, from the antibody (Catty, D. (1988) Antibodies, Volume I: A Practical Approach, IRL Press, Washington D.C.; Liddell, J. E. and A. Ciyer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of ECMCAD-antibody complexes. Procedures for evaluating antibody specificity, titer, and avidity, and guidelines for antibody quality and usage in various applications, are generally available. (See, e.g., Catty, supra, and Coligan et al. supra.)

In another embodiment of the invention, the polynucleotides encoding ECMCAD, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding ECMCAD. Such technology is well known in the all, and antisense oligonucleotides or larger fragments can be designed from various locations along the coding or control regions of sequences encoding ECMCAD. (See, e.g., Agrawal, S., ed. (1996) Antisense Therapeutics, Humana Press Inc., Totawa N.J.)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the taret protein. (See, e.g., Slater, J. E. et al. (1998) J. Allergy Cli. Immunol. 102(3):469-475; and Scanlon, K. J. et al. (1995) 9(13): 1288-1296.) Antisense sequences can also be introduced intracellularly through the use of viral vectors, such as retrovirus and adeno-associated virus vectors. (See, e.g., Miller, A. D. (1990) Blood 76:271; Ausubel, supra; Uckert, W. and W. Walther (1994) Pharmacol. Ther. 63(3):323-347.) Other gene delivery mechanisms include liposome-derived systems, artificial viral envelopes, and other systems known in the alt. (See, e.g., Rossi, J. J. (1995) Br. Med. Bull. 51(1):217-225; Boado, R. J. et al. (1998) J. Pharm. Sci. 87(11):1308-1315; and Morris, M. C. et al. (1997) Nucleic Acids Res. 25(14):2730-2736.)

In another embodiment of the invention, polynucleotides encoding ECMCAD may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon. C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703). thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA. 93:1 1395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in ECMCAD expression or regulation causes disease, the expression of ECMCAD from an appropriate population of transduced cells may alleviate the clinical manifestations caused by the genetic deficiency.

In a further embodiment of the invention, diseases or disorders caused by deficiencies in ECMCAD are treated by constructing mammalian expression vectors encoding ECMCAD and introducing these vectors by mechanical means into ECMCAD-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J-L. and H. Récipon (1998) Curr. Opin. Biotechnol. 9:445-450).

Expression vectors that may be effective for the expression of ECMCAD include, but are not limited to, the PCDNA 3.1, EPITAG, PRCCMV2, PREP, PVAX vectors (Invitrogen, Carlsbad Calif.), PCMV-SCRIPT, PCMV-TAG, PEGSLYPERV (Stratagene, La Jolla Calif.), and PTET-OFF, PTET-ON, PTRE2, PTRE2-LUC, PTK-HYG (Clontech, Palo Alto Calif.). ECMCAD may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycime-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and Blau, H. M. supra)), or (iii) a tissue-specific promoter or the native promoter of the endogenous gene encoding ECMCAD from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to ECMCAD expression are treated by constructing a retrovirus vector consisting of (i) the polynucleotide encoding ECMCAD under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano. D. et al. (1987) J. Virol. 61:1647-1650; Bender, M. A. et al. (1987) J. Virol. 61:1639-1646; Adam, M. A. and A. D. Miller(I988) J. Virol. 62:3802-3806: Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:47074716; Ranga, U. et al. (1998) Proc. Natl. Acad. Sci. USA 95:1201-1206; Su, L. (1997) Blood 89:2283-2290).

In the alternative, an adenovirus-based gene therapy delivery system is used to deliver polynucleotides encoding ECMCAD to cells which have one or more genetic abnormalities with respect to the expression of ECMCAD. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenoviius vectors for gene therapy”), hereby incorporated by reference. For adenoviral vectors, see also Antinozzi, P. A. et al. (1999) Annu. Rev. Nutr. 19:511-544 and Verma, I. M. and N. Somia (1997) Nature 18:389:239-242, both incorporated by reference herein.

In another alterative, a herpes-based, gene therapy delivery system is used to deliver polynucleotides encoding ECMCAD to target cells which have one or more genetic abnormalities with respect to the expression of ECMCAD. The use of herpes simplex virus (HSV)-based vectors may be especially valuable for introducing ECMCAD to cells of the central nervous system, for which HSV has a tropism. The construction and packaging of herpes-based vectors are well known to those with ordinary skill in the art. A replication-competent herpes simplex virus (HSV) type 1 -based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999) J. Virol. 73:519-532 and Xu, H. et al. (1994) Dev. Biol. 163:152-161, hereby incorporated by reference. The manipulation of cloned herpesvirus sequences the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another alternative, an alphavirus (positive, single-stranded RNA virus) vector is used to deliver polynucleotides encoding ECMCAD to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for ECMCAD into the alphavirus genome in place of the capsid-coding region results in the production of a large number of ECMCAD-coding RNAs and the synthesis of high levels of ECMCAD in vector transduced cells. While alphavirus infection is typically associated with cell lysis within a few days, the ability to establish a persistent infection in hamster normal kidney cells (BHK-21) with a variant of Sindbis virus (SET) indicates that the lytic replication of alphaviruses can be altered to suit the needs of the gene therapy application (Dryga, S. A. et al. (1997) Virology 228:74-83). The wide host range of alphaviruses will allow the introduction of ECMCAD into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee. J. E. et al. (1994) in Huber, B. E. and B. I. Carr, Molecular and Immunologic Approaches, Futura Publishing, Mt. Kisco N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA followed by endonucleolytic cleavage. For example, engineered hammerhead motif ribozyme molecules may specifically and efficiently catalyze endonucleolytic cleavage of sequences encoding ECMCAD.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the ant for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding ECMCAD. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′O-methyl rather than phospliodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding ECMCAD. Compounds which may be effective in altering expression of a specific polynucleotide may include, but are not limited to, oligonucleotides, antisense oligonucleotides, triple helix-forming oligonucleotides. transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased ECMCAD expression or activity, a compound which specifically inhibits expression of the polynucleotide encoding ECMCAD may be therapeutically useful, and in the treatment of disorders associated with decreased ECMCAD expression or activity, a compound which specifically promotes expression of the polynucleotide encoding ECMCAD may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a, library of chemical compounds created combinatorially or randomly. A sample comprising a polynucleotide encoding ECMCAD is exposed to at least one test compound thus obtained. The sample may comprise, for example, an intact or permeabilized cell, or an in vitro cellfree or reconstituted biochemical system. Alterations in the expression of a polynucleotide encoding ECMCAD are assayed by any method commonly known in the art. Typically, the expression of a specific nucleotide is detected by hybridization with a probe having a nucleotide sequence complementary to the sequence of the polynucleotide encoding ECMCAD. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435: Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino pollers may be achieved using methods which are well known in the art. (See, e.g., Goldman, C. K. et al. (1997) Nat. Biotechnol. 15:462-466.)

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of ECMCAD, antibodies to ECMCAD, and mimetics, agonists, antagonists, or inhibitors of ECMCAD.

The compositions utilized in this invention may be administered by any number of routes including, but not limited to, oral, intravenous, intramuscular, intra-aterial, intramedullaty, intrathecal, intravetitricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising ECMCAD or fragments thereof. For example, liposome preparations containing a cell-impermeable macromolecule may promote cell fusion and intracellular delivery of the macromolecule. Alternatively, ECMCAD or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al (1999) Science 285:1569-1572).

For any compound, the therapeutically effective dose can be estimated initially either in cell culture assays, e.g., of neoplastic cells, or in animal models such as mice. rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example ECMCAD or fragments thereof, antibodies of ECMCAD, and agonists, antagonists or inhibitors of ECMCAD, which ameliorates the symptoms or condition. Therapeutic efficacy and toxicity may be determined by standard pharmaceutical procedures in cell cultures or with experimental animals, such as by calculating the ED50 (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

### DIAGNOSTICS

In another embodiment, antibodies which specifically bind ECMCAD may be used for the diagnosis of disorders characterized by expression of ECMCAD, or in assays to monitor patients being treated with ECMCAD or agonists, antagonists, or inhibitors of ECMCAD. Antibodies useful for diagnostic purposes may be prepared in the same manner as described above for therapeutics. Diagnostic assays for ECMCAD include methods which utilize the antibody and a label to detect ECMCAD in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring ECMCAD, including ELISAs, RIAs, and FACS, are known in the art and provide a basis for diagnosing altered or abnormal levels of ECMCAD expression. Normal or standard values for ECMCAD expression are established by combining body fluids or cell extracts taken from normal mammalian subjects, for example, human subjects, with antibodies to ECMCAD under conditions suitable for complex formation. The amount of standard complex formation may be quantitated by various methods, such as photometric means. Quantities of ECMCAD expressed in subject, control, and disease samples from biopsied tissues are compared with the standard values. Deviation between standard and subject values establishes the parameters for diagnosing disease.

In another embodiment of the invention, the polynucleotides encoding ECMCAD may be used for diagnostic purposes. The polynucleotides which may be used include oligonucleotide sequences, complementary RNA and DNA molecules, and PNAs. The polynucleotides may be used to detect and quantify gene expression in biopsied tissues in which expression of ECMCAD may be correlated with disease. The diagnostic assay may be used to determine absence, presence, and excess expression of ECMCAD, and to monitor regulation of ECMCAD levels during therapeutic intervention.

In one aspect, hybridization with PCR probes which are capable of detecting polynucleotide sequences, including genomic sequences, encoding ECMCAD or closely related molecules may be used to identify nucleic acid sequences which encode ECMCAD. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding ECMCAD, allelic variants, or related sequences.

Probes may also be used for the detection of related sequences, and may have at least 50% sequence identity to any of the ECMCAD encoding sequences. The hybridization probes of the subject invention may be DNA or RNA and may be derived from the sequence of SEQ ID NO:37-72 or from genomic sequences including promoters, enhancers, and introns of the ECMCAD gene.

Means for producing specific hybridization probes for DNAs encoding ECMCAD include the cloning of polynucleotide sequences encoding ECMCAD or ECMCAD derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotide sequences encoding ECMCAD may be used for the diagnosis of disorders associated with expression of ECMCAD. Examples of such disorders include, but are not limited to, a genetic disorder such as adrenoleukodystrophy, Alport's syndrome, choroideremia, Duchenne and Becker muscular dystrophy, Down's syndrome, cystic fibrosis, chronic granulomatous disease, Gaucher's disease, Huntington's chorea, Marfan's syndrome, muscular dystrophy, myotonic dystrophy, pycnodysostosis, Refsum's syndrome, retinoblastoma, sickle cell anemia, thalassenia, Werner syndrome., von Willebrand's disease, Wilms' tumor, Zellweger syndrome, peroxisomal acyl-CoA oxidase deficiency, peroxisomal thiolase deficiency, peroxisomal bifunctional protein deficiency, mitochondrial carnitine palmitoyl transferase and carnitine deficiency, mitochondrial very-long-chain acyl-CoA dehydrogenase deficiency, mitochondrial medium-chain acyl-CoA dehydrogenase deficiency, mitochondrial short-chain acyl-CoA dehydrogenase deficiency, mitochondnial electron transport flavoprotein and electron transport flavoprotein:ubiquinone oxidoreductase deficiency, mitochondrial trifunctional protein deficiency, and mitochondrial short-chain 3-hydroxyacyl-CoA dehydrogenase deficiency; an immune/inflammatory disorder such as acquired immunodeficiency syndrome (AIDS), X-linked agammaglobinemia of Bruton, common variable immunodeficiency (CVI), DiGeorge's syndrome (thymic hypoplasia), thynic dysplasia, isolated IgA deficiency, severe combined immunodeficiency disease (SCID), immunodeficiency with thrombocytopenia and eczema (Wiskott-Aldrich syndrome), Chediak-Higashi syndrome, chronic granulomatous diseases, hereditary angioneurotic edema, immunodeficiency associated with Cushing's disease, Addison's disease, adult respiratory distress syndrome, allergies, ankrylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymuphocytotoxins, erythoblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjogren's syndrome, systemic anaphylaxis, systemic lupus erythematosus, systemic sclerosis, thrombocytopenia puipura, ulcerative colitis, uveitis, Werner syndrome, complications of cancer, hemodialysis, and extracorporeal circulation, viral, bacterial, fungal, parasitic, protozoal, and helminthic infections, and trauma; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, anriridia, geritourinary abnornalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anelncephaly, craniorachischisis, congenital glaucoma, cataract, and sensorineural hearing loss; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombopwlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotigerinal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, coilticobasal degeneration, and familial frontotemporal dementia; a connective tissue disorder such as osteogenesis imperfecta, Ehlers-Danlos syndrome, chondrodysplasias, Marfan syndrome, Alport syndrome, familial aortic aneurysm, achondroplasia, mucopolysaccharidoses, osteoporosis, osteopetrosis, Paget's disease, rickets, osteomalacia, hyperparathyroidism, renal osieodystrophy, osteonecrosis, osteomyelitis, osteoma, osteoid osteoma, osteoblastoma, osteosarcoma, osteochondroma, chondroma, chondroblastoma, chondromyxoid fibroma, chondrosarcoma, fibrous cortical defect, nonossifying fibroma, fibrous dysplasia, fibrosarcoma, malignant fibrous histiocetoma, Ewing's sarcoma, primitive neuroectodermal tumor, giant cell tumor, osteoartbritis, rheumatoid arthritis, ankylosing spondyloartluitis, Reiter's syndrome, psoriatic arthritis, enteropathic arthritis, infectious arthritis, gout, gouty arthritis, calcium pyrophosphate crystal deposition disease, ganglion, synovial cyst, villonodular synovitis, systemic sclerosis, Dupuytren's contracture, hepatic fibrosis, lupus erythematosus, mixed connective tissue disease, epidermolysis bullosa simplex, bullous congenital ichthyosiform eiythrodenna (epidermolytic hyperkeratosis), non-epidermlolytic and epidermolytic palmoplantar keratoderma, ichthyosis bullosa of Siemens, pachyonychia congenita, and white sponge nevus; and a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycytheinia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. The polynucleotide sequences encoding ECMCAD may be used in Southern or northern analysis, dot blot, or other membrane-based technologies; in PCR technologies; in dipstick, pin, and multiformat ELISA-like assays; and in microarrays utilizing fluids or tissues from patients to detect altered ECMCAD expression. Such qualitative or quantitative methods are well known in the art.

In a particular aspect, the nucleotide sequences encoding ECMCAD may be useful in assays that detect the presence of associated disorders, particularly those mentioned above. The nucleotide sequences encoding ECMCAD may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of nucleotide sequences encoding ECMCAD in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of ECMCAD, a normal or standard profile for expression is established. This may be accomplished by combining body fluids or cell extracts taken from normal subjects, either animal or human, with a sequence, or a fragment thereof, encoding ECMCAD, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding ECMCAD may involve the use of PCR. These oligomers may be chemically synthesized, generated enzymatically, or produced in vitro. Oligomers will preferably contain a fragment of a polynucleotide encoding ECMCAD, or a fragment of a polynucleotide complementary to the polynucleotide encoding ECMCAD, and will be employed under optimized conditions for identification of a specific gene or condition. Oligomers may also be employed under less stringent conditions for detection or quantification of closely related DNA or RNA sequences.

In a particular aspect, oligonucleotide primers derived from the polynucleotide sequences encoding ECMCAD may be used to detect single nucleotide polytheism (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from the polynucleotide sequences encoding ECMCAD are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplifiers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatogram. In the alterative. SNPs may be detected and characterized by mass spectrometry using, for example, the high throughput MASSARRAY system (Sequenom, Inc., San Diego Calif.).

Methods which may also be used to quantify the expression of ECMCAD include radiolabeling or biotinylating nucleotides, coamplification of a control nucleic acid, and interpolating results from standard curves. (See, e.g., Melby, P. C. et al. (1993) J. Immunol. Methods 159:235-244; Duplaa, C. et al. (1993) Anal. Biochem. 212:229-236.) The speed of quantitation of multiple samples may be accelerated by running the assay in a high-throughput format where the oligomer or polynucleotide of interest is presented in various dilutions and a spectrophotometic or calorimetric response gives rapid quantitation.

In further embodiments, oligonucleotides or longer fragments derived from any of the polynucleotide sequences described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, ECMCAD, fragments of ECMCAD, or antibodies specific for ECMCAD may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time. (See Seilhamer et al., “Comparative Gene Transcript Analysis,” U.S. Pat. No. 5,840,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471, expressly incorporated by reference herein). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released Feb. 29, 2000, available at http://www.niehs.nii.gov/oc/news/toxchip.htm.) Therefore, it is important and desirable in toxicological screening using toxicant signatures to include all expressed gene sequences.

In one embodiment, the toxicity of a test compound is assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another particular embodiment relates to the use of the polypeptide sequences of the present invention to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electropholesis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by compating its partial sequence, preferably of at least 5 contiguous amino acid residues, to the polypeptide sequences of the present invention. In some cases, further sequence data may be obtained for definitive protein identification.

A proteornic profile may also be generated using antibodies specific for ECMCAD to quantify the levels of ECMCAD expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array clement.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteotic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.) Various types of microarrays are well known and thoroughly described in DNA Microarrays: A Practical Approach, M. Schena, ed. (1999) Oxford University Press, London, hereby expressly incorporated by reference.

In another embodiment of the invention, nucleic acid sequences encoding ECMCAD may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet. 7:149-154.) Once mapped, the nucleic acid sequences of the invention may be used to develop genetic linkage maps, for example, which correlate the inheritance of a disease state with the inheritance of a particular chromosome region or restiction fragment length polymorphism (RFLP). (See, for example, Lander, E. S. and D. Botstein (1986) Proc. Natl. Acad. Sci. USA 83:7353-7357.)

Fluorescent in situ hybridization (FISH) may be correlated with other physical and genetic map data. (See, e.g., Heinz-Ulrich, et al. (1995) in Meyers, supra, pp. 965-968.) Examples of genetic map data can be found in various scientific journals or at the Online Mendelian Inheritance in Man (OMIM) World Wide Web site. Correlation between the location of the gene encoding ECMCAD on a physical map and a specific disorder, or a predisposition to a specific disorder, may help define the region of DNA associated with that disorder and thus may further positional cloning efforts. In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation (See, e.g., Gatti, R. A. et al. (1988) Nature 336:577-580.) The nucleotide sequence of the instant invention may also be used to detect differences in the chromosomal location due to translocation, inversion, etc., among normal, carrier, or affected individuals.

In another embodiment of the invention, ECMCAD, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between ECMCAD and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate. The test compounds are reacted with ECMCAD, or fragments thereof, and washed. Bound ECMCAD is then detected by methods well known in the art. Purified ECMCAD can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding ECMCAD specifically compete with a test compound for binding ECMCAD. In this manner, antibodies can be used to detect the presence of any peptide which shares one or more antigenic determinants with ECMCAD.

In additional embodiments, the nucleotide sequences which encode ECMCAD may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications, and publications mentioned above and below, including U.S. Ser. No. 60/215,454, U.S. Ser. No. 60/219,462, U.S. Ser. No. 60/240,111, U.S. Ser. No. 60/240,106, U.S. Ser. No. 60/244,021, U.S. Ser. No. 60/248,887, and U.S. Ser. No. 60/249,570 are hereby expressly incorporated by reference.

### EXAMPLES

### I. Construction of cDNA Libraries

Incyte cDNAs were derived from cDNA libraries described in the LIFESEQ GOLD database (Incyte Genomics, Palo Alto Calif.) and shown in Table 4, column 5. Some tissues were homogenized and lysed in guanidimiuin isothiocyanate, while others were homogenized and lysed in phenol or in a suitable mixture of denaturants, such as TRIZOL (Life Technologies), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Life Technologies), using the recommended procedures or similar methods known in the art. (See, e.g., Ausubel, 1997, supra, units 5.1-6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Pharmacia Biotech) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Life Technologies), PCDNA2.1 plasmid (Invitrogen, Carlsbad Calif.), PBK-CMV plasmid (Stratagene), or pINCY (Incyte Genomics, Palo Alto Calif.), or derivatives thereof. Recombinant plasmids were transformed into competent E. coli cells including XL1-Blue, XL1-BlueMRF, or SOLR from Stratagene or DH5α, DH10B, or ElectroMAX DH10 B from Life Technologies.

### II. Isolation of cDNA Clones

Plasmnids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

### III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Pharmacia Biotech or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Molecular Dynamics); the ABI PRISM 373 or 377 sequencing system (Applied Biosystems) in conjunction with standard ABI protocols and base calling software; or other sequence analysis systems known in the art. Reading frames within the cDNA sequences were identified using standard methods (reviewed in Ausubel, 1997, supra, unit 7.7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM, and hidden Markov model (HMM)-based protein family databases such as PFAM. (HMM is a probabilistic approach which analyzes consensus primary structures of gene families. See, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide of the invention may begin at any of the methionine residues of the full length translated polypeptide. Full length polypeptide sequences were subsequently analyzed by querying against databases such as the Genank protein databases (genpcpt), SwissProt, BLOCKS, PRINTS, DOMO, PRODOM, Prosite, and hidden Markov model (HMM)-based protein family databases such as PFAM. Full length polynucleotide sequences are also analyzed using MACDNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences wore also used to identify polynucleotide sequence fragments from SEQ ID NO:37-72 Fragments from about 20 to about 4000 nucleotides which are useful in hybridization and amplification technologies are described in Table 4, column 4.

### IV. Identification and Editing of Coding Sequences from Genomic DNA

Putative extracellular matrix and cell adhesion molecules were initially identified by running the Genscan gene identification program against public genomic sequence databases (e.g., gbpri and gbhtg). Genscan is a general-purpose gene identification program which analyzes genomic DNA sequences from a variety of organisms (See Burge, C. and S. Karlin (1997) J. Mol. Biol. 268:78-94, and Burge, C. and S. Karlin (1998) Curr. Opin. Struct. Biol. 8:346-354). The program concatenates predicted exons to form an assembled cDNA sequence extending from a methionine to a stop codoe The output of Genscan is a FASTA database of polynucleotide and polypeptide sequences. The maximum range of sequence for Genscan to analyze at once was set to 30 kb. To determine which of these Genscan predicted cDNA sequences encode extracellular matrix and cell adhesion molecules, the encoded polypeptides were analyzed by querying against PFAM models for extracellular matrix and cell adhesion molecules. Potential extracellular matrix and cell adhesion molecules were also identified by homology to Incyte cDNA sequences that had been annotated as extracellular matrix and cell adhesion molecules. These selected Genscan-predicted sequences were then compared by BLAST analysis to the genpept and gbpri public databases. Where necessary, the Genscan-predicted sequences wore then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

### V. Assembly of Genomic Sequence Data with cDNA Sequence Data

### “Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were farther extended with additional cDNA sequences, or by inspection of genomic DNA, when necessary.

### “Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and cukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

### VI. Chromosomal Mapping of ECMCAD Encoding Polynucleotides

The sequences which were used to assemble SEQ ID NO:37-72 were compared with sequences from the Incyte LIFESEQ database and public domain databases using BLAST and other implementations of the Smith-Waterman algorithm. Sequences from these databases that matched SEQ ID NO:37-72 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Ginethon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

In this manner, SEQ ID NO:47 was mapped to chromosome 3 within the interval from 162.00 to 168.30 centiMorgans. SEQ ID NO:49 was mapped to chromosome 4 within the interval from 63.90 to 88.50 centiMorgans.

### VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel (1995) supra, ch. 4 and 16.)

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and 4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, of 79% identity and 100% overlap.

Alternatively, polynucleotide sequences encoding ECMCAD are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding ECMCAD. cDNA sequences and cDNA library/tissue information are found in the LIFESEQ GOLD database (Incyte Genomics, Palo Alto Calif.).

### VIII. Extension of ECMCAD Encoding Polynucleotides

Full length polynucleotide sequences were also produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 nin; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to detemine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to relegation into pUC 18 vector (Amersham Pharmacia Biotech). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Pharmacia Biotech), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2×carb liquid media.

The cells were lysed, and DNA was amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Pharmacia Biotech) or the ABI PRISM BIGDYE Trminator cycle sequencing ready reaction kit (Applied Biosystems).

In like manner, full length polynucleotide sequences are verified using the above procedure or are used to obtain 5 regulatory sequences using the above procedure along with oligonucleotides designed for such extension, and an appropriate genomic library.

### IX. Labeling and Use of Individual Hybridization Probes

Hybridization probes derived from SEQ ID NO:37-72 are employed to screen cDNAs, gelioulic DNAs, or mRNAs. Although the labeling of oligonicleotides, consisting of about 20 base pairs, is specifically described, essentially the same procedure is used with larger nucleotide fragments. Oligonucleotides are designed using state-of-the-art software such as OLIGO 4.06 software (National Bioscicnces) and labeled by combining 50 pmol of each oligomer, 250 μCi of [γ-³²P] adenosine tiphosphate (Amersham Pharmacia Biotech), and T4 polynucleotide kinase (DuPont NEN, Boston Mass.). The labeled oligonucleotides are substantially purified using a SEPHADEX G-25 superfine size exclusion dextran bead column (Amersham Pharmacia Biotech). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

### X. Microarrays

The linkage or synthesis of array elements upon a microarray can be achieved utilizing photolithography, piezoelectric printing (ink-jet printing, See, e.g., Baldeschweiler, supra.), mechanical microspotting technologies, and derivatives thereof. The substrate in each of the aforementioned technologies should be uniform and solid with a non-porous surface (Schena (1999), supra). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements. (See, e.g., Schena, M. et al. (1995) Science 270:467-470; Shalon, D. et al. (1996) Genome Res. 6:639-645; Marshall, A. and J. Hodgson (1998) Nat. Biotechnol. 16:27-31.)

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorption and mass spectrometry may be used for detection of hybridization. The degree of complementarity and the relative abundance of each polynucleotide which hybridizes to an element on the microarray may be assessed. In one embodiment, microarray preparation and usage is described in detail below.

### Tissue or Cell Sample Preparation

Total RNA is isolated from tissue samples using the guanidinium thiocyanate method and poly(A)⁺RNA is purified using the oligo-(dT) cellulose method. Each poly(A)⁺RNA sample is reverse transcribed using MMLV reverse-transcriptase, 0.05 pg/μl oligo-(dT) primer (21mer), 1×first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Pharmacia Biotech). The reverse transcription reaction is performed in a 25 ml volume containing 200 ng poly(A)⁺RNA with GEMBRIGHT kits (Incyte). Specific control poly(A)⁺RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

### Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL400 (Amersham Pharmacia Biotech).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydroIluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

### Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

### Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte).

### XI. Complementary Polynucleotides

Sequences complementary to the ECMCAD-encoding sequences, or any parts thereof, are used to detect, decrease, or inhibit expression of naturally occurring ECMCAD. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of ECMCAD. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the ECMCAD-encoding transcript.

### XII. Expression of ECMCAD

Expression and purification of ECMCAD is achieved using bacterial or vitus-based expression systems. For expression of ECMCAD in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express ECMCAD upon induction with isopropyl beta-D-thiogalactopyranoside (IPTG). Expression of ECMCAD in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding ECMCAD by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus. (See Engelhard, E. K. et al. (1994) Proc. Natl. Acad. Sci. USA 91:3224-3227; Sandig, V. et al. (1996) Hum. Gene Ther. 7:1937-1945.)

In most expression systems, ECMCAD is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Pharmacia Biotech). Following purification, the GST moiety can be proteolytically cleaved from ECMCAD at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel (1995, supra, ch. 10 and 16). Purified ECMCAD obtained by these methods can be used directly in the assays shown in Examples XVI and XVI where applicable.

### XIII. Functional Assays

ECMCAD function is assessed by expressing the sequences encoding ECMCAD at physiologically elevated levels in mammalian cell culture systems. cDNA is subcloned into a mammalian expression vector containing a strong promoter that drives high levels of cDNA expression. Vectors of choice include PCMV SPORT (Life Technologies) and PCR3.1 (Invitrogen, Carlsbad Calif.), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of ECMCAD on gene expression can be assessed using highly purified populations of cells transfected with sequences encoding ECMCAD and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic heads coated with either human IgG or antibody against CD64 (DYNAL, Lake Success N.Y.). mRNA can be purified from the cells using methods well known by those of skill in the art. Expression of mRNA encoding ECMCAD and other genes of interest can be analyzed by northern analysis or microarray techniques.

### XIV. Production of ECMCAD Specific Antibodies

ECMCAD substantially purified using polyacrylamide gel electrophoresis (PAGE; see, e.g., Harrington. M. G. (1990) Methods Enzymol. 182:488-495), or other purification techniques, is used to immunize rabbits and to produce antibodies using standard protocols.

Alternatively, the ECMCAD amino acid sequence is analyzed using LASERGENE software (DNASTAR) to detentine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel, 1995, supra, ch. 11.)

Typically, oligopeptides of about 15 residues in length are synthesized using an ABI 431 A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity. (See, e.g., Ausubel, 1995, supra.) Rabbits are immunized with the oligopeptide-KLH complex in complete Freund's adjuvant. Resulting antisera are tested for antipeptide and anti-ECMCAD activity by, for example, binding the peptide or ECMCAD to a substrate, blocking with 1% BSA, reacting with rabbit antisera, washing, and reacting with radio-iodinated goat anti-rabbit IgG.

### XV. Purification of Naturally Occurring ECMCAD Using Specific Antibodies

Naturally occurring or recombinant ECMCAD is substantially purified by immunoaffinity chromatography using antibodies specific for ECMCAD. An immunoaffinity column is constructed by covalently coupling anti-ECMCAD antibody to an activated chromatographic resin, such as CNBr-activated SEPHAROSE (Amersham Pharmacia Biotech). After the coupling, the resin is blocked and washed according to the manufacturer's instructions.

Media containing ECMCAD are passed over the immunoaffinity column, and the column is washed under conditions that allow the preferential absorbance of ECMCAD (e.g., high ionic strength buffers in the presence of detergent). The column is eluted under conditions that disrupt antibody/ECMCAD binding (e.g., a buffer of pH 2 to pH 3, or a high concentration of a chaotrope, such as urea or thiocyanate ion), and ECMCAD is collected.

### XVI. Identification of Molecules Which Interact with ECMCAD

ECMCAD, or biologically active fragments thereof, are labeled with ¹²⁵Bolton-Hunter reagent. (See, e.g., Bolton A. E. and W. M. Hunter (1973) Biochem. J. 133:529-539.) Candidate molecules previously arrayed in the wells of a multi-well plate are incubated with the labeled ECMCAD, washed, and any wells with labeled ECMCAD complex are assayed. Data obtained using different concentrations of ECMCAD are used to calculate values for the number, affinity, and association of ECMCAD with the candidate molecules.

Alternatively, molecules interacting with ECMCAD are analyzed using the yeast two-hybrid system as described in Fields, S. and O. Song (1989) Nature 340:245-246, or using commercially available kits based on the two-hybrid system, such as the MATCHMAKER system (Clontech).

ECMCAD may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

### XVII. Demonstration of ECMCAD Activity

An assay for ECMCAD activity measures the expression of ECMCAD on the cell surface. cDNA encoding ECMCAD is transfected into a non-leukocytic cell line. Cell surface proteins are labeled with biotin (de la Fuente, M. A. et al. (1997) Blood 90:2398-2405). Immunoprecipitations are performed using ECMCAD-specific antibodies, and immunoprecipitated samples are analyzed using SDS-PAGE and immunoblotting techniques. The ratio of labeled immunoprecipitant to unlabeled immunoprecipitant is proportional to the amount of ECMCAD expressed on the cell surface.

Alternatively, an assay for ECMCAD activity measures the amount of cell aggregation induced by overexpression of ECMCAD. In this assay, cultured cells such as NIH3T3 are transfected with cDNA encoding ECMCAD contained within a suitable mammalian expression vector under control of a strong promoter. Cotransfection with cDNA encoding a fluorescent marker protein, such as Green Fluorescent Protein (CLONTECH), is useful for identifying stable transfectants. The amount of cell agglutination, or clumping, associated with transfected cells is compared with that associated with untransfected cells. The amount of cell agglutination is a direct measure of ECMCAD activity.

Alternatively, an assay for ECMCAD activity measures the disruption of cytoskeletal filament networks upon overexpression of ECMCAD in cultured cell lines (Rezniczek, G. A. et al. (1998) J. Cell Biol. 141:209-225). cDNA encoding ECMCAD is subcloned into a mammalian expression vector that drives high levels of cDNA expression. This construct is transfected into cultured cells, such as rat kangaroo PtK2 or rat bladder carcinoma 804G cells. Actin filaments and intermediate filaments such as keratin and vimentin are visualized by immunofluorescence microscopy using antibodies and techniques well known in the art. The configuration and abundance of cyoskeletal filaments can be assessed and quantified using confocal imaging techniques. In particular, the bundling and collapse of cytoskeletal filament networks is indicative of ECMCAD activity.

Alternatively, cell adhesion activity in ECMCAD is measured in a 96-well microtiter assay in which wells are first coated with ECMCAD by adding solutions of ECMCAD of varying concentrations to the wells. Excess ECMCAD is washed off with saline, and the wells incubated with a solution of 1% bovine serum albumin to block non-specific cell binding. Aliquots of a cell suspension of a suitable cell type are then added to the microtiter wells and incubated for a period of time at 37° C. Non-adhered cells are washed off with saline and the cells stained with a suitable cell stain such as Coomassie blue. The intensity of staining is measured using a variable wavelength microtiter plate reader and compared to a standard curve to determine the number of cells adhering to the ECMCAD coated plates. The degree of cell staining is proportional to the cell adhesion activity of ECMCAD in the sample.

Various modifications and variations of the described methods and systems of the invention wilt be apparent to those skilled in the art without departing from the scope and spirit of the invention. Although the invention has been described in collection with certain embodiments, it should be understood that the invention as claimed should not he unduly limited to such specific embodiments. Indeed, various modifications of the described modes for carrying out the invention which are obvious to those skilled in molecular biology or related fields are intended to be within the scope of the following claims.

TABLE 1

| | | | Polynucleo- | |
|---|---|---|---|---|
| Incyte | Polypeptide | Incyte | tide SEQ | Incyte Poly- |
| Project ID | SEQ ID NO: | Polypeptide ID | ID NO: | nucleotide ID |
| 1888682 | 1 | 1888682CD1 | 37 | 1888682CB1 |
| 1794980 | 2 | 1794980CD1 | 38 | 1794980CB1 |
| 5533958 | 3 | 5533958CD1 | 39 | 5533958CB1 |
| 60210196 | 4 | 60210196CD1 | 40 | 60210196CB1 |
| 815125 | 5 | 815125CD1 | 41 | 815125CB1 |
| 1386915 | 6 | 1386915CD1 | 42 | 1386915CB1 |
| 1344495 | 7 | 1344495CD1 | 43 | 1344495CB1 |
| 1485774 | 8 | 1485774CD1 | 44 | 1485774CB1 |
| 7289372 | 9 | 7289372CD1 | 45 | 7289372CB1 |
| 1672338 | 10 | 1672338CD1 | 46 | 1672338CB1 |
| 184661 | 11 | 184661CD1 | 47 | 184661CB1 |
| 3719737 | 12 | 3719737CD1 | 48 | 3719737CB1 |
| 5773251 | 13 | 5773251CD1 | 49 | 5773251CB1 |
| 5426470 | 14 | 5426470CD1 | 50 | 5426470CB1 |
| 7087904 | 15 | 7087904CD1 | 51 | 7087904CB1 |
| 7477312 | 16 | 7477312CD1 | 52 | 7477312CB1 |
| 2739431 | 17 | 2739431CD1 | 53 | 2739431CB1 |
| 7473606 | 18 | 7473606CD1 | 54 | 7473606CB1 |
| 3534918 | 19 | 3534918CD1 | 55 | 3534918CB1 |
| 2428715 | 20 | 2428715CD1 | 56 | 2428715CB1 |
| 3351332 | 21 | 3351332CD1 | 57 | 3351332CB1 |
| 6382722 | 22 | 6382722CD1 | 58 | 6382722CB1 |
| 55022490 | 23 | 55022490CD1 | 59 | 55022490CB1 |
| 6755002 | 24 | 6755002CD1 | 60 | 6755002CB1 |
| 7350907 | 25 | 7350907CD1 | 61 | 7350907CB1 |
| 7474411 | 26 | 7474411CD1 | 62 | 7474411CB1 |
| 4755911 | 27 | 4755911CD1 | 63 | 4755911CB1 |
| 379766 | 28 | 379766CD1 | 64 | 379766CB1 |
| 553744 | 29 | 553744CD1 | 65 | 553744CB1 |
| 1825473 | 30 | 1825473CD1 | 66 | 1825473CB1 |
| 7950094 | 31 | 7950094CD1 | 67 | 7950094CB1 |
| 7479484 | 32 | 7479484CD1 | 68 | 7479484CB1 |
| 6780147 | 33 | 6780147CD1 | 69 | 6780147CB1 |
| 7204554 | 34 | 7204554CD1 | 70 | 7204554CB1 |
| 6833247 | 35 | 6833247CD1 | 71 | 6833247CB1 |
| 4148119 | 36 | 4148119CD1 | 72 | 4148119CB1 |

TABLE 2

| Polypeptide | Incyte | GenBank | Probability | |
|---|---|---|---|---|
| SEQ ID NO: | Polypeptide ID | ID NO: | score | GenBank Homolog |
| 2 | 1794980CD1 | g192264 | 6.9e−46 | procollagen type I alpha chain |
| | | | | Grant, S. F. et al. (1996) |
| | | | | Nat. Genet. 14: 203-205 |
| 3 | 5533958CD1 | g4091819 | 2.2e−54 | glioma-inactivated protein precursor |
| | | | | Chernova, O. B. et al. (1998) |
| | | | | Oncogene 17: 2873-2882 |
| 4 | 60210196CD1 | g3132522 | 8.0e−144 | carcinogenesis-associated protein |
| | | | | ICB-1 (induced by cell-matrix |
| | | | | interactions) |
| | | | | Treeck, O. et al. (1998) FEBS Lett. |
| | | | | 425: 426-430 |
| 5 | 815125CD1 | g9652103 | 0 | [Mus musculus] netrin 4 |
| | | | | Yin, Y. et al. (2000) Identification |
| | | | | and expression of mouse netrin-4 |
| | | | | Mech. Dev. 96: 115-119 |
| | | g4388541 | 3.3e−96 | laminin B1 chain |
| | | | | Durkin, M. E. et al. (1988) |
| | | | | Biochemistry 27: 5198-5204 |
| 6 | 1386915CD1 | g1234793 | 4.2e−254 | [Xenopus laevis] MAM domain protein |
| | | | | Brown, D. D. et al. (1996) The |
| | | | | thyroid hormone-induced tail |
| | | | | resorption program during Xenopus |
| | | | | laevis metamorphosis. Proc. Natl. |
| | | | | Acad. Sci. U.S.A. 93, 1924-1929 |
| 9 | 7289372CD1 | g2554604 | 6.1e−88 | [Homo sapiens] ISLR |
| | | | | Nagasawa, A. et al. (1997) Cloning |
| | | | | of the cDNA for a new member of the |
| | | | | immunoglobulin superfamily (ISLR) |
| | | | | containing leucine-rich repeat |
| | | | | (LRR). Genomics 44, 273-279 |
| 10 | 1672338CD1 | g854326 | 6.0e−66 | [Mus musculus] semaphorin B |
| | | | | (Puschel, A. W. et al. (1995) Neuron |
| | | | | 14: 941-948) |
| 11 | 184661CD1 | g2367641 | 2.9e−26 | [Rattus norvegicus] neuropilin-2 |
| | | | | (Kolodkin, A. L. et al. (1997) Cell |
| | | | | 90: 753-762) |
| 12 | 3719737CD1 | g9622242 | 0 | [Homo sapiens] [5′ incom] |
| | | | | protocadherin 13 |
| | | g6978935 | 9.9e−170 | [Homo sapiens] protocadherin-Xa |
| | | | | (Yoshida, K. and Sugano, S. (1999) |
| | | | | Genomics 62: 540-543) |
| 13 | 5773251CD1 | g1304387 | 5.4e−27 | [Saccharomyces cerevisiae var. |
| | | | | diastaticus] Glucoamylase |
| | | | | (Lambrechts, M. G. et al. (1996) Proc. |
| | | | | Natl. Acad. Sci. U.S.A. 93: 8419-8424) |
| 14 | 5426470CD1 | g200057 | 0.0 | [Mus musculus] neuronal glycoprotein |
| | | | | Connelly, M. A. et al. (1994) Proc. |
| | | | | Natl. Acad. Sci. U.S.A. 91: 1337-1341 |
| 15 | 7087904CD1 | g4519558 | 1.4e−181 | [Rattus norvegicus] Kilon |
| | | | | Funatsu, N. et al. (1999) J. Biol. |
| | | | | Chem. 274: 8224-8230 |
| 16 | 7477312CD1 | g5262748 | 0.0 | [Rattus norvegicus] Proline rich |
| | | | | synapse associated protein 2 |
| | | | | Boeckers, T. M. et al. (1999) Biochem. |
| | | | | Biophys. Res. Commun. 264: 2476-2528; |
| | | | | Boeckers, T. M. et al. (1999) J. |
| | | | | Neurosci. 19: 6506-6518 |
| 17 | 2739431CD1 | g2708626 | 5.7e−34 | [Mus musculus] fibrinogen-like |
| | | | | protein |
| 18 | 7473606CD1 | g3928000 | 3.5e−07 | [Homo sapiens] procollagen I N- |
| | | | | proteinase |
| 19 | 3534918CD1 | g13872813 | 0 | [5′ incom] [Homo sapiens] (AJ306906) |
| | | | | fibulin-6 |
| | | g2947314 | 8.6e−63 | [Gallus gallus] fibulin-1, isoform D |
| | | | | precursor |
| 20 | 2428715CD1 | g10998440 | 0 | [Mus musculus] EGF-related protein |
| | | | | SCUBE1 |
| | | | | Grimmond, S. et al. (2000) Cloning, |
| | | | | Mapping, and Expression Analysis of |
| | | | | a Gene Encoding a Novel Mammalian |
| | | | | EGF-Related Protein (SCUBE1) |
| | | | | Genomics 70: 74-81 |
| | | g2072792 | 3.1e−64 | [Mus musculus] matrilin-2 precursor |
| | | | | Deak, F. et al. (1997) Primary |
| | | | | structure and expression of |
| | | | | matrilin-2, the closest relative of |
| | | | | cartilage matrix protein within the |
| | | | | von Willebrand |
| | | | | factor type A-like module |
| | | | | superfamily. J. Biol. Chem. 272, |
| | | | | 9268-9274 |
| 21 | 3351332CD1 | g3449294 | 0 | [Rattus norvegicus] MEGF6 |
| | | | | Nakayama, M. et al. (1998) |
| | | | | Identification of high-molecular- |
| | | | | weight proteins with multiple EGF- |
| | | | | like motifs by motif-trap screening |
| | | | | Genomics 51, 27-34 |
| | | g483581 | 1.1e−111 | [Mus musculus] Notch 3 |
| | | | | Lardelli, M. et al. (1994) The novel |
| | | | | Notch homologue mouse Notch 3 lacks |
| | | | | specific epidermal growth factor- |
| | | | | repeats and is expressed in |
| | | | | proliferating Neuroepithelium. Mech. |
| | | | | Dev. 46, 123-136 |
| 22 | 6382722CD1 | g2599232 | 0.0 | [Mus musculus] laminin alpha 5 chain |
| | | | | Miner, J. H. et al. (1995) Molecular |
| | | | | cloning of a novel laminin chain, |
| | | | | alpha 5, and widespread |
| | | | | expression in adult mouse tissues. |
| | | | | J. Biol. Chem. 270, 28523-28526 |
| 23 | 55022490CD1 | g8977890 | 9.3e−256 | [Homo sapiens] ADAMTS7, |
| | | | | alternatively spliced product |
| 24 | 6755002CD1 | g452821 | 0.0 | [Bos taurus] Brevican |
| | | | | Yamada, H. et al. (1994) J. Biol. |
| | | | | Chem. 269: 10119-10126 |
| 25 | 7350907CD1 | g442368 | 4.1e−246 | [Rattus norvegicus] Neuronal |
| | | | | olfactomedin-related ER localized |
| | | | | protein |
| | | | | Danielson, P. E. et al. (1994) J. |
| | | | | Neurosci. Res. 38: 468-478 |
| 26 | 7474411CD1 | g6164595 | 4.0e−95 | [Manduca sexta] Lacunin |
| | | | | (Extracellular matrix protein) |
| | | | | Nardi, J. B. et al. (1999) Insect |
| | | | | Biochem. Mol. Biol. 29: 883-897 |
| 27 | 4755911CD1 | g1504038 | 7.1e−41 | [Homo sapiens] Similar to human |
| | | | | ankyrin 1 |
| 28 | 379766CD1 | g13183078 | 1.00E−163 | [3′ incom] [Homo sapiens] a |
| | | | | disintegrin-like and metalloprotease |
| | | | | domain with thrombospondin type I |
| | | | | motifs-like 3 |
| | | g5923786 | 1.4e−44 | [Homo sapiens] Zinc metalloprotease |
| | | | | ADAMTS6 |
| | | | | Hurskainen, T. L. et al. (1999) J. |
| | | | | Biol. Chem. 274: 25555-25563 |
| 29 | 553744CD1 | g8572538 | 7.0e−14 | [Homo sapiens] Mucin |
| | | | | Gerard, C. et al. (1990) J. Clin. |
| | | | | Invest. 86: 1921-1927 |
| 30 | 1825473CD1 | g188864 | 9.2e−47 | [Homo sapiens] Mucin |
| | | | | Toribara, N. W. et al. (1991) J. |
| | | | | Clin. Invest. 88: 1005-1013 |
| 31 | 7950094CD1 | g1110599 | 0.0 | [Mus sp.] Semaphorin homolog |
| | | | | Inagaki, S. et al. (1995) FEBS Lett. |
| | | | | 370: 269-272 |
| 32 | 7479484CD1 | g4322670 | l.7e−28 | [Homo sapiens] Dentin phosphoryn |
| | | | | Gu, K. et al. (1998) Eur. J. Oral |
| | | | | Sci. 106: 1043-1047 |
| 33 | 6780147CD1 | g5805194 | 0.0 | [Rattus norvegicus] Leprecan |
| | | | | Wassenhove-McCarthy, D. J. and K. J. |
| | | | | McCarthy (1999) J. Biol. Chem. |
| | | | | 274: 25004-25017 |
| 34 | 7204554CD1 | g1665757 | 0.0 | [Mus musculus] Plexin 1 |
| | | | | Kameyama, T. et al. (1996) Biochem. |
| | | | | Biophys. Res. Commun. 226: 524-529 |
| 35 | 6833247CD1 | g4159801 | 2.9e−75 | [Mus musculus] C-type lectin |
| | | | | Balch, S. G. et al. (1998) J. Biol. |
| | | | | Chem. 273: 18656-18664 |
| 36 | 4148119CD1 | g6579191 | 6.5e−35 | [Rattus norvegicus] SLIT-2 |
| | | | | Liang Y. et al. (1999) J. Biol. |
| | | | | Chem. 274: 17885-17892 |

TABLE 3

| SEQ | Incyte | Amino | Potential | Potential | | Analytical |
|---|---|---|---|---|---|---|
| ID | Polypeptide | Acid | Phosphorylation | Glycosylation | Signature Sequences, | Methods and |
| NO: | ID | Residues | Sites | Sites | Domains and Motifs | Databases |
| 1 | 1888682CD1 | 234 | S141 S150 T137 | N147 N197 N52 | Signal cleavage: M1-A44 | SPSCAN |
| | | | T99 | N97 | Transmembrane domain: G168-F187 | HMMER |
| | | | | | Rgd cell surface interaction motif: R14-D16 | MOTIFS |
| | | | | | Fibronectin type III domain: P47-G130 | HMMER_PFAM |
| | | | | | Fibronectin type III PR00014: | BLIMPS_PRINTS |
| | | | | | N61-P70, V96-Y114, Y114-P128 | |
| 2 | 1794980CD1 | 443 | S103 S188 S31 | N138 N51 | Signal cleavage: M1-A21 | SPSCAN |
| | | | S438 S72 T122 | | Signal peptide: M1-A22 | HMMER |
| | | | T146 T160 T172 | | Glycosaminoglycan attchment site: S407-G410 | MOTIFS |
| | | | T242 T282 T80 | | Transmembrane domain: A8-F30 | HMMER |
| | | | T89 | | Collagen triple helix repeat: | HMMER_PFAM |
| | | | | | G208-P267 G299-R358 | |
| | | | | | COLLAGEN ALPHA PRECURSOR CHAIN | BLAST_PRODOM |
| | | | | | REPEAT PD000007: G223-G332 | |
| | | | | | FIBRILLAR COLLAGEN CARBOXYL-TERMINAL | BLAST_DOMO |
| | | | | | DM00019/P20908/1300-1524): P176-G374 | |
| 3 | 5533958CD1 | 261 | S243 S89 T151 | N177 | Leucine_Zipper: L129-L150 | MOTIFS |
| | | | T203 | | Atp_Gtp binding site: A239-T246 | MOTIFS |
| | | | | | Signal cleavage: M1-A19 | SPSCAN |
| | | | | | Signal peptide: M1-A19 | HMMER |
| | | | | | Leucine rich repeat: T53-Q171 | HMMER_PFAM |
| | | | | | Leucine rich repeat C-terminal domain: | HMMER_PFAM |
| | | | | | N158-R207 | |
| | | | | | Leucine-rich repeat signature PR00019: | BLIMPS_PRINTS |
| | | | | | L126-L139 | |
| | | | | | NEUROGENIC TRKB RECEPTOR DM01983\| | BLAST_DOMO |
| | | | | | B39667\|24-197: W20-L169 | |
| 4 | 60210196CD1 | 643 | S103 S143 S162 | | ICB1 membrane carcinogenesis-associated | BLAST_PRODOM |
| | | | S225 S237 S304 | | protein PD121395: M150-G403 | |
| | | | S33 S38 S388 | | | |
| | | | S429 S437 S441 | | | |
| | | | S45 S567 T114 | | | |
| | | | T333 T527 T593 | | | |
| | | | T92 | | | |
| 5 | 815125CD1 | 628 | S108 S136 S212 | N163 N353 | Signal cleavage: M1-L20 | SPSCAN |
| | | | S27 S28 S387 | N483 N56 | Egf (epidermal growth factor) motif: | MOTIFS |
| | | | S416 S465 S503 | | C293-C304, C420-C431 | |
| | | | S534 S554 S570 | | Signal peptide: M1-G25 | HMMER |
| | | | S98 T153 T182 | | Transmembrane domain: M1-V23 | HMMER |
| | | | T192 T331 T411 | | Laminin N-terminal (Domain VI) | HMMER_PFAM |
| | | | T467 T540 T572 | | laminin_Nterm: C34-G260 | |
| | | | T597 T71 | | Laminin EGF-like (Domains III and V): | HMMER_PFAM |
| | | | | | C262-C329, C332-C392, C395-C446 | |
| | | | | | Laminin-type EGF-like domain BL01248: | BLIMPS_BLOCKS |
| | | | | | C295-C307 | |
| | | | | | Type III EGF-like signature PR00011: | BLIMPS_PRINTS |
| | | | | | C413-C431 | |
| | | | | | EGFLIKE LAMININ PRECURSOR DOMAIN | BLAST_PRODOM |
| | | | | | REPEAT PD002082: C34-G260, C262-A319 | |
| | | | | | LAMININ CHAIN B1 DM01003\|P07942\|27-259: | BLAST_DOMO |
| | | | | | E31-K230 | |
| 6 | 1386915CD1 | 686 | S117 S135 S136 | N134 N329 | Signal peptide: | HMMER; SPSCAN |
| | | | S196 S336 S445 | N524 | M1-A18 | |
| | | | S449 S477 S497 | | MAM domains: | HMMER_PFAM |
| | | | S499 S578 S647 | | C26-E169; C170-N329; C342-S498; | |
| | | | S93 T209 T460 | | C509-E666 | |
| | | | T89 | | PRECURSOR GLYCOPROTEIN SIGNAL | BLAST_PRODOM |
| | | | | | TRANSMEMBRANE HYDROLASE PROTEIN | |
| | | | | | REPEAT RECEPTOR PHOSPHATASE | |
| | | | | | NEUROPILIN PD001482: C170-C327; C509-E666 | |
| | | | | | MAM DM01344\|A55620\|618-796: | BLAST_DOMO |
| | | | | | C509-R644 | |
| | | | | | MAM domain motif: | MOTIFS |
| | | | | | G551-F585 | |
| 7 | 1344495CD1 | 296 | S115 S38 S39 | N253 N259 N30 | Transmembrane domain: | HMMER |
| | | | S43 T129 T207 | | W218-F235 | |
| | | | T282 | | Putative peptidoglycan binding domain: | HMMER_PFAM |
| | | | | | R76-P119 | |
| 8 | 1485774CD1 | 575 | S240 S281 S402 | N257 N270 | Signal peptide: | SPSCAN |
| | | | S413 S511 S527 | N348 N509 | M1-P45 | |
| | | | T157 T170 T245 | N564 | WSC domain: | HMMER_PFAM |
| | | | T247 T301 T341 | | Y145-A224 | |
| | | | T500 | | | |
| 9 | 7289372CD1 | 592 | S151 S19 S198 | N121 N337 | Signal peptide: | HMMER; SPSCAN |
| | | | S202 S289 S346 | N364 N474 N52 | M1-P21 | |
| | | | S367 S395 S411 | N563 | Leucine rich repeat N-terminal domain: | HMMER_PFAM |
| | | | S440 S544 S587 | | S19-P50 | |
| | | | T267 T329 T354 | | Leucine Rich Repeats: | HMMER_PFAM |
| | | | T400 T431 T433 | | N52-T75; Q76-S99; Q100-S123; A124-P147; | |
| | | | | | D148-S171 | |
| | | | | | Leucine rich repeat C-terminal domain: | HMMER_PFAM |
| | | | | | N181-A231 | |
| | | | | | Immunoglobulin domain: | HMMER_PFAM |
| | | | | | G253-A357 | |
| | | | | | ISLR PRECURSOR SIGNAL PD103127: | BLAST_PRODOM |
| | | | | | P229-P286; P321-I417 | |
| | | | | | ISLR PRECURSOR SIGNAL PD167884: | BLAST_PRODOM |
| | | | | | S19-V74 | |
| 10 | 1672338CD1 | 255 | S106 S111 S119 | N120 N135 | signal peptide: M1-A31 | HMMER |
| | | | S180 T233 | | Sema domain: F64-V155 | HMMER_PFAM |
| | | | | | SEMAPHORIN B IMMUNOGLOBULIN FOLD | BLAST_PRODOM |
| | | | | | NEUROGENESIS DEVELOPMENTAL PROTEIN | |
| | | | | | PD107003: M1-D63 | |
| | | | | | SEMAPHORIN PROTEIN RECEPTOR KINASE | BLAST_PRODOM |
| | | | | | SIGNAL TYROSINE FAMILY HEPATOCYTE | |
| | | | | | PD001844: L67-H167 | |
| | | | | | SEMAPHORIN; FASCICLIN; COLLAPSIN; II | BLAST_DOMO |
| | | | | | DM01606\|I48745\|1-619: M1-I154, | |
| | | | | | C217-L255 | |
| | | | | | DM01606\|I48748\|1-589: G34-I154 | |
| | | | | | DM01606\|I48747\|1-646: L24-I154 | |
| | | | | | DM01606\|A49069\|1-646: F64-I154 | |
| 11 | 184661CD1 | 641 | S103 S139 S226 | N124 N277 | signal_cleavage: M1-A34 | SPSCAN |
| | | | S242 S244 S275 | N351 N418 | signal peptide: M1-A34 | HMMER |
| | | | S427 S433 S488 | N455 N64 | transmembrane domain: T458-F480 | HMMER |
| | | | S556 S615 S634 | | F5/8 type C domain (discoidin (DS) domain | HMMER_PFAM |
| | | | T129 T157 T325 | | family): S258-L409 | |
| | | | T357 T434 T46 | | CUB domain: C41-Y147 | HMMER_PFAM |
| | | | T527 T54 T552 | | GLYCOPROTEIN NEUROPILIN COAGULATION | BLAST_PRODOM |
| | | | T557 T563 T600 | | PD000875: D264-L409 | |
| | | | T66 | | GLYCOPROTEIN EGF-LIKE FACTORB12 | BLAST_PRODOM |
| | | | | | PD000165: C41-Y147 | |
| | | | | | DISCOIDIN I N-TERMINAL | BLAST_DOMO |
| | | | | | DM00516\|P12259\|2095-2223: H284-I414 | |
| | | | | | DM00516\|A42580\|2085-2210: P287-I414 | |
| | | | | | DM00516\|P00451\|2221-2347: W285-Q413 | |
| | | | | | DM00516\|A44258\|86-212: W285-Q413 | |
| 12 | 3719737CD1 | 924 | S116 S12 S144 | N108 N299 | signal_cleavage: M1-G33 | SPSCAN |
| | | | S333 S362 S366 | N305 N653 | transmembrane domain: L866-I884 | HMMER |
| | | | S44 S57 S609 | N721 N776 | Cadherin domain: | HMMER_PFAM |
| | | | S635 S767 S824 | N817 N822 | I187-S284, I298-I390, I513-L603, | |
| | | | T219 T428 T464 | | F617-L706, Y724-N817 | |
| | | | T516 T533 T568 | | Cadherin: | MOTIFS |
| | | | T581 T601 T637 | | I170-P180 I281-P291 V496-P506 | |
| | | | T662 T698 T778 | | L600-P610 I703-P713 | |
| | | | T82 T850 Y43 | | Cadherins extracellular repeated domain | PROFILESCAN |
| | | | Y436 Y580 Y802 | | signature: | |
| | | | | | V260-I312, T581-I631, V685-P733 | |
| | | | | | CADHERIN SIGNATURE | BLIMPS_PRINTS |
| | | | | | PR00205: Q678-P693, S696-P713, | |
| | | | | | V168-F182 | |
| | | | | | CADHERIN REPEAT | BLAST_DOMO |
| | | | | | DM00030\|P33450\|1079-1181: E539-D640 | |
| | | | | | DM00030\|P33450\|187-298: N215-L322 | |
| | | | | | DM00030\|P33450\|1952-2055: E539-A641 | |
| | | | | | DM00030\|P34616\|1682-1783: G642-D746 | |
| 13 | 5773251CD1 | 987 | S111 S115 S15 | N14 N213 N337 | KH domain: K113-G161 | HMMER_PFAM |
| | | | S16 S165 S32 | N391 N404 | OTOGELIN ALPHA POLYPEPTIDE ALPHANAC | BLAST_PRODOM |
| | | | S324 S33 S377 | N410 N478 | MUSCLESPECIFIC FORM GP220 | |
| | | | S443 S457 S459 | N581 N628 | PD147940: A206-S772 | |
| | | | S5 S52 S56 S662 | N729 N770 | MUCIN; MUC5; TRACHEOBRONCHIAL | BLAST_DOMO |
| | | | S669 S795 S816 | N800 N833 | DM05454\|S55316\|1-317: I287-P530, | |
| | | | S93 S959 T158 | | V384-S666, S317-P530, | |
| | | | T246 T249 T417 | | T355-S617, F311-S583 | |
| | | | T554 T62 T640 | | EPSTEIN; BARR; MEMBRANE | BLAST_DOMO |
| | | | T686 T813 | | DM06222\|P03200\|1-906: G203-S588, | |
| | | | | | S339-A768 | |
| 14 | 5426470CD1 | 1028 | S133 S164 S170 | N193 N375 | Fibronectin type III domain: | HMMER_PFAM |
| | | | S184 S270 S279 | N468 N489 N65 | P598-S687, P700-S790, P802-S891, | |
| | | | S342 S348 S377 | N765 N860 | P903-S986 | |
| | | | S397 S406 S436 | N895 N913 | Immunoglobulin domain: | HMMER_PFAM |
| | | | S442 S449 S507 | N931 N956 | D43-A102, G137-V198, G242-A299, | |
| | | | S512 S549 S558 | | C339-A388, G424-A481, G514-V579 | |
| | | | S572 S588 S617 | | CONTACTIN CELL ADHESION NEUROFASCIN | BLAST_PRODOM |
| | | | S67 S678 S690 | | GLYCOPROTEIN GP135 IMMUNOGLOBULIN | |
| | | | S713 S772 S797 | | PD001890: L688-P802 | |
| | | | S815 S817 S852 | | ADHESION IMMUNOGLOBULIN | BLAST_PRODOM |
| | | | S863 T244 T364 | | GLYCOPROTEIN GPI ANCHOR REPEAT | |
| | | | T47 T470 T581 | | CONTACTIN PD005229: V894-I991 | |
| | | | T648 T661 T754 | | FIBRONECTIN TYPEIII | BLAST_PRODOM |
| | | | T758 T897 T898 | | PD073047: N301-G560 | |
| | | | T955 T958 T984 | | NEURAL CELL ADHESION MOLECULE CLOSE | BLAST_PRODOM |
| | | | T995 Y98 | | HOMOLOGUE OF L1 L1LIKE PROTEIN | |
| | | | | | PD066559: E482-G596 | |
| | | | | | IMMUNOGLOBULIN | BLAST_DOMO |
| | | | | | DM00001\|A53449\|497-587: T497-S588 | |
| | | | | | DM00001\|A53449\|405-495: A405-V496 | |
| | | | | | DM00001\|A53449\|32-110: P32-S111 | |
| | | | | | DM00001\|A53449\|126-206: T126-V207 | |
| 15 | 7087904CD1 | 354 | S171 S178 S210 | N155 N275 | signal_cleavage: M1-P33 | SPSCAN |
| | | | S277 T125 T149 | N286 N294 | Immunoglobulin domain: | HMMER_PFAM |
| | | | T163 T226 Y187 | N307 N73 | G53-V120, G153-A205, G238-A299 | |
| | | | | | Receptor tyrosine kinase class III | PROFILESCAN |
| | | | | | signature: V135-A198 | |
| | | | | | PRECURSOR SIGNAL GLYCOPROTEIN | BLAST_PRODOM |
| | | | | | IMMUNOGLOBULIN FOLD CELL ADHESION GPI | |
| | | | | | ANCHOR ALTERNATIVE | |
| | | | | | PD005605: F40-T128 | |
| | | | | | IMMUNOGLOBULIN | BLAST_DOMO |
| | | | | | DM00001\|P32736\|39-125: A44-T128 | |
| | | | | | DM00001\|P32736\|139-212: D143-V214 | |
| | | | | | DM00001\|P32736\|226-306: I227-A308 | |
| 16 | 7477312CD1 | 1829 | S1050 S1124 | N122 N239 | signal_cleavage: M1-A15 | SPSCAN |
| | | | T948 S1150 S124 | N358 N531 | Ank repeat: | HMMER_PFAM |
| | | | S477 S1260 | | S226-R259 D260-S292 R293-E326 | |
| | | | S1285 S57 | | N327-A359 S360-Y392 | |
| | | | S1295 S484 T279 | | SAM domain (Sterile alpha motif): | HMMER_PFAM |
| | | | S1351 S667 | | Q1764-D1827 | |
| | | | S1370 S1371 | | SH3 domain: R580-V634 | HMMER_PFAM |
| | | | S844 S1389 | | CORTACTINBINDING PROTEIN 1 | BLAST_PRODOM |
| | | | S1390 S870 | | D0148775: P1333-L1765, V768-A1305, | |
| | | | S1391 S1436 | | P912-T1495, P17-R223, E16-A74, | |
| | | | S952 S1449 | | P27-A128, S454-P569, A6-P64, | |
| | | | S1453 S996 | | A60-P94, P1739-W1766, P478-L504, | |
| | | | S1462 S1496 | | P25-S38 | |
| | | | S880 S1536 | | LARGE STRUCTURAL PHOSPHOPROTEIN | BLAST_PRODOM |
| | | | S1596 S854 | | PD145465: R1021-R1560 | |
| | | | S1608 S1619 | | BAT2 (HLA-B-associated transcript 2) | BLAST_DOMO |
| | | | S889 S1637 | | DM05517\|S37671\|1-1870: P896-D1201, | |
| | | | S1660 S900 | | A12-A79, G1610-P1762 | |
| | | | S1664 S1729 | | PROLINE-RICH PROTEIN 3 | BLAST_DOMO |
| | | | S1767 S503 S608 | | DM00215\|S14972\|7-90: P912-P941, | |
| | | | T1011 T1086 | | P556-P566 | |
| | | | T1125 T415 | | | |
| | | | T1196 T1360 | | | |
| | | | T848 S38 | | | |
| | | | T1395 T443 | | | |
| | | | T1526 T1801 | | | |
| | | | T206 T323 T618 | | | |
| | | | T641 T645 T650 | | | |
| | | | T699 T771 T796 | | | |
| | | | T823 T837 T843 | | | |
| 17 | | | | N173 N264 N53 | Signal peptide: M1-A22 | SPSCAN |
| | | | | | Signal peptide: M1-A22 | HMMER |
| | | | | | Fibrinogen beta and gamma chains, C-term | HMMER_PFAM |
| | | | | | domain: | |
| | | | | | Q115-M317 | |
| | | | | | Fibrinogen beta and gamma chains C-terminal | BLIMPS_BLOCKS |
| | | | | | domain signature BL00514: | |
| | | | | | V116-G152, E157-V169, F206-A220 | |
| | | | | | Fibrinogen beta and gamma chains C-terminal | PROFILESCAN |
| | | | | | domain signature: | |
| | | | | | T237-S303 | |
| | | | | | PRECURSOR GLYCOPROTEIN SIGNAL | BLAST_PRODOM |
| | | | | | FIBRINOGEN BLOOD COAGULATION CHAIN | |
| | | | | | PLASMA PROTEIN PLATELET PD001241: | |
| | | | | | Q115-R315 FIBRINOGEN BETA/GAMMA | BLAST_DOMO |
| | | | | | DM00531\|P12804\|145-428: N45-P320 | |
| | | | | | S47273\|152-435: N26-P320 | |
| | | | | | JN0596\|27-305: V116-R315 | |
| | | | | | P12799\|106-414: S31-I314 | |
| | | | | | Fibrin_Ag_C_Domain motif: | MOTIFS |
| | | | | | W268-G280 | |
| 18 | 7473606CD1 | 644 | S171 S214 S270 | N574 | Transmembrane domain: | HMMER |
| | | | S290 S363 S405 | | M228-F248 | |
| | | | S425 S535 S581 | | Reprolysin family propeptide domain: | HMMER_PFAM |
| | | | S592 S630 T167 | | Q502-Q615 | |
| | | | T266 T315 T377 | | Glycosaminoglycan attachment sites: | MOTIFS |
| | | | T620 Y534 | | S86-G89, S125-G128 | |
| 19 | 3534918CD1 | 881 | S362 S387 S425 | N257 N403 | EGF-like domains: | HMMER_PFAM |
| | | | S447 S533 S548 | N630 N861 | C682-C716, C722-C762, C474-C508, | |
| | | | S552 S60 S658 | | C514-C553, C559-C591, C597-C633, | |
| | | | S8 S91 T199 | | C639-C676 | |
| | | | T259 T300 T322 | | Thrombospondin type 1 domains: | HMMER_PFAM |
| | | | T367 T39 T507 | | S124-C174, G181-C231 | |
| | | | T626 T632 T715 | | Calcium-binding EGF-like domain BL01187: | BLIMPS_BLOCKS |
| | | | T793 T839 T848 | | C633-H644, C692-Y707 | |
| | | | Y849 | | Type II EGF-like signature PR00010: | BLIMPS_PRINTS |
| | | | | | D593-P604, N630-D637, G697-Y707 | |
| | | | | | HEMICENTIN PRECURSOR SIGNAL | BLAST_PRODOM |
| | | | | | GLYCOPROTEIN EGFLIKE DOMAIN HIM4 | |
| | | | | | PROTEIN ALTERNATIVE SPLICING | |
| | | | | | PD083049: Q725-Y881 | |
| | | | | | EGF-LIKE DOMAIN DM00864\|I55476\|159-241: | BLAST_DOMO |
| | | | | | N649-C722, C480-E561, D525-N600 | |
| | | | | | THROMBOSPONDIN TYPE 1 REPEAT DM00275\| | BLAST_DOMO |
| | | | | | P35440\|485-548: Q165-C226 | |
| | | | | | P07996\|477-540: Q165-C226 | |
| | | | | | Q03350\|479-542: C169-C226 | |
| | | | | | Glycosaminoglycan attachment site: | MOTIFS |
| | | | | | S127-G130 | |
| | | | | | Aspartic acid and asparagine hydroxylation | MOTIFS |
| | | | | | sites: | |
| | | | | | C484-C495, C569-C580, C652-C663, | |
| | | | | | C692-C703 | |
| 20 | 2428715CD1 | 957 | S216 S258 S444 | N255 N400 | Signal peptide: M1-L22 | SPSCAN |
| | | | S525 S526 S56 | N674 N745 | Signal peptide: M1-G25 | HMMER |
| | | | S594 S728 S880 | N774 N784 | EGF-like domains: | HMMER_PFAM |
| | | | S894 T101 T180 | | C37-C72, C78-C115, C121-C156, | |
| | | | T240 T285 T309 | | C166-C202, C206-C241, C275-C310, | |
| | | | T350 T434 T505 | | C316-C351, C357-C390, C396-C431 | |
| | | | T520 T53 T551 | | CUB domain: C793-Y902 | HMMER_PFAM |
| | | | T579 T596 T648 | | Calcium-binding EGF-like signature BL01187: | BLIMPS_BLOCKS |
| | | | T721 T748 T775 | | C115-G126, C407-R422 | |
| | | | T777 T862 T877 | | THROMBOMODULIN SIGNATURE PR00907: | BLIMPS_PRINTS |
| | | | T904 | | C282-P298, L344-C367, G372-S397, | |
| | | | | | C50-D76 | |
| | | | | | GLYCOPROTEIN THYROGLOBULIN | BLAST_PRODOM |
| | | | | | PRECURSOR REPEAT THYROID HORMONE | |
| | | | | | IODINATION SIGNAL EGFLIKE PROTEIN | |
| | | | | | PD009765: C641-C788 | |
| | | | | | EGF-LIKE DOMAIN DM00864\|I55476\|159-241: | BLAST_DOMO |
| | | | | | N320-C396, N279-C357, N84-C156, | |
| | | | | | C363-C437, I49-N124 | |
| | | | | | EGF DM00003\| | BLAST_DOMO |
| | | | | | P98163\|1373-1460: C87-C156, | |
| | | | | | C323-L384 | |
| | | | | | P98063\|706-753: D117-C156 | |
| | | | | | Glycosaminoglycan attachment site: | MOTIFS |
| | | | | | S894-G897 | |
| | | | | | Aspartic acid and asparagine hydroxylation | MOTIFS |
| | | | | | sites: | |
| | | | | | C50-C61, C91-C102, C132-C143, | |
| | | | | | C327-C338, C367-C378, C407-C418 | |
| 21 | 3351332CD1 | 1393 | S1008 S1018 | N1098 N1109 | Signal peptide: M1-G26 | SPSCAN |
| | | | S1158 S1206 | N1169 N1204 | Signal peptide: M1-A22 | HMMER |
| | | | S1255 S1277 | N147 N447 | EGF-like domains: | HMMER_PFAM |
| | | | S1380 S201 S313 | N458 N634 | C60-C95, C101-C137, C143-C178, | |
| | | | S347 S49 S567 | N769 N856 | C184-C219, C230-C265, C271-C305, | |
| | | | T1179 T1214 | N867 N893 | C311-C346, C415-C446, C459-C489, | |
| | | | T1354 T1384 | | C502-C532, C536-C577, C635-C664, | |
| | | | T264 T399 T401 | | C677-C708, C721-C751, C764-C795, | |
| | | | T418 T487 T660 | | C808-C838, C851-C881, C894-C924, | |
| | | | T836 T953 | | C937-C967, C980-C1010, C1067-C1097, | |
| | | | | | C1110-C1140, C1153-C1183, C1196-C1226, | |
| | | | | | C1239-C1269, C1282-C1312, C1325-C1355, | |
| | | | | | Type III EGF-like signature PR00011: | BLIMPS_PRINTS |
| | | | | | C471-C489, C1337-C1355, Q797-G825, | |
| | | | | | C690-C708 | |
| | | | | | THROMBOMODULIN SIGNATURE PR00907: | BLIMPS_PRINTS |
| | | | | | G758-C777, C237-P253, C108-L130 | |
| | | | | | MEGF6 GLYCOPROTEIN EGFLIKE DOMAIN | BLAST_PRODOM |
| | | | | | PD169326: L349-D414 | |
| | | | | | PD165309: N507-C536 | |
| | | | | | SURFACE ANTIGEN PROTEIN PRECURSOR | BLAST_PRODOM |
| | | | | | SIGNAL REPEAT MEMBRANE GPIANCHOR | |
| | | | | | 156G 168G PD001714: H774-C1215 | |
| | | | | | EGF | BLAST_DOMO |
| | | | | | DM00003\|P98163\|1373-1460: S229-V308, | |
| | | | | | C143-E223 DM00003\|P35556\|2219-2292: | |
| | | | | | S274-C346 DM00003\|A57278\|2213-2286: | |
| | | | | | S274-C346 | |
| | | | | | EGF-LIKE DOMAIN DM00864\|I55476\|159-241: | BLAST_DOMO |
| | | | | | N234-C311 | |
| | | | | | EGF domain motifs: | MOTIFS |
| | | | | | C435-C446, C478-C489, C521-C532, | |
| | | | | | C566-C577, C653-C664, C697-C708, | |
| | | | | | C740-C751, C784-C795, C827-C838, | |
| | | | | | C870-C881, C913-C924, C956-C967, | |
| | | | | | C999-C1010, C1043-C1054, | |
| | | | | | C1086-C1097, C1129-C1140, | |
| | | | | | C1172-C1183, C1215-C1226, | |
| | | | | | C1258-C1269, C1301-C1312, | |
| | | | | | C1344-C1355 | |
| | | | | | Aspartic acid and asparagine hydroxylation | MOTIFS |
| | | | | | sites: | |
| | | | | | C71-C82, C195-206, C281-292, C322-333 | |
| 22 | 6382722CD1 | 3695 | S1093 S111 | N1330 N143 | Signal peptide: M1-A35 | SPSCAN |
| | | | S1172 S164 | N1529 N1555 | Signal peptide: M1-A35 | HMMER |
| | | | S1731 S1779 | N2019 N2196 | Laminin N-terminal (Domain VI): L45-G298 | HMMER_PFAM |
| | | | S1807 S1826 | N2209 N2303 | | |
| | | | S1836 S1841 | N2423 N243 | Laminin EGF-like (Domains III and V): | HMMER_PFAM |
| | | | S1870 S190 | N2501 N2568 | C300-C356, C359-C426, | |
| | | | S1901 S1902 | N2707 N3107 | C429-C471, C494-C538, C541-C584, | |
| | | | S191 S1982 | N3209 N3257 | C587-C629, C632-C674, C677-C720, | |
| | | | S2211 S2358 | N3287 N3626 | E721-C773, C776-C826, C829-D870, | |
| | | | S2626 S2684 | N452 N479 | C1438-C1481, C1484-C1525, C1528-C1574, | |
| | | | S2698 S2798 | N900 N921 N95 | C1577-C1625, C1864-C1910, | |
| | | | S2819 S290 | N959 | C1913-C1966, C1969-C2020, C2023-C2067, | |
| | | | S2934 S2944 | | C2070-C2114, C2117-C2167 | |
| | | | S3035 S3076 | | Laminin B (Domain IV): Y1693-E1829 | HMMER_PFAM |
| | | | S3086 S3155 | | Laminin G domain; | HMMER_PFAM |
| | | | S3316 S3349 | | F2876-S2911, L2970-D3102, | |
| | | | S3374 S3429 | | V3370-G3502, V3549-A3676 | |
| | | | S3478 S352 S380 | | Laminin-type EGF-like signature BL01248: | BLIMPS_BLOCKS |
| | | | S477 S697 S73 | | C1883-C1895 | |
| | | | S768 S810 S828 | | Type III EGF-like signature PR00011: | BLIMPS_PRINTS |
| | | | S902 S943 S947 | | C1589-C1607, C2033-C2051, C1596-G1624, | |
| | | | T1032 T1091 | | C1543-C1561 | |
| | | | T1154 T124 | | LAMININ DOMAIN | BLAST_PRODOM |
| | | | T1269 T1355 | | PD035152: S2731-C3292, P3301-F3353 | |
| | | | T1362 T1557 | | PD025440: L871-A1435 | |
| | | | T1634 T1643 | | PD002082: H46-G298 | |
| | | | T1658 T1711 | | PD155637: E1687-L1858 | |
| | | | T1720 T1745 | | LAMININ CHAIN B1 DM01003\|P25391\|14-258: | BLAST_DOMO |
| | | | T2021 T2047 | | L45-Y289 | |
| | | | T208 T2128 | | DM01003\|S53868\|27-271: L45-Y289 | |
| | | | T2288 T2315 | | DM01003\|I49077\|27-271: L45-Y289 | |
| | | | T2515 T2570 | | DM01003\|S50829\|1-208: P94-Y289 | |
| | | | T2625 T2749 | | TNFR/NGFR motif: C2051-C2090 | MOTIFS |
| | | | | | Glycosaminoglycan attachment sites: | MOTIFS |
| | | | | | S1531-G1534, S1972-G1975, S3149-G3152 | |
| | | | | | RGD motifs: | MOTIFS |
| | | | | | R1722-D1724, R1838-D1840 | |
| | | | | | EGF domain motifs: | MOTIFS |
| | | | | | C322-C333, C447-C458, C515-C526, C560-C571, | |
| | | | | | C605-C616, C650-C661, C696-C707, | |
| | | | | | C744-C755, C797-C808, C848-C859, C1457-C1468, | |
| | | | | | C1550-C1561, C1596-C1607, C1831-C1842 | |
| | | | | | C1937-C1948, C2040-C2051, C2070-C2081, | |
| | | | | | C2088-C2099, C2131-C2142 | |
| 23 | 55022490CD1 | 1255 | S1009 S1052 | N1039 N1129 | Signal peptide: M1-G27 | SPSCAN |
| | | | S1097 S1188 | N262 N347 | Thrombospondin type 1 domain: | HMMER_PFAM |
| | | | S167 S174 S444 | N519 N540 | S111-C161, W394-C448, G518-C563, | |
| | | | S470 S486 S580 | N981 N988 | W984-C1032, W1035-C1090, | |
| | | | S604 S74 S761 | | W1093-C1139, T1140-C1197, | |
| | | | S877 S939 S954 | | PROTEIN F25H8.3 F53B6.2 KIAA0605 | BLAST_PRODOM |
| | | | S966 T1115 | | PROCOLLAGEN C37C3.6 SERINE | |
| | | | T1130 T1217 | | PROTEASE INHIBITOR ALTERNATIVE | |
| | | | T1228 T138 T272 | | PD007018: W394-P512, W1093-P1203 | |
| | | | T31 T526 T531 | | PROTEIN PROCOLLAGEN THROMBOSPONDIN | BLAST_PRODOM |
| | | | Y316 | | MOTIFS NPROTEINASE A DISINTEGRIN | |
| | | | | | METALLOPROTEASE WITH ADAMTS1 | |
| | | | | | PD011654: C198-C266 | |
| | | | | | PD014161: V269-E380 | |
| | | | | | Glycosaminoglycan attachment site: | MOTIFS |
| | | | | | S575-G578 | |
| 24 | 6755002CD1 | 911 | S116 S165 S29 | N130 N337 | Signal peptide: M1-A22 | HMMER |
| | | | S310 S319 S419 | | Signal cleavage: M1-A22 | SPSCAN |
| | | | S446 S615 S67 | | C_Type_Lectin C784-C808 | MOTIFS |
| | | | S704 S708 S727 | | Egf C670-C681 | MOTIFS |
| | | | S74 T212 T219 | | Ig_Mhc Y135-H141 | MOTIFS |
| | | | T269 T382 T386 | | Lectin C-type domain lectin_c: T714-C808 | HMMER_PFAM |
| | | | T397 T420 T430 | | Extracellular link domain Xlink: | HMMER_PFAM |
| | | | T545 T558 T660 | | G156-Y251, G257-F353 | |
| | | | T705 T728 T807 | | Immunoglobulin domain ig: G50-V139 | HMMER_PFAM |
| | | | Y135 Y459 | | EGF-like domain EGF: C650-C681 | HMMER_PFAM |
| | | | | | Sushi domain (SCR repeat) sushi: C815-C871 | HMMER_PFAM |
| | | | | | C-type lectin domain signature | PROFILESCAN |
| | | | | | c_type_lectin.prf: P763-G828 | |
| | | | | | C-type lectin domain protein BL00615: | BLIMPS_BLOCKS |
| | | | | | C699-C716, W795-C808 | |
| | | | | | Link domain proteins BL01241: E272-G324 | BLIMPS_BLOCKS |
| | | | | | TYPE II ANTIFREEZE PROTEIN (lectin-like) | BLIMPS_PRINTS |
| | | | | | PR00356: | |
| | | | | | F687-C699, C699-C716, R717-F734, | |
| | | | | | W744-D760, W795-C808 | |
| | | | | | C-TYPE LECTIN DM00035\|A54423\|689-811: | BLAST_DOMO |
| | | | | | C688-G811 | |
| | | | | | COMPLEMENT FACTOR H REPEAT DM00260 | BLAST_DOMO |
| | | | | | A54423\|142-252: G142-E253 | |
| | | | | | BREVICAN CORE PROTEIN PRECURSOR (brain- | BLAST_PRODOM |
| | | | | | specific lectican) | |
| | | | | | PD022317: L479-V651 PD021260: R354-E455 | |
| | | | | | EGFLIKE DOMAIN REPEAT | BLAST_PRODOM |
| | | | | | PD150847: D28-V154, PD000918: K267-F353 | |
| 25 | 7350907CD1 | 467 | S124 S194 S261 | N169 N270 | Signal cleavage: M1-T31 | SPSCAN |
| | | | S273 S335 S362 | N289 N376 | GLYCOPROTEIN OLFACTOMEDIN | BLAST_PRODOM |
| | | | S383 S415 S432 | N413 N455 N85 | MESHWORK-INDUCED RESPONSE SIGNAL | |
| | | | S47 T220 T36 | | PROTEIN PRECURSOR PD006897: E258-I452 | |
| | | | T97 Y103 Y323 | | NEURONAL OLFACTOMEDIN RELATED | BLAST_PRODOM |
| | | | | | ER LOCALIZED PRECURSOR | |
| | | | | | PD037534: A135-R257, PD020721: M11-K134 | |
| 26 | 7474411CD1 | 1018 | S117 S195 S206 | N373 N441 | Signal cleavage: M1-P25 | SPSCAN |
| | | | S309 S312 S394 | N709 | Receptor_Cytokines_2 G57-S63 | MOTIFS |
| | | | S458 S557 S648 | | Transmembrane domain: M1-F20 | HMMER |
| | | | S656 S661 S803 | | Thrombospondin type 1 domain tsp_1: | HMMER_PFAM |
| | | | S84 S896 T368 | | S737-C793 | |
| | | | T483 T493 T649 | | PROCOLLAGEN THROMBOSPONDIN MOTIFS | BLAST_PRODOM |
| | | | T665 T843 T851 | | PD011654: K344-C411, PD014161: Q412-I527 | |
| | | | | | PROCOLLAGEN SERINE PROTEASE INHIBITOR | BLAST_PRODOM |
| | | | | | PD007018: W856-P969 | |
| 27 | 4755911CD1 | 1458 | S1014 S1027 | N1199 N215 | Ankyrin repeat ank: P108-D140, E141-N173, | HMMER_PFAM |
| | | | S104 S1073 | N354 N958 | S174-L204, N215-K247, S248-T279 | |
| | | | S1131 S1254 | | (SAM) protein interaction domain SAM: | HMMER_PFAM |
| | | | S1274 S1298 | | E497-S560, H568-A630 | |
| | | | S1391 S1395 | | PROLINE-RICH PROTEIN DM03894\|A39066\| | BLAST_DOMO |
| | | | S1429 S1437 | | 1-159: P1216-P1358 | |
| | | | S1444 S174 S367 | | | |
| | | | S447 S450 S560 | | | |
| | | | S673 S732 S777 | | | |
| | | | S782 S79 S846 | | | |
| | | | S916 S918 S946 | | | |
| | | | S981 T1056 | | | |
| | | | T1092 T1103 | | | |
| | | | T1122 T1150 | | | |
| | | | T1170 T1174 | | | |
| | | | T1203 T1273 | | | |
| | | | T177 T283 T337 | | | |
| | | | T356 T534 T604 | | | |
| | | | T667 T678 T702 | | | |
| | | | T712 T716 T717 | | | |
| | | | T80 T828 T841 | | | |
| | | | T855 Y363 Y594 | | | |
| 28 | 379766CD1 | 323 | | | Signal peptide: M1-T24 | HMMER |
| | | | | | Thrombospondin type 1 domain: D79-C123 | HMMER-PFAM |
| | | | | | Thrombospondin, procollagen, N-proteinase | BLAST-PRODOM |
| | | | | | A, disintegrin, metalloprotease with | |
| | | | | | ADAMTS1 PD011654: | |
| | | | | | P157-C227, Q133-G203 | |
| 29 | 553744CD1 | 234 | S231 T140 | | | |
| 30 | 1825473CD1 | 377 | S120 S140 S18 | N128 N135 | Signal peptide: M1-S18 | HMMER |
| | | | S38 S41 S62 | N146 N97 | Signal peptide: M1-G22 | SPScan |
| | | | T267 T343 Y48 | | Mucin, MUC5, tracheobronchial: | BLAST-DOMO |
| | | | | | DM05454\|S55316\|1-317: | |
| | | | | | P91-A350, C70-T348, S150-P351, | |
| | | | | | Q163-A350, P203-A350 | |
| | | | | | Salivary glue protein: | BLAST-DOMO |
| | | | | | DM02055\|P02840\|17-234: | |
| | | | | | S149-K355, S120-T330, P85-T291 | |
| 31 | 7950094CD1 | 833 | S200 S34 S364 | N106 N121 | Signal peptide: M1-V23 | HMMER |
| | | | S382 S46 S480 | N310 N419 | Transmembrane domain: L667-R687 | HMMER |
| | | | S505 S555 S685 | N522 N564 | Plexin repeat: D499-N551 | HMMER-PFAM |
| | | | S742 S826 T229 | | Sema domain: F53-K481 | HMMER-PFAM |
| | | | T276 T302 T418 | | Semaphorin, fasciclin, collapsin: | BLAST-DOMO |
| | | | T429 T523 T561 | | DM01606\|I48747\|1-646: L10-W519 | |
| | | | T57 T701 Y249 | | Semaphorin, fasciclin, collapsin: | BLAST-DOMO |
| | | | Y345 Y736 | | DM01606\|A49069\|1-646: N26-W519 | |
| | | | | | Semaphorin, fasciclin, collapsin: | BLAST-DOMO |
| | | | | | DM01606\|I48744\|1-639: A12-G592 | |
| | | | | | Semaphorin, fasciclin, collapsin: | BLAST-DOMO |
| | | | | | DM01606\|I48748\|1-589: D52-G530 | |
| | | | | | Semaphorin I (neural development factor): | BLAST-PRODOM |
| | | | | | PD129812: H540-V833 | |
| | | | | | Semaphorin, receptor, kinase, tyrosin | BLAST-PRODOM |
| | | | | | protein PD001844: | |
| | | | | | F145-E351, R242-S453, L56-E204, | |
| | | | | | P754-G764 | |
| | | | | | Semaphorin I: | BLAST-PRODOM |
| | | | | | PD166847: M1-D52 | |
| 32 | 7479484CD1 | 1291 | S1037 S113 | N394 N500 N54 | Cell attachment sequence: R844-D846 | MOTIFS |
| | | | S1279 S216 S224 | | Tumor recognition, prolyl: | BLAST-DOMO |
| | | | S225 S272 S324 | | DM08077\|P30414\|230-1403: | |
| | | | S380 S396 S434 | | S613-P1098, E398-P896 | |
| | | | S445 S450 S453 | | Acidic serine cluster repeat: | BLAST-DOMO |
| | | | S465 S466 S480 | | DM03496\|P32583\|57-405: | |
| | | | S481 S489 S508 | | S539-Q824, N500-K819, S557-V878, | |
| | | | S535 S547 S558 | | S421-S754, A474-R765, S465-W800, | |
| | | | S563 S585 S589 | | A378-Y718 | |
| | | | S595 S596 S606 | | Type B repeat: | BLAST-DOMO |
| | | | S618 S635 S652 | | DM05511\|S26650\|1-1203: | |
| | | | S655 S656 S662 | | E400-P826, D534-Q791, Q593-D842 | |
| | | | S678 S683 S688 | | Type B repeat: | BLAST- DOMO |
| | | | S691 S695 S708 | | DM05511\|P18583\|113-1296: | |
| | | | S713 S719 S720 | | S585-P826, D475-D842, D534-S783 | |
| | | | S721 S760 S764 | | | |
| | | | S785 S790 S795 | | | |
| | | | S799 S804 S810 | | | |
| | | | S831 S866 S871 | | | |
| | | | S889 S947 S954 | | | |
| | | | S965 S971 T1102 | | | |
| | | | T118 T175 T243 | | | |
| | | | T268 T341 T371 | | | |
| | | | T41 T439 T862 | | | |
| | | | T879 T944 | | | |
| 33 | 6780147CD1 | 736 | S131 S144 S20 | N316 N467 | Signal peptide: M1-A22 | HMMER |
| | | | S361 S369 S409 | N540 | Signal peptide: M1-A22 | SPScan |
| | | | S469 S479 S653 | | Cell attachment sequence: R48-D50 | MOTIFS |
| | | | S683 S699 S726 | | Leucine zipper pattern: L445-L466 | MOTIFS |
| | | | T394 T430 T495 | | CD4, GNB3, mouse BAC library PD043366: | BLAST-PRODOM |
| | | | T508 T542 T570 | | L445-K733, H176-E412, C79-E234, | |
| | | | T608 T630 Y250 | | Q97-E135 | |
| | | | | | CASP, cartilage-associated PD023886: | BLAST-PRODOM |
| | | | | | Y46-C282, E201-S361 | |
| 34 | 7204554CD1 | 1896 | S1004 S1115 S45 | N1043 N1098 | Signal peptide: M1-A26 | SPScan |
| | | | S1271 S1382 | N1140 N1187 | Signal peptide: M1-A28 | HMMER |
| | | | S1435 S1487 | N1212 N1609 | Transmembrane domains: | HMMER |
| | | | S1546 S1621 | N1612 N572 | V10-A28, P1244-Y1267 | |
| | | | S1631 S1635 | N597 N660 | Plexin repeat: | HMMER-PFAM |
| | | | S1767 S1785 | N672 N701 | S514-V564, N660-P707, K808-T862 | |
| | | | S1797 S1811 | N761 N769 N77 | Sema domain: | HMMER-PFAM |
| | | | S1827 S1883 | N785 | L51-N495 | |
| | | | S202 S203 S249 | | IPT/TIG domain: | HMMER-PFAM |
| | | | S294 S454 S542 | | P864-V959, P961-T1045, P1048-Y1147, | |
| | | | S599 S608 S621 | | P1150-Y1236 | |
| | | | S838 S858 S908 | | ATP/GTP-binding site motif A (P-loop): | MOTIFS |
| | | | S929 S952 T1036 | | G188-S195 | |
| | | | S162 T1075 | | Hepatocyte tyrosine kinase: | BLAST-DOMO |
| | | | T1200 T1220 | | DM03653\|P08581\|14-526: | |
| | | | T1277 T1363 | | L51-C521, T647-C667 | |
| | | | T1574 T1575 | | Tyrosine kinase: | BLAST-DOMO |
| | | | T1739 T1779 | | DM01368\|P51805\|796-899: | |
| | | | T189 T268 T279 | | C819-I924 | |
| | | | T361 T503 T519 | | Tyrosine kinase: | BLAST-DOMO |
| | | | T604 T647 T957 | | DM02937\|P51805\|991-1085: | |
| | | | Y1540 Y1817 | | P1021-V1109 | |
| | | | | | Hepatocyte tyrosine kinase: | BLAST-DOMO |
| | | | | | DM03653\|A48196\|13-528: | |
| | | | | | L19-E522 | |
| | | | | | Plexin precursor PD008852: | BLAST-PRODOM |
| | | | | | A1262-S1672, T1504-S1896, E482-N495 | |
| | | | | | Receptor, tyrosine kinase PD003981: | BLAST-PRODOM |
| | | | | | R892-N1212, M502-H531 | |
| | | | | | Plexin precursor PD010132: | BLAST-PRODOM |
| | | | | | P570-C843 | |
| | | | | | Plexin precursor PD003973: | BLAST-PRODOM |
| | | | | | R372-Y498 | |
| 35 | 6833247CD1 | 215 | S108 S140 S177 | N102 N111 N45 | signal peptide: | SPSCAN |
| | | | S8 T104 T124 | | M1-C39 | |
| | | | T53 | | Transmembrane domain: | HMMER |
| | | | | | Q17-A37 | |
| | | | | | Lectin C-type domain: | HMMER_PFAM |
| | | | | | R110-C207 | |
| | | | | | C-type lectin domain protein | BLIMPS_BLOCKS |
| | | | | | BL00615A: C95-C112 | |
| | | | | | BL00615B: W194-C207 | |
| | | | | | C-type lectin domain signature: | PROFILESCAN |
| | | | | | Q159-T213 | |
| | | | | | TYPE II ANTIFREEZE PROTEIN | BLIMPS_PRINTS |
| | | | | | PR00356: S113-F130, F142-D158, | |
| | | | | | W194-C207, C83-C95, | |
| | | | | | C95-C112 | |
| | | | | | C-TYPE LECTIN | BLAST_DOMO |
| | | | | | DM00035\|A54423\|689-811: C83-I209 | |
| | | | | | DM00035\|P10716\|405-536: D87-K208 | |
| | | | | | DM00035\|P16112\|2205-2327: C84-K208 | |
| | | | | | DM00035\|A46274\|248-377: C84-K208 | |
| 36 | 4148119CD1 | 579 | S144 S253 S394 | N69 | signal_cleavage: | SPSCAN |
| | | | S485 T162 T472 | | M1-G24 | |
| | | | T521 T570 | | signal peptide: | HMMER |
| | | | | | M1-G24 | |
| | | | | | Leucine Rich Repeat: | HMMER_PFAM |
| | | | | | Q239-H264, S265-A288, G310-R335, | |
| | | | | | G336-R359, G381-R406, A407-P426, | |
| | | | | | G428-D451, Q452-Q477, A478-P497, | |
| | | | | | A499-P522, R523-P548, A73-S96, | |
| | | | | | G97-T122, Q123-R146, V168-E193, | |
| | | | | | A194-P213, S215-T238 | |
| | | | | | Leucine zipper pattern: | MOTIFS |
| | | | | | L198-L219 L269-L290 L340-L361 | |
| | | | | | L411-L432 L418-L439 L482-L503 | |

TABLE 4

| Polynucleotide | Incyte | Sequence | Selected | | | |
|---|---|---|---|---|---|---|
| SEQ ID NO: | Polynucleotide ID | Length | Fragment(s) | Sequence Fragments | 5′ Position | 3′ Position |
| 37 | 1888682CB1 | 1211 | | 7347284H1 (LUNLTUE01) | 315 | 905 |
| | | | | 2110746R6 (BRAITUT03) | 749 | 1211 |
| | | | | 7016843H1 (KIDNNOC01) | 1 | 625 |
| 38 | 1794980CB1 | 1523 | 1403-1523, 1-121, | 6775891H1 (OVARDIR01) | 1 | 700 |
| | | | 409-454, | 4955572H1 (ENDVUNT01) | 1202 | 1523 |
| | | | 833-907 | 6149683H1 (BRANDIT03) | 780 | 1450 |
| | | | | 2149263F6 (BRAINOT09) | 678 | 1313 |
| 39 | 5533958CB1 | 1368 | 1-589 | 6552411H1 (BRAFNON02) | 878 | 1368 |
| | | | | 7237564H1 (BRAINOY02) | 850 | 1358 |
| | | | | 6976222H1 (BRAHTDR04) | 579 | 1333 |
| | | | | 7182163H1 (BONRFEC01) | 1 | 604 |
| 40 | 60210196CB1 | 3157 | 1-2311 | 71699406V1 | 2592 | 3157 |
| | | | | 71699506V1 | 2085 | 2898 |
| | | | | 71699453V1 | 1611 | 2165 |
| | | | | 7050851H1 (BRACNOK02) | 1 | 792 |
| | | | | 70810715V1 | 2310 | 2955 |
| | | | | 71699537V1 | 1393 | 2080 |
| | | | | 3767657F7 (BRSTNOT24) | 769 | 1492 |
| | | | | 5436183F6 (SPLNNOT17) | 882 | 1623 |
| 41 | 815125CB1 | 3264 | 1879-1968, 1-938 | 70506843V1 | 512 | 1131 |
| | | | | 71182375V1 | 1999 | 2553 |
| | | | | 6764263H1 (BRAUNOR01) | 1 | 646 |
| | | | | 71149313V1 | 2520 | 3264 |
| | | | | 60205342U1 | 1305 | 1932 |
| | | | | 1376122F1 (LUNGNOT10) | 2143 | 2654 |
| | | | | 6488119H1 (MIXDUNB01) | 1453 | 2026 |
| | | | | 70483405V1 | 702 | 1342 |
| 42 | 1386915CB1 | 3383 | 1-1510 | 6841587H1 (BRSTNON02) | 3124 | 3376 |
| | | | | 70772013V1 | 1247 | 1875 |
| | | | | 70773009V1 | 663 | 1266 |
| | | | | 1350440F1 (LATRTUT02) | 2451 | 3007 |
| | | | | 5797946H1 (PLACFET04) | 2547 | 3041 |
| | | | | 6428723H1 (LUNGNON07) | 1 | 338 |
| | | | | 6481347H1 (PROSTMC01) | 319 | 991 |
| | | | | 3326918T6 (HEAONOT04) | 2914 | 3333 |
| | | | | 843210H1 (PROSTUT05) | 3173 | 3383 |
| | | | | 70772645V1 | 1958 | 2562 |
| | | | | 70771297V1 | 1319 | 1951 |
| | | | | 70772890V1 | 1852 | 2483 |
| 43 | 1344495CB1 | 2741 | 1-361, 2693-2741 | 70267334V1 | 657 | 1209 |
| | | | | 1860069T6 (PROSNOT18) | 1944 | 2667 |
| | | | | g2577445 | 2437 | 2741 |
| | | | | 1344495F6 (PROSNOT11) | 1 | 566 |
| | | | | 1860069F6 (PROSNOT18) | 1083 | 1873 |
| | | | | 70269146V1 | 2051 | 2687 |
| | | | | 70266807V1 | 1211 | 1944 |
| | | | | 70270212V1 | 539 | 1172 |
| | | | | 70267638V1 | 1803 | 2365 |
| 44 | 1485774CB1 | 2076 | 524-976, 303-332 | 1617862H1 (BRAITUT12) | 1776 | 2006 |
| | | | | 744054R6 (BRAITUT01) | 1413 | 1972 |
| | | | | 1485774F6 (CORPNOT02) | 1028 | 1467 |
| | | | | 6904024H1 (MUSLTDR02) | 1 | 703 |
| | | | | g3043569_CD | 356 | 1842 |
| | | | | 1290195H1 (BRAINOT11) | 562 | 837 |
| | | | | g1242437 | 1607 | 2076 |
| 45 | 7289372CB1 | 2957 | 1214-1324, 1-236, | 7289157H1 (BRAIFER06) | 2467 | 2957 |
| | | | 991-1098, | 7252620H2 (BRAIFEE04) | 1964 | 2575 |
| | | | 1803-2480, | 7675562J2 (NOSETUE01) | 972 | 1527 |
| | | | 464-920 | g772391 | 1 | 495 |
| | | | | 7290371H1 (BRAIFER06) | 1579 | 2105 |
| | | | | 7288441H1 (BRAIFER06) | 342 | 788 |
| | | | | 7292572R8 (BRAIFER06) | 747 | 1443 |
| | | | | 5090004R8 (UTRSTMR01) | 1440 | 1948 |
| 46 | 1672338CB1 | 1223 | 1196-1223, | 6609653H1 (EPIGTMC01) | 1 | 609 |
| | | | 652-678 | 71743918V1 | 558 | 1223 |
| 47 | 184661CB1 | 2888 | 1331-1592, 1-596, | 70160946V1 | 2386 | 2888 |
| | | | 2355-2401 | 7703219J1 (UTRETUE01) | 1520 | 2162 |
| | | | | 70160174V1 | 2176 | 2810 |
| | | | | 71401492V1 | 620 | 1168 |
| | | | | 70154040V1 | 2020 | 2637 |
| | | | | 6153426H1 (ENDMUNT04) | 1 | 544 |
| | | | | 7192494H2 (BRATDIC01) | 1062 | 1709 |
| | | | | 71142234V1 | 449 | 1137 |
| 48 | 3719737CB1 | 3142 | 3067-3142, 1-409, | 71046117V1 | 2277 | 2807 |
| | | | 1301-1659, | 71047416V1 | 1375 | 1784 |
| | | | 761-822 | 71046670V1 | 1676 | 2317 |
| | | | | 70064096V1 | 2614 | 3142 |
| | | | | 4027661F8 (BRAINOT23) | 916 | 1327 |
| | | | | 7455730H1 (LIVRTUE01) | 1156 | 1582 |
| | | | | 7431827H1 (UTRMTMR02) | 543 | 1071 |
| | | | | 7189788H2 (BRATDIC01) | 1 | 603 |
| 49 | 5773251CB1 | 4749 | 3194-3442, 1-1545, | 71699127V1 | 1649 | 2386 |
| | | | 4114-4749, | 7091322H1 (BRAUTDR03) | 963 | 1558 |
| | | | 2497-2543 | 71698165V1 | 2430 | 3198 |
| | | | | 6981926H1 (BRAIFER05) | 1221 | 1633 |
| | | | | 594160T6 (BRAVUNT02) | 3989 | 4604 |
| | | | | 7733307H2 (COLDDIE01) | 3460 | 4056 |
| | | | | g3927714 | 326 | 712 |
| | | | | 71698388V1 | 2348 | 3102 |
| | | | | 7733307J2 (COLDDIE01) | 4181 | 4749 |
| | | | | 70089831V1 | 3135 | 3978 |
| | | | | 71699024V1 | 1512 | 2271 |
| | | | | 7754525J1 (SPLNTUE01) | 1 | 570 |
| | | | | 7285547H1 (BRAIFEJ01) | 650 | 1140 |
| 50 | 5426470CB1 | 4155 | 2222-2681, | 6991563H1 (BRAIFER05) | 1 | 478 |
| | | | 4067-4086, 1-171, | 6122067H1 (BRAHNON05) | 3604 | 4155 |
| | | | 3250-3606, | 5814755F8 (PROSTUS23) | 2969 | 3555 |
| | | | 301-1873 | 7177748H1 (BRAXDIC01) | 2416 | 3023 |
| | | | | g7959252_CD | 442 | 3284 |
| | | | | 5426470T6 (PROSTMT07) | 3210 | 4048 |
| | | | | 5426470F6 (PROSTMT07) | 2219 | 2938 |
| | | | | 7035583R8 (SINTFER03) | 1610 | 1786 |
| | | | | 4329672H1 (KIDNNOT32) | 2110 | 2361 |
| | | | | 7178436H1 (BRAXDIC01) | 949 | 1498 |
| 51 | 7087904CB1 | 1327 | 943-1327, | 7946383H1 (BRABNOE02) | 151 | 976 |
| | | | 267-490 | 7087904H1 (BRAUTDR03) | 1 | 392 |
| | | | | 6312090H1 (NERDTDN03) | 605 | 1327 |
| 52 | 7477312CB1 | 5529 | 4005-4070, 1-246, | GNN.g7923864_002 | 90 | 2347 |
| | | | 3475-3746, | 7102261F8 (BRAWTDR02) | 5148 | 5529 |
| | | | 688-2835, | 7314180H1 (LIVRNOE07) | 1803 | 2384 |
| | | | 4856-5529 | 7719744J1 (SINTFEE02) | 2885 | 3567 |
| | | | | 8023704J1 (BRABDIE02) | 2342 | 3058 |
| | | | | 7398367H1 (KIDEUNE02) | 596 | 1224 |
| | | | | 6953114H1 (BRAITDR02) | 3429 | 4052 |
| | | | | 7231729H1 (BRAXTDR15) | 4267 | 4889 |
| | | | | 6772109J1 (BRAUNOR01) | 4599 | 5255 |
| | | | | 7228637H1 (BRAXTDR15) | 3676 | 4218 |
| | | | | 6880723J1 (BRAHTDR03) | 4193 | 4837 |
| | | | | GBI: g7923864.edit | 1 | 503 |
| | | | | 7070679R8 (BRAUTDR02) | 239 | 650 |
| | | | | 6034612H1 (PITUNOT06) | 4974 | 5501 |
| | | | | 7647642J1 (UTRSTUE01) | 1362 | 1642 |
| 53 | 2739431CB1 | 1623 | 1-1302 | 2819460F6 (BRSTNOT14) | 1503 | 1623 |
| | | | | 70563142V1 | 627 | 1142 |
| | | | | 1388139T6 (CARGDIT02) | 303 | 1023 |
| | | | | 1388139F6 (CARGDIT02) | 1 | 587 |
| | | | | 2737908T6 (OVARNOT09) | 975 | 1599 |
| 54 | 7473606CB1 | 2242 | 1053-1326, | 2618950T6 (GBLANOT01) | 666 | 863 |
| | | | 252-776, 1-167, | 5624160R8 (THYMNOR02) | 1892 | 2242 |
| | | | 864-955, | 2618950F6 (GBLANOT01) | 1 | 667 |
| | | | 1401-1490 | 6770895J1 (BRAUNOR01) | 1610 | 1865 |
| | | | | g5545559 | 1602 | 2085 |
| | | | | GNN.g6454068_000018_002.edit | 29 | 1865 |
| 55 | 3534918CB1 | 3751 | 854-1195, | 6483020H1 (MIXDUNB01) | 1355 | 1880 |
| | | | 593-646, 1-129, | 70882296V1 | 2517 | 3054 |
| | | | 3709-3751, | 70880032V1 | 3139 | 3751 |
| | | | 1706-2129 | 6819410H1 (OVARDIR01) | 513 | 1170 |
| | | | | 3736613T6 (SMCCNOS01) | 3127 | 3701 |
| | | | | 70879201V1 | 1925 | 2526 |
| | | | | GNN.g6634914_000002_002 | 1 | 1672 |
| | | | | 70879032V1 | 2483 | 3011 |
| | | | | 70879778V1 | 1833 | 2470 |
| | | | | 1404901T6 (LATRTUT02) | 2984 | 3670 |
| 56 | 2428715CB1 | 3579 | 1-94, 2957-3167 | 71902796V1 | 2452 | 3184 |
| | | | 599-2309 | 71907111V1 | 2725 | 3295 |
| | | | | 7362261H1 (BRAIFEE05) | 2407 | 2663 |
| | | | | GNN.g5911819_008.edit | 1051 | 2966 |
| | | | | 2428715H1 (SCORNON02) | 2359 | 2590 |
| | | | | 6925281R8 (PLACFER06) | 2767 | 3579 |
| | | | | 55037058H2 | 1632 | 2403 |
| | | | | 8186336H1 (EYERNON01) | 1 | 648 |
| | | | | GBI.g5911819_000001.edit | 600 | 1412 |
| | | | | 7287205F8 (BRAIFER06) | 984 | 1517 |
| | | | | FL2428715_g6815043_000026_g8052237_1_3-4.edit | 466 | 726 |
| | | | | 1736320T6 (COLNNOT22) | 4260 | 4888 |
| 57 | 3351332CB1 | 5178 | 4912-5178, 1-3036 | 71990942V1 | 3479 | 4113 |
| | | | 3143-3274, | 72036025V1 | 2484 | 3377 |
| | | | 3855-4182 | 71992140V1 | 2434 | 3066 |
| | | | | 8006270H1 (PENIFEC01) | 1743 | 2484 |
| | | | | 7715524H1 (SINTFEE02) | 1197 | 1736 |
| | | | | 1736320F6 (COLNNOT22) | 4066 | 4603 |
| | | | | 8037780H1 (SMCRUNE01) | 1715 | 2099 |
| | | | | 7715524J1 (SINTFEE02) | 666 | 1078 |
| | | | | GNN.g9187279_000012_002.edit | 644 | 1978 |
| | | | | 1437833F1 (PANCNOT08) | 4690 | 5178 |
| | | | | GBI.g9844022_000020_000016_000015.edit | 272 | 766 |
| | | | | 8037316H1 (SMCRUNE01) | 854 | 1588 |
| | | | | 71990329V1 | 3204 | 4032 |
| | | | | 8037316J1 (SMCRUNE01) | 1 | 639 |
| 58 | 6382722CB1 | 11367 | 1-5178 | 7104534H1 (BRAWTDR02) | 4529 | 5108 |
| | | | | 6808001J1 (SKIRNOR01) | 5941 | 6661 |
| | | | | 7000837H1 (HEALDIR01) | 3133 | 3749 |
| | | | | 7324406H1 (COLRTUE01) | 2301 | 2916 |
| | | | | 2778756T6 (OVARTUT03) | 10822 | 11353 |
| | | | | 1675050F6 (BLADNOT05) | 10293 | 10903 |
| | | | | 6938286R8 (FTUBTUR01) | 834 | 1498 |
| | | | | 2951540F6 (KIDNFET01) | 10075 | 10675 |
| | | | | 6942017H1 (FTUBTUR01) | 2091 | 2548 |
| | | | | 7663312J1 (UTRSTME01) | 8747 | 9415 |
| | | | | 7705507H1 (UTRETUE01) | 3523 | 4235 |
| | | | | 7716415J1 (SINTFEE02) | 8632 | 9160 |
| | | | | 7755720H1 (SPLNTUE01) | 4863 | 5504 |
| | | | | 8037265J1 (SMCRUNE01) | 3740 | 4494 |
| | | | | 8045466H1 (OVARTUE01) | 1434 | 2165 |
| | | | | 7641932J1 (SEMVTDE01) | 9355 | 10078 |
| | | | | 7751563H1 (HEAONOE01) | 6986 | 7768 |
| | | | | 7076128F6 (BRAUTDR04) | 121 | 881 |
| | | | | 7970390H1 (MIXDDIA01) | 10912 | 11367 |
| | | | | 7713737H1 (TESTTUE02) | 9483 | 10150 |
| | | | | 7755720J1 (SPLNTUE01) | 4429 | 5069 |
| | | | | 8045466J1 (OVARTUE01) | 1075 | 1528 |
| | | | | 7763930J1 (URETTUE01) | 7298 | 8008 |
| | | | | 6975271H1 (BRAHTDR04) | 6667 | 7261 |
| | | | | GNN.g8670608_000002_002.edit | 1 | 297 |
| | | | | 7644864J1 (UTRSTUE01) | 7987 | 8616 |
| | | | | 7699541H1 (KIDPTDE01) | 5176 | 5894 |
| | | | | 6765766H1 (BRAUNOR01) | 2543 | 3057 |
| | | | | 6948079H1 (BRAITDR02) | 2765 | 3434 |
| | | | | 6759347J1 (HEAONOR01) | 5801 | 6513 |
| | | | | 7699541J1 (KIDPTDE01) | 6468 | 7236 |
| | | | | 55145718J1 | 8102 | 8728 |
| 59 | 55022490CB1 | 4255 | 2644-2898, 1-436, | 8110806H1 (OSTEUNC01) | 1231 | 1882 |
| | | | 1179-1891 | 7201271F8 (LUNGFER04) | 2265 | 2964 |
| | | | | 8213356H1 (FIBRTXC01) | 3680 | 4255 |
| | | | | 7953052H1 (SYNONOC01) | 2062 | 2558 |
| | | | | 55022495H1 | 1 | 645 |
| | | | | 55022496H1 | 1316 | 1961 |
| | | | | 2939061F6 (THYMFET02) | 3185 | 3774 |
| | | | | 55022495J1 | 608 | 1248 |
| | | | | 55022496J1 | 661 | 1310 |
| | | | | 7704179H1 (UTRETUE01) | 1886 | 2338 |
| | | | | 7346573H1 (SYNODIN02) | 2630 | 3219 |
| | | | | 7577135H1 (ADIPUNS02) | 3426 | 4023 |
| | | | | g1382343 | 3648 | 4255 |
| 60 | 6755002CB1 | 3438 | 1-37, 1548-1618, | 5879324F6 (BRAUNOT01) | 617 | 1285 |
| | | | 2405-2610 | 7630423H1 (BRAFTUE03) | 177 | 711 |
| | | | | 6755102H1 (SINTFER02) | 84 | 702 |
| | | | | 6337127H1 (BRANDIN01) | 1351 | 1904 |
| | | | | 5968891H1 (BRAZNOT01) | 2185 | 2887 |
| | | | | 8126594H1 (SCOMDIC01) | 863 | 1538 |
| | | | | 1290035H1 (BRAINOT11) | 3190 | 3438 |
| | | | | 6555447H1 (BRAFNON02) | 2732 | 3317 |
| | | | | 1305462H1 (PLACNOT02) | 1 | 182 |
| | | | | 71389138V1 | 1524 | 2121 |
| | | | | 71185765V1 | 2086 | 2672 |
| 61 | 7350907CB1 | 1683 | | 7350907H1 (COLNNON05) | 1299 | 1683 |
| | | | | 6631669J1 (BMARTXR02) | 567 | 1302 |
| | | | | 7750841J1 (HEAONOE01) | 1 | 603 |
| | | | | 6443310H1 (BRAENOT02) | 718 | 1366 |
| 62 | 7474411CB1 | 6886 | 6622-6886, | 7222381H1 (PLACFEC01) | 2499 | 3017 |
| | | | 5389-5509, 1-1582, | 7737011J1 (BRAITUE01) | 6219 | 6886 |
| | | | 6439-6474, | 70870035V1 | 3823 | 4452 |
| | | | 1894-1951, | 7633045H1 (SINTDIE01) | 2306 | 2579 |
| | | | 2850-4036 | 8037283J1 (SMCRUNE01) | 1014 | 1741 |
| | | | | 2483734F6 (SMCANOT01) | 4465 | 5002 |
| | | | | 7755056H1 (SPLNTUE01) | 3653 | 4411 |
| | | | | 1603055T6 (BLADNOT03) | 5780 | 6443 |
| | | | | 70743936V1 | 5077 | 5667 |
| | | | | 6822892H1 (SINTNOR01) | 1766 | 2503 |
| | | | | 7931495H1 (COLNDIS02) | 4915 | 5652 |
| | | | | 8053929J1 (FTUBTUE01) | 132 | 720 |
| | | | | 7689270H1 (PROSTME06) | 1 | 458 |
| | | | | 6822892J1 (SINTNOR01) | 1481 | 2273 |
| | | | | 7644647J1 (UTRSTUE01) | 632 | 1115 |
| | | | | 7738773H1 (BRAITUE01) | 3004 | 3655 |
| | | | | 7738657H1 (BRAITUE01) | 4306 | 4946 |
| | | | | 7688349J1 (PROSTME06) | 2589 | 3035 |
| | | | | 71230141V1 | 3125 | 3768 |
| | | | | 2695328T6 (UTRSNOT12) | 5562 | 6252 |
| 63 | 4755911CB1 | 4457 | 1-324, 750-1798, | 6773194J1 (BRAUNOR01) | 336 | 1095 |
| | | | 3657-3747, | 4755911H1 (BRAHNOT01) | 1769 | 2034 |
| | | | 2407-3438, | g7242966_CD | 913 | 4457 |
| | | | 2273-2326, | 6880647J1 (BRAHTDR03) | 3285 | 4044 |
| | | | 3912-4457 | 6773172H1 (BRAUNOR01) | 3132 | 3908 |
| | | | | 6765836H1 (BRAUNOR01) | 1902 | 2429 |
| | | | | 7076957H1 (BRAUTDR04) | 689 | 1321 |
| | | | | g953650 | 311 | 699 |
| | | | | 7371978H2 (BRAIFEE04) | 1207 | 1591 |
| | | | | GNN.g7454228_000009_004.edit | 1 | 4172 |
| 64 | 379766CB1 | 1943 | 1-260, 922-1565 | 71913768V1 | 1049 | 1755 |
| | | | | 71225395V1 | 529 | 1072 |
| | | | | 71912043V1 | 1122 | 1769 |
| | | | | 71910219V1 | 1291 | 1943 |
| | | | | 1672013H1 (BLADNOT05) | 1 | 221 |
| | | | | 7082633R8 (STOMTMR02) | 192 | 957 |
| 65 | 553744CB1 | 4111 | 1-580, 2777-2939 | 3918424H1 (BRAINOT14) | 3588 | 3876 |
| | | | | 6610359H2 (MUSTTMC01) | 2323 | 2934 |
| | | | | 71249585V1 | 1666 | 2217 |
| | | | | 3294472T6 (TLYJINT01) | 3163 | 3801 |
| | | | | 6338521F7 (BRANDIN01) | 1 | 427 |
| | | | | 8016583J1 (BMARTXE01) | 531 | 895 |
| | | | | 7467954H1 (LUNGNOE02) | 1059 | 1601 |
| | | | | 553744R6 (SCORNOT01) | 3799 | 4110 |
| | | | | 1799062T6 (COLNNOT27) | 2193 | 2814 |
| | | | | 7167059H1 (PLACNOR01) | 147 | 755 |
| | | | | 1304490H1 (PLACNOT02) | 3030 | 3251 |
| | | | | 71066496V1 | 1496 | 2151 |
| | | | | 2219161H1 (LUNGNOT18) | 3947 | 4111 |
| | | | | 71065885V1 | 2537 | 3206 |
| | | | | 7608285J1 (COLRTUE01) | 807 | 1308 |
| 66 | 1825473CB1 | 1604 | 1186-1222, 1-662, | 71671748V1 | 957 | 1604 |
| | | | 697-1075 | 7977810H1 (LSUBDMC01) | 1 | 614 |
| | | | | 7978864H1 (LSUBDMC01) | 642 | 1276 |
| | | | | 7978667H1 (LSUBDMC01) | 482 | 1181 |
| 67 | 7950094CB1 | 2646 | 1887-1962, | 7751102H1 (HEAONOE01) | 477 | 1191 |
| | | | 1358-1427, 1-716, | 71824138V1 | 1889 | 2577 |
| | | | 2117-2176 | 1674661F6 (BLADNOT05) | 1 | 503 |
| | | | | 71495047V1 | 608 | 1350 |
| | | | | 7721119H2 (THYRDIE01) | 1991 | 2646 |
| | | | | 7675220H1 (NOSETUE01) | 1248 | 1912 |
| | | | | 6252635H1 (LUNPTUT02) | 529 | 1205 |
| 68 | 7479484CB1 | 3876 | 1855-1892, | GBI.g8569175_000028.edit | 580 | 3876 |
| | | | 1380-1714, | GNN.g8569175_000028_002.edit | 1 | 3579 |
| | | | 2489-2791 | | | |
| 69 | 6780147CB1 | 2583 | 720-1210, | 6081718H1 (LUNLTUT11) | 1909 | 2583 |
| | | | 2237-2583 | 2417167F6 (HNT3AZT01) | 674 | 1192 |
| | | | | 7220328H1 (SPLNDIC01) | 586 | 1176 |
| | | | | 4603304F8 (BRSTNOT07) | 1242 | 1889 |
| | | | | 6780147H1 (OVARDIR01) | 1111 | 1709 |
| | | | | 3282558T6 (HEAONOT05) | 1836 | 2558 |
| | | | | 8116139H1 (TONSDIC01) | 1 | 630 |
| 70 | 7204554CB1 | 6147 | 4831-5200, | 6321562F6 (LUNGDIN02) | 3284 | 3732 |
| | | | 2698-3091, 1-557, | 7655014H1 (UTREDME06) | 2035 | 2634 |
| | | | 3609-4067, | 8036632H1 (SMCRUNE01) | 1 | 593 |
| | | | 1239-2254 | 7655014J1 (UTREDME06) | 1286 | 1880 |
| | | | | 6804453H1 (COLENOR03) | 762 | 1352 |
| | | | | 7631942H1 (BLADTUE01) | 5282 | 5852 |
| | | | | 7292462H1 (BRAIFER06) | 2334 | 2853 |
| | | | | 4099025F8 (BRAITUT26) | 5241 | 5762 |
| | | | | 6832366H1 (BRSTNON02) | 3821 | 4567 |
| | | | | GBI.g10518389_000002_CDS_5.edit | 162 | 5852 |
| | | | | 6814168J1 (ADRETUR01) | 4696 | 5261 |
| | | | | 7721587H2 (THYRDIE01) | 5641 | 6147 |
| | | | | 6883267J1 (BRAHTDR03) | 3504 | 4216 |
| | | | | 7698650J1 (KIDPTDE01) | 572 | 1196 |
| | | | | 8239939J1 (LIVRTMR01) | 2589 | 3255 |
| | | | | 8076958J1 (ADRETUE02) | 4407 | 5152 |
| | | | | 7383235R8 (FTUBTUE01) | 1551 | 2326 |
| 71 | 6833247CB1 | 888 | 331-430 | 498875H1 (NEUTLPT01) | 692 | 888 |
| | | | | 6128926H1 (BRAHNON05) | 1 | 432 |
| | | | | 6833247T8 (BRSTNON02) | 265 | 879 |
| 72 | 4148119CB1 | 3582 | 1681-1740, 1-1470, | g6465050 | 2274 | 2694 |
| | | | 2944-3582 | 7412335H1 (BONMTUE02) | 2052 | 2514 |
| | | | | 70776200V1 | 1409 | 2028 |
| | | | | 1539263R6 (SINTTUT01) | 3199 | 3582 |
| | | | | 2212958H1 (SINTFET03) | 2452 | 2684 |
| | | | | 4148119F6 (SINITUT04) | 2704 | 3290 |
| | | | | 5984846F6 (MCLDTXT02) | 1 | 732 |
| | | | | 70776601V1 | 1053 | 1616 |
| | | | | 7765150J1 (URETTUE01) | 614 | 1285 |
| | | | | 70776341V1 | 1632 | 2115 |

TABLE 5

| Polynucleotide | Incyte | Representative |
|---|---|---|
| SEQ ID NO: | Project ID | Library |
| 37 | 1888682CB1 | BRAITUT08 |
| 38 | 1794980CB1 | BRAINOT09 |
| 39 | 5533958CB1 | CONNTUT04 |
| 40 | 60210196CB1 | BRACNOK02 |
| 41 | 815125CB1 | BRAENOK01 |
| 42 | 1386915CB1 | LATRTUT02 |
| 43 | 1344495CB1 | SINTFET03 |
| 44 | 1485774CB1 | BRAITUT01 |
| 45 | 7289372CB1 | BRAIFER06 |
| 46 | 1672338CB1 | CONNNOT01 |
| 47 | 184661CB1 | CARDNOT01 |
| 48 | 3719737CB1 | KIDETXS02 |
| 49 | 5773251CB1 | PLACFER06 |
| 50 | 5426470CB1 | PROSTUS23 |
| 51 | 7087904CB1 | NERDTDN03 |
| 52 | 7477312CB1 | BRABDIE02 |
| 53 | 2739431CB1 | OVARNOT09 |
| 54 | 7473606CB1 | GBLADIT03 |
| 55 | 3534918CB1 | BONRTUT01 |
| 56 | 2428715CB1 | PLACFER06 |
| 57 | 3351332CB1 | ENDCNOT03 |
| 58 | 6382722CB1 | FTUBTUR01 |
| 59 | 55022490CB1 | BRAIFEE05 |
| 60 | 6755002CB1 | BRAITUT12 |
| 61 | 7350907CB1 | BRAENOT02 |
| 62 | 7474411CB1 | BRAITUT26 |
| 63 | 4755911CB1 | BRAUNOR01 |
| 64 | 379766CB1 | NEUTFMT01 |
| 65 | 553744CB1 | SEMVNOT01 |
| 66 | 1825473CB1 | LSUBDMC01 |
| 67 | 7950094CB1 | BLADNOT05 |
| 68 | 7479484CB1 | LUNPTUT02 |
| 69 | 6780147CB1 | HNT3AZT01 |
| 70 | 7204554CB1 | COLENOR03 |
| 71 | 6833247CB1 | BRSTNON02 |
| 72 | 4148119CB1 | CARGDIT01 |

TABLE 6

| Library | Vector | Library Description |
|---|---|---|
| BLADNOT05 | pINCY | Library was constructed using RNA isolated from bladder tissue removed from a 60-year- |
| | | old Caucasian male during a radical cystectomy, prostatectomy, and vasectomy. Pathology |
| | | for the associated tumor tissue indicated grade 3 transitional cell carcinoma. Carcinoma |
| | | in-situ was identified in the dome and trigone. Patient history included tobacco use. |
| BONRTUT01 | pINCY | Library was constructed using RNA isolated from rib tumor tissue removed from a 16-year- |
| | | old Caucasian male during a rib osteotomy and a wedge resection of the lung. Pathology |
| | | indicated a metastatic grade 3 (of 4) osteosarcoma, forming a mass involving the chest |
| | | wall. |
| BRABDIE02 | pINCY | This 5′ biased random primed library was constructed using RNA isolated from diseased |
| | | cerebellum tissue removed from the brain of a 57-year-old Caucasian male who died from a |
| | | cerebrovascular accident. Serologies were negative. Patient history included |
| | | Huntington's disease, emphysema, and tobacco abuse (3-4 packs per day, for 40 years). |
| BRACNOK02 | PSPORT1 | This amplified and normalized library was constructed using RNA isolated from posterior |
| | | cingulate tissue removed from an 85-year-old Caucasian female who died from myocardial |
| | | infarction and retroperitoneal hemorrhage. Pathology indicated atherosclerosis, moderate |
| | | to severe, involving the circle of Willis, middle cerebral, basilar and vertebral |
| | | arteries; infarction, remote, left dentate nucleus; and amyloid plaque deposition |
| | | consistent with age. There was mild to moderate leptomeningeal fibrosis, especially over |
| | | the convexity of the frontal lobe. There was mild generalized atrophy involving all |
| | | lobes. The white matter was mildly thinned. Cortical thickness in the temporal lobes, |
| | | both maximal and minimal, was slightly reduced. The substantia nigra pars compacta |
| | | appeared mildly depigmented. Patient history included COPD, hypertension, and recurrent |
| | | deep venous thrombosis. 6.4 million independent clones from this amplified library were |
| | | normalized in one round using conditions adapted Soares et al., PNAS (1994) 91: 9228-9232 |
| | | and Bonaldo et al., Genome Research 6 (1996): 791. |
| BRAENOK01 | PSPORT1 | This amplified and normalized library was constructed using RNA isolated from inferior |
| | | parietal cortex tissue removed from a 35-year-old Caucasian male who died from cardiac |
| | | failure. Pathology indicated moderate leptomeningeal fibrosis and multiple |
| | | microinfarctions of the cerebral neocortex. There was evidence of shrunken and slightly |
| | | eosinophilic pyramidal neurons throughout the cerebral hemispheres. There were multiple |
| | | small microscopic areas of cavitation with surrounding gliosis scattered throughout the |
| | | cerebral cortex. Patient history included dilated cardiomyopathy, congestive heart |
| | | failure, and cardiomegaly. Patient medications included simethicone, Lasix, Digoxin, |
| | | Colace, Zantac, captopril, and Vasotec. 1.08 million independent clones from this |
| | | amplified library were normalized in one round using conditions adapted from Soares et |
| | | al., PNAS (1994) 91: 9228-9232 and Bonaldo et al., Genome Research 6 (1996): 791, except |
| | | that a significantly longer (48 hours/round) reannealing hybridization was used. |
| BRAENOT02 | pINCY | Library was constructed using RNA isolated from posterior parietal cortex tissue removed |
| | | from the brain of a 35-year-old Caucasian male who died from cardiac failure. |
| BRAIFEE05 | PCDNA2.1 | This 5′ biased random primed library was constructed using RNA isolated from brain |
| | | tissue removed from a Caucasian male fetus who was stillborn with a hypoplastic left |
| | | heart at 23 weeks' gestation. |
| BRAIFER06 | PCDNA2.1 | This random primed library was constructed using RNA isolated from brain tissue removed |
| | | from a Caucasian male fetus who was stillborn with a hypoplastic left heart at 23 weeks' |
| | | gestation. Serologies were negative. |
| BRAINOT09 | pINCY | Library was constructed using RNA isolated from brain tissue removed from a Caucasian |
| | | male fetus, who died at 23 weeks' gestation. |
| BRAITUT01 | PSPORT1 | Library was constructed using RNA isolated from brain tumor tissue removed from a 50- |
| | | year-old Caucasian female during a frontal lobectomy. Pathology indicated recurrent |
| | | grade 3 oligoastrocytoma with focal necrosis and extensive calcification. Patient |
| | | history included a speech disturbance and epilepsy. The patient's brain had also been |
| | | irradiated with a total dose of 5,082 cyg (Fraction 8). Family history included a brain |
| | | tumor. |
| BRAITUT08 | pINCY | Library was constructed using RNA isolated from brain tumor tissue removed from the left |
| | | frontal lobe of a 47-year-old Caucasian male during excision of cerebral meningeal |
| | | tissue. Pathology indicated grade 4 fibrillary astrocytoma with focal tumoral |
| | | radionecrosis. Patient history included cerebrovascular disease, deficiency anemia, |
| | | hyperlipidemia, epilepsy, and tobacco use. Family history included cerebrovascular |
| | | disease and a malignant prostate neoplasm. |
| BRAITUT12 | pINCY | Library was constructed using RNA isolated from brain tumor tissue removed from the left |
| | | frontal lobe of a 40-year-old Caucasian female during excision of a cerebral meningeal |
| | | lesion. Pathology indicated grade 4 gemistocytic astrocytoma. |
| BRAITUT26 | pINCY | Library was constructed using RNA isolated from brain tumor tissue removed from the |
| | | right posterior fossa, occipital convexity of a 70-year-old Caucasian male during |
| | | cerebral meninges lesion excision. Pathology indicated meningioma. Patient history |
| | | included a benign colon neoplasm and unspecified personality disorder. Family history |
| | | included chronic proliferative nephritis, acute myocardial infarction, atherosclerotic |
| | | coronary artery disease, and chronic proliferative nephritis. |
| BRAUNOR01 | pINCY | This random primed library was constructed using RNA isolated from striatum, globus |
| | | pallidus and posterior putamen tissue removed from an 81-year-old Caucasian female who |
| | | died from a hemorrhage and ruptured thoracic aorta due to atherosclerosis. Pathology |
| | | indicated moderate atherosclerosis involving the internal carotids, bilaterally; |
| | | microscopic infarcts of the frontal cortex and hippocampus; and scattered diffuse |
| | | amyloid plaques and neurofibrillary tangles, consistent with age. Grossly, the |
| | | leptomeninges showed only mild thickening and hyalinization along the superior sagittal |
| | | sinus. The remainder of the leptomeninges was thin and contained some congested blood |
| | | vessels. Mild atrophy was found mostly in the frontal poles and lobes, and temporal |
| | | lobes, bilaterally. Microscopically, there were pairs of Alzheimer type II astrocytes |
| | | within the deep layers of the neocortex. There was increased satellitosis around neurons |
| | | in the deep gray matter in the middle frontal cortex. The amygdala contained rare |
| | | diffuse plaques and neurofibrillary tangles. The posterior hippocampus contained a |
| | | microscopic area of cystic cavitation with hemosiderin-laden macrophages surrounded by |
| | | reactive gliosis. Patient history included sepsis, cholangitis, post-operative |
| | | atelectasis, pneumonia CAD, cardiomegaly due to left ventricular hypertrophy, |
| | | splenomegaly, arteriolonephrosclerosis, nodular colloidal goiter, emphysema, CHF, |
| | | hypothyroidism, and peripheral vascular disease. |
| BRSTNON02 | pINCY | This normalized breast tissue library was constructed from 6.2 million independent |
| | | clones from a pool of two libraries from two different donors. Starting RNA was made |
| | | from breast tissue removed from a 46-year-old Caucasian female during a bilateral |
| | | reduction mammoplasty (donor A), and from breast tissue removed from a 60-year-old |
| | | Caucasian female during a bilateral reduction mammoplasty (donor B). Pathology indicated |
| | | normal breast parenchyma, bilaterally (A) and bilateral mammary hypertrophy (B). Patient |
| | | history included hypertrophy of breast, obesity, lumbago, and glaucoma (A) and joint |
| | | pain in the shoulder, thyroid cyst, colon cancer, normal delivery and cervical cancer |
| | | (B). Family history included cataract, osteoarthritis, uterine cancer, benign |
| | | hypertension, hyperlipidemia, and alcoholic cirrhosis of the liver, cerebrovascular |
| | | disease, and type II diabetes (A) and cerebrovascular accident, atherosclerotic coronary |
| | | artery disease, colon cancer, type II diabetes, hyperlipidemia, depressive disorder, and |
| | | Alzheimer's Disease. The library was normalized in two rounds using conditions adapted |
| | | from Soares et al., PNAS (1994) 91: 9228-9232 and Bonaldo et al., Genome Research 6 |
| | | (1996): 791, except that a significantly longer (48 hours/round) reannealing |
| | | hybridization was used. |
| CARDNOT01 | PBLUESCRIPT | Library was constructed using RNA isolated from the cardiac muscle of a 65-year-old |
| | | Caucasian male, who died from a gunshot wound. |
| | | CARGDIT01 pINCY Library was constructed using RNA isolated from diseased cartilage |
| | | tissue. Patient history included osteoarthritis. |
| COLENOR03 | PCDNA2.1 | Library was constructed using RNA isolated from colon epithelium tissue removed from a |
| | | 13-year-old Caucasian female who died from a motor vehicle accident. |
| CONNNOT01 | pINCY | Library was constructed using RNA isolated from mesentery fat tissue obtained from a 71- |
| | | year-old Caucasian male during a partial colectomy and permanent colostomy. Family |
| | | history included atherosclerotic coronary artery disease, myocardial infarction, and |
| | | extrinsic asthma. |
| CONNTUT04 | pINCY | Library was constructed using RNA isolated from tumorous spinal tissue removed from a |
| | | 35-year-old Caucasian male during an exploratory laparotomy. Pathology indicated |
| | | schwannoma with degenerative changes. Patient history included anxiety, depression, |
| | | neurofibromatosis and benign neoplasm of the scrotum. Previously the patient had a |
| | | spinal fusion. Family history included brain cancer, liver disease, and multiple |
| | | sclerosis. |
| ENDCNOT03 | pINCY | Library was constructed using RNA isolated from dermal microvascular endothelial cells |
| | | removed from a neonatal Caucasian male. |
| FTUBTUR01 | PCDNA2.1 | This random primed library was constructed using RNA isolated from fallopian tube tumor |
| | | tissue removed from an 85-year-old Caucasian female during bilateral salpingo- |
| | | oophorectomy and hysterectomy. Pathology indicated poorly differentiated mixed |
| | | endometrioid (80%) and serous (20%) adenocarcinoma, which was confined to the mucosa |
| | | without mural involvement. Endometrioid carcinoma in situ was also present. Pathology |
| | | for the associated uterus tumor indicated focal endometrioid adenocarcinoma in situ and |
| | | moderately differentiated invasive adenocarcinoma arising in an endometrial polyp. |
| | | Metastatic endometrioid and serous adenocarcinoma was present at the cul-de-sac tumor. |
| | | Patient history included medullary carcinoma of the thyroid and myocardial infarction. |
| GBLADIT03 | pINCY | Library was constructed using RNA isolated from diseased gallbladder tissue removed from |
| | | a 53-year-old Caucasian female during cholecystectomy. Pathology indicated mild chronic |
| | | cholecystitis and cholelithiasis with approximately 150 mixed stones ranging in size |
| | | from 0.1 cm to 0.5 cm. The patient presented with abdominal pain and nausea and |
| | | vomiting. Patient history included hyperlipidema and tobacco and alcohol abuse. Previous |
| | | surgeries included adenotonsillectomy. Patient medications included Zantac, Provera, |
| | | Premarin, and calcium. Family history included benign hypertension in the mother and the |
| | | father. |
| HNT3AZT01 | pINCY | Library was constructed using RNA isolated from the hNT2 cell line (derived from a human |
| | | teratocarcinoma that exhibited properties characteristic of a committed neuronal |
| | | precursor). Cells were treated for three days with 0.35 micromolar 5-aza-2′- |
| | | deoxycytidine (AZ). |
| KIDETXS02 | pINCY | This subtracted, transformed embryonal cell line library was constructed using 9 million |
| | | clones from a treated, transformed embryonal cell line (293-EBNA) derived from kidney |
| | | epithelial tissue and was subjected to two rounds of subtraction hybridization with 1.9 |
| | | million clones from an untreated transformed embryonal cell line (293-EBNA) derived from |
| | | a kidney epithelial tissue library. The starting library for subtraction was constructed |
| | | using RNA isolated from the treated, transformed embryonal cell line (293-EBNA). The |
| | | cells were treated with 5-aza-2′-deoxycytidine and transformed with adenovirus 5 DNA. |
| | | The hybridization probe for subtraction was derived from a similarly constructed library |
| | | from RNA isolated from untreated 293-EBNA cells from the same cell line. Subtractive |
| | | hybridization conditions were based on the methodologies of Swaroop et al., NAR 19 |
| | | (1991): 1954 and Bonaldo, et al. Genome Research (1996) 6: 791. |
| LATRTUT02 | pINCY | Library was constructed using RNA isolated from a myxoma removed from the left atrium of |
| | | a 43-year-old Caucasian male during annuloplasty. Pathology indicated atrial myxoma. |
| | | Patient history included pulmonary insufficiency, acute myocardial infarction, |
| | | atherosclerotic coronary artery disease, hyperlipidemia, and tobacco use. Family history |
| | | included benign hypertension, acute myocardial infarction, atherosclerotic coronary |
| | | artery disease, and type II diabetes. |
| LSUBDMC01 | PSPORT1 | This large size fractionated library was constructed using RNA isolated from |
| | | submandibular gland tissue removed from a 49-year-old Caucasian female during |
| | | sialoadenectomy. Pathology indicated unremarkable gland. The patient presented with |
| | | sialoadenitis. Patient history included vericose veins and normal delivery. Previous |
| | | surgeries included cholecystectomy and total abdominal hysterectomy. Patient medications |
| | | included vitamins, phentermine HCL, and Pondimin. Family history included |
| | | atherosclerotic coronary artery disease and acute myocardial infarction in the mother; |
| | | benign hypertension, cerebrovascular accident, atherosclerotic coronary artery disease, |
| | | and hyperlipidemia in the sibling(s); and alcohol abuse and depressive disorder in the |
| | | grandparent(s). |
| LUNPTUT02 | pINCY | Library was constructed using RNA isolated from pleura tumor tissue removed from a 55- |
| | | year-old Caucasian female during complete pneumonectomy. Pathology indicated grade 3 |
| | | sarcoma most consistent with leiomyosarcoma, uterine primary, forming a bosellated mass |
| | | replacing the right lower lobe and a portion of the middle lobe. The tumor involved the |
| | | adjacent parietal pleura and pericardium. Multiple nodules comprising the tumor show |
| | | near total necrosis. The right upper lobe was atelectic but uninvolved by tumor. |
| | | Microsections of cellular nodules show brisk mitotic activity. The pericardium shows |
| | | direct involvement but its margins were tumor free. Smooth muscle actin was positive. |
| | | Estrogen receptor was negative and progesterone receptor was positive. Patient history |
| | | included shortness of breath, peptic ulcer disease, lung cancer, uterine cancer, normal |
| | | delivery, tobacco abuse, and deficiency anemia. Previous surgeries included endoscopic |
| | | excision of a lung lesion. Family history included atherosclerotic coronary artery |
| | | disease, breast cancer, type II diabetes, and multiple sclerosis. |
| NERDTDN03 | pINCY | This normalized dorsal root ganglion tissue library was constructed from 1.05 million |
| | | independent clones from a dorsal root ganglion tissue library. Starting RNA was made |
| | | from dorsal root ganglion tissue removed from the cervical spine of a 32-year-old |
| | | Caucasian male who died from acute pulmonary edema, acute bronchopneumonia, bilateral |
| | | pleural effusions, pericardial effusion, and malignant lymphoma (natural killer cell |
| | | type). The patient presented with pyrexia of unknown origin, malaise, fatigue, and |
| | | gastrointestinal bleeding. Patient history included probable cytomegalovirus infection, |
| | | liver congestion, and steatosis, splenomegaly, hemorrhagic cystitis, thyroid hemorrhage, |
| | | respiratory failure, pneumonia of the left lung, natural killer cell lymphoma of the |
| | | pharynx, Bell's palsy, and tobacco and alcohol abuse. Previous surgeries included |
| | | colonoscopy, closed colon biopsy, adenotonsillectomy, and nasopharyngeal endoscopy and |
| | | biopsy. Patient medications included Diflucan (fluconazole), Deltasone (prednisone), |
| | | hydrocodone, Lortab, Alprazolam, Reazodone, ProMace-Cytabom, Etoposide, Cisplatin, |
| | | Cytarabine, and dexamethasone. The patient received radiation therapy and multiple blood |
| | | transfusions. The library was normalized in 2 rounds using conditions adapted from |
| | | Soares et al., PNAS (1994) 91: 9228-9232 and Bonaldo et al., Genome Research 6 |
| | | (1996): 791, except that a significantly longer (48 hours/round) reannealing |
| | | hybridization was used. |
| NEUTFMT01 | PBLUESCRIPT | Library was constructed using total RNA isolated from peripheral blood granulocytes |
| | | collected by density gradient centrifugation through Ficoll-Hypaque. The cells were |
| | | isolated from buffy coat units obtained from unrelated male and female donors. Cells |
| | | were cultured in 10 nM fMLP for 30 minutes, lysed in GuSCN, and spun through CsCl to |
| | | obtain RNA for library construction. Because this library was made from total RNA, it |
| | | has an unusually high proportion of unique singleton sequences, which may not all come |
| | | from polyA RNA species. |
| OVARNOT09 | pINCY | Library was constructed using RNA isolated from ovarian tissue removed from a 28-year- |
| | | old Caucasian female during a vaginal hysterectomy and removal of the fallopian tubes |
| | | and ovaries. Pathology indicated multiple follicular cysts ranging in size from 0.4 to |
| | | 1.5 cm in the right and left ovaries, chronic cervicitis and squamous metaplasia of the |
| | | cervix, and endometrium in weakly proliferative phase. Family history included benign |
| | | hypertension, hyperlipidemia, and atherosclerotic coronary artery disease. |
| PLACFER06 | pINCY | This random primed library was constructed using RNA isolated from placental tissue |
| | | removed from a Caucasian fetus who died after 16 weeks' gestation from fetal demise and |
| | | hydrocephalus. Patient history included umbilical cord wrapped around the head (3 times) |
| | | and the shoulders (1 time). Serology was positive for anti-CMV. Family history included |
| | | multiple pregnancies and live births, and an abortion. |
| PROSTUS23 | pINCY | This subtracted prostate tumor library was constructed using 10 million clones from a |
| | | pooled prostate tumor library that was subjected to 2 rounds of subtractive |
| | | hybridization with 10 million clones from a pooled prostate tissue library. The starting |
| | | library for subtraction was constructed by pooling equal numbers of clones from 4 |
| | | prostate tumor libraries using mRNA isolated from prostate tumor removed from Caucasian |
| | | males at ages 58 (A), 61 (B), 66 (C) , and 68 (D) during prostatectomy with lymph node |
| | | excision. Pathology indicated adenocarcinoma in all donors. History included elevated |
| | | PSA, induration and tobacco abuse in donor A; elevated PSA, induration, prostate |
| | | hyperplasia, renal failure, osteoarthritis, renal artery stenosis, benign HTN, |
| | | thrombocytopenia, hyperlipidemia, tobacco/alcohol abuse and hepatitis C (carrier) in |
| | | donor B; elevated PSA, induration, and tobacco abuse in donor C; and elevated PSA, |
| | | induration, hypercholesterolemia, and kidney calculus in donor D. The hybridization |
| | | probe for subtraction was constructed by pooling equal numbers of cDNA clones from 3 |
| | | prostate tissue libraries derived from prostate tissue, prostate epithelial cells, and |
| | | fibroblasts from prostate stroma from 3 different donors. Subtractive hybridization |
| | | conditions were based on the methodologies of Swaroop et al., NAR 19 (1991): 1954 and |
| | | Bonaldo, et al. Genome Research 6 (1996): 791. |
| SEMVNOT01 | pINCY | Library was constructed using RNA isolated from seminal vesicle tissue removed from a |
| | | 58-year-old Caucasian male during radical prostatectomy. Pathology for the associated |
| | | tumor tissue indicated adenocarcinoma (Gleason grade 3 + 2) of the prostate. |
| | | Adenofibromatous hyperplasia was also present. The patient presented with elevated |
| | | prostate specific antigen (PSA). Family history included a malignant breast neoplasm. |
| SINTFET03 | pINCY | Library was constructed using RNA isolated from small intestine tissue removed from a |
| | | Caucasian female fetus, who died at 20 weeks' gestation. |
| CARGDIT01 | pINCY | Library was constructed using RNA isolated from diseased cartilage tissue. Patient |
| | | history included osteoarthritis. |

TABLE 7

| Program | Description | Reference | Parameter Threshold |
|---|---|---|---|
| ABI | A program that removes vector sequences and | Applied Biosystems, Foster City, CA. | |
| FACTURA | masks ambiguous bases in nucleic acid sequences. | | |
| ABI/ | A Fast Data Finder useful in comparing and | Applied Biosystems, Foster City, CA; | Mismatch < 50% |
| PARACEL | annotating amino acid or nucleic acid sequences. | Paracel Inc., Pasadena, CA. | |
| FDF | | | |
| ABI Auto- | A program that assembles nucleic acid sequences. | Applied Biosystems, Foster City, CA. | |
| Assembler | | | |
| BLAST | A Basic Local Alignment Search Tool useful in | Altschul, S. F. et al. (1990) J. Mol. Biol. | ESTs: Probability value = |
| | sequence similarity search for amino acid and | 215: 403-410; Altschul, S. F. et al. (1997) | 1.0E−8 or less |
| | nucleic acid sequences. BLAST includes five | Nucleic Acids Res. 25: 3389-3402. | Full Length sequences: |
| | functions: blastp, blastn, blastx, tblastn, and tblastx. | | Probability value = 1.0E−10 |
| | | | or less |
| FASTA | A Pearson and Lipman algorithm that searches for | Pearson, W. R. and D. J. Lipman (1988) Proc. | ESTs: fasta E value = |
| | similarity between a query sequence and a group of | Natl. Acad Sci. USA 85: 2444-2448; Pearson, | 1.06E−6 Assembled ESTs: |
| | sequences of the same type. FASTA comprises as | W. R. (1990) Methods Enzymol. 183: 63-98; | fasta Identity = 95% or |
| | least five functions: fasta, tfasta, fastx, tfastx, and | and Smith, T. F. and M. S. Waterman (1981) | greater and Match length = |
| | ssearch. | Adv. Appl. Math. 2: 482-489. | 200 bases or greater; |
| | | | fastx E value = 1.0E−8 or less |
| | | | Full Length sequences: |
| | | | fastx score = 100 or greater |
| BLIMPS | A BLocks IMProved Searcher that matches a | Henikoff, S. and J. G. Henikoff (1991) Nucleic | Probability value = 1.0E−3 |
| | sequence against those in BLOCKS, PRINTS, | Acids Res. 19: 6565-6572; Henikoff, J. G. and | or less |
| | DOMO, PRODOM, and PFAM databases to search | S. Henikoff (1996) Methods Enzymol. | |
| | for gene families, sequence homology, and | 266: 88-105; and Attwood, T. K. et al. (1997) | |
| | structural fingerprint regions. | J. Chem. Inf. Comput. Sci. 37: 417-424. | |
| HMMER | An algorithm for searching a query sequence against | Krogh, A. et al. (1994) J. Mol. Biol. | PFAM hits: Probability |
| | hidden Markov model (HMM)-based databases of | 235: 1501-1531; Sonnhammer, E. L. L. et al. | value = 1.0E−3 or less |
| | protein family consensus sequences, such as PFAM. | (1988) Nucleic Acids Res. 26: 320-322; | Signal peptide hits: Score = 0 |
| | | Durbin, R. et al. (1998) Our World View, in a | or greater |
| | | Nutshell, Cambridge Univ. Press, pp. 1-350. | |
| ProfileScan | An algorithm that searches for structural and sequence | Gribskov, M. et al. (1988) CABIOS 4: 61-66; | Normalized quality score ≧ |
| | motifs in protein sequences that match sequence patterns | Gribskov, M. et al. (1989) Methods Enzymol. | GCG-specified “HIGH” value |
| | defined in Prosite. | 183: 146-159; Bairoch, A. et al. (1997) | for that particular Prosite |
| | | Nucleic Acids Res. 25: 217-221. | motif. Generally, score = |
| | | | 1.4-2.1. |
| Phred | A base-calling algorithm that examines automated | Ewing, B. et al. (1998) Genome Res. | |
| | sequencer traces with high sensitivity and probability. | 8: 175-185; Ewing, B. and P. Green | |
| | | (1998) Genome Res. 8: 186-194. | |
| Phrap | A Phils Revised Assembly Program including SWAT and | Smith, T. F. and M. S. Waterman (1981) Adv. | Score = 120 or greater; |
| | CrossMatch, programs based on efficient implementation | Appl. Math. 2: 482-489; Smith, T. F. and M. | Match length = 56 or greater |
| | of the Smith-Waterman algorithm, useful in searching | S. Waterman (1981) J. Mol. Biol. 147: 195- | |
| | sequence homology and assembling DNA sequences. | 197; and Green, P., University of Washington, | |
| | | Seattle, WA. | |
| Consed | A graphical tool for viewing and editing Phrap | Gordon, D. et al. (1998) Genome Res. 8: | |
| | assemblies. | 195-202. | |
| SPScan | A weight matrix analysis program that scans protein | Nielson, H. et al. (1997) Protein Engineering | Score = 3.5 or greater |
| | sequences for the presence of secretory signal peptides. | 10: 1-6; Claverie, J. M. and S. Audic (1997) | |
| | | CABIOS 12: 431-439. | |
| TMAP | A program that uses weight matrices to delineate | Persson, B. and P. Argos (1994) J. Mol. Biol. | |
| | transmembrane segments on protein sequences and | 237: 182-192; Persson, B. and P. Argos (1996) | |
| | determine orientation. | Protein Sci. 5: 363-371. | |
| TMHMMER | A program that uses a hidden Markov model (HMM) to | Sonnhammer, E. L. et al. (1998) Proc. Sixth | |
| | delineate transmembrane segments on protein sequences | Intl. Conf. on Intelligent Systems for Mol. | |
| | and determine orientation. | Biol., Glasgow et al., eds., The Am. Assoc. | |
| | | for Artificial Intelligence Press, Menlo Park, | |
| | | CA, pp. 175-182. | |
| Motifs | A program that searches amino acid sequences for | Bairoch, A. et al. (1997) Nucleic Acids Res. | |
| | patterns that matched those defined in Prosite. | 25: 217-221; Wisconsin Package Program | |
| | | Manual, version 9, page M51-59, Genetics | |
| | | Computer Group, Madison, WI. | |

What is claimed is:

1. An isolated polypeptide selected from the group consisting of:
a) a polypeptide comprising an amino acid sequence selected from the group consisting of SEQ ID NO:1-36,
b) a naturally occurring polypeptide comprising an amino acid sequence at least 90%o identical to an amino acid sequence selected from the group consisting of SEQ ID NO:1-36,
c) a biologically active fragment of a polypeptide having an amino acid sequence selected from the group consisting of SEQ ID NO:1-36, and
d) an immunogenic fragment of a polypeptide having an amino acid sequence selected from the group consisting of SEQ ID NO:1-36.

2. An isolated polypeptide of claim 1 selected from the group consisting of SEQ ID NO:1-36.

3. An isolated polynucleotide encoding a polypeptide of claim 1.

4. An isolated polynucleotide encoding a polypeptide of claim 2.

5. An isolated polynucleotide of claim 4 selected from the group consisting of SEQ ID NO:37-72.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method for producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. An isolated antibody which specifically binds to a polypeptide of claim 1.

11. An isolated polynucleotide selected from the group consisting of:
a) a polynucleotide comprising a polynucleotide sequence selected from the group consisting of SEQ ID NO:37-72,
b) a naturally occurring polynucleotide comprising a polynucleotide sequence at least 90% identical to a polynucleotide sequence selected from the group consisting of SEQ ID NO:37-72,
c) a polynucleotide complementary to a polynucleotide of a),
d) a polynucleotide complementary to a polynucleotide of b), and
e) an RNA equivalent of a)-d).

12. An isolated polynucleotide comprising at least 60 contiguous nucleotides of a polynucleotide of claim 11.

13. A method for detecting a target polynucleotide in a sample, said target polynucleotide having a sequence of a polynucleotide of claim 11, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

14. A method of claim 13, wherein the probe comprises at least 60 contiguous nucleotides.

15. A method for detecting a target polynucleotide in a sample, said target polynucleotide having a sequence of a polynucleotide of claim 11, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

16. A composition comprising a polypeptide of claim 1 and a pharmaceutically acceptable excipient.

17. A composition of claim 16, wherein the polypeptide has an amino acid sequence selected from the group consisting of SEQ ID NO:1-36.

18. A method for treating a disease or condition associated with decreased expression of functional ECMCAD, comprising administering to a patient in need of such treatment the composition of claim 16.

19. A method for screening a compound for effectiveness as an agonist of a polypeptide of claim 1, the method comprising:
a) exposing a sample comprising a polypeptide of claim 1 to a compound, and
b) detecting agonist activity in the sample.

20. A composition comprising an agonist compound identified by a method of claim 19 and a pharmaceutically acceptable excipient.

21. A method for treating a disease or condition associated with decreased expression of functional ECMCAD, comprising administering to a patient in need of such treatment a composition of claim 20.

22. A method for screening a compound for effectiveness as an antagonist of a polypeptide of claim 1, the method comprising:
a) exposing a sample comprising a polypeptide of claim 1 to a compound, and
b) detecting antagonist activity in the sample.

23. A composition comprising an antagonist compound identified by a method of claim 22 and a pharmaceutically acceptable excipient.

24. A method for treating a disease or condition associated with overexpression of functional ECMCAD, comprising administering to a patient in need of such treatment a composition of claim 23.

25. A method of screening for a compound that specifically binds to the polypeptide of claim 1, said method comprising the steps of:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

26. A method of screening for a compound that modulates the activity of the polypeptide of claim 1, said method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

27. A method for screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

28. A method for assessing toxicity of a test compound, said method comprising:
a) treating a biological sample containing nucleic acids with the test compound;
b) hybridizing the nucleic acids of the treated biological sample with a probe comprising at least 20 contiguous nucleotides of a polynucleotide of claim 11 under conditions whereby a specific hybridization complex is formed between said probe and a target polynucleotide in the biological sample, said target polynucleotide comprising a polynucleotide sequence of a polynucleotide of claim 11 or fragment thereof;
c) quantifying the amount of hybridization complex; and
d) comparing the amount of hybridization complex in the treated biological sample with the amount of hybridization complex in an untreated biological sample, wherein a difference in the amount of hybridization complex in the treated biological sample is indicative of toxicity of the test compound.

29. A diagnostic test for a condition or disease associated with the expression of ECMCAD in a biological sample comprising the steps of:
a) combining the biological sample with an antibody of claim 10, under conditions suitable for the antibody to bind the polypeptide and form an antibody:polypeptide complex; and
b) detecting the complex, wherein the presence of the complex correlates with the presence of the polypeptide in the biological sample.

30. The antibody of claim 10, wherein the antibody is:
a) a chimeric antibody,
b) a single chain antibody,
c) a Fab fragment,
d) a F(ab′)₂ fragment, or
e) a humanized antibody.

31. A composition comprising an antibody of claim 10 and an acceptable excipient.

32. A method of diagnosing a condition or disease associated with the expression of ECMCAD in a subject, comprising administering to said subject an effective amount of the composition of claim 31.

33. A composition of claim 31, wherein the antibody is labeled.

34. A method of diagnosing a condition or disease associated with the expression of ECMCAD in a subject, comprising administering to said subject an effective amount of the composition of claim 33.

35. A method of preparing a polyclonal antibody with the specificity of the antibody of claim 10 comprising:
a) immunizing an animal with a polypeptide having an amino acid sequence selected from the group consisting of SEQ ID NO:1-36, or an immunogenic fragment thereof, under conditions to elicit an antibody response:
b) isolating antibodies from said animal; and
c) screening the isolated antibodies with the polypeptide, thereby identifying a polyclonal antibody which binds specifically to a polypeptide having an amino acid sequence selected from the group consisting of SEQ ID NO:1-36.

36. An antibody produced by a method of claim 35.

37. A composition comprising the antibody of claim 36 and a suitable carrier.

38. A method of making a monoclonal antibody with the specificity of the antibody of claim 10 comprising:
a) immunizing an animal with a polypeptide having an amino acid sequence selected from the group consisting of SEQ ID NO:1-36, or an immunogenic fragment thereof, under conditions to elicit an antibody response;
b) isolating antibody producing cells from the animal;
c) fusing the antibody producing cells with immortalized cells to form monoclonal antibody-producing hybridoma cells;
d) culturing the hybridoma cells: and
e) isolating from the culture monoclonal antibody which binds specifically to a polypeptide having an amino acid sequence selected from the group consisting of SEQ ID NO:1-36.

39. A monoclonal antibody produced by a method of claim 38.

40. A composition comprising the antibody of claim 39 and a suitable carrier.

41. The antibody of claim 10, wherein the antibody is produced by screening a Fab expression library.

42. The antibody of claim 10, wherein the antibody is produced by screening a recombinant immunoglobulin library.

43. A method for detecting a polypeptide having an amino acid sequence selected from the group consisting of SEQ ID NO:1-36 in a sample, comprising the steps of:
a) incubating the antibody of claim 10 with a sample under conditions to allow specific binding of the antibody and the polypeptide; and
b) detecting specific binding, wherein specific binding indicates the presence of a polypeptide having an amino acid sequence selected from the group consisting of SEQ ID NO:1-36 in the sample.

44. A method of purifying a polypeptide having an amino acid sequence selected from the group consisting of SEQ ID NO:1-36 from a sample, the method comprising:
a) incubating the antibody of claim 10 with a sample under conditions to allow specific binding of the antibody and the polypeptide; and
b) separating the antibody from the sample and obtaining the purified polypeptide having an amino acid sequence selected from the group consisting of SEQ ID NO:1-36.

45. A polypeptide of claim 1, comprising the amino acid sequence of SEQ ID NO:1.

46. A polypeptide of claim 1, comprising the amino acid sequence of SEQ ID NO:2.

47. A polypeptide of claim 1, comprising the amino acid sequence of SEQ ID NO:3.

48. A polypeptide of claim 1, comprising the amino acid sequence of SEQ ID NO:4.

49. A polypeptide of claim 1, comprising the amino acid sequence of SEQ ID NO:5.

50. A polypeptide of claim 1, comprising the amino acid sequence of SEQ ID NO:6.

51. A polypeptide of claim 1, comprising the amino acid sequence of SEQ ID NO:7.

52. A polypeptide of claim 1, comprising the amino acid sequence of SEQ ID NO:8.

53. A polypeptide of claim 1, comprising the amino acid sequence of SEQ ID NO:9.

54. A polypeptide of claim 1, comprising the amino acid sequence of SEQ ID NO:10.

55. A polypeptide of claim 1, comprising the amino acid sequence of SEQ ID NO:11.

56. A polypeptide of claim 1, comprising the amino acid sequence of SEQ ID NO:12.

57. A polypeptide of claim 1, comprising the amino acid sequence of SEQ ID NO:13.

58. A polypeptide of claim 1, comprising the amino acid sequence of SEQ ID NO:14.

59. A polypeptide of claim 1, comprising the amino acid sequence of SEQ ID NO:15.

60. A polypeptide of claim 1, comprising the amino acid sequence of SEQ ID NO:16.

61. A polypeptide of claim 1, comprising the amino acid sequence of SEQ ID NO:17.

62. A polypeptide of claim 1, comprising the amino acid sequence of SEQ ID NO18.

63. A polypeptide of claim 1, comprising the amino acid sequence of SEQ ID NO19.

64. A polypeptide of claim 1, comprising the amino acid sequence of SEQ ID NO:20.

65. A polypeptide of claim 1, comprising the amino acid sequence of SEQ ID NO:21.

66. A polypeptide of claim 1, comprising the amino acid sequence of SEQ ID NO:22.

67. A polypeptide of claim 1, comprising the amino acid sequence of SEQ ID NO:23.

68. A polypeptide of claim 1, comprising the amino acid sequence of SEQ ID NO:24.

69. A polypeptide of claim 1, comprising the amino acid sequence of SEQ ID NO:25.

70. A polypeptide of claim 1, comprising the amino acid sequence of SEQ ID NO:26.

71. A polypeptide of claim 1, comprising the amino acid sequence of SEQ ID NO:27.

72. A polypeptide of claim 1, comprising the amino acid sequence of SEQ ID NO:28.

73. A polypeptide of claim 1, comprising the amino acid sequence of SEQ ID NO:29.

74. A polypeptide of claim 1, comprising the amino acid sequence of SEQ ID NO:30.

75. A polypeptide of claim 1, comprising the amino acid sequence of SEQ ID NO:31.

76. A polypeptide of claim 1, comprising the amino acid sequence of SEQ ID NO:32.

77. A polypeptide of claim 1, comprising the amino acid sequence of SEQ ID NO:33.

78. A polypeptide of claim 1, comprising the amino acid sequence of SEQ ID NO:34.

79. A polypeptide of claim 1, comprising the amino acid sequence of SEQ ID NO:35.

80. A polypeptide of claim 1, comprising the amino acid sequence of SEQ ID NO:36.

81. A polynucleotide of claim 11, comprising the polynucleotide sequence of SEQ ID NO:37.

82. A polynucleotide of claim 11, comprising the polynucleotide sequence of SEQ ID NO:38.

83. A polynucleotide of claim 11, comprising the polynucleotide sequence of SEQ ID NO:39.

84. A polynucleotide of claim 11, comprising the polynucleotide sequence of SEQ ID NO:40.

85. A polynucleotide of claim 11, comprising the polynucleotide sequence of SEQ ID NO:41.

86. A polynucleotide of claim 11, comprising the polynucleotide sequence of SEQ ID NO:42.

87. A polynucleotide of claim 11, comprising the polynucleotide sequence of SEQ ID NO:43.

88. A polynucleotide of claim 11, comprising the polynucleotide sequence of SEQ ID NO:44.

89. A polynucleotide of claim 11, comprising the polynucleotide sequence of SEQ ID NO:45.

90. A polynucleotide of claim 11, comprising the polynucleotide sequence of SEQ ID NO:46.

91. A polynucleotide of claim 11, comprising the polynucleotide sequence of SEQ ID NO:47.

92. A polynucleotide of claim 11, comprising the polynucleotide sequence of SEQ ID NO:48.

93. A polynucleotide of claim 11, comprising the polynucleotide sequence of SEQ ID NO:49.

94. A polynucleotide of claim 11, comprising the polynucleotide sequence of SEQ ID NO:50.

95. A polynucleotide of claim 11, comprising the polynucleotide sequence of SEQ ID NO:51.

96. A polynucleotide of claim 11, comprising the polynucleotide sequence of SEQ ID NO:52.

97. A polynucleotide of claim 11, comprising the polynucleotide sequence of SEQ ID NO:53.

98. A polynucleotide of claim 11, comprising the polynucleotide sequence of SEQ ID NO:54.

99. A polynucleotide of claim 11, comprising the polynucleotide sequence of SEQ ID NO:55.

100. A polynucleotide of claim 11, comprising the polynucleotide sequence of SEQ ID NO:56.

101. A polynucleotide of claim 11, comprising the polynucleotide sequence of SEQ ID NO:57.

102. A polynucleotide of claim 11, comprising the polynucleotide sequence of SEQ ID NO:58.

103. A polynucleotide of claim 11, comprising the polynucleotide sequence of SEQ ID NO:59.

104. A polynucleotide of claim 11, comprising the polynucleotide sequence of SEQ ID NO:60.

105. A polynucleotide of claim 11, comprising the polynucleotide sequence of SEQ ID NO:61.

106. A polynucleotide of claim 11, comprising the polynucleotide sequence of SEQ ID NO:62.

107. A polynucleotide of claim 11, comprising the polynucleotide sequence of SEQ ID NO:63.

108. A polynucleotide of claim 11, comprising the polynucleotide sequence of SEQ ID NO:64.

109. A polynucleotide of claim 11, comprising the polynucleotide sequence of SEQ ID NO:65.

110. A polynucleotide of claim 11, comprising the polynucleotide sequence of SEQ ID NO:66.

111. A polynucleotide of claim 11, comprising the polynucleotide sequence of SEQ ID NO:67.

112. A polynucleotide of claim 11, comprising the polynucleotide sequence of SEQ ID NO:68.

113. A polynucleotide of claim 11, comprising the polynucleotide sequence of SEQ ID NO:69.

114. A polynucleotide of claim 11, comprising the polynucleotide sequence of SEQ ID NO:70.

115. A polynucleotide of claim 11, comprising the polynucleotide sequence of SEQ ID NO:71.

116. A polynucleotide of claim 11, comprising the polynucleotide sequence of SEQ ID NO:72.
